(19) United States
(12) Patent Application Publication
(10) Pub. No.: US 2024/0329032 A1
(43) Pub. Date: Oct. 3, 2024

(54) METHODS OF DIFFERENTIATING NEURAL CELLS AND PREDICTING ENGRAFTMENT THEREOF AND RELATED COMPOSITIONS

(71) Applicant: Aspen Neuroscience, Inc., San Diego, CA (US)

(72) Inventors: Andres BRATT-LEAL, San Jose, CA (US); Ai ZHANG, San Diego, CA (US); Roy WILLIAMS, San Diego, CA (US); Jim MOSSMAN, San Diego, CA (US); Derren BARKEN, San Diego, CA (US)

(73) Assignee: Aspen Neuroscience, Inc., San Diego, CA (US)

(21) Appl. No.: 18/580,557

(22) PCT Filed: Jul. 20, 2022
(86) PCT No.: PCT/US2022/073974

§ 371 Date: Jan. 18, 2024

Related U.S. Application Data
(60) Provisional application No. 63/224,404, filed on Jul. 21, 2021.

(51) Int. Cl. G01N 33/50; C12N 5/0793; G16B 25/10; G16B 40/20

(52) U.S. Cl. CPC G01N 33/5023; C12N 5/0619; G01N 33/5088; G16B 25/10; G16B 40/20; C12N 2503/00; C12N 2506/45; C12N 2533/52

(57) ABSTRACT

Provided herein are methods of differentiating neural cells as well as methods of predicting cell engraftment of populations of cells using gene expression, for instance populations of neuronal progenitor cells, following implantation in a subject. Also provided herein are related compositions, articles of manufacture, and kits, including for use in methods of treating a subject having neurodegenerative disease, for instance Parkinson's disease.

### CROSS-REFERENCE TO RELATED APPLICATIONS

This application claims priority to U.S. Provisional Application No. U.S. 63/224,404, filed Jul. 21, 2021, entitled “METHODS OF DIFFERENTIATING NEURAL CELLS AND PREDICTING ENGRAFTMENT THEREOF AND RELATED COMPOSITIONS,” the contents of which are incorporated by reference in their entirety for all purposes.

### FIELD

The present disclosure relates to methods of differentiating neural cells as well as to methods of predicting cell engraftment of populations of cells, for instance populations of neuronal progenitor cells, following implantation in a subject. Also provided herein are related compositions, articles of manufacture, and kits, including for use in methods of treating a subject having a neurodegenerative disease, for instance Parkinson's disease.

### BACKGROUND

Various methods for differentiating pluripotent stem cells into lineage specific cell populations and the resulting cellular compositions are contemplated to find use in cell replacement therapies for patients with diseases resulting in a loss of function of a defined cell population. However, in some cases, such methods are limited in their ability to produce cells that engraft and innervate other cells in vivo. Improved methods and cellular compositions thereof are needed, including to provide for methods of predicting and identifying the engraftment fitness of differentiated cells.

### SUMMARY

Provided herein in some embodiments is a method of predicting cell engraftment of a population of neuronal progenitor cells, the method comprising: (a) obtaining gene expression levels of a plurality of genes for one or more cells of a population of neuronal progenitor cells, wherein: the population of neuronal progenitor cells is from a culture of cells differentiated from pluripotent stem cells under conditions to neurally differentiate the cells; and the plurality of genes comprise one or more cell cycle genes and/or one or more maturity genes; and (b) applying the gene expression levels as input to a process configured to predict if the population of neuronal progenitor cells will engraft in a brain region of a subject following implantation of the population of neuronal progenitor cells into the brain region, wherein the predicting is based on gene expression levels of one or more of the plurality of genes.

Provided herein in some embodiments is a method of assessing a population of neuronal progenitor cells for implantation in a subject to treat a neurodegenerative disease, the method comprising: (a) obtaining gene expression levels of a plurality of genes for one or more cells of a population of neuronal progenitor cells, wherein: the population of neuronal progenitor cells is from a culture of cells differentiated from pluripotent stem cells under conditions to neurally differentiate the cells; and the plurality of genes comprise one or more cell cycle genes and/or one or more maturity genes; and (b) applying the gene expression levels as input to a process configured to predict if the population of neuronal progenitor cells will engraft in a brain region of a subject following implantation of the population of neuronal progenitor cells into the brain region, wherein the predicting is based on gene expression levels of one or more of the plurality of genes.

In some of any embodiments, the population of neuronal progenitor cells are for implantation in a brain region of the subject if the population of neuronal progenitor cells is predicted to engraft.

Provided herein in some embodiments is a method of selecting a population of neuronal progenitor cells for implantation in a subject for treating a neurodegenerative disease, the method comprising: (a) obtaining gene expression levels of a plurality of genes for one or more cells of a population of neuronal progenitor cells, wherein: the population of neuronal progenitor cells is from a culture of cells differentiated from pluripotent stem cells under conditions to neurally differentiate the cells; and the plurality of genes comprise one or more cell cycle genes and/or one or more maturity genes; and (b) applying the gene expression levels as input to a process configured to predict if the population of neuronal progenitor cells will engraft in a brain region of a subject following implantation of the population of neuronal progenitor cells into the brain region, wherein the predicting is based on gene expression levels of one or more of the plurality of genes; and (c) selecting the population of neuronal progenitor cells for implantation in the subject if the population of neuronal progenitor cells are predicted to engraft.

In some embodiments, the predicting is based on the gene expression levels of the plurality of genes.

In some of any embodiments, the process comprises a machine learning model. In some of any embodiments, the machine learning model is trained using gene expression levels of the one or more of the plurality of genes for a plurality of reference populations of neuronal progenitor cells that have been differentiated from pluripotent stem cells under conditions to neurally differentiate the cells.

In some of any embodiments, the machine learning model is trained using gene expression levels of the plurality of genes for a plurality of reference populations of neuronal progenitor cells that have been differentiated from pluripotent stem cells under conditions to neurally differentiate the cells.

In some of any embodiments, the machine learning model is or comprises a supervised machine learning model.

In some of any embodiments, the machine learning model is trained using (i) gene expression levels of the one or more of the plurality of genes for a plurality of reference populations of neuronal progenitor cells that have been differentiated from pluripotent stem cells under conditions to neurally differentiate the cells and (ii) engraftment fitness of the plurality of reference populations, wherein the engraftment fitness of a reference population indicates whether or not, or the degree to which, the reference population engrafted in a brain region of a subject following implantation of the reference population into the brain region.

In some of any embodiments, the machine learning model is trained using (i) the gene expression levels for the plurality of reference populations and (ii) engraftment fitness of the plurality of reference populations, wherein the engraftment fitness of a reference population indicates whether or not, or the degree to which, the reference population engrafted in a brain region of a subject following implantation of the reference population into the brain region.

Provided herein in some embodiments is a method of training a machine learning model, comprising: (a) obtaining gene expression levels of one or more of a plurality of genes for a plurality of reference populations of neuronal progenitor cells that are from cultures of cells that have been differentiated from pluripotent stem cells under conditions to neurally differentiate the cells, wherein the plurality of genes comprise one or more cell cycle genes and/or one or more maturity genes; and (b) applying the gene expression levels of the plurality of reference populations as input to train a machine learning model.

In some of any embodiments, gene expression levels of the plurality of genes are obtained for the plurality of reference populations, and the gene expression levels of the plurality of genes for the plurality of reference populations are applied as input to train the machine learning model.

In some of any embodiments, the machine learning model is or comprises a supervised machine learning model.

In some of any embodiments, the method further comprises: (a) receiving engraftment fitness of the plurality of reference populations, wherein the engraftment fitness of a reference population indicates whether or not, or the degree to which, the reference population engrafted in a brain region of a subject following implantation of the reference population into the brain region; and (b) applying the engraftment fitness of the plurality of reference populations as input to train the machine learning model, wherein the machine learning model is trained to predict based on gene expression levels of one or more of the plurality of genes if a population of neuronal progenitor cells that is from a culture of cells differentiated from pluripotent stem cells under conditions to neurally differentiate the cells will engraft in a brain region of a subject following implantation of the population of neuronal progenitor cells into the brain region.

In some of any embodiments, the machine learning model is trained to predict based on the gene expression levels of the plurality of genes if a population of neuronal progenitor cells that is from a culture of cells differentiated from pluripotent stem cells under conditions to neurally differentiate the cells will engraft in a brain region of a subject following implantation of the population of neuronal progenitor cells into the brain region.

In some of any embodiments, the engraftment fitness of a reference population is determined based on the number of cells of the reference population that are present in the brain region following the implantation. In some of any embodiments, the number of cells is counted at, about, at least, or at least about 7 days, 14 days, or 21 days following the implantation.

In some of any embodiments, a reference population is considered fit for engraftment if at least a predetermined number of cells are present in the brain region following the implantation. In some of any embodiments, the predetermined number of cells is greater than or greater than about 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9%, or 10% of the number of cells implanted in the brain region.

In some of any embodiments, the machine learning model is or comprises a classification model. In some of any embodiments, the machine learning model is or comprises a regression model. In some of any embodiments, the machine learning model is or comprises a logistic regression model.

In some of any embodiments, the machine learning model is or comprises a penalized model. In some of any embodiments, the machine learning model is or comprises a ridge regression model, a lasso regression model, and/or an elastic net regression model. In some of any embodiments, the machine learning model is or comprises a lasso regression model. In some of any embodiments, the machine learning model is or comprises a lasso logistic regression model.

In some of any embodiments, the plurality of genes comprises, comprises about, comprises greater than, or comprises greater than about 2, 3, 4, 5, 6, 7, 8, 9, 10, 12, 14, 16, 18, 20, 25, 30, 35, 40, 45, 50, 55, 60, 62, 64, 66, 68, or 70 genes. In some of any embodiments, the plurality of genes comprises between or between about 2 and 100, 2 and 95, 2 and 90, 2 and 85, 2 and 80, 2 and 75, or 2 and 70 genes. In some of any embodiments, the plurality of genes comprises between or between about 5 and 100, 5 and 95, 5 and 90, 5 and 85, 5 and 80, 5 and 75, or 5 and 70 genes.

In some of any embodiments, the gene expression levels of the plurality of genes are gene expression levels of genes associated with the ability of a population of neuronal progenitor cells to engraft in a brain region of a subject.

In some of any embodiments, the plurality of genes comprises one or more of AC104083.1, ACE, ACSL1, ACSS3, ADSS, AFAP1, ANLN, ANP32A, ANXA11, APBA1, ARHGDIG, ARL8A, ASPH, ASPM, AURKA, AURKB, BDNF, BICDL1, BIRC5, BRINP1, BUB1, BUB1B, CAMK2B, CCDC112, CCDC160, CCDC60, CCNA2, CCNB1, CCNB2, CDC20, CDC25C, CDCA2, CDCA8, CDK1, CDK5R2, CDKN1A, CENPE, CENPF, CEP170B, CEP55, CHGB, CIT, COL23A1, CTSC, CYFIP1, DAAM2, DIRAS1, DLGAP5, DMTN, DNAJB5, DPY19L1, DUSP26, ECT2, ESPL1, FABP7, FAM71E2, FAM83D, FAM86C2P, FANCD2, FBXL16, FNBP1L, FOXM1, FZD2, GEM, GFOD2, GTSE1, GUCY1A1, HAPLN3, HCN3, HJURP, HLA-E, HMMR, HTATIP2, IKZF2, IL4R, IQGAP3, ITGA5, JPT1, KCNB1, KCNC1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MGST1, MIR100HG, MKI67, MRVI1, MYCBP, NAALAD2, NACAD, NAV2, NCAM1, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SALL4, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TOP2A, TPH1, TPX2, TRIM46, TTK, TUBA1C, UBE2C, and YBX3.

In some of any embodiments, the plurality of genes comprises one or more of AC104083.1, ACE, ACSL1, ACSS3, ADSS, AFAP1, ANLN, ANP32A, ANXA11, ARHGDIG, ARL8A, ASPH, AURKA, AURKB, BICDL1, BIRC5, BRINP1, BUB1, BUB1B, CCDC112, CCDC160, CCDC60, CCNA2, CCNB1, CCNB2, CDC20, CDC25C, CDCA2, CDCA8, CDK1, CDKN1A, CENPE, CENPF, CEP170B, CEP55, CHGB, CIT, CYFIP1, DAAM2, DIRAS1, DLGAP5, DNAJB5, DPY19L1, DUSP26, ECT2, ESPL1, FAM71E2, FAM83D, FAM86C2P, FANCD2, FBXL16, FNBP1L, FOXM1, FZD2, GEM, GFOD2, GTSE1, GUCY1A1, HCN3, HJURP, HLA-E, HMMR, HTATIP2, IKZF2, IQGAP3, ITGA5, JPT1, KCNB1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MIR100HG, MKI67, MRVI1, MYCBP, NAALAD2, NACAD, NAV2, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TPH1, TPX2, TRIM46, TTK, TUBA1C, and UBE2C.

In some of any embodiments, the plurality of genes comprises one or more of ACE, ACSL1, ANP32A, ARL8A, AURKB, BDNF, BIRC5, CCDC112, CCDC160, CCDC60, CCNB1, CCNB2, CDC20, CDC25C, CDCA8, CDK1, CEP170B, CHGB, COL23A1, DAAM2, DLGAP5, DMTN, ESPL1, FABP7, FAM71E2, FAM83D, FNBP1L, GTSE1, HAPLN3, HJURP, HTATIP2, IQGAP3, KCNB1, KIF1A, KIF20A, KIF2C, KIFC1, LINC01128, LRIG1, MAP3K9, MGST1, MIR100HG, MRVI1, NAALAD2, NACAD, NDC80, NEK2, NFIC, NFIX, NR6A1, NT5DC1, NUF2, NUSAP1, PLAG1, PRKACB, PRTG, PTCH1, PTPN13, PTTG1, SALL4, SAPCD2, SEMA5B, SLC35D2, SLC6A17, STOX1, SUCLG2, SYT13, TACC3, TGFBR3, TOP2A, TPH1, TPX2, UBE2C, and YBX3.

In some of any embodiments, the plurality of genes comprises one or more of ACE, ACSL1, ANP32A, ARL8A, AURKB, BIRC5, CCDC112, CCDC160, CCDC60, CCNB1, CCNB2, CDC20, CDC25C, CDCA8, CDK1, CEP170B, CHGB, COL23A1, DAAM2, DLGAP5, ESPL1, FABP7, FAM71E2, FAM83D, FNBP1L, GTSE1, HAPLN3, HJURP, HTATIP2, IQGAP3, KCNB1, KIF1A, KIF20A, KIF2C, KIFC1, LINC01128, LRIG1, MAP3K9, MGST1, MIR100HG, MRVI1, NAALAD2, NACAD, NDC80, NEK2, NFIC, NFIX, NR6A1, NT5DC1, NUF2, NUSAP1, PLAG1, PRKACB, PRTG, PTCH1, PTPN13, PTTG1, SALL4, SAPCD2, SEMA5B, SLC35D2, SLC6A17, STOX1, SUCLG2, SYT13, TACC3, TGFBR3, TOP2A, TPH1, TPX2, UBE2C, and YBX3.

In some of any embodiments, the plurality of genes comprise one or more cell cycle genes. In some of any embodiments, the one or more cell cycle genes comprise one or more of ANLN, ASPM, AURKA, AURKB, BIRC5, BRINP1, BUB1, BUB1B, CCNA2, CCNB1, CCNB2, CDC20, CDC25C, CDCA2, CDCA8, CDK1, CDKN1A, CENPE, CENPF, CEP55, CIT, DLGAP5, ECT2, ESPL1, FAM83D, FANCD2, FOXM1, GEM, GTSE1, HJURP, HMMR, IQGAP3, KIF11, KIF14, KIF15, KIF18A, KIF20A, KIF23, KIF2C, KIF4A, KIFC1, KNL1, MELK, MKI67, NCAPG, NCAPH, NDC80, NEK2, NUF2, NUSAP1, PBK, PIMREG, PLK1, PLK2, POC1A, PRC1, PRR11, PTTG1, RACGAP1, SAPCD2, SKA3, SPAG5, TACC3, TOP2A, TPX2, TTK, TUBA1C, and UBE2C. In some of any embodiments, the one or more cell cycle genes comprise BRINP1, CDKN1A, FAM83D, FANCD2, GEM, PLK2, and SAPCD2. In some of any embodiments, the one or more cell cycle genes consist of BRINP1, CDKN1A, FAM83D, FANCD2, GEM, PLK2, and SAPCD2.

In some of any embodiments, the plurality of genes comprises one or more of ANLN, ASPM, AURKA, AURKB, BIRC5, BRINP1, BUB1, BUB1B, CCNA2, CCNB1, CCNB2, CDC20, CDC25C, CDCA2, CDCA8, CDK1, CDKN1A, CENPE, CENPF, CEP55, CIT, DLGAP5, ECT2, ESPL1, FAM83D, FANCD2, FOXM1, GEM, GTSE1, HJURP, HMMR, IQGAP3, KIF11, KIF14, KIF15, KIF18A, KIF20A, KIF23, KIF2C, KIF4A, KIFC1, KNL1, MELK, MKI67, NCAPG, NCAPH, NDC80, NEK2, NUF2, NUSAP1, PBK, PIMREG, PLK1, PLK2, POC1A, PRC1, PRR11, PTTG1, RACGAP1, SAPCD2, SKA3, SPAG5, TACC3, TPX2, TTK, TUBA1C, and UBE2C.

In some of any embodiments, the plurality of genes comprises one or more of AURKB, BIRC5, CCNB1, CCNB2, CDC20, CDC25C, CDCA8, CDK1, DLGAP5, ESPL1, FAM83D, GTSE1, HJURP, IQGAP3, KIF20A, KIF2C, KIFC1, NDC80, NEK2, NUF2, NUSAP1, PTTG1, SAPCD2, TACC3, TOP2A, TPX2, and UBE2C.

In some of any embodiments, the plurality of genes comprises one or more of AURKB, BIRC5, CCNB1, CCNB2, CDC20, CDC25C, CDCA8, CDK1, DLGAP5, ESPL1, FAM83D, GTSE1, HJURP, IQGAP3, KIF20A, KIF2C, KIFC1, NDC80, NEK2, NUF2, NUSAP1, PTTG1, SAPCD2, TACC3, TPX2, and UBE2C.

In some of any embodiments, the plurality of genes comprise one or more maturity genes.

In some of any embodiments, the plurality of genes comprise one or more of AC104083.1, ACE, ACSL1, AFAP1, APBA1, ARHGDIG, ARL8A, BDNF, BICDL1, CAMK2B, CCDC112, CDK5R2, CEP170B, CHGB, DIRAS1, DMTN, DNAJB5, DUSP26, FBXL16, FNBP1L, GFOD2, GUCY1A1, HCN3, JPT1, KCNB1, KCNC1, KCNH6, KIF1A, KLF7, LINC01128, MACO1, MAP3K9, MAPRE3, MIR100HG, NACAD, NCAM1, NFIC, NFIX, PARP6, PRKACB, RIMS1, SBK1, SHISA7, SLC6A17, SPTBN1, SRGAP2, SYT13, TMEM151B, TPH1, TRIM46, ACSS3, ADSS, ANP32A, ANXA11, ASPH, CCDC160, CCDC60, COL23A1, CTSC, CYFIP1, DAAM2, DPY19L1, FABP7, FAM71E2, FAM86C2P, FZD2, HAPLN3, HLA-E, HTATIP2, IKZF2, IL4R, ITGA5, KCNJ2-AS1, LPIN3, LRIG1, LRIG3, MGST1, MRVI1, MYCBP, NAALAD2, NAV2, NR6A1, NT5DC1, PLAG1, POFUT2, PRTG, PTCH1, PTPN13, SALL4, SAV1, SELENOP, SEMA5B, SLC35D2, SLC66A3, STOX1, SUCLG2, TGFBR3, TM6SF2, TOB1, and YBX3.

In some of any embodiments, the plurality of genes comprise one or more of AC104083.1, ACE, ACSL1, ACSS3, ADSS, AFAP1, ANP32A, ANXA11, ARHGDIG, ARL8A, ASPH, BICDL1, CCDC112, CCDC160, CCDC60, CEP170B, CHGB, CYFIP1, DAAM2, DIRAS1, DNAJB5, DPY19L1, DUSP26, FAM71E2, FAM86C2P, FBXL16, FNBP1L, FZD2, GFOD2, GUCY1A1, HCN3, HLA-E, HTATIP2, IKZF2, ITGA5, JPT1, KCNB1, KCNH6, KCNJ2-AS1, KIF1A, KLF7, LINC01128, LPIN3, LRIG1, LRIG3, MACO1, MAP3K9, MAPRE3, MIR100HG, MRVI1, MYCBP, NAALAD2, NACAD, NAV2, NFIC, NFIX, NR6A1, NT5DC1, PARP6, PLAG1, POFUT2, PRKACB, PRTG, PTCH1, PTPN13, RIMS1, SAV1, SBK1, SELENOP, SEMA5B, SHISA7, SLC35D2, SLC66A3, SLC6A17, SPTBN1, SRGAP2, STOX1, SUCLG2, SYT13, TGFBR3, TM6SF2, TMEM151B, TOB1, TPH1, and TRIM46.

In some of any embodiments, the plurality of genes comprises one or more of ACE, ACSL1, ANP32A, ARL8A, BDNF, CCDC112, CCDC160, CCDC60, CEP170B, CHGB, COL23A1, DAAM2, DMTN, FABP7, FAM71E2, FNBP1L, HAPLN3, HTATIP2, KCNB1, KIF1A, LINC01128, LRIG1, MAP3K9, MGST1, MIR100HG, MRVI1, NAALAD2, NACAD, NFIC, NFIX, NR6A1, NT5DC1, PLAG1, PRKACB, PRTG, PTCH1, PTPN13, SALL4, SEMA5B, SLC35D2, SLC6A17, STOX1, SUCLG2, SYT13, TGFBR3, TPH1, and YBX3.

In some of any embodiments, the plurality of genes comprises one or more of ACE, ACSL1, ANP32A, ARL8A, CCDC112, CCDC160, CCDC60, CEP170B, CHGB, DAAM2, FAM71E2, FNBP1L, HTATIP2, KCNB1, KIF1A, LINC01128, LRIG1, MAP3K9, MIR100HG, MRVI1, NAALAD2, NACAD, NFIC, NFIX, NR6A1, NT5DC1, PLAG1, PRKACB, PRTG, PTCH1, PTPN13, SEMA5B, SLC35D2, SLC6A17, STOX1, SUCLG2, SYT13, TGFBR3, and TPH1.

In some of any embodiments, the one or more maturity genes comprise genes whose gene expression levels increase during differentiation, optionally during days 17-22 of differentiation of the culture of cells. In some of any embodiments, the one or more maturity genes comprise genes whose gene expression levels increase substantially monotonically during differentiation, optionally during days 17-22 of differentiation of the culture of cells. In some of any embodiments, the one or more maturity genes comprise genes whose gene expression levels increase during days 17-22 of differentiation of the culture of cells. In some of any embodiments, the one or more maturity genes comprise genes whose gene expression levels increase substantially monotonically during days 17-22 of differentiation of the culture of cells.

In some of any embodiments, the one or more maturity genes comprise one or more of AC104083.1, ACE, ACSL1, AFAP1, APBA1, ARHGDIG, ARL8A, BDNF, BICDL1, CAMK2B, CCDC112, CDK5R2, CEP170B, CHGB, DIRAS1, DMTN, DNAJB5, DUSP26, FBXL16, FNBP1L, GFOD2, GUCY1A1, HCN3, JPT1, KCNB1, KCNC1, KCNH6, KIF1A, KLF7, LINC01128, MACO1, MAP3K9, MAPRE3, MIR100HG, NACAD, NCAM1, NFIC, NFIX, PARP6, PRKACB, RIMS1, SBK1, SHISA7, SLC6A17, SPTBN1, SRGAP2, SYT13, TMEM151B, TPH1, and TRIM46.

In some of any embodiments, the plurality of genes comprises one or more of AC104083.1, ACE, ACSL1, AFAP1, ARHGDIG, ARL8A, BICDL1, CCDC112, CEP170B, CHGB, DIRAS1, DNAJB5, DUSP26, FBXL16, FNBP1L, GFOD2, GUCY1A1, HCN3, JPT1, KCNB1, KCNH6, KIF1A, KLF7, LINC01128, MACO1, MAP3K9, MAPRE3, MIR100HG, NACAD, NFIC, NFIX, PARP6, PRKACB, RIMS1, SBK1, SHISA7, SLC6A17, SPTBN1, SRGAP2, SYT13, TMEM151B, TPH1, and TRIM46.

In some of any embodiments, the plurality of genes comprises one or more of ACE, ACSL1, ARL8A, BDNF, CCDC112, CEP170B, CHGB, DMTN, FNBP1L, KCNB1, KIF1A, LINC01128, MAP3K9, MIR100HG, NACAD, NFIC, NFIX, PRKACB, SLC6A17, SYT13, and TPH1.

In some of any embodiments, the plurality of genes comprises one or more of ACE, ACSL1, ARL8A, CCDC112, CEP170B, CHGB, FNBP1L, KCNB1, KIF1A, LINC01128, MAP3K9, MIR100HG, NACAD, NFIC, NFIX, PRKACB, SLC6A17, SYT13, and TPH1.

In some of any embodiments, the one or more maturity genes comprise genes whose gene expression levels decrease during differentiation, optionally during days 17-22 of differentiation of the culture of cells. In some of any embodiments, the one or more maturity genes comprise genes whose gene expression levels decrease substantially monotonically during differentiation, optionally during days 17-22 of differentiation of the culture of cells. In some of any embodiments, the one or more maturity genes comprise genes whose gene expression levels decrease during days 17-22 of differentiation of the culture of cells. In some of any embodiments, the one or more maturity genes comprise genes whose gene expression levels decrease substantially monotonically during days 17-22 of differentiation of the culture of cells. In some of any embodiments, the one or more maturity genes comprise one or more of ACSS3, ADSS, ANP32A, ANXA11, ASPH, CCDC160, CCDC60, COL23A1, CTSC, CYFIP1, DAAM2, DPY19L1, FABP7, FAM71E2, FAM86C2P, FZD2, HAPLN3, HLA-E, HTATIP2, IKZF2, IL4R, ITGA5, KCNJ2-AS1, LPIN3, LRIG1, LRIG3, MGST1, MRVI1, MYCBP, NAALAD2, NAV2, NR6A1, NT5DC1, PLAG1, POFUT2, PRTG, PTCH1, PTPN13, SALL4, SAV1, SELENOP, SEMA5B, SLC35D2, SLC66A3, STOX1, SUCLG2, TGFBR3, TM6SF2, TOB1, and YBX3.

In some of any embodiments, the plurality of genes comprises one or more of ACSS3, ADSS, ANP32A, ANXA11, ASPH, CCDC160, CCDC60, CYFIP1, DAAM2, DPY19L1, FAM71E2, FAM86C2P, FZD2, HLA-E, HTATIP2, IKZF2, ITGA5, KCNJ2-AS1, LPIN3, LRIG1, LRIG3, MRVI1, MYCBP, NAALAD2, NAV2, NR6A1, NT5DC1, PLAG1, POFUT2, PRTG, PTCH1, PTPN13, SAV1, SELENOP, SEMA5B, SLC35D2, SLC66A3, STOX1, SUCLG2, TGFBR3, TM6SF2, and TOB1.

In some of any embodiments, the plurality of genes comprises one or more of ANP32A, CCDC160, CCDC60, COL23A1, DAAM2, FABP7, FAM71E2, HAPLN3, HTATIP2, LRIG1, MGST1, MRVI1, NAALAD2, NR6A1, NT5DC1, PLAG1, PRTG, PTCH1, PTPN13, SALL4, SEMA5B, SLC35D2, STOX1, SUCLG2, TGFBR3, and YBX3.

In some of any embodiments, the plurality of genes comprises one or more of ANP32A, CCDC160, CCDC60, DAAM2, FAM71E2, HTATIP2, LRIG1, MRVI1, NAALAD2, NR6A1, NT5DC1, PLAG1, PRTG, PTCH1, PTPN13, SEMA5B, SLC35D2, STOX1, SUCLG2, and TGFBR3.

In some of any embodiments, if the population of neuronal progenitor cells is predicted to not engraft, the method further comprises repeating steps (a) and (b) for the same or a different population of neuronal progenitor cells.

In some of any embodiments, the method further comprises selecting based on an output of the process the population of neuronal progenitor cells as a population of neuronal progenitor cells that is predicted to engraft.

In some of any embodiments, the process is configured to predict the presence or absence of engraftment, and the population of neuronal progenitor cells is predicted to engraft if the process predicts the presence of engraftment. In some of any embodiments, the process is configured to predict the probability of engraftment, and the population of neuronal progenitor cells is predicted to engraft if the predicted probability of engraftment exceeds a predetermined threshold level. In some of any embodiments, the predetermined probability threshold level is, is about, is greater than, or is greater than about 0.5. In some of any embodiments, the predetermined probability threshold level is, is about, is greater than, or is greater than about 0.55, 0.6, 0.65, 0.7, 0.75, 0.8, 0.85, 0.9, 0.91, 0.92, 0.93, 0.94, 0.95, 0.96, 0.97, 0.98, or 0.99. In some of any embodiments, the process is configured to predict the degree of engraftment, and the population of neuronal progenitor cells is predicted to engraft if the predicted degree of engraftment exceeds a predetermined threshold level.

In some of any embodiments, the method further comprises harvesting the selected population of neuronal progenitor cells.

Provided herein in some embodiments is a method of assessing engraftment fitness of a population of neuronal progenitor cells, the method comprising (a) measuring gene expression levels of a plurality of genes for one or more cells of a population of neuronal progenitor cells, wherein: the population of neuronal progenitor cells is from a culture of cells differentiated from pluripotent stem cells under conditions to neurally differentiate the cells; and the plurality of genes comprise one or more of AC104083.1, ACE, ACSL1, ACSS3, ADSS, AFAP1, ANLN, ANP32A, ANXA11, APBA1, ARHGDIG, ARL8A, ASPH, ASPM, AURKA, AURKB, BDNF, BICDL1, BIRC5, BRINP1, BUB1, BUB1B, CAMK2B, CCDC112, CCDC160, CCDC60, CCNA2, CCNB1, CCNB2, CDC20, CDC25C, CDCA2, CDCA8, CDK1, CDK5R2, CDKN1A, CENPE, CENPF, CEP170B, CEP55, CHGB, CIT, COL23A1, CTSC, CYFIP1, DAAM2, DIRAS1, DLGAP5, DMTN, DNAJB5, DPY19L1, DUSP26, ECT2, ESPL1, FABP7, FAM71E2, FAM83D, FAM86C2P, FANCD2, FBXL16, FNBP1L, FOXM1, FZD2, GEM, GFOD2, GTSE1, GUCY1A1, HAPLN3, HCN3, HJURP, HLA-E, HMMR, HTATIP2, IKZF2, IL4R, IQGAP3, ITGA5, JPT1, KCNB1, KCNC1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MGST1, MIR100HG, MKI67, MRVI1, MYCBP, NAALAD2, NACAD, NAV2, NCAM1, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SALL4, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TOP2A, TPH1, TPX2, TRIM46, TTK, TUBA1C, UBE2C, and YBX3.

In some of any embodiments, the plurality of genes comprises one or more of AC104083.1, ACE, ACSL1, ACSS3, ADSS, AFAP1, ANLN, ANP32A, ANXA11, ARHGDIG, ARL8A, ASPH, AURKA, AURKB, BICDL1, BIRC5, BRINP1, BUB1, BUB1B, CCDC112, CCDC160, CCDC60, CCNA2, CCNB1, CCNB2, CDC20, CDC25C, CDCA2, CDCA8, CDK1, CDKN1A, CENPE, CENPF, CEP170B, CEP55, CHGB, CIT, CYFIP1, DAAM2, DIRAS1, DLGAP5, DNAJB5, DPY19L1, DUSP26, ECT2, ESPL1, FAM71E2, FAM83D, FAM86C2P, FANCD2, FBXL16, FNBP1L, FOXM1, FZD2, GEM, GFOD2, GTSE1, GUCY1A1, HCN3, HJURP, HLA-E, HMMR, HTATIP2, IKZF2, IQGAP3, ITGA5, JPT1, KCNB1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MIR100HG, MKI67, MRVI1, MYCBP, NAALAD2, NACAD, NAV2, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TPH1, TPX2, TRIM46, TTK, TUBA1C, and UBE2C.

In some of any embodiments, the plurality of genes comprises one or more of ACE, ACSL1, ANP32A, ARL8A, AURKB, BDNF, BIRC5, CCDC112, CCDC160, CCDC60, CCNB1, CCNB2, CDC20, CDC25C, CDCA8, CDK1, CEP170B, CHGB, COL23A1, DAAM2, DLGAP5, DMTN, ESPL1, FABP7, FAM71E2, FAM83D, FNBP1L, GTSE1, HAPLN3, HJURP, HTATIP2, IQGAP3, KCNB1, KIF1A, KIF20A, KIF2C, KIFC1, LINC01128, LRIG1, MAP3K9, MGST1, MIR100HG, MRVI1, NAALAD2, NACAD, NDC80, NEK2, NFIC, NFIX, NR6A1, NT5DC1, NUF2, NUSAP1, PLAG1, PRKACB, PRTG, PTCH1, PTPN13, PTTG1, SALL4, SAPCD2, SEMA5B, SLC35D2, SLC6A17, STOX1, SUCLG2, SYT13, TACC3, TGFBR3, TOP2A, TPH1, TPX2, UBE2C, and YBX3.

In some of any embodiments, the plurality of genes comprises one or more of ACE, ACSL1, ANP32A, ARL8A, AURKB, BIRC5, CCDC112, CCDC160, CCDC60, CCNB1, CCNB2, CDC20, CDC25C, CDCA8, CDK1, CEP170B, CHGB, COL23A1, DAAM2, DLGAP5, ESPL1, FABP7, FAM71E2, FAM83D, FNBP1L, GTSE1, HAPLN3, HJURP, HTATIP2, IQGAP3, KCNB1, KIF1A, KIF20A, KIF2C, KIFC1, LINC01128, LRIG1, MAP3K9, MGST1, MIR100HG, MRVI1, NAALAD2, NACAD, NDC80, NEK2, NFIC, NFIX, NR6A1, NT5DC1, NUF2, NUSAP1, PLAG1, PRKACB, PRTG, PTCH1, PTPN13, PTTG1, SALL4, SAPCD2, SEMA5B, SLC35D2, SLC6A17, STOX1, SUCLG2, SYT13, TACC3, TGFBR3, TOP2A, TPH1, TPX2, UBE2C, and YBX3.

In some of any embodiments, the plurality of genes comprise one or more of ANLN, ASPM, AURKA, AURKB, BIRC5, BRINP1, BUB1, BUB1B, CCNA2, CCNB1, CCNB2, CDC20, CDC25C, CDCA2, CDCA8, CDK1, CDKN1A, CENPE, CENPF, CEP55, CIT, DLGAP5, ECT2, ESPL1, FAM83D, FANCD2, FOXM1, GEM, GTSE1, HJURP, HMMR, IQGAP3, KIF11, KIF14, KIF15, KIF18A, KIF20A, KIF23, KIF2C, KIF4A, KIFC1, KNL1, MELK, MKI67, NCAPG, NCAPH, NDC80, NEK2, NUF2, NUSAP1, PBK, PIMREG, PLK1, PLK2, POC1A, PRC1, PRR11, PTTG1, RACGAP1, SAPCD2, SKA3, SPAG5, TACC3, TOP2A, TPX2, TTK, TUBA1C, and UBE2C.

In some of any embodiments, the plurality of genes comprises one or more of ANLN, ASPM, AURKA, AURKB, BIRC5, BRINP1, BUB1, BUB1B, CCNA2, CCNB1, CCNB2, CDC20, CDC25C, CDCA2, CDCA8, CDK1, CDKN1A, CENPE, CENPF, CEP55, CIT, DLGAP5, ECT2, ESPL1, FAM83D, FANCD2, FOXM1, GEM, GTSE1, HJURP, HMMR, IQGAP3, KIF11, KIF14, KIF15, KIF18A, KIF20A, KIF23, KIF2C, KIF4A, KIFC1, KNL1, MELK, MKI67, NCAPG, NCAPH, NDC80, NEK2, NUF2, NUSAP1, PBK, PIMREG, PLK1, PLK2, POC1A, PRC1, PRR11, PTTG1, RACGAP1, SAPCD2, SKA3, SPAG5, TACC3, TPX2, TTK, TUBA1C, and UBE2C.

In some of any embodiments, the plurality of genes comprises one or more of AURKB, BIRC5, CCNB1, CCNB2, CDC20, CDC25C, CDCA8, CDK1, DLGAP5, ESPL1, FAM83D, GTSE1, HJURP, IQGAP3, KIF20A, KIF2C, KIFC1, NDC80, NEK2, NUF2, NUSAP1, PTTG1, SAPCD2, TACC3, TOP2A, TPX2, and UBE2C.

In some of any embodiments, the plurality of genes comprises one or more of AURKB, BIRC5, CCNB1, CCNB2, CDC20, CDC25C, CDCA8, CDK1, DLGAP5, ESPL1, FAM83D, GTSE1, HJURP, IQGAP3, KIF20A, KIF2C, KIFC1, NDC80, NEK2, NUF2, NUSAP1, PTTG1, SAPCD2, TACC3, TPX2, and UBE2C.

In some of any embodiments, the plurality of genes comprises BRINP1, CDKN1A, FAM83D, FANCD2, GEM, PLK2, and SAPCD2.

In some of any embodiments, the plurality of genes comprise one or more of AC104083.1, ACE, ACSL1, AFAP1, APBA1, ARHGDIG, ARL8A, BDNF, BICDL1, CAMK2B, CCDC112, CDK5R2, CEP170B, CHGB, DIRAS1, DMTN, DNAJB5, DUSP26, FBXL16, FNBP1L, GFOD2, GUCY1A1, HCN3, JPT1, KCNB1, KCNC1, KCNH6, KIF1A, KLF7, LINC01128, MACO1, MAP3K9, MAPRE3, MIR100HG, NACAD, NCAM1, NFIC, NFIX, PARP6, PRKACB, RIMS1, SBK1, SHISA7, SLC6A17, SPTBN1, SRGAP2, SYT13, TMEM151B, TPH1, TRIM46, ACSS3, ADSS, ANP32A, ANXA11, ASPH, CCDC160, CCDC60, COL23A1, CTSC, CYFIP1, DAAM2, DPY19L1, FABP7, FAM71E2, FAM86C2P, FZD2, HAPLN3, HLA-E, HTATIP2, IKZF2, IL4R, ITGA5, KCNJ2-AS1, LPIN3, LRIG1, LRIG3, MGST1, MRVI1, MYCBP, NAALAD2, NAV2, NR6A1, NT5DC1, PLAG1, POFUT2, PRTG, PTCH1, PTPN13, SALL4, SAV1, SELENOP, SEMA5B, SLC35D2, SLC66A3, STOX1, SUCLG2, TGFBR3, TM6SF2, TOB1, and YBX3.

In some of any embodiments, the plurality of genes comprise one or more of AC104083.1, ACE, ACSL1, ACSS3, ADSS, AFAP1, ANP32A, ANXA11, ARHGDIG, ARL8A, ASPH, BICDL1, CCDC112, CCDC160, CCDC60, CEP170B, CHGB, CYFIP1, DAAM2, DIRAS1, DNAJB5, DPY19L1, DUSP26, FAM71E2, FAM86C2P, FBXL16, FNBP1L, FZD2, GFOD2, GUCY1A1, HCN3, HLA-E, HTATIP2, IKZF2, ITGA5, JPT1, KCNB1, KCNH6, KCNJ2-AS1, KIF1A, KLF7, LINC01128, LPIN3, LRIG1, LRIG3, MACO1, MAP3K9, MAPRE3, MIR100HG, MRVI1, MYCBP, NAALAD2, NACAD, NAV2, NFIC, NFIX, NR6A1, NT5DC1, PARP6, PLAG1, POFUT2, PRKACB, PRTG, PTCH1, PTPN13, RIMS1, SAV1, SBK1, SELENOP, SEMA5B, SHISA7, SLC35D2, SLC66A3, SLC6A17, SPTBN1, SRGAP2, STOX1, SUCLG2, SYT13, TGFBR3, TM6SF2, TMEM151B, TOB1, TPH1, and TRIM46.

In some of any embodiments, the plurality of genes comprises one or more of ACE, ACSL1, ANP32A, ARL8A, BDNF, CCDC112, CCDC160, CCDC60, CEP170B, CHGB, COL23A1, DAAM2, DMTN, FABP7, FAM71E2, FNBP1L, HAPLN3, HTATIP2, KCNB1, KIF1A, LINC01128, LRIG1, MAP3K9, MGST1, MIR100HG, MRVI1, NAALAD2, NACAD, NFIC, NFIX, NR6A1, NT5DC1, PLAG1, PRKACB, PRTG, PTCH1, PTPN13, SALL4, SEMA5B, SLC35D2, SLC6A17, STOX1, SUCLG2, SYT13, TGFBR3, TPH1, and YBX3.

In some of any embodiments, the plurality of genes comprises one or more of ACE, ACSL1, ANP32A, ARL8A, CCDC112, CCDC160, CCDC60, CEP170B, CHGB, DAAM2, FAM71E2, FNBP1L, HTATIP2, KCNB1, KIF1A, LINC01128, LRIG1, MAP3K9, MIR100HG, MRVI1, NAALAD2, NACAD, NFIC, NFIX, NR6A1, NT5DC1, PLAG1, PRKACB, PRTG, PTCH1, PTPN13, SEMA5B, SLC35D2, SLC6A17, STOX1, SUCLG2, SYT13, TGFBR3, and TPH1.

In some of any embodiments, the plurality of genes comprise one or more of AC104083.1, ACE, ACSL1, AFAP1, APBA1, ARHGDIG, ARL8A, BDNF, BICDL1, CAMK2B, CCDC112, CDK5R2, CEP170B, CHGB, DIRAS1, DMTN, DNAJB5, DUSP26, FBXL16, FNBP1L, GFOD2, GUCY1A1, HCN3, JPT1, KCNB1, KCNC1, KCNH6, KIF1A, KLF7, LINC01128, MACO1, MAP3K9, MAPRE3, MIR100HG, NACAD, NCAM1, NFIC, NFIX, PARP6, PRKACB, RIMS1, SBK1, SHISA7, SLC6A17, SPTBN1, SRGAP2, SYT13, TMEM151B, TPH1, and TRIM46.

In some of any embodiments, the plurality of genes comprises one or more of AC104083.1, ACE, ACSL1, AFAP1, ARHGDIG, ARL8A, BICDL1, CCDC112, CEP170B, CHGB, DIRAS1, DNAJB5, DUSP26, FBXL16, FNBP1L, GFOD2, GUCY1A1, HCN3, JPT1, KCNB1, KCNH6, KIF1A, KLF7, LINC01128, MACO1, MAP3K9, MAPRE3, MIR100HG, NACAD, NFIC, NFIX, PARP6, PRKACB, RIMS1, SBK1, SHISA7, SLC6A17, SPTBN1, SRGAP2, SYT13, TMEM151B, TPH1, and TRIM46.

In some of any embodiments, the plurality of genes comprises one or more of ACE, ACSL1, ARL8A, BDNF, CCDC112, CEP170B, CHGB, DMTN, FNBP1L, KCNB1, KIF1A, LINC01128, MAP3K9, MIR100HG, NACAD, NFIC, NFIX, PRKACB, SLC6A17, SYT13, and TPH1.

In some of any embodiments, the plurality of genes comprises one or more of ACE, ACSL1, ARL8A, CCDC112, CEP170B, CHGB, FNBP1L, KCNB1, KIF1A, LINC01128, MAP3K9, MIR100HG, NACAD, NFIC, NFIX, PRKACB, SLC6A17, SYT13, and TPH1.

In some of any embodiments, the plurality of genes comprise one or more of ACSS3, ADSS, ANP32A, ANXA11, ASPH, CCDC160, CCDC60, COL23A1, CTSC, CYFIP1, DAAM2, DPY19L1, FABP7, FAM71E2, FAM86C2P, FZD2, HAPLN3, HLA-E, HTATIP2, IKZF2, IL4R, ITGA5, KCNJ2-AS1, LPIN3, LRIG1, LRIG3, MGST1, MRVI1, MYCBP, NAALAD2, NAV2, NR6A1, NT5DC1, PLAG1, POFUT2, PRTG, PTCH1, PTPN13, SALL4, SAV1, SELENOP, SEMA5B, SLC35D2, SLC66A3, STOX1, SUCLG2, TGFBR3, TM6SF2, TOB1, and YBX3.

In some of any embodiments, the plurality of genes comprises one or more of ACSS3, ADSS, ANP32A, ANXA11, ASPH, CCDC160, CCDC60, CYFIP1, DAAM2, DPY19L1, FAM71E2, FAM86C2P, FZD2, HLA-E, HTATIP2, IKZF2, ITGA5, KCNJ2-AS1, LPIN3, LRIG1, LRIG3, MRVI1, MYCBP, NAALAD2, NAV2, NR6A1, NT5DC1, PLAG1, POFUT2, PRTG, PTCH1, PTPN13, SAV1, SELENOP, SEMA5B, SLC35D2, SLC66A3, STOX1, SUCLG2, TGFBR3, TM6SF2, and TOB1.

In some of any embodiments, the plurality of genes comprises one or more of ANP32A, CCDC160, CCDC60, COL23A1, DAAM2, FABP7, FAM71E2, HAPLN3, HTATIP2, LRIG1, MGST1, MRVI1, NAALAD2, NR6A1, NT5DC1, PLAG1, PRTG, PTCH1, PTPN13, SALL4, SEMA5B, SLC35D2, STOX1, SUCLG2, TGFBR3, and YBX3.

In some of any embodiments, the plurality of genes comprises one or more of ANP32A, CCDC160, CCDC60, DAAM2, FAM71E2, HTATIP2, LRIG1, MRVI1, NAALAD2, NR6A1, NT5DC1, PLAG1, PRTG, PTCH1, PTPN13, SEMA5B, SLC35D2, STOX1, SUCLG2, and TGFBR3.

In some of any embodiments, the method further comprises comparing one or more of the gene expression levels or one or more combinations thereof to one or more predetermined first threshold levels, wherein gene expression levels or combinations thereof that are greater than the first threshold levels are associated with a population of neuronal progenitor cells that is predicted to engraft in a brain region of a subject following implantation of the population of neuronal progenitor cells in the brain region.

In some of any embodiments, the method further comprises comparing one or more of the gene expression levels or one or more combinations thereof to one or more predetermined second threshold levels, wherein gene expression levels or combinations thereof that are less than the second threshold levels are associated with a population of neuronal progenitor cells that is predicted to engraft in a brain region of a subject following implantation of the population of neuronal progenitor cells in the brain region.

In some of any embodiments, the gene expression levels are RNA expression levels. In some of any embodiments, the gene expression levels are obtained by RNA sequencing.

In some of any embodiments, the brain region is the substantia nigra.

In some of any embodiments, the population of neuronal progenitor cells comprises determined dopaminergic neuron progenitor cells.

In some of any embodiments, prior to (a), the method further comprises differentiating the culture of cells comprising the population of neuronal progenitor cells.

In some of any embodiments, the culture of cells comprising the population of neuronal progenitor cells is differentiated from pluripotent stem cells by a process comprising: (a) performing a first incubation comprising culturing the pluripotent stem cells (PSCs) in a first culture vessel, wherein beginning at the initiation of the first incubation (day 0) the cells are exposed to (i) an inhibitor of TGF-3/activin-Nodal signaling; and (ii) an inhibitor of bone morphogenetic protein (BMP) signaling; and (b) performing a second incubation comprising culturing cells produced by the first incubation in a second culture vessel under conditions to neurally differentiate the cells.

In some of any embodiments, the second culture vessel is an adherent culture vessel. In some of any embodiments, the adherent culture vessel is coated with laminin or a fragment thereof.

Provided herein in some embodiments is a method of differentiating neural cells, the method comprising: (a) performing a first incubation comprising culturing pluripotent stem cells (PSCs) in a first culture vessel, wherein beginning at the initiation of the first incubation (day 0) the cells are exposed to (i) an inhibitor of TGF-β/activin-Nodal signaling; and (ii) an inhibitor of bone morphogenetic protein (BMP) signaling; and (b) performing a second incubation comprising culturing cells produced by the first incubation in a second culture vessel under conditions to neurally differentiate the cells, wherein the second culture vessel is an adherent culture vessel coated with laminin or a fragment thereof.

In some of any embodiments, the laminin is or comprises Laminin-111, Laminin-211, Laminin-121, Laminin-221, Laminin-332, Laminin-3A32, Laminin-3B32, Laminin-311, Laminin-3A11, Laminin-321, Laminin-3A21, Laminin-411, Laminin-421, Laminin-511, Laminin-521, Laminin-213, Laminin-423, Laminin-522, Laminin-523, or a fragment of any of the foregoing, optionally wherein the laminin is or comprises Laminin-521, Laminin-111, Laminin-511, or a fragment of any of the foregoing. In some of any embodiments, the laminin is or comprises Laminin-521, Laminin-111, Laminin-511, or a fragment of any of the foregoing. In some of any embodiments, the laminin is or comprises Laminin-511 or a fragment thereof. In some of any embodiments, the laminin is or comprises a Laminin-511 E8 fragment.

In some of any embodiments, the first culture vessel is a non-adherent culture vessel.

In some of any embodiments, beginning on day 0, the cells are also exposed to (iii) at least one activator of Sonic Hedgehog (SHH) signaling and (iv) an inhibitor of glycogen synthase kinase 3β(GSK3β). In some of any embodiments, the second incubation begins on about day 7. In some of any embodiments, the cells are exposed to the inhibitor of TGF-β/activin-Nodal signaling up to a day at or before day 7. In some of any embodiments, the cells are exposed to the inhibitor of TGF-β/activin-Nodal beginning at day 0 and through day 6, inclusive of each day. In some of any embodiments, the cells are exposed to the at least one activator of SHH signaling up to a day at or before day 7. In some of any embodiments, the cells are exposed to the at least one activator of SHH signaling beginning at day 0 and through day 6, inclusive of each day. In some of any embodiments, the cells are exposed to the inhibitor of BMP signaling up to a day at or before day 11. In some of any embodiments, the cells are exposed to the inhibitor of BMP signaling beginning at day 0 and through day 10, inclusive of each day. In some of any embodiments, the cells are exposed to the inhibitor of GSK3β signaling up to a day at or before day 13. In some of any embodiments, the cells are exposed to the inhibitor of GSK3β signaling beginning at day 0 and through day 12, inclusive of each day.

In some of any embodiments, the first incubation produces a spheroid of cells, and prior to performing the second incubation, the spheroid is dissociated to produce a cell suspension, and cells of the cell suspension are cultured in the second culture vessel. In some of any embodiments, the spheroid is dissociated by enzymatic dissociation. In some of any embodiments, the spheroid is dissociated by enzymatic dissociation comprising use of an enzyme selected from among the group consisting of accutase, dispase, collagenase, and combinations thereof. In some of any embodiments, the spheroid is dissociated by enzymatic dissociation comprising use of accutase. In some of any embodiments, the dissociating is carried out at a time when the cells of the spheroid express at least one of PAX6 and OTX2. In some of any embodiments, the dissociating is carried out on about day 7.

In some of any embodiments, the first culture vessel is an adherent culture vessel coated with laminin or a fragment thereof, optionally wherein the laminin or a fragment thereof is or comprises: Laminin-111, Laminin-211, Laminin-121, Laminin-221, Laminin-332, Laminin-3A32, Laminin-3B32, Laminin-311, Laminin-3A11, Laminin-321, Laminin-3A21, Laminin-411, Laminin-421, Laminin-511, Laminin-521, Laminin-213, Laminin-423, Laminin-522, Laminin-523, or a fragment of any of the foregoing, further optionally wherein the laminin is or comprises Laminin-511 or Laminin-511 E8 fragment. In some of any embodiments, the first culture vessel is an adherent culture vessel coated with Laminin-111, Laminin-211, Laminin-121, Laminin-221, Laminin-332, Laminin-3A32, Laminin-3B32, Laminin-311, Laminin-3A11, Laminin-321, Laminin-3A21, Laminin-411, Laminin-421, Laminin-511, Laminin-521, Laminin-213, Laminin-423, Laminin-522, Laminin-523, or a fragment of any of the foregoing. In some of any embodiments, the first culture vessel is an adherent culture vessel coated with Laminin-511 or Laminin-511 E8 fragment.

In some of any embodiments, beginning on day 1, the cells are exposed to (iii) at least one activator of Sonic Hedgehog (SHH) signaling; and beginning on day 2, the cells are exposed to an (iv) an inhibitor of glycogen synthase kinase 33 (GSK33) signaling. In some of any embodiments, the second incubation begins on about day 11. In some of any embodiments, the cells are exposed to the inhibitor of TGF-3/activin-Nodal signaling up to a day at or before day 5. In some of any embodiments, the cells are exposed to the inhibitor of TGF-3/activin-Nodal beginning at day 0 and through day 4, inclusive of each day. In some of any embodiments, the cells are exposed to the at least one activator of SHH signaling up to a day at or before day 7. In some of any embodiments, the cells are exposed to the at least one activator of SHH signaling beginning at day 0 and through day 6, inclusive of each day. In some of any embodiments, the cells are exposed to the inhibitor of BMP signaling up to a day at or before day 11. In some of any embodiments, the cells are exposed to the inhibitor of BMP signaling beginning at day 0 and through day 10, inclusive of each day. In some of any embodiments, the cells are exposed to the inhibitor of GSK3β signaling up to a day at or before day 13. In some of any embodiments, the cells are exposed to the inhibitor of GSK3β signaling beginning at day 0 and through day 12, inclusive of each day.

In some of any embodiments, the cells are cultured to differentiate the cells to determined dopaminergic neuron progenitor cells.

In some of any embodiments, culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF33) (collectively, “BAGCT”); and (vi) an inhibitor of Notch signaling.

In some of any embodiments, the cells are exposed to BAGCT and the inhibitor of Notch signaling beginning on day 11. In some of any embodiments, the cells are exposed to BAGCT and the inhibitor of Notch signaling beginning at day 11 and until harvest of the neurally differentiated cells, optionally until day 20. In some embodiments, the day of harvest is between at or about day 19 and day 24. In some embodiments, the day of harvest is at or about day 19, at or about day 20, at or about day 21, at or about day 22, at or about day 23 or at or about day 24. In some embodiments, the day of harvest is day 20. In some of any embodiments, the cells are exposed to BAGCT and the inhibitor of Notch signaling beginning at day 11 and until day 20.

In some of any embodiments, the inhibitor of TGF-3/activin-Nodal signaling is SB431542. In some of any embodiments, the cells are exposed to SB431542 at a concentration of between about 1 μM and about 20 μM, between about 5 μM and about 15 PM, or between about 8 μM and about 12 μM, optionally about 10 μM. In some of any embodiments, the cells are exposed to SB431542 at a concentration of about 10 μM.

In some of any embodiments, the at least one activator of SHH signaling is SHH or purmorphamine. In some of any embodiments, the at least one activator of SHH signaling comprises two activators of SHH signaling selected from SHH protein and purmorphamine. In some of any embodiments, the cells are exposed to SHH at a concentration of between about 10 ng/mL and 500 ng/mL, between about 20 ng/mL and about 400 ng/mL, between about 50 ng/mL and about 200 ng/mL, or between about 75 ng/mL and about 150 ng/mL, optionally about 100 ng/mL. In some of any embodiments, the cells are exposed to SHH at a concentration of about 100 ng/mL. In some of any embodiments, the cells are exposed to purmorphamine at a concentration of between about 1 μM and about 20 μM, between about 5 μM and about 15 μM, or between about 8 μM and about 12 μM, optionally about 10 μM. In some of any embodiments, the cells are exposed to purmorphamine at a concentration of about 10 PM.

In some of any embodiments, the inhibitor of BMP signaling is LDN193189. In some of any embodiments, the cells are exposed to LDN193189 at a concentration of between about 10 nM and 500 nM, between about 20 nM and about 400 nM, between about 50 nM and about 200 nM, or between about 75 nM and about 150 nM, optionally about 100 nM. In some of any embodiments, the cells are exposed to LDN193189 at a concentration of about 100 nM.

In some of any embodiments, the inhibitor of GSK3β signaling is CHIR99021. In some of any embodiments, the cells are exposed to CHIR99021 at a concentration of between about 0.1 μM and about 5 μM, between about 0.5 μM and about 4 μM, or between about 1 μM and about 3 μM, optionally about 2 μM. In some of any embodiments, the cells are exposed to CHIR99021 at a concentration of about 2 μM.

In some of any embodiments, the cells are exposed to GDNF at a concentration of between about 1 ng/mL and about 100 ng/mL, between about 5 ng/mL and about 80 ng/mL, between about 10 ng/mL and about 60 ng/mL, or between about 15 ng/mL and about 30 ng/mL, optionally about 20 ng/mL. In some of any embodiments, the cells are exposed to GDNF at a concentration of about 20 ng/mL.

In some of any embodiments, the cells are exposed to BDNF at a concentration of between about 1 ng/mL and about 100 ng/mL, between about 5 ng/mL and about 80 ng/mL, between about 10 ng/mL and about 60 ng/mL, or between about 15 ng/mL and about 30 ng/mL, optionally about 20 ng/mL. In some of any embodiments, the cells are exposed to BDNF at a concentration of about 20 ng/mL.

In some of any embodiments, the cells are exposed to dbcAMP at a concentration of between about 0.1 mM and 5 mM, between about 0.2 mM and about 4 mM, between about 0.3 mM and about 3 mM, or between about 0.4 mM and about 2 mM, optionally about 0.5 mM. In some of any embodiments, the cells are exposed to dbcAMP at a concentration of about 0.5 mM.

In some of any embodiments, the cells are exposed to ascorbic acid at a concentration of between about 0.05 mM and about 5 mM, between about 0.1 mM and about 1 mM, or between about 0.2 mM and about 0.5 mM, optionally about 0.2 mM. In some of any embodiments, the cells are exposed to ascorbic acid at a concentration of about 0.2 mM.

In some of any embodiments, the cells are exposed to TGFβ3 at a concentration of between about 0.1 ng/mL and about 5 ng/mL, between about 0.3 ng/mL and about 3 ng/mL, or between about 0.5 ng/mL and about 2 ng/mL, optionally about 1 ng/mL. In some of any embodiments, the cells are exposed to TGFβ3 at a concentration of about 1 ng/mL.

In some of any embodiments, the inhibitor of Notch signaling is DAPT. In some of any embodiments, the cells are exposed to DAPT at a concentration of between about 1 μM and about 20 μM, between about 5 μM and about 15 μM, or between about 8 μM and about 12 μM, optionally about 10 μM. In some of any embodiments, the cells are exposed to DAPT at a concentration of about 10 μM.

In some of any embodiments, the culturing in the first incubation and/or the second incubation is carried out in media comprising serum or a serum replacement. In some of any embodiments, the cells are cultured in a media comprising serum or a serum replacement from about day 0 to about day 10. In some of any embodiments, the serum or serum replacement comprises about 5% of the media (v/v) or about 2% of the media (v/v). In some of any embodiments, the media comprises about 5% serum or serum replacement (v/v) from about day 0 to about day 1 about 2% serum replacement (v/v) from about day 2 to about day 10.

In some of any embodiments, the method further comprises harvesting the neurally differentiated cells, optionally wherein the harvesting comprises enzymatic dissociation comprising use of Accutase. In some of any embodiments, the method further comprises harvesting the neurally differentiated cells, wherein the harvesting comprises enzymatic dissociation comprising use of Accutase.

In some of any embodiments, the method comprises harvesting the cells between at or about day 19 and day 24. In some embodiments, the day of harvest is at or about day 19. In some embodiments, the day of harvest is at or about day 20. In some embodiments, the day of harvest is at or about day 21. In some embodiments, the day of harvest is at or about day 22. In some embodiments, the day of harvest is at or about day 23. In some embodiments, the day of harvest is at or about day 24.

In some of any embodiments, the harvesting is carried out at about day 16 or later, optionally between about day 18 and about day 23, optionally wherein the harvesting comprises enzymatic dissociation comprising use of Accutase. In some of any embodiments, the harvesting is carried out between about day 18 and about day 23. In some of any embodiments, the harvesting is carried out at or about at day 18, day 19, day 20, day 21, day 22, or day 23, optionally wherein the harvesting comprises enzymatic dissociation comprising use of Accutase.

In some of any embodiments, the harvesting is carried out at or about at day 20, optionally wherein the harvesting comprises enzymatic dissociation comprising use of Accutase. In some embodiments, the harvesting comprises enzymatic dissociation comprising use of Accutase.

In some of any embodiments, the harvested cells comprise determined dopaminergic neuronal progenitor cells (DDPCs).

In some of any embodiments, at least 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, or 90% of the harvested cells are DDPCs.

In some of any embodiments, the DDPCs express one or more genes selected from (a) ASPM; (b) AURKB; (c) BRINP1; (d) BUB1; (e) CCNB2; (f) CDC20; (g) CDC25C; (h) CDKN1A; (i) CENPF; (j) DLGAP5; (k) FAM83D; (1) FANCD2; (m) GEM; (n) HMMR; (o) IQGAP3; (p) KIF20A; (q) KIF2C; (r) KIFC1; (s) MKI67; (t) PIMREG; (u) PLK2; (v) PTTG1; (w) SAPCD2; (x) TOP2A; (y) TPX2; and (z) TTK.

In some of any embodiments, the DDPCs express one or more genes selected from (b) AURKB; (c) BRINP1; (d) BUB1; (e) CCNB2; (f) CDC20; (g) CDC25C; (h) CDKN1A; (i) CENPF; (j) DLGAP5; (k) FAM83D; (1) FANCD2; (m) GEM; (n) HMMR; (o) IQGAP3; (p) KIF20A; (q) KIF2C; (r) KIFC1; (s) MKI67; (t) PIMREG; (u) PLK2; (v) PTTG1; (w) SAPCD2; (y) TPX2; and (z) TTK.

In some of any embodiments, the DDPCs express one or more genes selected from (b) AURKB; (e) CCNB2; (g) CDC25C; (j) DLGAP5; (k) FAM83D; (o) IQGAP3; (p) KIF20A; (q) KIF2C; (r) KIFC1; (v) PTTG1; (w) SAPCD2; (x) TOP2A; and (y) TPX2.

In some of any embodiments, the DDPCs express one or more genes selected from (b) AURKB; (e) CCNB2; (g) CDC25C; (j) DLGAP5; (k) FAM83D; (o) IQGAP3; (p) KIF20A; (q) KIF2C; (r) KIFC1; (v) PTTG1; (w) SAPCD2; and (y) TPX2.

In some of any embodiments, expression of at least one of the one or more genes by the DDPCs is or is on average 1.1, 1.2, 1.3, 1.4, 1.5, 1.6, 1.7, 1.8, 1.9, or 2.0 fold greater than expression by a reference population of cells. In some of any embodiments, expression of each of the one or more genes by the DDPCs is or is on average 1.1, 1.2, 1.3, 1.4, 1.5, 1.6, 1.7, 1.8, 1.9, or 2.0 fold greater than expression by a reference population of cells.

In some embodiments, the reference population of cells comprises reference cells. In some embodiments, the reference population of cells is enriched for reference cells. In some embodiments, at least 50, 55, 60, 65, 70, 75, 80, 85, 90, 95, 96, 97, 98, or 99 percent of the reference population of cells is reference cells. In some embodiments, the reference population of cells is reference cells.

In some embodiments, the reference cells are not determined dopaminergic neuronal progenitor cells. In some embodiments, the reference cells are pluripotent stem cells. In some embodiments, the reference cells are floor plate midbrain progenitor cells. In some embodiments, the reference cells are differentiated dopaminergic neurons.

In some embodiments, the reference cells are cells differentiated from pluripotent stem cells under conditions to neurally differentiate the cells. In some embodiments, the cells are differentiated according to any of the methods described herein.

In some embodiments, the reference cells are cells at a particular timepoint of the differentiation method. In some embodiments, the timepoint is before the timepoint at which the harvested cells are harvested. In some embodiments, the timepoint is after the timepoint at which the harvested cells are harvested. In some embodiments, the timepoint is day 13. In some embodiments, the timepoint is day 14. In some embodiments, the timepoint is day 15. In some embodiments, the timepoint is day 16. In some embodiments, the timepoint is day 17. In some embodiments, the timepoint is day 16. In some embodiments, the timepoint is day 19. In some embodiments, the timepoint is day 20. In some embodiments, the timepoint is day 21. In some embodiments, the timepoint is day 22. In some embodiments, the timepoint is day 23. In some embodiments, the timepoint is day 24. In some embodiments, the timepoint is day 25.

In some of any embodiments, expression of at least one of the one or more genes by the DDPCs is at a ratio with respect to GAPDH expression. In some of any embodiments, expression of each of the one or more genes by the DDPCs is at a ratio with respect to GAPDH expression.

In some of any embodiments, the DDPCs exhibit or exhibit on average one or more of: (a) a ratio of ASPM to GAPDH expression of greater than about 7×10⁻⁴; (b) a ratio of AURKB to GAPDH expression of greater than about 9×10⁻⁴; (c) a ratio of BRINP1 to GAPDH expression of greater than about 9×10⁻⁵; (d) a ratio of BUB1 to GAPDH expression of greater than about 3×10⁻³; (e) a ratio of CCNB2 to GAPDH expression of greater than about 3×10⁻³; (f) a ratio of CDC20 to GAPDH expression of greater than about 3×10⁻³; (g) a ratio of CDC25C to GAPDH expression of greater than about 5×10⁻⁴; (h) a ratio of CDKN1A to GAPDH expression of greater than about 1×10⁻³; (i) a ratio of CENPF to GAPDH expression of greater than about 7×10⁻³; (j) a ratio of DLGAP5 to GAPDH expression of greater than about 2×10⁻³; (k) a ratio of FAM83D to GAPDH expression of greater than about 6×10⁻⁴; (1) a ratio of FANCD2 to GAPDH expression of greater than about 3×10⁻³; (m) a ratio of GEM to GAPDH expression of greater than about 6×10⁻⁴; (n) a ratio of HMMR to GAPDH expression of greater than about 8×10⁻⁴; (o) a ratio of IQGAP3 to GAPDH expression of greater than about 1×10⁻³; (p) a ratio of KIF20A to GAPDH expression of greater than about 1×10⁻³; (q) a ratio of KIF2C to GAPDH expression of greater than about 3×10⁻³; (r) a ratio of KIFC1 to GAPDH expression of greater than about 2×10⁻³; (s) a ratio of MKI67 to GAPDH expression of greater than about 2 ×10⁻³; (t) a ratio of PIMREG to GAPDH expression of greater than about 1×10⁻³; (u) a ratio of PLK2 to GAPDH expression of greater than about 4×10⁻³; (v) a ratio of PTTG1 to GAPDH expression of greater than about 3×10⁻³; (w) a ratio of SAPCD2 to GAPDH expression of greater than about 1×10⁻³; (x) a ratio of TOP2A to GAPDH expression of greater than about 3×10⁻²; (y) a ratio of TPX2 to GAPDH expression of greater than about 7×10⁻³; and (z) a ratio of TTK to GAPDH expression of greater than about 2×10⁻³.

In some of any embodiments, (a) the ratio of ASPM to GAPDH expression is between about 7×10⁻⁴ and about 2×10⁻¹; (b) the ratio of AURKB to GAPDH expression is between about 9×10⁻⁴ and about 4×10⁻²; (c) the ratio of BRINP1 to GAPDH expression is between about 9×10⁻⁵ and about 5×10⁻²; (d) the ratio of BUB1 to GAPDH expression is between about 3×10⁻³ and about 7×10⁻²; (e) the ratio of CCNB2 to GAPDH expression is between about 3×10⁻³ and about 7×10⁻²; (f) the ratio of CDC20 to GAPDH expression is between about 3×10⁻³ and about 1×10⁻¹; (g) the ratio of CDC25C to GAPDH expression is between about 5×10⁻⁴ and about 3×10⁻²; (h) the ratio of CDKN1A to GAPDH expression is between about 1×10⁻³ and about 9×10⁻²; (i) the ratio of CENPF to GAPDH expression is between about 7×10⁻³ and about 5×10⁻¹; (j) the ratio of DLGAP5 to GAPDH expression is between about 2×10⁻³ and about 9×10⁻²; (k) the ratio of FAM83D to GAPDH expression is between about 6×10⁻⁴ and about 3×10⁻²; (1) the ratio of FANCD2 to GAPDH expression is between about 3×10⁻³ and about 4×10⁻²; (m) the ratio of GEM to GAPDH expression is between about 6×10⁻⁴ and about 3×10⁻²; (n) the ratio of HMMR to GAPDH expression is between about 8×10⁻⁴ and about 6×10⁻²; (o) the ratio of IQGAP3 to GAPDH expression is between about 1×10⁻³ and about 6×10⁻²; (p) the ratio of KIF20A to GAPDH expression is between about 1×10⁻³ and about 8×10⁻²; (q) the ratio of KIF2C to GAPDH expression is between about 3×10⁻³ and about 7×10⁻²; (r) the ratio of KIFC1 to GAPDH expression is between about 2×10⁻³ and about 8×10⁻²; (s) the ratio of MKI67 to GAPDH expression is between about 2×10⁻³ and about 4×10⁻¹; (t) the ratio of PIMREG to GAPDH expression is between about 1×10⁻³ and about 4×10⁻²; (u) the ratio of PLK2 to GAPDH expression is between about 4×10⁻³ and about 6×10⁻²; (v) the ratio of PTTG1 to GAPDH expression is between about 3×10⁻³ and about 9×10⁻²; (w) the ratio of SAPCD2 to GAPDH expression is between about 1×10⁻³ and about 3×10⁻²; (x) the ratio of TOP2A to GAPDH expression is between about 3×10⁻² and about 7×10⁻¹; (y) the ratio of TPX2 to GAPDH expression is between about 7×10⁻³ and about 2×10⁻¹; and/or (z) the ratio of TTK to GAPDH expression is between about 2×10⁻³ and about 8×10⁻². In some of any embodiments, the ratio is on average across the DDPCs.

In some of any embodiments, the ratio of BRINP1 to GAPDH expression is between about 9×10⁻⁵ and about 5×10⁻²; the ratio of CDKN1A to GAPDH expression is between about 1×10⁻³ and about 9×10⁻²; the ratio of FAM83D to GAPDH expression is between about 6×10⁻⁴ and about 3×10⁻²; the ratio of FANCD2 to GAPDH expression is between about 3×10⁻³ and about 4×10⁻²; the ratio of GEM to GAPDH expression is between about 6×10⁻⁴ and about 3×10⁻²; the ratio of PLK2 to GAPDH expression is between about 4×10⁻³ and about 6×10⁻²; and/or the ratio of SAPCD2 to GAPDH expression is between about 1×10⁻³ and about 3×10⁻². In some of any embodiments, the ratio is on average across the DDPCs.

In some of any embodiments, the cells are passaged during the first incubation and/or during the second incubation by enzymatic dissociation comprising use of Accutase.

In some of any embodiments, the method further comprises formulating the harvested cells with a cryoprotectant. In some of any embodiments, the cryoprotectant is selected from among the group consisting of glycerol, propylene glycol, and dimethyl sulfoxide (DMSO).

In some of any embodiments, the method further comprises cryopreserving the formulated cells. In some of any embodiments, the cryopreserving comprises controlled rate freezing.

In some of any embodiments, the pluripotent stem cells are embryonic stem (ES) cells, induced pluripotent stem cells (iPSCs), or a combination thereof. In some of any embodiments, the pluripotent stem cells are induced pluripotent stem cells, optionally human induced pluripotent stem cells. In some of any embodiments, the pluripotent stem cells are human induced pluripotent stem cells. In some of any embodiments, the pluripotent stem cells are autologous to the subject. In some of any embodiments, the pluripotent stem cells are allogeneic to the subject. In some of any embodiments, the pluripotent stem cells are from a healthy human subject. In some of any embodiments, the pluripotent stem cells are from a human subject with a neurodegenerative disease or condition. In some of any embodiments, the neurodegenerative disease or condition comprises the loss of dopaminergic neurons. In some of any embodiments, the neurodegenerative disease or condition is a Parkinsonism. In some of any embodiments, the neurodegenerative disease or condition is Parkinson's disease. In some of any embodiments, the pluripotent stem cells are hypoimmunogenic. In some of any embodiments, the pluripotent stem cells are engineered to (a) remove genes encoding one or more of polymorphic HLA-A/-B/-C and HLA class II molecules; and (b) to provide genes encoding one or more of PD-L1, HLA-G, and CD47, optionally into a AAVS1 safe harbor locus.

Provided herein in some embodiments is a therapeutic composition produced by any of the provided methods. Provided herein is a population of neuronal progenitor cells that is selected as a population of neuronal progenitor cells that is predicted to engraft by any of the provided methods. Provided herein in some embodiments is a therapeutic composition comprising any of the provided populations of neuronal progenitor cells.

Provided herein in some embodiments is a therapeutic composition comprising determined dopaminergic neuronal progenitor cells (DDPCs) derived from pluripotent stem cells, wherein DDPCs of the therapeutic composition express one or more genes selected from (a) ASPM; (b) AURKB; (c) BRINP1; (d) BUB1; (e) CCNB2; (f) CDC20; (g) CDC25C; (h) CDKN1A; (i) CENPF; (j) DLGAP5; (k) FAM83D; (1) FANCD2; (m) GEM; (n) HMMR; (o) IQGAP3; (p) KIF20A; (q) KIF2C; (r) KIFC1; (s) MKI67; (t) PIMREG; (u) PLK2; (v) PTTG1; (w) SAPCD2; (x) TOP2A; (y) TPX2; and (z) TTK.

In some of any embodiments, the DDPCs express one or more genes selected from (b) AURKB; (c) BRINP1; (d) BUB1; (e) CCNB2; (f) CDC20; (g) CDC25C; (h) CDKN1A; (i) CENPF; (j) DLGAP5; (k) FAM83D; (1) FANCD2; (m) GEM; (n) HMMR; (o) IQGAP3; (p) KIF20A; (q) KIF2C; (r) KIFC1; (s) MKI67; (t) PIMREG; (u) PLK2; (v) PTTG1; (w) SAPCD2; (y) TPX2; and (z) TTK.

In some of any embodiments, the DDPCs express one or more genes selected from (b) AURKB; (e) CCNB2; (g) CDC25C; (j) DLGAP5; (k) FAM83D; (o) IQGAP3; (p) KIF20A; (q) KIF2C; (r) KIFC1; (v) PTTG1; (w) SAPCD2; (x) TOP2A; and (y) TPX2.

In some of any embodiments, the DPPCs express one or more genes selected from (b) AURKB; (e) CCNB2; (g) CDC25C; (j) DLGAP5; (k) FAM83D; (o) IQGAP3; (p) KIF20A; (q) KIF2C; (r) KIFC1; (v) PTTG1; (w) SAPCD2; and (y) TPX2.

In some of any embodiments, expression of at least one of the one or more genes by the DDPCs is or is on average 1.1, 1.2, 1.3, 1.4, 1.5, 1.6, 1.7, 1.8, 1.9, or 2.0 fold greater than expression by a reference population of cells. In some of any embodiments, expression of each of the one or more genes by the DDPCs is or is on average 1.1, 1.2, 1.3, 1.4, 1.5, 1.6, 1.7, 1.8, 1.9, or 2.0 fold greater than expression by a reference population of cells.

In some embodiments, the reference population of cells comprises reference cells. In some embodiments, the reference population of cells is enriched for reference cells. In some embodiments, at least 50, 55, 60, 65, 70, 75, 80, 85, 90, 95, 96, 97, 98, or 99 percent of the reference population of cells is reference cells. In some embodiments, the reference population of cells is reference cells.

In some embodiments, the reference cells are not determined dopaminergic neuronal progenitor cells. In some embodiments, the reference cells are pluripotent stem cells. In some embodiments, the reference cells are floor plate midbrain progenitor cells. In some embodiments, the reference cells are differentiated dopaminergic neurons.

In some embodiments, the reference cells are cells differentiated from pluripotent stem cells under conditions to neurally differentiate the cells. In some embodiments, the cells are differentiated according to any of the methods described herein.

In some embodiments, the reference cells are cells at a particular timepoint of the differentiation method. In some embodiments, the timepoint is before the timepoint at which the harvested cells are harvested. In some embodiments, the timepoint is after the timepoint at which the harvested cells are harvested. In some embodiments, the timepoint is day 13. In some embodiments, the timepoint is day 14. In some embodiments, the timepoint is day 15. In some embodiments, the timepoint is day 16. In some embodiments, the timepoint is day 17. In some embodiments, the timepoint is day 16. In some embodiments, the timepoint is day 19. In some embodiments, the timepoint is day 20. In some embodiments, the timepoint is day 21. In some embodiments, the timepoint is day 22. In some embodiments, the timepoint is day 23. In some embodiments, the timepoint is day 24. In some embodiments, the timepoint is day 25.

In some of any embodiments, expression of at least one of the one or more genes by the DDPCs is at a ratio with respect to GAPDH expression. In some of any embodiments, expression of each of the one or more genes by the DDPCs is at a ratio with respect to GAPDH expression.

In some of any embodiments, the DDPCs exhibit or exhibit on average one or more of: (a) a ratio of ASPM to GAPDH expression of greater than about 7×10⁻⁴; (b) a ratio of AURKB to GAPDH expression of greater than about 9×10⁻⁴; (c) a ratio of BRINP1 to GAPDH expression of greater than about 9×10⁻⁵; (d) a ratio of BUB1 to GAPDH expression of greater than about 3×10⁻³; (e) a ratio of CCNB2 to GAPDH expression of greater than about 3×10⁻³; (f) a ratio of CDC20 to GAPDH expression of greater than about 3×10⁻³; (g) a ratio of CDC25C to GAPDH expression of greater than about 5×10⁻⁴; (h) a ratio of CDKN1A to GAPDH expression of greater than about 1×10⁻³; (i) a ratio of CENPF to GAPDH expression of greater than about 7×10⁻³; (j) a ratio of DLGAP5 to GAPDH expression of greater than about 2×10⁻³; (k) a ratio of FAM83D to GAPDH expression of greater than about 6×10⁻⁴; (1) a ratio of FANCD2 to GAPDH expression of greater than about 3×10⁻³; (m) a ratio of GEM to GAPDH expression of greater than about 6×10⁻⁴; (n) a ratio of HMMR to GAPDH expression of greater than about 8×10⁻⁴; (o) a ratio of IQGAP3 to GAPDH expression of greater than about 1×10⁻³; (p) a ratio of KIF20A to GAPDH expression of greater than about 1×10⁻³; (q) a ratio of KIF2C to GAPDH expression of greater than about 3×10⁻³; (r) a ratio of KIFC1 to GAPDH expression of greater than about 2×10⁻³; (s) a ratio of MKI67 to GAPDH expression of greater than about 2 ×10⁻³; (t) a ratio of PIMREG to GAPDH expression of greater than about 1×10⁻³; (u) a ratio of PLK2 to GAPDH expression of greater than about 4×10⁻³; (v) a ratio of PTTG1 to GAPDH expression of greater than about 3×10⁻³; (w) a ratio of SAPCD2 to GAPDH expression of greater than about 1×10⁻³; (x) a ratio of TOP2A to GAPDH expression of greater than about 3×10⁻²; (y) a ratio of TPX2 to GAPDH expression of greater than about 7×10⁻³; and (z) a ratio of TTK to GAPDH expression of greater than about 2×10⁻³.

Provided herein in some embodiments is a therapeutic composition comprising determined dopaminergic neuronal progenitor cells (DDPCs) derived from pluripotent stem cells, wherein the therapeutic composition exhibits one or more of: (a) a ratio of ASPM to GAPDH expression of greater than about 7×10⁻⁴; (b) a ratio of AURKB to GAPDH expression of greater than about 9×10⁻⁴; (c) a ratio of BRINP1 to GAPDH expression of greater than about 9×10⁻⁵; (d) a ratio of BUB1 to GAPDH expression of greater than about 3×10⁻³; (e) a ratio of CCNB2 to GAPDH expression of greater than about 3×10⁻³; (f) a ratio of CDC20 to GAPDH expression of greater than about 3×10⁻³; (g) a ratio of CDC25C to GAPDH expression of greater than about 5×10⁻⁴; (h) a ratio of CDKN1A to GAPDH expression of greater than about 1×10⁻³; (i) a ratio of CENPF to GAPDH expression of greater than about 7×10⁻³; (j) a ratio of DLGAP5 to GAPDH expression of greater than about 2×10⁻³; (k) a ratio of FAM83D to GAPDH expression of greater than about 6×10⁻⁴; (1) a ratio of FANCD2 to GAPDH expression of greater than about 3×10⁻³; (m) a ratio of GEM to GAPDH expression of greater than about 6×10⁻⁴; (n) a ratio of HMMR to GAPDH expression of greater than about 8×10⁻⁴; (o) a ratio of IQGAP3 to GAPDH expression of greater than about 1×10⁻³; (p) a ratio of KIF20A to GAPDH expression of greater than about 1×10⁻³; (q) a ratio of KIF2C to GAPDH expression of greater than about 3×10⁻³; (r) a ratio of KIFC1 to GAPDH expression of greater than about 2×10⁻³; (s) a ratio of MKI67 to GAPDH expression of greater than about 2×10⁻³; (t) a ratio of PIMREG to GAPDH expression of greater than about 1×10⁻³; (u) a ratio of PLK2 to GAPDH expression of greater than about 4×10⁻³; (v) a ratio of PTTG1 to GAPDH expression of greater than about 3×10⁻³; (w) a ratio of SAPCD2 to GAPDH expression of greater than about 1×10⁻³; (x) a ratio of TOP2A to GAPDH expression of greater than about 3×10⁻²; (y) a ratio of TPX2 to GAPDH expression of greater than about 7×10⁻³; and (z) a ratio of TTK to GAPDH expression of greater than about 2×10⁻³.

In some of any embodiments, (a) the ratio of ASPM to GAPDH expression is between about 7×10⁻⁴ and about 2×10⁻¹; (b) the ratio of AURKB to GAPDH expression is between about 9×10⁻⁴ and about 4×10⁻²; (c) the ratio of BRINP1 to GAPDH expression is between about 9×10⁻⁵ and about 5×10⁻²; (d) the ratio of BUB1 to GAPDH expression is between about 3×10⁻³ and about 7×10⁻²; (e) the ratio of CCNB2 to GAPDH expression is between about 3×10⁻³ and about 7×10⁻²; (f) the ratio of CDC20 to GAPDH expression is between about 3×10⁻³ and about 1×10⁻¹; (g) the ratio of CDC25C to GAPDH expression is between about 5×10⁻⁴ and about 3×10⁻²; (h) the ratio of CDKN1A to GAPDH expression is between about 1×10⁻³ and about 9×10⁻²; (i) the ratio of CENPF to GAPDH expression is between about 7×10⁻³ and about 5×10⁻¹; (j) the ratio of DLGAP5 to GAPDH expression is between about 2×10⁻³ and about 9×10⁻²; (k) the ratio of FAM83D to GAPDH expression is between about 6×10⁻⁴ and about 3×10⁻²; (1) the ratio of FANCD2 to GAPDH expression is between about 3×10⁻³ and about 4×10⁻²; (m) the ratio of GEM to GAPDH expression is between about 6×10⁻⁴ and about 3×10⁻²; (n) the ratio of HMMR to GAPDH expression is between about 8×10⁻⁴ and about 6×10⁻²; (o) the ratio of IQGAP3 to GAPDH expression is between about 1×10⁻³ and about 6×10⁻²; (p) the ratio of KIF20A to GAPDH expression is between about 1×10⁻³ and about 8×10⁻²; (q) the ratio of KIF2C to GAPDH expression is between about 3×10⁻³ and about 7×10⁻²; (r) the ratio of KIFC1 to GAPDH expression is between about 2×10⁻³ and about 8×10⁻²; (s) the ratio of MKI67 to GAPDH expression is between about 2×10⁻³ and about 4×10⁻¹; (t) the ratio of PIMREG to GAPDH expression is between about 1×10⁻³ and about 4×10⁻²; (u) the ratio of PLK2 to GAPDH expression is between about 4×10⁻³ and about 6×10⁻²; (v) the ratio of PTTG1 to GAPDH expression is between about 3×10⁻³ and about 9×10⁻²; (w) the ratio of SAPCD2 to GAPDH expression is between about 1×10⁻³ and about 3×10⁻²; (x) the ratio of TOP2A to GAPDH expression is between about 3×10⁻² and about 7×10⁻¹; (y) the ratio of TPX2 to GAPDH expression is between about 7×10⁻³ and about 2×10⁻¹; and/or (z) the ratio of TTK to GAPDH expression is between about 2×10⁻³ and about 8×10⁻².

In some of any embodiments, the composition exhibits between two and 26 of (a)-(z).

In some of any embodiments, the ratio of BRINP1 to GAPDH expression is between about 9×10⁻⁵ and about 5×10⁻²; the ratio of CDKN1A to GAPDH expression is between about 1×10⁻³ and about 9×10⁻²; the ratio of FAM83D to GAPDH expression is between about 6×10⁴ and about 3×10⁻²; the ratio of FANCD2 to GAPDH expression is between about 3×10⁻³ and about 4×10⁻²; the ratio of GEM to GAPDH expression is between about 6×10⁻⁴ and about 3×10⁻²; the ratio of PLK2 to GAPDH expression is between about 4×10⁻³ and about 6×10⁻²; and/or the ratio of SAPCD2 to GAPDH expression is between about 1×10⁻³ and about 3×10⁻².

In some embodiments, the ratio is on average across the DDPCs.

In some of any embodiments, at least 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, or 90% of cells of the therapeutic composition is DDPCs.

In some of any embodiments, cells in the therapeutic composition express EN1 and CORIN. In some of any embodiments, the composition exhibits: (a) a ratio of EN1 to GAPDH expression of greater than about 1×10⁻⁴; and/or (b) a ratio of CORIN to GAPDH expression of greater than about 2×10⁻². In some of any embodiments, the composition exhibits: a ratio of EN1 to GAPDH expression of between about 1.5×10⁻³ and 1×10⁻²; and/or a ratio of CORIN to GAPDH expression of between about 5×10⁻² and 5×10^{-1.}

In some of any embodiments, cells in the composition express TH. In some of any embodiments, less than 10% of the total cells in the composition express TH. In some of any embodiments, the composition exhibits a ratio of TH to GAPDH expression of less than about 3×10⁻². In some of any embodiments, between about 2% and about 10%, between about 2% and about 8%, between about 2% and about 6%, between about 2% and about 4%, between about 4% and about 10%, between about 4% and about 8%, between about 4% and about 6%, between about 6% and about 10%, between about 6% and about 8%, or between about 8% and 10% of the total cells in the composition express TH.

In some of any embodiments, the expression is RNA expression. In some of any embodiments, the RNA expression is measured by RNA sequencing.

In some of any embodiments, cells in the composition are capable of engrafting in and innervating other cells in vivo.

In some of any embodiments, cells in the composition are capable of producing dopamine and optionally do not produce or do not substantially produce norepinephrine. In some of any embodiments, cells in the composition are capable of producing dopamine and do not produce or do not substantially produce norepinephrine

In some of any embodiments, the composition comprises at least 5 million total cells, at least 10 million total cells, at least 15 million total cells, at least 20 million total cells, at least 30 million total cells, at least 40 million total cells, at least 50 million total cells, at least 100 million total cells, at least 150 million total cells, or at least 200 million total cells. In some of any embodiments, the composition comprises between at or about 5 million total cells and at or about 200 million total cells, between at or about 5 million total cells and at or about 150 million total cells, between at or about 5 million total cells and at or about 100 million total cells, between at or about 5 million total cells and at or about 50 million total cells, between at or about 5 million total cells and at or about 25 million total cells, between at or about 5 million total cells and at or about 10 million total cells, between at or about 10 million total cells and at or about 200 million total cells, between at or about 10 million total cells and at or about 150 million total cells, between at or about 10 million total cells and at or about 100 million total cells, between at or about 10 million total cells and at or about 50 million total cells, between at or about 10 million total cells and at or about 25 million total cells, between at or about 25 million total cells and at or about 200 million total cells, between at or about 25 million total cells and at or about 150 million total cells, between at or about 25 million total cells and at or about 100 million total cells, between at or about 25 million total cells and at or about 50 million total cells, between at or about 50 million total cells and at or about 200 million total cells, between at or about 50 million total cells and at or about 150 million total cells, between at or about 50 million total cells and at or about 100 million total cells, between at or about 100 million total cells and at or about 200 million total cells, between at or about 100 million total cells and at or about 150 million total cells, or between at or about 150 million total cells and at or about 200 million total cells.

In some of any embodiments, at least about 70%, 75%, 80%, 85%, 90%, or 95% of the total cells in the composition are viable.

In some of any embodiments, the composition comprises a cryoprotectant. In some of any embodiments, the cryoprotectant is selected from among the group consisting of glycerol, propylene glycol, and dimethyl sulfoxide (DMSO).

In some of any embodiments, the composition is for use in the manufacture of a medicament for treatment of a neurodegenerative disease or condition in a subject, optionally wherein the neurodegenerative disease or condition comprises a loss of dopaminergic neurons.

In some of any embodiments, the composition is for use in treatment of a neurodegenerative disease or condition in a subject, optionally wherein the neurodegenerative disease or condition comprises a loss of dopaminergic neurons. In some of any embodiments, the neurodegenerative disease or condition comprises a loss of dopaminergic neurons in the substantia nigra, optionally in the SNc. In some of any embodiments, the neurodegenerative disease or condition is Parkinson's disease. In some of any embodiments, the neurodegenerative disease or condition is a Parkinsonism.

Provided herein in some embodiments is a method of treatment, comprising implanting in a brain region of a subject in need thereof a therapeutically effective amount of any of the provided therapeutic compositions.

In some of any embodiments, the number of cells implanted in the subject is between about 0.25×10⁶ cells and about 20×10⁶ cells, between about 0.25×10⁶ cells and about 15×10⁶ cells, between about 0.25×10⁶ cells and about 10×10⁶ cells, between about 0.25×10⁶ cells and about 5×10⁶ cells, between about 0.25×10⁶ cells and about 1×10⁶ cells, between about 0.25×10⁶ cells and about 0.75×10⁶ cells, between about 0.25×10⁶ cells and about 0.5×10⁶ cells, between about 0.5×10⁶ cells and about 20×10⁶ cells, between about 0.5×10⁶ cells and about 15×10⁶ cells, between about 0.5×10⁶ cells and about 10×10⁶ cells, between about 0.5×10⁶ cells and about 5×10⁶ cells, between about 0.5×10⁶ cells and about 1×10⁶ cells, between about 0.5×10⁶ cells and about 0.75×10⁶ cells, between about 0.75×10⁶ cells and about 20×10⁶ cells, between about 0.75×10⁶ cells and about 15×10⁶ cells, between about 0.75×10⁶ cells and about 10×10⁶ cells, between about 0.75×10⁶ cells and about 5×10⁶ cells, between about 0.75×10⁶ cells and about 1×10⁶ cells, between about 1×10⁶ cells and about 20×10⁶ cells, between about 1×10⁶ cells and about 15×10⁶ cells, between about 1×10⁶ cells and about 10×10⁶ cells, between about 1×10⁶ cells and about 5×10⁶ cells, between about 5×10⁶ cells and about 20×10⁶ cells, between about 5×10⁶ cells and about 15×10⁶ cells, between about 5×10⁶ cells and about 10×10⁶ cells, between about 10×10⁶ cells and about 20×10⁶ cells, between about 10×10⁶ cells and about 15×10⁶ cells, or between about 15×10⁶ cells and about 20×10⁶ cells.

In some of any embodiments, the subject has a neurodegenerative disease or condition. In some of any embodiments, the neurodegenerative disease or condition comprises the loss of dopaminergic neurons. In some of any embodiments, the subject has lost at least 50%, at least 60%, at least 70%, or at least 80% of dopaminergic neurons. In some of any embodiments, the subject has lost at least 50%, at least 60%, at least 70%, or at least 80% of dopaminergic neurons in the substantia nigra (SN), optionally in the SN pars compacta (SNc). In some of any embodiments, the neurodegenerative disease or condition is a Parkinsonism. In some of any embodiments, the neurodegenerative disease or condition is Parkinson's disease.

In some of any embodiments, the brain region is the substantia nigra.

In some of any embodiments, the implanting is by stereotactic injection.

In some of any embodiments, the cells of the therapeutic composition are autologous to the subject. In some of any embodiments, the cells of the therapeutic composition are allogeneic to the subject.

In some of any embodiments, the cells of the therapeutic composition are hypoimmunogenic. In some of any embodiments, the cells of the therapeutic composition are engineered to (a) remove genes encoding one or more of polymorphic HLA-A/-B/-C and HLA class II molecules; and (b) to provide genes encoding one or more of PD-L1, HLA-G, and CD47, optionally into a AAVS1 safe harbor locus.

### BRIEF DESCRIPTION OF THE DRAWINGS

FIG. 1 shows amphetamine-induced rotation over 24 weeks following transplantation of differentiated dopaminergic neurons in Lister-Hooded rats that were lesioned via unilateral stereotaxic injection of 6-hydroxydopamine (6-OHDA).

FIG. 2A-2B show fiber outgrowth in the transplanted rats.

FIG. 3A-3G show lateral and medial fiber outgrowth in the transplanted rats.

FIG. 4A-4B show fiber density in the transplanted rats.

FIG. 5 shows the number of surviving implanted cells following implantation in rats of cells harvested on day 18 or day 20 of different dopaminergic differentiation methods.

FIG. 6A shows quantification of fiber outgrowth of the cells following implantation.

FIG. 6B shows images of fiber outgrowth of the cells following implantation (240 μM between images x 20=4.8 mm anterior-posterior fiber spread; ˜2 mm+/−from ZOI).

FIG. 7 shows predicted engraftment scores for samples of differentiated dopaminergic neurons. The predicted engraftment scores were output from a machine learning model trained to predict engraftment using expression levels of cell cycle genes.

FIG. 8 shows predicted engraftment scores throughout differentiation (day 16 to day 22) for the samples.

FIG. 9A-9B show exemplary genes whose expression levels were associated with differentiation time. FIG. 9A shows the top 5 significantly up-regulated genes ranked by increasing P-value. FIG. 9B shows the top 5 significant down-regulated genes ranked by increasing P-value.

FIG. 10 shows the results of Principal Component Analysis (PCA) performed using expression levels of 50 genes identified as up-regulated during differentiation (maturity genes). Principal Component 1 (PC1) is shown vs. differentiation time.

FIG. 11 shows for cells differentiated using different experimental protocols the results of PCA performed using expression levels of the 50 identified up-regulated genes. PCA of the maturity genes revealed a transcriptional time delay between the experimental protocols. RNA-seq libraries from Day 18, Day 20, and Day 25 were all made from cells that underwent the same differentiation protocol, and each datapoint represents cells from a different individual (n=6 for each of Day 18, Day 20, and Day 25). Cells that were developed for 18 days under a different differentiation protocol are shown in white with a ‘G’ letter (n=7). While the Geltrex samples (white) were differentiated for the same duration as the Day 18 samples, they were transcriptionally more similar to the Day 20 cells.

FIG. 12 shows that PC1 of four samples, wherein PC1 is based on maturity gene expression, was associated with engraftment success. The plot shows a within-timepoint (Day 18) distribution of samples that were used in an engraftment study. The top 50 up-regulated maturity genes were used, and ordination was performed on the transcriptomes from the four samples (HDF109iPS601, HDF111iPS602, HDF468iPS609, HDF410iPS602). The rank order of PC1 corresponds with the rank order of a visual estimate of engraftment success. PC1 explained 48.9% of the variance of the 50 up-regulated clock genes.

FIG. 13 shows a conceptual model of engraftment success. The x-axis represents time, while the y-axis represents a measure of engraftment success. A threshold of engraftment success is shown that can be captured by a proxy of time or transcriptional (biological) age.

### DETAILED DESCRIPTION

Provided herein are methods of predicting cell engraftment of a population of neuronal progenitor cells. In some embodiments, the neuronal progenitor cells are cells that have been differentiated from pluripotent stem cells, such as induced pluripotent stem cells. Also provided herein are methods of differentiating neural cells.

In some embodiments, cell engraftment is predicted using gene expression levels of a plurality of genes for one or more cells of the population of neuronal progenitor cells. In some embodiments, the plurality of genes include one or more cell cycle genes and/or one or more maturity genes. In some embodiments, the gene expression levels of the plurality of genes is associated with the ability of the population of neuronal progenitor cells to engraft in a brain region of a subject following implantation of the population of neuronal progenitor cells in the brain region. In some embodiments, a population of neuronal progenitor cells is selected for implantation based on the gene expression levels of the plurality of genes. Also provided herein are compositions comprising populations of cells produced or selected according to the provided methods. Also provided herein are methods of treating a subject having a neurodegenerative disease, wherein the subject is treated by implanting any of the provided compositions.

The provided embodiments related to methods for producing a cell therapy of differentiated neuronal progenitor cells that are suitable for administration to a subject for treating a neurodegenerative disease. In particular embodiments, the methods provided herein improve the ability to produce therapeutic cell compositions of differentiated neuronal progenitor cells that are more likely to engraft, such as also innervate, the brainn region of a subject with the neurodegenerative disease. In some particular embodiments, the neurodegenerative disease is Parkinson's disease (PD). In some embodiments, the provided methods address problems related to characteristics of Parkinson's disease (PD), including the selective degeneration of midbrain dopamine (mDA) neurons in patients' brains. Because PD symptoms are primarily due to the selective loss of DA neurons in the substantia nigra of the ventral midbrain, PD is considered suitable for cell replacement therapeutic strategies.

A challenge in developing a cell based therapy for PD has been the identification of an appropriate cell source for use in neuronal replacement. The search for an appropriate cell source is decades-long, and many potential sources for DA neuron replacement have been proposed. Kriks, Protocols for generating ES cell-derived dopamine neurons in Development and engineering of dopamine neurons (eds. Pasterkamp, R. J., Smidt, & Burbach) Landes Biosciences (2008); Fitzpatrick, et al., Antioxid. Redox. Signal. (2009) 11:2189-2208. Several of these sources progressed to early stage clinical trials including catecholaminergic cells from the adrenal medulla, carotid body transplants, or encapsulated retinal pigment epithelial cells. Madrazo, et al., N. Engl. J. Med. (1987) 316: 831-34; Arjona, et al., Neurosurgery (2003) 53: 321-28; Spheramine trial Bakay, et al., Front Biosci. (2004) 9:592-602. However, those trials largely failed to show clinical efficacy and resulted in poor long-term survival and low DA release from the grafted cells.

Another approach was the transplantation of fetal midbrain DA neurons, such as was performed in over 300 patients worldwide. Brundin, et al., Prog. Brain Res. (2010) 184:265-94; Lindvall, & Kokaia, J. Clin. Invest (2010) 120:29-40. Therapy using human fetal tissue in these patients demonstrated evidence of DA neuron survival and in vivo DA release up to 10 or 20 years after transplantation in some patients. In many patients, though, fetal tissue transplantation fails to replace DA neuronal function. Further, fetal tissue transplantation is plagued by challenges including low quantity and quality of donor tissue, ethical and practical issues surrounding tissue acquisition, and the poorly defined heterogeneous nature of transplanted cells, which are some of the factors contributing to the variable clinical outcomes. Mendez, et al. Nature Med. (2008); Kordower, et al. N. Engl. J. Med. (1995) 332:1118-24; and Piccini, et al. Nature Neuroscience (1999) 2:1137-40. Hypotheses as to the limited efficacy observed in the human fetal grafting trials include that fetal grafting may not provide a sufficient number of cells at the correct developmental stage and that fetal tissue is quite poorly defined by cell type and variable with regard to the stage and quality of each tissue sample. Bjorklund, et al. Lancet Neurol. (2003) 2:437-45. A further contributing factor may be inflammatory host response to the graft. Id.

Stem cell-derived cells, such as pluripotent stem cells (PSCs), are contemplated as a source of cells for applications in regenerative medicine. Pluripotent stem cells have the ability to undergo self-renewal and give rise to all cells of the issues of the body. PSCs include two broad categories of cells: embryonic stem (ES) cells and induced pluripotent stem cells (iPSCs). ES cells are derived from the inner cell mass of preimplantation embryos and can be maintained indefinitely and expanded in their pluripotent state in vitro. Romito and Cobellis, Stem Cells Int. (2016) 2016:9451492. iPSCs can be obtained by reprogramming (“dedifferentiating”) adult somatic cells to become more ES cell-like, including having the ability to expand indefinitely and differentiate into all three germ layers. Id.

Pluripotent stem cells such as ES cells have been tested as sources for generating engraftable cells. Early studies in the 1990s using mouse ES cells demonstrated the feasibility of deriving specific lineages from pluripotent cells in vitro, including neurons. Okabe, et al., Mech. Dev. (1996) 59:89-102; Bain, et al., Dev. Biol. (1995) 168v342-357. Midbrain DA neurons were generated using a directed differentiation strategy based on developmental insights from early explants studies. Lee, et al., Nat. Biotechnol. (2000) 18v675-679; Ye, et al., Cell (1998) 93:755-66. However, these efforts did not result in cell populations containing high percentages of midbrain DA neurons or cells capable of restoring neuronal function in vivo. Additionally, the resulting populations contained a mixture of cell types in addition to midbrain DA neurons.

Existing strategies for using human PSCs (hPSCs) for cell therapy has not been entirely satisfactory. DA neurons derived from human PSCs generally have displayed poor in vivo performance, failing to compensate for the endogenous loss of neuronal function. Tabar, et al. Nature Med. (2008) 14:379-81; Lindvall and Kokaia, J. Clin. Invest (2010) 120: 29-40.

More recently, preclinical studies in which human ES cells were first differentiated into midbrain floor intermediates, and then further into DA neurons, exhibited in vivo survival and led to motor deficit recovery in animal models. Krik et al., Nature (2011) 480:547-51; Kirkeby et al., Cell Rep. (2012) 1:703-14. Despite these advances, the use of embryonic stem cells is plagued by ethical concerns, as well as the possibility that such cells may form tumors in patients. Finally, ES cell-derived transplants may cause immune reactions in patients in the context of allogeneic stem cell transplant.

The use of induced pluripotent stem cells (iPSCs), rather than ES-derived cells, has the advantages of avoiding ethical concerns. Further, derivation of iPSCs from a patient to be treated (i.e. the patient receives an autologous cell transplant) avoids risks of immune rejection inherent in the use of embryonic stem cells. As previous studies revealed that poor standardization of transplanted cell material contributes to high variability, new methods of producing substantial numbers of standardized cells, such as for autologous stem cell transplant, are needed. Lindvall and Kokaia, J. Clin. Invest (2010) 120: 29-40.

A study is currently underway in which human iPSCs were differentiated into DA neuron precursors and transplanted into the striatum of a human. However, the ability of these cells to survive, engraft, and innervate other cells in vivo has not yet been reported. Takahashi, Brain Res. (2017) 230:213-26 (2017); Cyranoski, D., Nature News (2018).

Thus, existing strategies have not yet proved to be successful in producing a population of differentiated cells for use in engraftment procedures for restoring neuronal function in vivo.

Provided herein are methods of assessing, selecting or predicting whether a cell population of differentiated neuronal progenitor cells is likely to exhibit cell engraftment when implanted or administered to a brain region of a subject. For instance, the provided methods relate to differentiation of neuronal progenitor cells from pluripotent stem cells, such as induced pluripotent stem cells, and selecting (e.g. harvesting) from a differentiation culture a population of such cells at such time that are ready for for implantation in a subject, such as because they are predicted to, or have features that are associated with, the ability to engraft, such as innervate, the brain region of the subject. In aspects of the provided methods, populations of cells, including cells produced by the differentiation methods described herein, are selected as suitable for transplant, engraftment, and innervation of other cells in vivo based on their differential expression of certain genes, including one or more cell cycle genes and/or one or more maturity genes (e.g., genes that increase or decrease in expression during differentiation).

In some aspects, the provided methods are based on findings shown herein that particular gene expression profiles are associated with or predictive of the ability of a population of differentiating cells to engraft following implantation in a brain region. In some aspects, the provided methods allow for selection of cells suitable for implantation by the identification of cells having these gene expression profiles.

In some aspects, the provided methods are also based on findings shown herein that neuronal progenitor cells that are at different stages of differentiation have varying levels of engraftment following implantation in a brain region. In some aspects, it is shown herein that cells that are within a particular time window of differentiation (e.g., cells that are of a particular biological maturity or biological age) are more likely to engraft following implantation than are cells that are outside this time window. Moreover, it is shown herein that this time window can vary based on cell culture conditions, including conditions in which the cells are differentiated. For instance, in some cases harvesting the cells at later time points resulted in the cells exhibiting engraftment whereas engraftnment was not achieved if the cells from the same differentiation process were harvested at earlier time points. This was suprising because, for instance, it had previously been expected that cells harvested earlier during differentiation would be more likely to engraft, for instance due to younger cells being less differentiated and/or having more proliferative capacity.

For instance, it is shown herein that for certain differentiation conditions, cells harvested at a later timepoint, e.g., at or about day 20, were more likely to engraft than were cells harvested earlier, e.g., at or about day 18. As shown herein, transcriptional analysis revealed that cells harvested at day 20 of one differentiation method had gene expression profiles similar to cells harvested at day 18 of an alternative differentiation method, suggesting that different conditions may affect the rate at which the differentiating cells mature or age. For instance, the methods differed in that the alternative differentiation method included culture of the cells on an adherent vessel composed of a Geltrex™ matrix whereas the other method including culture of cells on an adherent vessel on a substrate containing laminin or a fragment. Moreover, the ability of cells harvested at day 18 from both methods did not exhibit the same ability to engraft or innervate; it is found herein that the ability to engraft is correlated or associated with certain gene expression, which may differ depending on the particular differentiation method. By selecting cells for implantation based on particular gene expression profiles, the provided methods allow for the identification of suitable cells of particular biological age or maturity. In some aspects, such identification is achieved for a population of cells regardless of, or independent from, the specific conditions in which the cells are cultured.

Also provided herein are methods of differentiating PSCs into determined dopaminergic neuron progenitor cells (DDPCs) and/or DA neurons cells. In particular, the provided methods are based on findings that cells differentiated in accord with the provided methods are suitable for transplant, engraftment, and innervation of other cells in vivo. In some embodiments, the cells are harvested at a time in which they are predicted or determined to engraft, such as in accord with the provided methods of predicting, assessing or selecting cells likely to engraft in a brain region of a subject following implantation. In some embodiments of the provided methods, such cells are cells that are differentiated in accord with the provided methods and are harvested between at or about day 18 and day 25. In some embodiments of the provided methods, such cells are cells that are differentiated in accord with the provided methods and are harvested between at or about day 19 and day 24. In some embodiments of the provided methods, such cells are cells that are differentiated in accord with the provided methods and are harvested at or about day 20.

In some embodiments, the methods of differentiation include culture of pluripotent stem cells, such as induced pluripotent stem cells, under conditions to induce differention of the cells to neuronal progenitor cells that are determined dopaminergic neurons. In some embodiments, the methods of differentiating neural cells, include (a) performing a first incubation comprising culturing pluripotent stem cells (PSCs) in a first culture vessel, wherein beginning at the initiation of the first incubation (day 0) the cells are exposed to (i) an inhibitor of TGF-3/activin-Nodal signaling; and (ii) an inhibitor of bone morphogenetic protein (BMP) signaling; and (b) performing a second incubation comprising culturing cells produced by the first incubation in a second culture vessel under conditions to neurally differentiate the cells. In some aspects, the first culture vessel is an adherent culture vessel and the cells are plated under conditions for adherence of the cells during the incubation. In other aspects, the first culture vessel is a non-adherent vessel and the first incubation produces a spheroid of cells, in which prior to performing the second incubation, the spheroid is dissociated to produce a cell suspension (e.g. by enzymatic dissociation), and cells of the cell suspension are cultured in the second culture vessel. In embodiments of the methods, the the second culture vessel is an adherent culture vessel. In particular embodiments herein, the second culture vessel is an adherent culture vessel and is coated with laminin or a fragment thereof. Exemplary methods are described herein.

In some embodiments, cells exhibiting the gene expression profile identified herein or therapeutic compositions containing same described herein may exhibit an improved ability to engraft and/or innvervate other cells compared to cells that do not exhibit the gene expression profile identified herein. In some embodiments, cells exhibiting the gene expression profile identified herein may also exhibit improved efficacy in vivo, due to their state of differentiation and neuronal committement. For example, cells exhibiting the gene expression profile identified herein may demonstrate improved engraftment and/or innvervation, improved efficacy, or both, as compared to cells harvested not exhibiting the gene expression profile identified herein.

In some embodiments, cells identified by the gene expression profile and produced by the provided differentiation method exhibit therapeutic effect(s) to treat a neurodegenerative disease. In some embodiments, the ability for differentiated cells to treat a neurodegenerative disease can be determined in an animal model of a neurodegenerative disease. In some embodiments, the neurodegenerative disease is Parkinson's disease. In some embodiments, differentiated cells harvested by provided method are screened using an animal model of Parkinson's disease. Any known and available animal model of Parkinson's disease can be used for screening. In some embodiments, the animal model is a lesion model wherein animals receive unilateral stereotaxic injection of 6-hydroxydopamine (6-OHDA) into the substantia nigra. In some embodiments, the animal model is a lesion model wherein animals receive unilateral stereotaxic injection of 6-OHDA into the medial forebrain bundle. In some embodiments, a therapeutic composition containing differentiated cells produced by the provided method, e.g. harvested cells, such as harvested at a time in which they are predicted to engraft by the methods provided herien, are implanted into the substantia nigra of the animal model. In some embodiments, a behavioral assay is performed to screen for therapeutic effects of the implantation on the animal model. In some embodiments, the behavioral assay comprises monitoring amphetamine-induced circling behavior. In some embodiments, differentiated cells exhibit a therapeutic effect to treat a neurodegenerative disease if it such cells are determined to reduce, decrease or reverse a Parkinsonian model brain lesion in this model.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker. When referring to a marker in the cell, such as a transcriptional or translational product, the term refers to the presence of detectable transcriptional or translational product, for example, wherein the product is detected at a level substantially above the level detected carrying out the same procedure with a control under otherwise identical conditions and/or at a level substantially similar to that for a cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker. When referring to a marker in the cell, such as a transcriptional or translational product, the term refers to the absence of detectable transcriptional or translational product, for example, wherein the product is not detected at a level substantially above the level detected carrying out the same procedure with a control under otherwise identical conditions, and/or at a level substantially lower than that for cell known to be positive for the marker, and/or at a level substantially similar as compared to that for a cell known to be negative for the marker.

The term “expression” or “expressed” as used herein in reference to a gene refers to the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

As used herein, the term “stem cell” refers to a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

As used herein, the term “adult stem cell” refers to an undifferentiated cell found in an individual after embryonic development. Adult stem cells multiply by cell division to replenish dying cells and regenerate damaged tissue. An adult stem cell has the ability to divide and create another cell like itself or to create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers.

As used herein, the terms “induced pluripotent stem cell,” “iPS” and “iPSC” refer to a pluripotent stem cell artificially derived (e.g., through man-made manipulation) from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells.

As used herein, the term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism.

As used herein, the term “pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

As used herein, the term “reprogramming” refers to the process of dedifferentiating a non-pluripotent cell into a cell exhibiting pluripotent stem cell characteristics.

As used herein, the term “adherent culture vessel” refers to a culture vessel to which a cell may attach via extracellular matrix molecules and the like, and requires the use of an enzyme (e.g., trypsin, dispase, etc.) for detaching cells from the culture vessel. An “adherent culture vessel” is opposed to a culture vessel to which cell attachment is reduced and does not require the use of an enzyme for removing cells from the culture vessel.

As used herein, the term “non-adherent culture vessel” refers to a culture vessel to which cell attachment is reduced or limited, such as for a period of time. A non-adherent culture vessel may contain a low attachment or ultra-low attachment surface, such as may be accomplished by treating the surface with a substance to prevent cell attachment, such as a hydrogel (e.g. a neutrally charged and/or hydrophilic hydrogel) and/or a surfactant (e.g. pluronic acid). A non-adherent culture vessel may contain rounded or concave wells, and/or microwells (e.g. Aggrewells™). In some embodiments, a non-adherent culture vessel is an Aggrewell™ plate. For non-adherent culture vessels, use of an enzyme to remove cells from the culture vessel may not be required.

As used herein, the term “cell culture” may refer to an in vitro population of cells residing outside of an organism. The cell culture can be established from primary cells isolated from a cell bank or animal, or secondary cells that are derived from one of these sources and immortalized for long-term in vitro cultures.

As used herein, the terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division.

As used herein, a composition refers to any mixture of two or more products, substances, or compounds, including cells. It may be a solution, a suspension, liquid, powder, a paste, aqueous, non-aqueous or any combination thereof.

The term “pharmaceutical composition” refers to a composition suitable for pharmaceutical use, such as in a mammalian subject (e.g., a human). A pharmaceutical composition typically comprises an effective amount of an active agent (e.g., cells) and a carrier, excipient, or diluent. The carrier, excipient, or diluent is typically a pharmaceutically acceptable carrier, excipient or diluent, respectively.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, a “subject” is a mammal, such as a human or other animal, and typically is human.

### II. Method for Differentiating Cells

Provided herein are methods of differentiating neural cells involving (1) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-3/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to neurally differentiate the cells.

Also provided herein are methods of differentiating neural cells involving (1) plating pluripotent stem cells on an adherent culture vessel on about day −1; (b) initiating a first incubation on about day 0, wherein the first incubation includes culturing cells on an adherent culture vessel and exposing the cells to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (c) initiating a second incubation on about day 11, wherein the second incubation includess culturing the cells on an adherent culture vessel under conditions to neurally differentiate the cells. In some embodiments, the conditions to neurally differentiate the cells include exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF33) (collectively, “BAGCT”); and (vi) an inhibitor of Notch signaling.

The provided methods of differentiating neural cells, such as by subjecting iPSCs to cell culture methods that induce their differentiation into floor plate midbrain progenitor cells, determined dopamine (DA) neuron progenitor cells, and/or, dopamine (DA) neurons.

As described herein, iPSCs were generated from fibroblasts of human patients with Parkinson's disease. In a first incubation, the iPSCs were then differentiated to midbrain floor plate precursors and grown in non-adherent or adherent culture by exposure to small molecules, such as LDN, SB, PUR, SHH, CHIR, and combinations thereof, such as beginning on day 0.

Where the iPSCs were first grown in non-adherent culture, the resulting spheroids were transferred to an adherent culture as part of a second incubation, optionally following dissociation of the spheroid, before being exposed to additional small molecules (e.g., LDN, CHIR, BDNF, GDNF, ascorbic acid, dbcAMP, TGF33, DAPT, and combinations thereof) to induce further differentiation into engraftable determined DA neuron progenitor cells or DA neurons.

### A. Samples and Cell Preparation

In embodiments of the provided method, pluripotent stem cells are differentiated into floor plate midbrain progenitor cells, determined dopamine (DA) neuron progenitor cells, and/or, dopamine (DA) neurons. Various sources of pluripotent stem cells can be used in the method, including embryonic stem (ES) cells and induced pluripotent stem cells (iPSCs).

In some aspects, pluripotency refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population.

However, identification of various pluripotent stem cell characteristics can also be used to identify pluripotent cells. In some aspects, pluripotent stem cells can be distinguished from other cells by particular characteristics, including by expression or non-expression of certain combinations of molecular markers. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. In some aspects, a pluripotent stem cell characteristic is a cell morphologies associated with pluripotent stem cells.

In some embodiments, pluripotent stem cells are induced pluripotent stem cells (iPSCs), artificially derived from a non-pluripotent cell. In some aspects, a non-pluripotent cell is a cell of lesser potency to self-renew and differentiate than a pluripotent stem cell. iPSCs may be generated by a process known as reprogramming, wherein non-pluripotent cells are effectively “dedifferentiated” to an embryonic stem cell-like state by engineering them to express genes such as OCT4, SOX2, and KLF4. Takahashi and Yamanaka Cell (2006) 126: 663-76.

Methods for generating iPSCs are known. For example, mouse iPSCs were reported in 2006 (Takahashi and Yamanaka), and human iPSCs were reported in late 2007 (Takahashi et al. and Yu et al.). Mouse iPSCs demonstrate important characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers.

In some embodiments, the PSCs (e.g. iPSCs) are autologous to the subject to be treated, i.e. the PSCs are derived from the same subject to whom the differentiated cells are administered. In some embodiments, non-pluripotent cells (e.g., fibroblasts) derived from patients having Parkinson's disease (PD) are reprogrammed to become iPSCs before differentiation into neural and/or neuronal cells. In some embodiments, fibroblasts may be reprogrammed to iPSCs by transforming fibroblasts with genes (OCT4, SOX2, NANOG, LIN28, and KLF4) cloned into a plasmid (for example, see, Yu, et al., Science DOI: 10.1126/science.1172482). In some embodiments, non-pluripotent fibroblasts derived from patients having PD are reprogrammed to become iPSCs before differentiation into determined DA neuron progenitors cells and/or DA neurons, such as by use of the non-integrating Sendai virus to reprogram the cells (e.g., use of CTS™ CytoTune™-iPS 2.1 Sendai Reprogramming Kit). In some embodiments, the resulting differentiated cells are then administered to the patient from whom they are derived in an autologous stem cell transplant. In some embodiments, the PSCs (e.g., iPSCs) are allogeneic to the subject to be treated, i.e. the PSCs are derived from a different individual than the subject to whom the differentiated cells will be administered. In some embodiments, non-pluripotent cells (e.g., fibroblasts) derived from another individual (e.g. an individual not having a neurodegenerative disorder, such as Parkinson's disease) are reprogrammed to become iPSCs before differentiation into determined DA neuron progenitor cells and/or DA neurons. In some embodiments, reprogramming is accomplished, at least in part, by use of the non-integrating Sendai virus to reprogram the cells (e.g., use of CTS™ CytoTune™-iPS 2.1 Sendai Reprogramming Kit). In some embodiments, the resulting differentiated cells are then administered to an individual who is not the same individual from whom the differentiated cells are derived (e.g. allogeneic cell therapy or allogeneic cell transplantation).

In any of the provided embodiments, the PSCs described herein (e.g. allogeneic cells) may be genetically engineered to be hypoimmunogenic. Methods for reducing the immunogenicity are known, and include ablating polymorphic HLA-A/-B/-C and HLA class II molecule expression and introducing the immunomodulatory factors PD-L1, HLA-G, and CD47 into the AAVS1 safe harbor locus in differentiated cells. Han et al., PNAS (2019) 116(21):10441-46. Thus, in some embodiments, the PSCs described herein are engineered to delete highly polymorphic HLA-A/-B/-C genes and to introduce immunomodulatory factors, such as PD-L1, HLA-G, and/or CD47, into the AAVS1 safe harbor locus.

In some embodiments, PSC (e.g., iPSCs) are cultured in the absence of feeder cells, until they reach 80-90% confluency, at which point they are harvested and further cultured for differentiation (day 0). In one aspect of the method described herein, once iPSCs reach 80-90% confluence, they are washed in phosphate buffered saline (PBS) and subjected to enzymatic dissociation, such as with Accutase™, until the cells are easily dislodged from the surface of a culture vessel. The dissociated iPSCs are then re-suspended in media for downstream differentiation into determined DA neuron progenitor cells and/or DA neurons.

In some embodiments, the PSCs are resuspended in a basal induction media. In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with serum replacement, a Rho-associated protein kinase (ROCK) inhibitor, and various small molecules, for differentiation. In some embodiments, the PSCs are resuspended in the same media they will be cultured in for at least a portion of the first incubation.

### B. Non-Adherent Culture

### 1. First Incubation

The provided methods include culturing PSCs (e.g. iPSCs) by incubation with certain molecules (e.g. small molecules) to induce their differentiation into floor plate midbrain progenitor cells, determined dopamine (DA) neuron progenitor cells, and/or, dopamine (DA) neurons. In particular, the provided embodiments include a first incubation of PSCs under non-adherent conditions to produce spheroids, in the presence of certain molecules (e.g., small molecules). In some embodiments, the methods include performing a first incubation involving culturing pluripotent stem cells in a non-adherent culture vessel under conditions to produce a cell spheroid, wherein beginning at the initiation of the first incubation (day 0) the cells are exposed to (i) an inhibitor of TGF-3/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling.

In some embodiments, a non-adherent culture vessel is a culture vessel with a low or ultra-low attachment surface, such as to inhibit or reduce cell attachment. In some embodiments, culturing cells in a non-adherent culture vessel does not prevent all cells of the culture from attaching the surface of the culture vessel.

In some embodiments, a non-adherent culture vessel is a culture vessel with an ultra-low attachment surface. In some aspects, an ultra-low attachment surface may inhibit cell attachment for a period of time. In some embodiments, an ultra-low attachment surface may inhibit cell attachment for the period of time necessary to obtain confluent growth of the same cell type on an adherent surface. In some embodiments, the ultra-low attachment surface is coated or treated with a substance to prevent cell attachment, such as a hydrogel layer (e.g., a neutrally charged and/or hydrophilic hydrogel layer). In some embodiments, a non-adherent culture vessel is coated or treated with a surfactant prior to the first incubation. In some embodiments, the surfactant is pluronic acid.

In some embodiments, the non-adherent culture vessel is a plate, a dish, a flask, or a bioreactor. In some embodiments, the non-adherent culture vessel is a plate, such as a multi-well plate. In some embodiments, the non-adherent culture vessel is a 6-well or 24-well plate. In some embodiments, the wells of the multi-well plate further include micro-wells. In some any of the provided embodiments, a non-adherent culture vessel, such as a multi-well plate, has round or concave wells and/or microwells. In any of the provided embodiments, a non-adherent culture vessel, such as a multi-well plate, does not have corners or seams.

In some embodiments, a non-adherent culture vessel allows for three-dimensional formation of cell aggregates. In some embodiments, iPSCs are cultured in a non-adherent culture vessel, such as a multi-well plate, to produce cell aggregates (e.g., spheroids). In some embodiments, iPSCs are cultured in a non-adherent culture vessel, such as a multi-well plate, to produce cell aggregates (e.g., spheroids) on about day 7 of the method. In some embodiments, the cell aggregate (e.g., spheroid) expresses at least one of PAX6 and OTX2 on or by about day 7 of the method.

In some embodiments, the first incubation includes culturing pluripotent stem cells in a non-adherent culture vessel under conditions to produce a cellular spheroid.

In some embodiments, the number of PSCs plated on day 0 of the method is between about between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.4×10⁶ cells/cm² and about 0.8×10⁶ cells/cm².

In some embodiments, the number of PSCs plated on day 0 of the method is between about 1×10⁵ pluripotent stem cells per well and about 20×10⁶ pluripotent stem cells per well, between about 1×10⁵ pluripotent stem cells per well and about 15×10⁶ pluripotent stem cells per well, between about 1×10⁵ pluripotent stem cells per well and about 10×10⁶ pluripotent stem cells per well, between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 20×10⁶ pluripotent stem cells per well, between about 5×10⁵ pluripotent stem cells per well and about 15×10⁶ pluripotent stem cells per well, between about 5×10⁵ pluripotent stem cells per well and about 10×10⁶ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 6-well plate on day 0 of the method is between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 24-well plate on day 0 of the method is between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, or between about 1×10⁶ pluripotent stem cells per well and about 5×10⁶ pluripotent stem cells per well.

In some days, the number of PSCs plated on day 0 of the method is a number of cells sufficient to produce a cellular spheroid containing between about 1,000 cells and about 5,000 cells, or between about 2,000 cells and about 3,000 cells. In some days, the number of PSCs plated on day 0 of the method is a number of cells sufficient to produce a cellular spheroid containing between about 1,000 cells and about 5,000 cells. In some days, the number of PSCs plated on day 0 of the method is a number of cells sufficient to produce a cellular spheroid containing between about 2,000 cells and about 3,000 cells. In some days, the number of PSCs plated on day 0 of the method is a number of cells sufficient to produce a cellular spheroid containing about 2,000 cells. In some days, the number of PSCs plated on day 0 of the method is a number of cells sufficient to produce a cellular spheroid containing about 3,000 cells. In some embodiments, the spheroids containing the desired number is produced by the method on or by about day 7.

In some embodiments of the method provided herein, the first incubation includes culturing pluripotent stem cells in a non-adherent culture vessel under conditions to produce a cellular spheroid. In some embodiments, the first incubation is from about day 0 through about day 6. In some embodiments, the first incubation comprises culturing pluripotent stem cells in a culture media (“media”). In some embodiments, the first incubation comprises culturing pluripotent stem cells in the media from about day 0 through about day 6. In some embodiments, the first incubation comprises culturing pluripotent stem cells in the media to induce differentiation of the PSCs into floor plate midbrain progenitor cells.

In some embodiments, the media is also supplemented with a serum replacement containing minimal non-human-derived components (e.g., KnockOut™ serum replacement). In some embodiments, the serum replacement is provided in the media at 5% (v/v) for at least a portion of the first incubation. In some embodiments, the serum replacement is provided in the media at 5% (v/v) on day 0 and day 1. In some embodiments, the serum replacement is provided in the media at 2% (v/v) for at least a portion of the first incubation. In some embodiments, the serum replacement is provided in the media at 2% (v/v) from day 2 through day 6. In some embodiments, the serum replacement is provided in the media at 5% (v/v) on day 0 and day 1, and at 2% (v/v) from day 2 through day 6.

In some embodiments, the media is further supplemented with small molecules, such as any described above. In some embodiments, the small molecules are selected from among the group consisting of: a Rho-associated protein kinase (ROCK) inhibitor, an inhibitor of TGF-β/activin-Nodal signaling, at least one activator of Sonic Hedgehog (SHH) signaling, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 0.

In some embodiments, cells are exposed to the ROCK inhibitor at a concentration of between about 1 μM and about 20 μM, between about 5 μM and about 15 PM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 PM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is selected from among the group consisting of: Fasudil, Ripasudil, Netarsudil, RKI-1447, Y-27632, GSK429286A, Y-30141, and combinations thereof. In some embodiments, the ROCK inhibitor is a small molecule. In some embodiments, the ROCK inhibitor selectively inhibits p160ROCK. In some embodiments, the ROCK inhibitor is Y-27632, having the formula:

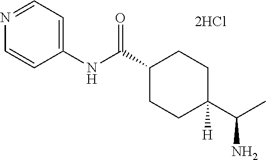 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 0.

In some embodiments the media is supplemented with an inhibitor of TGF-3/activin-Nodal signaling. In some embodiments the media is supplemented with an inhibitor of TGF-β/activin-Nodal signaling up to about day 7 (e.g. day 6 or day 7). In some embodiments the media is supplemented with an inhibitor of TGF-β/activin-Nodal signaling from about day 0 through day 6, each day inclusive.

In some embodiments, cells are exposed to the inhibitor of TGF-β/activin-Nodal signaling at a concentration of between about 1 μM and about 20 μM, between about 5 μM and about 15 μM, or between about 8 μM and about 12 μM. In some embodiments, cells are exposed to the inhibitor of TGF-β/activin-Nodal signaling at a concentration of between about 1 μM and about 20 μM. In some embodiments, cells are exposed to the inhibitor of TGF-3/activin-Nodal signaling at a concentration of between about 5 μM and about 15 PM. In some embodiments, cells are exposed to the inhibitor of TGF-β/activin-Nodal signaling at a concentration of between about 8 μM and about 12 μM. In some embodiments, cells are exposed to the inhibitor of TGF-3/activin-Nodal signaling at a concentration of about 10 μM.

In some embodiments, the inhibitor of TGF-β/activin-Nodal signaling is a small molecule. In some embodiments, the inhibitor of TGF-β/activin-Nodal signaling is capable of lowering or blocking transforming growth factor beta (TGFP)/Activin-Nodal signaling. In some embodiments, the inhibitor of TGF-β/activin-Nodal signaling inhibits ALK4, ALK5, ALK7, or combinations thereof. In some embodiments, the inhibitor of TGF-β/activin-Nodal signaling inhibits ALK4, ALK5, and ALK7. In some embodiments, the inhibitor of TGF-β/activin-Nodal signaling does not inhibit ALK2, ALK3, ALK6, or combinations thereof. In some embodiments, the inhibitor does not inhibit ALK2, ALK3, or ALK6. In some embodiments, the inhibitor of TGF-β/activin-Nodal signaling is SB431542 (e.g., CAS 301836-41-9, molecular formula of C22H18N4O3, and name of 4-[4-(1,3-benzodioxol-5-yl)-5-(2-pyridinyl)-1H-imidazol-2-yl]-benzamide), having the formula:

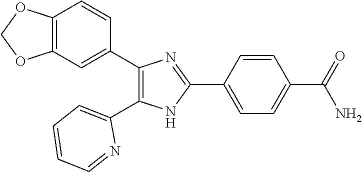 [SMILES: NC(=O)c1ccc(-c2nc(-c3ccc4c(c3)OCO4)c(-c3ccccn3)[nH]2)cc1]

In some embodiments, cells are exposed to SB431542 at a concentration of about 10 μM. In some embodiments, cells are exposed to SB431542 at a concentration of about 10 μM until about day 7. In some embodiments, cells are exposed to SB431542 at a concentration of about 10 μM from about day 0 through about day 6, inclusive of each day.

In some embodiments the media is supplemented with at least one activator of sonic hedghehog (SHH) signaling. SHH refers to a protein that is one of at least three proteins in the mammalian signaling pathway family called hedgehog, another is desert hedgehog (DHH) while a third is Indian hedgehog (IHH). Shh interacts with at least two transmembrane proteins by interacting with transmembrane molecules Patched (PTC) and Smoothened (SMO). In some embodiments the media is supplemented with the at least one activator of SHH signaling up to about day 7 (e.g., day 6 or day 7). In some embodiments the media is supplemented with the at least one activator of SHH signaling from about day 0 through day 6, each day inclusive.

In some embodiments, the at least one activator of SHH signaling is SHH protein. In some embodiments, the at least one activator of SHH signaling is recombinant SHH protein. In some embodiments, the at least one activator of SHH signaling is recombinant mouse SHH protein. In some embodiments, the at least one activator of SHH signaling is recombinant human SHH protein. In some embodiments, the least one activator of SHH signaling is a recombinant N-Terminal fragment of a full-length murine sonic hedgehog protein capable of binding to the SHH receptor for activating SHH. In some embodiments, the at least one activator of SHH signaling is C25II SHH protein.

In some embodiments, cells are exposed to the at least one activator of SHH signaling at a concentration of between about 10 ng/mL and about 500 ng/mL, between about 20 ng/mL and 400 μg/mL, between about 30 ng/mL and about 300 ng/mL, between about 40 ng/mL and about 200 ng/mL, or between about 50 ng/mL and about 100 ng/mL, each inclusive. In some embodiments, cells are exposed to the at least one activator of SHH signaling at a concentration of between about 50 ng/mL and about 100 ng/mL, each inclusive. In some embodiments, cells are exposed to the at least one activator of SHH signaling at a concentration of about 100 ng/mL. In some embodiments, the cells are exposed to SHH protein at about 100 ng/mL. In some embodiments, the cells are exposed to recombinant SHH protein at about 100 ng/mL. In some embodiments, the cells are exposed to recombinant mouse SHH protein at about 100 ng/mL. In some embodiments, the cells are exposed to C25II SHH protein at about 100 ng/mL.

In some embodiments, cells are exposed to recombinant SHH protein at a concentration of about 10 ng/mL. In some embodiments, cells are exposed to recombinant SHH protein at a concentration of about 10 ng/mL up to about day 7 (e.g., day 6 or day 7). In some embodiments, cells are exposed to recombinant SHH protein at a concentration of about 10 ng/mL from about day 0 through about day 6, inclusive of each day.

In some embodiments, cells are exposed to the at least one activator of SHH signaling at a concentration of between about 1 μM and about 20 μM, between about 5 μM and about 15 μM, or between about 8 μM and about 12 μM. In some embodiments, cells are exposed to the at least one activator of SHH signaling at a concentration of between about 1 μM and about 20 μM. In some embodiments, cells are exposed to the at least one activator of SHH signaling at a concentration of between about 5 μM and about 15 μM. In some embodiments, cells are exposed to the at least one activator of SHH signaling at a concentration of between about 8 μM and about 12 μM. In some embodiments, cells are exposed to the at least one activator of SHH signaling at a concentration of about 10 μM.

In some embodiments, the at least one activator of SHH signaling is an activator of the Hedgehog receptor Smoothened. It some embodiments, the at least one activator of SHH signaling is a small molecule. In some embodiments, the least one activator of SHH signaling is purmorphamine (e.g. CAS 483367-10⁻⁸), having the formula below:

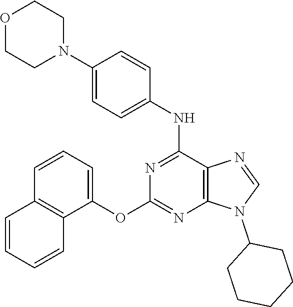 [SMILES: c1ccc2c(Oc3nc(Nc4ccc(N5CCOCC5)cc4)c4ncn(C5CCCCC5)c4n3)cccc2c1]

In some embodiments, cells are exposed to purmorphamine at a concentration of about 10 μM. In some embodiments, cells are exposed to purmorphamine at a concentration of about 10 μM up to day 7 (e.g., day 6 or day 7). In some embodiments, cells are exposed to purmorphamine at a concentration of about 10 μM from about day 0 through about day 6, inclusive of each day.

In some embodiments, the at least one activator of SHH signaling is SHH protein and purmorphamine. In some embodiments, cells are exposed to SHH protein and purmorphamine at a concentration up to about day 7 (e.g., day 6 or day 7). In some embodiments, cells are exposed to SHH protein and purpomorphamine from about day 0 through about day 6, inclusive of each day. In some embodiments, cells are exposed to 100 ng/mL SHH protein and 10 μM purmorphamine at a concentration up to about day 7 (e.g., day 6 or day 7). In some embodiments, cells are exposed to 100 ng/mL SHH protein and 10 μM purpomorphamine from about day 0 through about day 6, inclusive of each day.

In some embodiments the media is supplemented with an inhibitor of BMP signaling. In some embodiments the media is supplemented with an inhibitor of BMP signaling up to about day 7 (e.g., day 6 or day 7). In some embodiments the media is supplemented with an inhibitor of BMP signaling from about day 0 through day 6, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is selected from LDN193189 or K02288. In some embodiments, the inhibitor of BMP signaling is capable of inhibiting “Small Mothers Against Decapentaplegic” SMAD signaling. In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

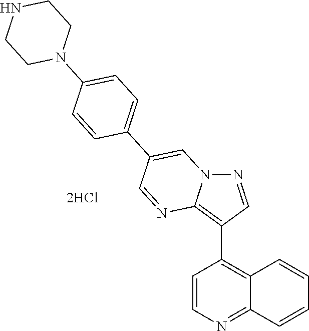 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM up to about day 7 (e.g., day 6 or day 7). In some embodiments, cells are exposed to LDN193189 at a concentration of about 0.1 μM from about day 0 through about day 6, inclusive of each day.

In some embodiments the media is supplemented with an inhibitor of GSK3β signaling. In some embodiments the media is supplemented with an inhibitor of GSK3β signaling up to about day 7 (e.g., day 6 or day 7). In some embodiments the media is supplemented with an inhibitor of GSK3β signaling from about day 0 through day 6, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of BMP signaling at a concentration of between about 1 μM and about 4 μM. In some embodiments, cells are exposed to the inhibitor of BMP signaling at a concentration of between about 2 μM and about 3 PM. In some embodiments, cells are exposed to the inhibitor of GSK3β signaling at a concentration of about 2 μM.

In some embodiments, the inhibitor of GSK3β signaling is selected from among the group consisting of: lithium ion, valproic acid, iodotubercidin, naproxen, famotidine, curcumin, olanzapine, CHIR99012, and combinations thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3β. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

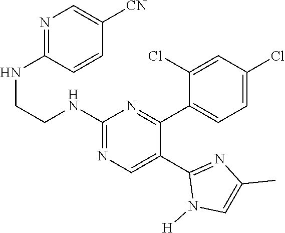 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM up to about day 7 (e.g., day 6 or day 7). In some embodiments, cells are exposed to CHIR99021 at a concentration of about 2.0 μM from about day 0 through about day 6, inclusive of each day.

In some embodiments, from day about 2 to about day 6, at least about 50% of the media is replaced daily. In some embodiments, from about day 2 to about day 6, about 50% of the media is replaced daily, every other day, or every third day. In some embodiments, from about day 2 to about day 6, about 50% of the media is replaced daily. In some embodiments, at least about 75% of the media is replaced on day 1. In some embodiments, about 100% of the media is replaced on day 1. In some embodiments, the replacement media contains small molecules about twice as concentrated as compared to the concentration of the small molecules in the media on day 0.

In some embodiments, the first incubation comprises culturing pluripotent stem cells in a “basal induction media.” In some embodiments, the first incubation comprises culturing pluripotent stem cells in the basal induction media from about day 0 through about day 6. In some embodiments, the first incubation comprises culturing pluripotent stem cells in the basal induction media to induce differentiation of the PSCs into floor plate midbrain progenitor cells.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, 0-mercaptoethanol, and insulin. In some embodiments, the basal induction media is further supplemented with any of the small molecules as described above.

### 2. Transfer or Dissociation of Spheroids

In some embodiments, cell aggregates (e.g. spheroids) that are produced following the first incubation of culturing pluripotent stem cells in a non-adherent culture vessel are transferred or dissociated, prior to carrying out a second incubation of the cells on a substrate (adherent culture).

In some embodiments, the first incubation is carried out to produce a cell aggregate (e.g. a spheroid) that expresses at least one of PAX6 and OTX2. In some embodiments, the first incubation produces a cell aggregate (e.g. a spheroid) that expresses PAX6 and OTX2. In some embodiments, the first incubation produces a cell aggregate (e.g. a spheroid) on or by about day 7 of the methods provided herein. In some embodiments, the first incubation produces a cell aggregate (e.g. a spheroid) that expresses at least one of PAX6 and OTX2 on or by about day 7 of the methods provided herein. In some embodiments, the first incubation produces a cell aggregate (e.g. a spheroid) that expresses PAX6 and OTX2 on or by about day 7 of the methods provided herein.

In some embodiments, the cell aggregate (e.g. spheroid) produced by the first incubation is dissociated prior to the second incubation of the cells on a substrate. In some embodiments, the cell aggregate (e.g. spheroid) produced by the first incubation is dissociated to produce a cell suspension. In some embodiments, the cell suspension produced by the dissociation is a single cell suspension. In some embodiments, the dissociation is carried out at a time when the spheroid cells express at least one of PAX6 and OTX2. In some embodiments, the dissociation is carried out at a time when the spheroid cells express PAX6 and OTX2. In some embodiments, the dissociation is carried out on about day 7. In some embodiments, the cell aggregate (e.g. spheroid) is dissociated by enzymatic dissociation. In some embodiments, the enzyme is selected from among the group consisting of: accutase, dispase, collagenase, and combinations thereof. In some embodiments, the enzyme comprises accutase. In some embodiments, the enzyme is accutase. In some embodiments, the enzyme is dispase. In some embodiments, the enzyme is collagenase. In some embodiments, the enzyme is dispase and collagenase.

In some embodiments, the cell aggregate or cell suspension produced therefrom is transferred to a substrate-coated culture vessel for a second incubation. In some embodiments, the cell aggregate (e.g. spheroid) or cell suspension produced therefrom is transferred to a substrate-coated culture vessel following dissociation of the cell aggregate (e.g. spheroid). In some embodiments, the transferring is carried out immediately after the dissociating. In some embodiments, the transferring is carried out on about day 7.

In some embodiments, the cell aggregate (e.g., spheroid) is not dissociated prior to a second incubation. In some embodiments, a cell aggregate (e.g. spheroid) is transferred in its entirety to a substrate-coated culture vessel for a second incubation. In some embodiments, the transferring is carried out at a time when the spheroid cells express at least one of PAX6 and OTX2. In some embodiments, the transferring is carried out at a time when the spheroid cells express PAX6 and OTX2. In some embodiments, the transferring is carried out on about day 7.

In some embodiments, the transferring is to an adherent culture vessel. In some embodiments, the culture vessel is a plate, a dish, a flask, or a bioreactor. In some embodiments, the culture vessel is substrate-coated. In some embodiments, the substrate is a basement membrane protein. In some embodiments, the substrate is selected from laminin or a fragment thereof, collagen, entactin, heparin sulfate proteoglycans, and combinations thereof. In some embodiments, the substrate is laminin or a fragment thereof. In some embodiments, the substrate is recombinant. In some embodiments, the substrate is recombinant laminin or a fragment thereof. In some embodiments, the substrate is xeno-free. In some embodiments, the substrate is xeno-free laminin or a fragment thereof.

In some embodiments, the laminin or fragment thereof comprises an alpha chain, a beta chain, and a gamma chain. In some embodiments, the alpha chain is LAMA1, LAMA2, LAMA3, LAMA4, LAMA5, or a combination thereof. In some embodiments, the beta chain is LAMB1, LAMB2, LAMB3, LAMB4, or a combination thereof. In some embodiments, the gamma chain is LAMC1, LAMC2, LAMC3, or a combination thereof. In some embodiments, the laminin or a fragment thereof comprises any alpha, beta, and/or gamma chains as described in Aumailley, Cell Adh Migra (2013) 7(1):48-55 (see e.g. Table 1).

In some embodiments, the laminin or a fragment thereof is selected from the group consisting of: laminin 111, laminin 121, laminin 211, laminin 213, laminin 221, laminin 3A32, laminin 3B32, laminin 3A11, laminin 3A21, laminin 411, laminin 421, laminin 423, laminin 511, laminin 521, laminin 522, laminin 523, or a fragment of any of the foregoing. In some embodiments, the laminin is selected from laminin 521, laminin 111, laminin 511, and laminin 511-E8.

In some embodiments, the laminin or a fragment thereof comprises LAMA1, LAMB1, and LAMC1. In some embodiments, the laminin or a fragment thereof is laminin 111.

In some embodiments, the laminin or a fragment thereof comprises LAMA1, LAMB2, and LAMC1. In some embodiments, the laminin or a fragment thereof is laminin 121.

In some embodiments, the laminin or a fragment thereof comprises LAMA2, LAMB1, and LAMC1. In some embodiments, the laminin or a fragment thereof is laminin 211.

In some embodiments, the laminin or a fragment thereof comprises LAMA2, LAMB1, and LAMC3. In some embodiments, the laminin or a fragment thereof is laminin 213.

In some embodiments, the laminin or a fragment thereof comprises LAMA2, LAMB2, and LAMC1. In some embodiments, the laminin or a fragment thereof is laminin 221.

In some embodiments, the laminin or a fragment thereof comprises LAMA3A, LAMB3, and LAMC2. In some embodiments, the laminin or a fragment thereof is laminin 3A32.

In some embodiments, the laminin or a fragment thereof comprises LAMA3B, LAMB3, and LAMC2. In some embodiments, the laminin or a fragment thereof is laminin 3B32.

In some embodiments, the laminin or a fragment thereof comprises LAMA3A, LAMB1, and LAMC1. In some embodiments, the laminin or a fragment thereof is laminin 3A11.

In some embodiments, the laminin or a fragment thereof comprises LAMA3A, LAMB2, and LAMC1. In some embodiments, the laminin or a fragment thereof is laminin 3A21.

In some embodiments, the laminin or a fragment thereof comprises LAMA4, LAMB1, and LAMC1. In some embodiments, the laminin or a fragment thereof is laminin 411.

In some embodiments, the laminin or a fragment thereof comprises LAMA4, LAMB2, and LAMC1. In some embodiments, the laminin or a fragment thereof is laminin 421.

In some embodiments, the laminin or a fragment thereof comprises LAMA4, LAMB2, and LAMC3. In some embodiments, the laminin or a fragment thereof is laminin 423.

In some embodiments, the laminin or a fragment thereof comprises LAMA5, LAMB1, and LAMC1. In some embodiments, the laminin or a fragment thereof is laminin 511.

In some embodiments, the laminin or a fragment thereof is a fragment of laminin 511. In some embodiments, the laminin or a fragment thereof comprises a fragment of LAMA5, a fragment of LAMB1, and a fragment of LAMC1. In some embodiments, the laminin or a fragment thereof comprises a truncated C-terminal fragment of LAMA5, a truncated, C-terminal fragment of LAMB1, and a truncated, C-terminal fragment of LAMC1. In some embodiments, the laminin or a fragment thereof comprises an E8 fragment of LAMA5, an E8 fragment of LAMB1, and an E8 fragment of LAMC1. In some embodiments, the laminin or a fragment thereof is laminin 511-E8 fragment. See Miyazaki et al., Nat Commun (2012) 3:1236.

In some embodiments, the laminin or a fragment thereof comprises LAMA5, LAMB2, and LAMC1. In some embodiments, the laminin or a fragment thereof is laminin 521.

In some embodiments, the laminin or a fragment thereof comprises LAMA5, LAMB2, and LAMC2. In some embodiments, the laminin or a fragment thereof is laminin 522.

In some embodiments, the laminin or a fragment thereof comprises LAMA5, LAMB2, and LAMC3. In some embodiments, the laminin or a fragment thereof is laminin 523.

In some embodiments, the substrate-coated culture vessel is exposed to poly-L-ornithine, optionally prior to being used for culturing cells. In some embodiments, the substrate-coated culture vessel is a 6-well or 24-well plate. In some embodiments, the substrate-coated culture vessel is a 6-well plate. In some embodiments, the substrate-coated culture vessel is a 24-well plate.

### 3. Second Incubation

In some embodiments, the methods include performing a second incubation of the spheroid cells transferred to the substrate-coated culture vessel. In some embodiments, culturing the cells of the spheroid in the substrate-coated culture vessel under adherent conditions induces their differentiation into floor plate midbrain progenitor cells, determined dopamine (DA) neuron progenitor cells, and/or, dopamine (DA) neurons.

In some embodiments, the second incubation involves culturing cells of the spheroid in a culture vessel coated with a substrate including laminin, collagen, entactin, heparin sulfate proteoglycans, or a combination thereof, wherein beginning on day 7, the cells are exposed to (i) an inhibitor of BMP signaling and (ii) an inhibitor of GSK3β signaling; and beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF33); and (vi) an inhibitor of Notch signaling.

In some embodiments, the method further includes harvesting the differentiated cells. In some embodiments, the differentiated cells are harvested at a time in which it is determined that the cells of the second incubation will be predicted to engraft, such as determined using the methods of predicting cell engraftment or assessing cells for engraftment as described in Section III. In some embodiments, the provided methods of predicting cell engraftment or assessing cells for engraftment are performed during the second incubation. In some embodiments, the provided methods of predicting cell engraftment or assessing cells for engraftment are to determine, based on gene expression levels, if cells of the second incubation have reached a biological age or maturity that is associated with the ability of cells to engraft following implantation.

In some embodiments, the substrate-coated culture vessel is a culture vessel with a surface to which cells can attach. In some embodiments, the substrate-coated culture vessel is a culture vessel with a surface to which a substantial number of cells attach. In some embodiments, the substrate is a basement membrane protein. In some embodiments, the substrate is laminin or a fragment thereof, collagen, entactin, heparin sulfate proteoglycans, or a combination thereof. In some embodiments, the substrate is laminin or a fragment thereof. In some embodiments, the substrate is collagen. In some embodiments, the substrate is entactin. In some embodiments, the substrate is heparin sulfate proteoglycans. In some embodiments, the substrate is a recombinant protein. In some embodiments, the substrate is recombinant laminin or a fragment thereof. In some embodiments, the laminin is selected from laminin 521, laminin 111, laminin 511, and laminin 511-E8. In some embodiments, the laminin is laminin 511-E8.

In some embodiments, the substrate-coated culture vessel is exposed to poly-L-ornithine. In some embodiments, the substrate-coated culture vessel is exposed to poly-L-ornithine prior to being used for cell culture.

In some embodiments, thesubstrate-coated culture vessel is a plate, a dish, a flask, or a bioreactor. In some embodiments, the substrate-coated culture vessel is a plate, such as a multi-well plate. In some embodiments, the substrate-coated culture vessel is a plate. In some embodiments, the substrate-coated culture vessel is a 6-well or 24-well plate. In some embodiments, the substrate-coated culture vessel is a dish. In some embodiments, the substate-coated culture vessel is a flask. In some embodiments, the substrate-coated culture vessel is a bioreactor.

In some embodiments, the substrate-coated culture vessel allows for a monolayer cell culture. In some embodiments, cells derived from the cell aggregate (e.g. spheroid) produced by the first incubation are cultured in a monolayer culture on the substrate-coated plates. In some embodiments, cells derived from the cell aggregate (e.g. spheroid) produced by the first incubation are cultured to produce a monolayer culture of cells positive for one or more of LMX1A, FOXA2, EN1, CORIN, and combinations thereof. In some embodiments, cells derived from the cell aggregate (e.g. spheroid) produced by the first incubation are cultured to produce a monolayer culture of cells, wherein at least some of the cells are positive for EN1 and CORIN.

In some embodiments, cells derived from the cell aggregate (e.g. spheroid) produced by the first incubation are cultured to produce a monolayer culture of cells, wherein at least some of the cells are TH+. In some embodiments, at least some cells are TH+ by or on about day 25. In some embodiments, cells derived from the cell aggregate (e.g. spheroid) produced by the first incubation are cultured to produce a monolayer culture of cells, wherein at least some of the cells are TH+FOXA2+. In some embodiments, at least some cells are TH+FOXA2+ by or on about day 25.

In the methods provided herein, the second incubation involves culturing cells of the spheroid in a substrate-coated culture vessel under conditions to induce neural differentiation of the cells. In some embodiments, the cells of the spheroid are plated on the substrate-coated culture vessel on about day 7.

In some embodiments, the number of cells plated on the substrate-coated culture vessel is between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.4×10⁶ cells/cm² and about 0.8×10⁶ cells/cm².

In some embodiments, the second incubation is from about day 7 until harvesting of the cells. In some embodiments, the cells are harvested on about day 16 or later. In some embodiments, the cells are harvested between about day 16 and about day 30. In some embodiments, the cells are harvested between about day 18 and about day 25. In some embodiments, the cells are harvested on about day 18. In some embodiments, the cells are harvested on about day 25. In some embodiments, the second incubation is from about day 7 until about day 18. In some embodiments, the second incubation is from about day 7 until about day 25.

In some embodiments, the second incubation involves culturing cells derived from the cell aggregate (e.g. spheroid) in a culture media (“media”).

In some embodiments, the second incubation involves culturing the cells in the media from about day 7 until harvest or collection. In some embodiments, cells are cultured in the media to produce determined dopamine (DA) neuron progenitor cells, or dopamine (DA) neurons.

In some embodiments, the media is also supplemented with a serum replacement containing minimal non-human-derived components (e.g., KnockOut™ serum replacement). In some embodiments, the media is supplemented with the serum replacement from about day 7 through about day 10. In some embodiments, the media is supplemented with about 2% (v/v) of the serum replacement. In some embodiments, the media is supplemented with about 2% (v/v) of the serum replacement from about day 7 through about day 10.

In some embodiments, the media is further supplemented with small molecules. In some embodiments, the small molecules are selected from among the group consisting of: a Rho-associated protein kinase (ROCK) inhibitor, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 7, day 16, day 20, or a combination thereof. In some embodiments the media is supplemented with a ROCK inhibitor on day 7. In some embodiments the media is supplemented with a ROCK inhibitor on day 16.

In some embodiments the media is supplemented with a ROCK inhibitor on day 20. In some embodiments the media is supplemented with a ROCK inhibitor on day 7 and day 16. In some embodiments the media is supplemented with a ROCK inhibitor on day 16 and day 20. In some embodiments the media is supplemented with a ROCK inhibitor on day 7, day 16, and day 20.

In some embodiments, cells are exposed to the ROCK inhibitor at a concentration of between about 1 μM and about 20 μM, between about 5 μM and about 15 PM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 PM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is Fasudil, Ripasudil, Netarsudil, RKI-1447, Y-27632, GSK429286A, Y-30141, or a combination thereof. In some embodiments, the ROCK inhibitor is a small molecule. In some embodiments, the ROCK inhibitor selectively inhibits p160ROCK. In some embodiments, the ROCK inhibitor is Y-27632, having the formula:

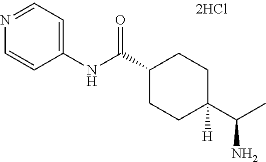 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 7, day 16, day 20, or a combination thereof. In some embodiments, cells are exposed to Y-27632 at a concentration of about 10 μM on day 7. In some embodiments, cells are exposed to Y-27632 at a concentration of about 10 μM on day 16. In some embodiments, cells are exposed to Y-27632 at a concentration of about 10 μM on day 20. In some embodiments, cells are exposed to Y-27632 at a concentration of about 10 μM on day 7 and day 16. In some embodiments, cells are exposed to Y-27632 at a concentration of about 10 μM on day 16 and day 20. In some embodiments, cells are exposed to Y-27632 at a concentration of about 10 μM on day 7, day 16, and day 20.

In some embodiments the media is supplemented with an inhibitor of BMP signaling. In some embodiments the media is supplemented with an inhibitor of BMP signaling from about day 7 up to about day 11 (e.g., day 10 or day 11). In some embodiments the media is supplemented with an inhibitor of BMP signaling from about day 7 through day 10, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is LDN193189 or K02288. In some embodiments, the inhibitor of BMP signaling is capable of inhibiting “Small Mothers Against Decapentaplegic” SMAD signaling. In In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

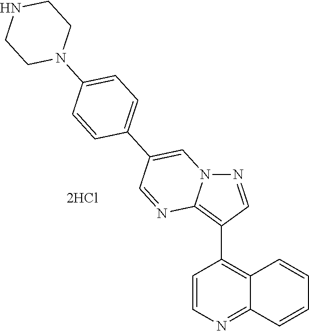 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM from about day 7 up to about day 11 (e.g., day 10 or day 11). In some embodiments, cells are exposed to LDN193189 at a concentration of about 0.1 μM from about day 7 through about day 10, inclusive of each day.

In some embodiments the media is supplemented with an inhibitor of GSK3β signaling. In some embodiments the media is supplemented with an inhibitor of GSK3β signaling from about day 7 up to about day 13 (e.g., day 12 or day 13). In some embodiments the media is supplemented with an inhibitor of GSK3β signaling from about day 7 through day 12, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of GSK3β signaling at a concentration of between about 1 μM and about 4 μM. In some embodiments, cells are exposed to the inhibitor of GSK3β signaling at a concentration of between about 2 μM and about 3 PM. In some embodiments, cells are exposed to the inhibitor of GSK3β signaling at a concentration of about 2 μM.

In some embodiments, the inhibitor of GSK3β signaling is selected from lithium ion, valproic acid, iodotubercidin, naproxen, famotidine, curcumin, olanzapine, CHIR99012, or a combination thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3β. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

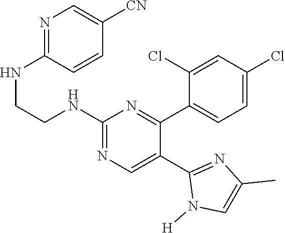 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM from about day 7 up to about day 13 (e.g., day 12 or day 13). In some embodiments, cells are exposed to CHIR99021 at a concentration of about 2.0 μM from about day 7 through about day 12, inclusive of each day.

In some embodiments the media is supplemented with brain-derived neurotrophic factor (BDNF). In some embodiments the media is supplemented with BDNF beginning on about day 11. In some embodiments the media is supplemented with BDNF from about day 11 until harvest or collection. In some embodiments the media is supplemented with BDNF from about day 11 through day 18. In some embodiments the media is supplemented with BDNF from about day 11 through day 20. In some embodiments the media is supplemented with BDNF from about day 11 through day 25.

In some embodiments, cells are exposed to BDNF at a concentration of between about 1 ng/mL and 100 ng/mL, between about 5 ng/mL and about 50 ng/mL, between about 10 ng/mL and about 30 ng/mL. In some embodiments, cells are exposed to BDNF at a concentration of between about 10 ng/mL and about 30 ng/mL. In some embodiments, cells are exposed to BDNF at a concentration of about 20 ng/mL.

In some embodiments, the media is supplemented with about 20 ng/mL BDNF beginning on about day 11. In some embodiments the media is supplemented with 20 ng/mL BDNF from about day 11 until harvest or collection. In some embodiments the media is supplemented with about 20 ng/mL BDNF from about day 11 through day 18. In some embodiments the media is supplemented with about 20 ng/mL BDNF from about day 11 through day 20. In some embodiments the media is supplemented with about 20 ng/mL BDNF from about day 11 through day 25.

In some embodiments the media is supplemented with glial cell-derived neurotrophic factor (GDNF). In some embodiments the media is supplemented with GDNF beginning on about day 11. In some embodiments the media is supplemented with GDNF from about day 11 until harvest or collection. In some embodiments the media is supplemented with GDNF from about day 11 through day 18. In some embodiments the media is supplemented with GDNF from about day 11 through day 20. In some embodiments the media is supplemented with GDNF from about day 11 through day 25.

In some embodiments, cells are exposed to GDNF at a concentration of between about 1 ng/mL and 100 ng/mL, between about 5 ng/mL and about 50 ng/mL, between about 10 ng/mL and about 30 ng/mL. In some embodiments, cells are exposed to GDNF at a concentration of between about 10 ng/mL and about 30 ng/mL. In some embodiments, cells are exposed to GDNF at a concentration of about 20 ng/mL.

In some embodiments, the media is supplemented with about 20 ng/mL GDNF beginning on about day 11. In some embodiments the media is supplemented with 20 ng/mL GDNF from about day 11 until harvest or collection. In some embodiments the media is supplemented with about 20 ng/mL GDNF from about day 11 through day 18. In some embodiments the media is supplemented with about 20 ng/mL GDNF from about day 11 through day 20. In some embodiments the media is supplemented with about 20 ng/mL GDNF from about day 11 through day 25.

In some embodiments the media is supplemented with ascorbic acid. In some embodiments the media is supplemented with ascorbic acid beginning on about day 11. In some embodiments the media is supplemented with ascorbic acid from about day 11 until harvest or collection. In some embodiments the media is supplemented with ascorbic acid from about day 11 through day 18. In some embodiments the media is supplemented with ascorbic acid from about day 11 through day 20. In some embodiments the media is supplemented with ascorbic acid from about day 11 through day 25.

In some embodiments, cells are exposed to ascorbic acid at a concentration of between about 0.05 mM and 5 mM, between about 0.1 mM and about 1 mM, between about 0.2 mM and about 0.5 mM, each inclusive. In some embodiments, cells are exposed to ascorbic acid at a concentration of between about 0.05 mM and about 5 mM, each inclusive. In some embodiments, cells are exposed to ascorbic acid at a concentration of between about 0.1 mM and about 1 mM, each inclusive. In some embodiments, cells are exposed to ascorbic acid at a concentration of about 0.2 mM.

In some embodiments, the media is supplemented with about 0.2 mM ascorbic acid beginning on about day 11. In some embodiments the media is supplemented with 0.2 mM ascorbic acid from about day 11 until harvest or collection. In some embodiments the media is supplemented with about 0.2 mM ascorbic acid from about day 11 through day 18. In some embodiments the media is supplemented with about 0.2 mM ascorbic acid from about day 11 through day 20. In some embodiments the media is supplemented with about 0.2 mM ascorbic acid from about day 11 through day 25.

In some embodiments the media is supplemented with dibutyryl cyclic AMP (dbcAMP). In some embodiments the media is supplemented with dbcAMP beginning on about day 11. In some embodiments the media is supplemented with dbcAMP from about day 11 until harvest or collection. In some embodiments the media is supplemented with dbcAMP from about day 11 through day 18. In some embodiments the media is supplemented with dbcAMP from about day 11 through day 20. In some embodiments the media is supplemented with dbcAMP from about day 11 through day 25.

In some embodiments, cells are exposed to dbcAMP at a concentration of between about 0.05 mM and 5 mM, between about 0.1 mM and about 3 mM, between about 0.2 mM and about 1 mM, each inclusive. In some embodiments, cells are exposed to dbcAMP at a concentration of between about 0.1 mM and about 3 mM, each inclusive. In some embodiments, cells are exposed to dbcAMP at a concentration of between about 0.2 mM and about 1 mM, each inclusive. In some embodiments, cells are exposed to dbcAMP at a concentration of about 0.5 mM.

In some embodiments, the media is supplemented with about 0.5 mM dbcAMP beginning on about day 11. In some embodiments the media is supplemented with 0.5 mM dbcAMP from about day 11 until harvest or collection. In some embodiments the media is supplemented with about 0.5 mM dbcAMP from about day 11 through day 18. In some embodiments the media is supplemented with about 0.5 mM dbcAMP from about day 11 through day 20. In some embodiments the media is supplemented with about 0.5 mM dbcAMP from about day 11 through day 25.

In some embodiments the media is supplemented with transforming growth factor beta 3 (TGF33). In some embodiments the media is supplemented with TGFβ3 beginning on about day 11. In some embodiments the media is supplemented with TGFβ3 from about day 11 until harvest or collection. In some embodiments the media is supplemented with TGFβ3 from about day 11 through day 18. In some embodiments the media is supplemented with TGFβ3 from about day 11 through day 20. In some embodiments the media is supplemented with TGFβ3 from about day 11 through day 25.

In some embodiments, cells are exposed to TGFβ3 at a concentration of between about 0.1 ng/mL and 10 ng/mL, between about 0.5 ng/mL and about 5 ng/mL, or between about 1.0 ng/mL and about 2.0 ng/mL. In some embodiments, cells are exposed to TGFβ3 at a concentration of between about 1.0 ng/mL and about 2.0 ng/mL, each inclusive. In some embodiments, cells are exposed to TGFβ3 at a concentration of about 1 ng/mL.

In some embodiments, the media is supplemented with about 1 ng/mL TGFβ3 beginning on about day 11. In some embodiments the media is supplemented with 1 ng/mL TGFβ3 from about day 11 until harvest or collection. In some embodiments the media is supplemented with about 1 ng/mL TGFβ3 from about day 11 through day 18. In some embodiments the media is supplemented with about 1 ng/mL TGFβ3 from about day 11 through day 20. In some embodiments the media is supplemented with about 1 ng/mL TGFβ3 from about day 11 through day 25.

In some embodiments the media is supplemented with an inhibitor of Notch signaling. In some embodiments the media is supplemented with an inhibitor of Notch signaling beginning on about day 11. In some embodiments the media is supplemented with an inhibitor of Notch signaling from about day 11 until harvest or collection. In some embodiments the media is supplemented with an inhibitor of Notch signaling from about day 11 through day 18. In some embodiments the media is supplemented with an inhibitor of Notch signaling from about day 11 through day 20. In some embodiments the media is supplemented with an inhibitor of Notch signaling from about day 11 through day 25.

In some embodiments, an inhibitor of Notch signaling is selected from cowanin, PF-03084014, L685458, LY3039478, DAPT, or a combination thereof. In some embodiments, the inhibitor of Notch signaling inhibits gamma secretase. In some embodiments, the inhibitor of Notch signaling is a small molecule. In some embodiments, the inhibitor of Notch signaling is DAPT, having the following formula:

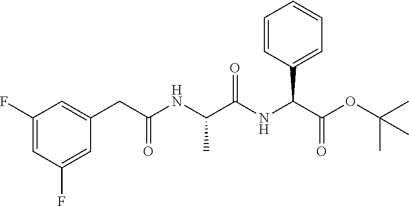 [SMILES: C[C@H](NC(=O)Cc1cc(F)cc(F)c1)C(=O)N[C@H](C(=O)OC(C)(C)C)c1ccccc1]

In some embodiments, cells are exposed to DAPT at a concentration of between about 1 μM and about 20 μM, between about 5 μM and about 15 μM, or between about 8 μM and about 12 μM. In some embodiments, cells are exposed to DAPT at a concentration of between about 1 μM and about 20 μM. In some embodiments, cells are exposed to DAPT at a concentration of between about 5 μM and about 15 μM. In some embodiments, cells are exposed to DAPT at a concentration of between about 8 μM and about 12 μM. In some embodiments, cells are exposed to DAPT at a concentration of about 10 PM.

In some embodiments, the media is supplemented with about 10 μM DAPT beginning on about day 11. In some embodiments the media is supplemented with 10 μM DAPT from about day 11 until harvest or collection. In some embodiments the media is supplemented with about 10 μM DAPT from about day 11 through day 18. In some embodiments the media is supplemented with about 10 μM DAPT from about day 11 through day 20. In some embodiments the media is supplemented with about 10 μM DAPT from about day 11 through day 25.

In some embodiments, beginning on about day 11, the media is supplemented with about 20 ng/mL BDNF, about 20 ng/mL GDNF, about 0.2 mM ascorbic acid, about 0.5 mM dbcAMP, about 1 ng/mL TGF33, and about 10 μM DAPT. In some embodiments, from about day 11 until harvest or collection, the media is supplemented with about 20 ng/mL BDNF, about 20 ng/mL GDNF, about 0.2 mM ascorbic acid, about 0.5 mM dbcAMP, about 1 ng/mL TGF33, and about 10 μM DAPT. In some embodiments, from about day 11 until day 18, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until day 20, the media is supplemented with about 20 ng/mL BDNF, about 20 ng/mL GDNF, about 0.2 mM ascorbic acid, about 0.5 mM dbcAMP, about 1 ng/mL TGF33, and about 10 μM DAPT. In some embodiments, from about day 11 until day 25, the media is supplemented with about 20 ng/mL BDNF, about 20 ng/mL GDNF, about 0.2 mM ascorbic acid, about 0.5 mM dbcAMP, about 1 ng/mL TGF33, and about 10 μM DAPT.

In some embodiments, a serum replacement is provided in the media from about day 7 through about day 10. In some embodiments, the serum replacement is provided at 2% (v/v) in the media on day 7 through day 10.

In some embodiments, from day about 7 to about day 16, at least about 50% of the media is replaced daily. In some embodiments, from about day 7 to about day 16, about 50% of the media is replaced daily, every other day, or every third day. In some embodiments, from about day 7 to about day 16, about 50% of the media is replaced daily. In some embodiments, beginning on about day 17, at least about 50% of the media is replaced daily, every other day, or every third day. In some embodiments, beginning on about day 17, at least about 50% of the media is replaced every other day. In some embodiments, beginning on about day 17, about 50% of the media is replaced daily, every other day, or every third day. In some embodiments, beginning on about day 17, about 50% of the media is replaced every other day. In some embodiments, the replacement media contains small molecules about twice as concentrated as compared to the concentration of the small molecules in the media on day 0.

In some embodiments, the second incubation involves culturing cells derived from the cell aggregate (e.g. spheroid) in a “basal induction media.” In some embodiments, the second incubation involves culturing cells derived from the cell aggregate (e.g. spheroid) in a “maturation media.” In some embodiments, the second incubation involves culturing cells derived from the cell aggregate (e.g. spheroid) in the basal induction media, and then in the maturation media.

In some embodiments, the second incubation involves culturing the cells in the basal induction media from about day 7 through about day 10. In some embodiments, the second incubation involves comprises culturing the cells in the maturation media beginning on about day 11. In some embodiments, the second incubation involves culturing the cells in the basal induction media from about day 7 through about day 10, and then in the maturation media beginning on about day 11. In some embodiments, cells are cultured in the maturation media to produce determined dopamine (DA) neuron progenitor cells, or dopamine (DA) neurons.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, 0-mercaptoethanol, and insulin. In some embodiments, the basal induction media is further supplemented with any of the molecules described in Section II.

In some embodiments, the maturation media is formulated to contain Neurobasal™ media, supplemented with N-2 and B27 supplements, non-essential amino acids (NEAA), and GlutaMAX™. In some embodiments, the maturation media is further supplemented with any of the molecules described in Section II.

In some embodiments, the cells are cultured in the basal induction media from about day 7 up to about day 11 (e.g., day 10 or day 11). In some embodiments, the cells are cultured in the basal induction media from about day 7 through day 10, each day inclusive. In some embodiments, the cells are cultured in the maturation media beginning on about day 11. In some embodiments, the cells are cultured in the basal induction media from about day 7 through about day 10, and then the cells are cultured in the maturation media beginning on about day 11. In some embodiments, the cells are cultured in the maturation media from about day 11 until harvest or collection of the cells. In some embodiments, cells are harvested between day 16 and 27. In some embodiments, cells are harvested between day 18 and day 25. In some embodiments, cells are harvested on day 18. In some embodiments, cells are harvested on day 20. In some embodiments, cells are harvested on day 25.

### C. Adherent Culture

### 1. First Incubation

The provided methods include culturing PSCs (e.g. iPSCs) by incubation with certain molecules (e.g. small molecules) to induce their differentiation into floor plate midbrain progenitor cells, determined dopamine (DA) neuron progenitor cells, and/or, dopamine (DA) neurons. In particular, the provided embodiments include a first incubation of PSCs under adherent conditions in the presence of certain molecules (e.g., small molecules). In some embodiments, the methods include performing a first incubation involving culturing pluripotent stem cells in an adherent culture vessel, wherein: beginning at the initiation of the first incubation (day 0) the cells are exposed to (i) an inhibitor of TGF-3/activin-Nodal signaling; and (ii) an inhibitor of bone morphogenetic protein (BMP) signaling; beginning on day 1, the cells are exposed to at least one activator of Sonic Hedgehog (SHH) signaling; and beginning on day 2, the cells are exposed to (iv) an inhibitor of glycogen synthase kinase 3β (GSK3β) signaling.

In some embodiments, an adherent culture vessel is a culture vessel to which a cell may attach via extracellular matrix molecules and the like, and requires the use of an enzyme (e.g., trypsin, dispase, accutase etc.) for detaching cells from the culture vessel.

In some embodiments, the adherent culture vessel is a plate, a dish, a flask, or a bioreactor. In some embodiments, the adherent culture vessel is a plate, such as a multi-well plate. In some embodiments, the adherent culture vessel is a 6-well or 24-well plate. In some embodiments, the wells of the multi-well plate further include micro-wells. In some any of the provided embodiments, an adherent culture vessel, such as a multi-well plate, has round or concave wells and/or microwells. In any of the provided embodiments, an adherent culture vessel, such as a multi-well plate, does not have corners or seams.

In some embodiments, the number of PSCs plated on day 0 of the method is between about between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.2×10⁶ cells/cm² and about 0.4×10⁶ cells/cm².

In some embodiments, the number of PSCs plated on day −1 of the method is between about 1×10⁵ pluripotent stem cells per well and about 20×10⁶ pluripotent stem cells per well, between about 1×10⁵ pluripotent stem cells per well and about 15×10⁶ pluripotent stem cells per well, between about 1×10⁵ pluripotent stem cells per well and about 10×10⁶ pluripotent stem cells per well, between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 20×10⁶ pluripotent stem cells per well, between about 5×10⁵ pluripotent stem cells per well and about 15×10⁶ pluripotent stem cells per well, between about 5×10⁵ pluripotent stem cells per well and about 10×10⁶ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 6-well plate on day −1 of the method is between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 24-well plate on day −1 of the method is between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, or between about 1×10⁶ pluripotent stem cells per well and about 5×10⁶ pluripotent stem cells per well.

In some embodiments, the first incubation is from about day 0 through about day 10. In some embodiments, the first incubation comprises culturing pluripotent stem cells in a culture media (“media”). In some embodiments, the first incubation comprises culturing pluripotent stem cells in the media from about day 0 through about day10. In some embodiments, the first incubation comprises culturing pluripotent stem cells in the media to induce differentiation of the PSCs into floor plate midbrain progenitor cells.

In some embodiments, the media is also supplemented with a serum replacement containing minimal non-human-derived components (e.g., KnockOut™ serum replacement). In some embodiments, the serum replacement is provided in the media at 5% (v/v) for at least a portion of the first incubation. In some embodiments, the serum replacement is provided in the media at 5% (v/v) on day 0 and day 1. In some embodiments, the serum replacement is provided in the media at 2% (v/v) for at least a portion of the first incubation. In some embodiments, the serum replacement is provided in the media at 2% (v/v) from day 2 through day 10. In some embodiments, the serum replacement is provided in the media at 5% (v/v) on day 0 and day 1, and at 2% (v/v) from day 2 through day 10.

In some embodiments, the media is further supplemented with small molecules, such as any described above. In some embodiments, the small molecules are selected from among the group consisting of: a Rho-associated protein kinase (ROCK) inhibitor, an inhibitor of TGF-β/activin-Nodal signaling, at least one activator of Sonic Hedgehog (SHH) signaling, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day −1.

In some embodiments, cells are exposed to the ROCK inhibitor at a concentration of between about 1 μM and about 20 μM, between about 5 μM and about 15 PM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 PM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is selected from among the group consisting of: Fasudil, Ripasudil, Netarsudil, RKI-1447, Y-27632, GSK429286A, Y-30141, and combinations thereof. In some embodiments, the ROCK inhibitor is a small molecule. In some embodiments, the ROCK inhibitor selectively inhibits p160ROCK. In some embodiments, the ROCK inhibitor is Y-27632, having the formula:

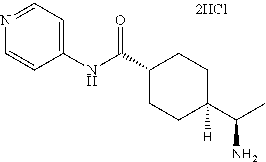 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day −1.

In some embodiments the media is supplemented with an inhibitor of TGF-3/activin-Nodal signaling. In some embodiments the media is supplemented with an inhibitor of TGF-β/activin-Nodal signaling up to about day 5. In some embodiments the media is supplemented with an inhibitor of TGF-β/activin-Nodal signaling from about day 0 through day 4, each day inclusive.

In some embodiments, cells are exposed to the inhibitor of TGF-3/activin-Nodal signaling at a concentration of between about 1 μM and about 20 μM, between about 5 μM and about 15 μM, or between about 8 μM and about 12 μM. In some embodiments, cells are exposed to the inhibitor of TGF-β/activin-Nodal signaling at a concentration of between about 1 μM and about 20 μM. In some embodiments, cells are exposed to the inhibitor of TGF-3/activin-Nodal signaling at a concentration of between about 5 μM and about 15 PM. In some embodiments, cells are exposed to the inhibitor of TGF-β/activin-Nodal signaling at a concentration of between about 8 μM and about 12 μM. In some embodiments, cells are exposed to the inhibitor of TGF-β/activin-Nodal signaling at a concentration of about 10 μM.

In some embodiments, the inhibitor of TGF-β/activin-Nodal signaling is a small molecule. In some embodiments, the inhibitor of TGF-β/activin-Nodal signaling is capable of lowering or blocking transforming growth factor beta (TGFP)/Activin-Nodal signaling. In some embodiments, the inhibitor of TGF-β/activin-Nodal signaling inhibits ALK4, ALK5, ALK7, or combinations thereof. In some embodiments, the inhibitor of TGF-β/activin-Nodal signaling inhibits ALK4, ALK5, and ALK7. In some embodiments, the inhibitor of TGF-β/activin-Nodal signaling does not inhibit ALK2, ALK3, ALK6, or combinations thereof. In some embodiments, the inhibitor does not inhibit ALK2, ALK3, or ALK6. In some embodiments, the inhibitor of TGF-β/activin-Nodal signaling is SB431542 (e.g., CAS 301836-41-9, molecular formula of C22H18N4O3, and name of 4-[4-(1,3-benzodioxol-5-yl)-5-(2-pyridinyl)-1H-imidazol-2-yl]-benzamide), having the formula:

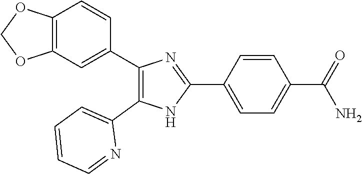 [SMILES: NC(=O)c1ccc(-c2nc(-c3ccc4c(c3)OCO4)c(-c3ccccn3)[nH]2)cc1]

In some embodiments, cells are exposed to SB431542 at a concentration of about 10 μM. In some embodiments, cells are exposed to SB431542 at a concentration of about 10 μM until about day 4. In some embodiments, cells are exposed to SB431542 at a concentration of about 10 μM from about day 0 through about day 4, inclusive of each day.

In some embodiments the media is supplemented with at least one activator of sonic hedghehog (SHH) signaling. SHH refers to a protein that is one of at least three proteins in the mammalian signaling pathway family called hedgehog, another is desert hedgehog (DHH) while a third is Indian hedgehog (IHH). Shh interacts with at least two transmembrane proteins by interacting with transmembrane molecules Patched (PTC) and Smoothened (SMO). In some embodiments the media is supplemented with the at least one activator of SHH signaling beginning on day 1 and up to about day 7 (e.g., day 6 or day 7). In some embodiments the media is supplemented with the at least one activator of SHH signaling from about day 1 through day 6, each day inclusive.

In some embodiments, the at least one activator of SHH signaling is SHH protein. In some embodiments, the at least one activator of SHH signaling is recombinant SHH protein. In some embodiments, the at least one activator of SHH signaling is recombinant mouse SHH protein. In some embodiments, the at least one activator of SHH signaling is recombinant human SHH protein. In some embodiments, the least one activator of SHH signaling is a recombinant N-Terminal fragment of a full-length murine sonic hedgehog protein capable of binding to the SHH receptor for activating SHH. In some embodiments, the at least one activator of SHH signaling is C25II SHH protein.

In some embodiments, cells are exposed to the at least one activator of SHH signaling at a concentration of between about 10 ng/mL and about 500 ng/mL, between about 20 ng/mL and 400 μg/mL, between about 30 ng/mL and about 300 ng/mL, between about 40 ng/mL and about 200 ng/mL, or between about 50 ng/mL and about 100 ng/mL, each inclusive. In some embodiments, cells are exposed to the at least one activator of SHH signaling at a concentration of between about 50 ng/mL and about 100 ng/mL, each inclusive. In some embodiments, cells are exposed to the at least one activator of SHH signaling at a concentration of about 100 ng/mL. In some embodiments, the cells are exposed to SHH protein at about 100 ng/mL. In some embodiments, the cells are exposed to recombinant SHH protein at about 100 ng/mL. In some embodiments, the cells are exposed to recombinant mouse SHH protein at about 100 ng/mL. In some embodiments, the cells are exposed to C25II SHH protein at about 100 ng/mL.

In some embodiments, cells are exposed to recombinant SHH protein at a concentration of about 10 ng/mL. In some embodiments, cells are exposed to recombinant SHH protein at a concentration of about 10 ng/mL beginning at about day 1 and up to about day 7 (e.g., day 6 or day 7). In some embodiments, cells are exposed to recombinant SHH protein at a concentration of about 10 ng/mL from about day 1 through about day 6, inclusive of each day.

In some embodiments, cells are exposed to the at least one activator of SHH signaling at a concentration of between about 1 μM and about 20 μM, between about 5 μM and about 15 μM, or between about 8 μM and about 12 μM. In some embodiments, cells are exposed to the at least one activator of SHH signaling at a concentration of between about 1 μM and about 20 μM. In some embodiments, cells are exposed to the at least one activator of SHH signaling at a concentration of between about 5 μM and about 15 μM. In some embodiments, cells are exposed to the at least one activator of SHH signaling at a concentration of between about 8 μM and about 12 μM. In some embodiments, cells are exposed to the at least one activator of SHH signaling at a concentration of about 10 μM.

In some embodiments, the at least one activator of SHH signaling is an activator of the Hedgehog receptor Smoothened. It some embodiments, the at least one activator of SHH signaling is a small molecule. In some embodiments, the least one activator of SHH signaling is purmorphamine (e.g. CAS 483367-10⁻⁸), having the formula below:

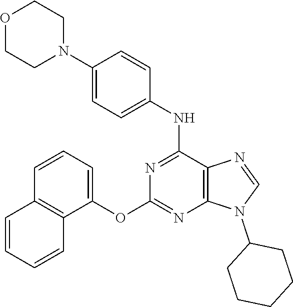 [SMILES: c1ccc2c(Oc3nc(Nc4ccc(N5CCOCC5)cc4)c4ncn(C5CCCCC5)c4n3)cccc2c1]

In some embodiments, cells are exposed to purmorphamine at a concentration of about 10 μM. In some embodiments, cells are exposed to purmorphamine at a concentration of about 10 μM beginning at about day 1 and up to day 7 (e.g., day 6 or day 7). In some embodiments, cells are exposed to purmorphamine at a concentration of about 10 μM from about day 1 through about day 6, inclusive of each day.

In some embodiments, the at least one activator of SHH signaling is SHH protein and purmorphamine. In some embodiments, cells are exposed to SHH protein and purmorphamine at a concentration beginning at about day 1 and up to about day 7 (e.g., day 6 or day 7). In some embodiments, cells are exposed to SHH protein and purpomorphamine from about day 1 through about day 6, inclusive of each day. In some embodiments, cells are exposed to 100 ng/mL SHH protein and 10 μM purmorphamine beginning at about day 1 and up to about day 7 (e.g., day 6 or day 7). In some embodiments, cells are exposed to 100 ng/mL SHH protein and 10 μM purpomorphamine from about day 1 through about day 6, inclusive of each day.

In some embodiments the media is supplemented with an inhibitor of BMP signaling. In some embodiments the media is supplemented with an inhibitor of BMP signaling up to about day 11 (e.g., day 10 or 11). In some embodiments the media is supplemented with an inhibitor of BMP signaling from about day 0 through day 10, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is selected from LDN193189 or K02288. In some embodiments, the inhibitor of BMP signaling is capable of inhibiting “Small Mothers Against Decapentaplegic” SMAD signaling. In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

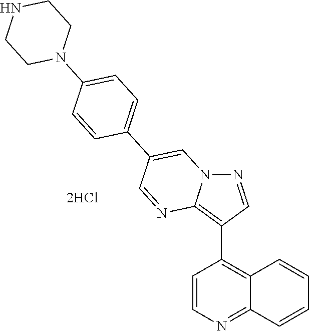 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 pM up to about day 11 (e.g., day 10 or day 22). In some embodiments, cells are exposed to LDN193189 at a concentration of about 0.1 μM from about day 0 through about day 10, inclusive of each day.

In some embodiments the media is supplemented with an inhibitor of GSK3β signaling. In some embodiments the media is supplemented with an inhibitor of GSK3β signaling beginning on about day 2 up to about day 11 (e.g., day 10 or day 11). In some embodiments the media is supplemented with an inhibitor of GSK3β signaling from about day 2 through day 10, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of BMP signaling at a concentration of between about 1 μM and about 4 μM. In some embodiments, cells are exposed to the inhibitor of BMP signaling at a concentration of between about 2 μM and about 3 PM. In some embodiments, cells are exposed to the inhibitor of GSK3β signaling at a concentration of about 2 μM.

In some embodiments, the inhibitor of GSK3β signaling is selected from among the group consisting of: lithium ion, valproic acid, iodotubercidin, naproxen, famotidine, curcumin, olanzapine, CHIR99012, and combinations thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3β. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

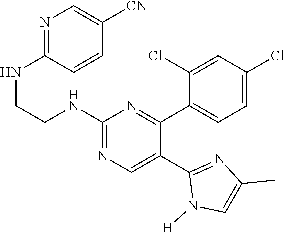 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM beginning at about day 2 and up to about day 11 (e.g., day 10 or 11). In some embodiments, cells are exposed to CHIR99021 at a concentration of about 2.0 μM from about day 2 through about day 10, inclusive of each day.

In some embodiments, from about day 0 to about day 17, at least about 50% of the media is replaced daily. In some embodiments, from about day 0 to about day 17, at leaset about 75% of the media is replaced daily. In some embodiments, from about day 0 to about day 17, about 100% of the media is replaced daily. In some embodiments, at least about 50% of the media is replaced on day 19. In some embodiments, at least about 75% of the media is replaced on day 19. In some embodiments, about 100% of the media is replaced on day 19. In some embodiments, about 100% of the media is replaced daily from days 0 to 17 and on day 19.

In some embodiments, the first incubation comprises culturing pluripotent stem cells in a “basal induction media.” In some embodiments, the first incubation comprises culturing pluripotent stem cells in the basal induction media from about day 0 through about day 10. In some embodiments, the first incubation comprises culturing pluripotent stem cells in the basal induction media to induce differentiation of the PSCs into floor plate midbrain progenitor cells.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, 0-mercaptoethanol, and insulin. In some embodiments, the basal induction media is further supplemented with any of the small molecules as described above

### 2. Second Incubation

In some embodiments, the methods include performing a second incubation of the cells produced by the first incubation. In some embodiments, culturing the cells produced by the first incubation under adherent conditions induces their differentiation into floor plate midbrain progenitor cells, determined dopamine (DA) neuron progenitor cells, and/or, dopamine (DA) neurons.

In some embodiments, the second incubation involves culturing cells in a culture vessel coated with a substrate including laminin or a fragment thereof, collagen, entactin, heparin sulfate proteoglycans, or a combination thereof, wherein, beginning on day 11, the cells are exposed to (i) brain-derived neurotrophic factor (BDNF); (ii) glial cell-derived neurotrophic factor (GDNF); (iii) transforming growth factor beta-3 (TGF33); (iv) an inhibitor of Notch signaling; (v) ascorbic acid; and (vi) dibutyryl cyclic AMP (dbcAMP). In some embodiments, the method further includes harvesting the differentiated cells.

In some embodiments, the substrate-coated culture vessel is a culture vessel with a surface to which cells can attach. In some embodiments, the substrate-coated culture vessel is a culture vessel with a surface to which a substantial number of cells attach. In some embodiments, the substrate is a basement membrane protein. In some embodiments, the substrate is laminin or a fragment thereof, collagen, entactin, heparin sulfate proteoglycans, or a combination thereof. In some embodiments, the substrate is laminin or a fragment thereof. In some embodiments, the substrate is collagen. In some embodiments, the substrate is entactin. In some embodiments, the substrate is heparin sulfate proteoglycans. In some embodiments, the substrate is a recombinant protein. In some embodiments, the substrate is recombinant laminin or a fragment thereof. In some embodiments, the laminin is selected from laminin 521, laminin 111, laminin 511, and laminin 511-E8. In some embodiments, the laminin is laminin 511-E8.

In some embodiments, the substrate-coated culture vessel is exposed to poly-L-ornithine. In some embodiments, the substrate-coated culture vessel is exposed to poly-L-ornithine prior to being used for cell culture.

In some embodiments, thesubstrate-coated culture vessel is a plate, a dish, a flask, or a bioreactor. In some embodiments, the substrate-coated culture vessel is a plate, such as a multi-well plate. In some embodiments, the substrate-coated culture vessel is a plate. In some embodiments, the substrate-coated culture vessel is a 6-well or 24-well plate. In some embodiments, the substrate-coated culture vessel is a dish. In some embodiments, the substrate-coated culture vessel is a flask. In some embodiments, the substrate-coated culture vessel is a bioreactor.

In some embodiments, the substrate-coated culture vessel allows for a monolayer cell culture. In some embodiments, cells produced by the first incubation are cultured in a monolayer culture on the substrate-coated plates. In some embodiments, cells produced by the first incubation are cultured to produce a monolayer culture of cells positive for one or more of LMX1A, FOXA2, EN1, CORIN, and combinations thereof. In some embodiments, cells derived from the cells produced by the first incubation are cultured to produce a monolayer culture of cells, wherein at least some of the cells are positive for EN1 and CORIN. In some embodiments, cells produced by the first incubation are cultured to produce a monolayer culture of cells, wherein at least some of the cells are TH+. In some embodiments, at least some cells are TH+ by or on about day 25. In some embodiments, cells produced by the first incubation are cultured to produce a monolayer culture of cells, wherein at least some of the cells are TH+FOXA2+. In some embodiments, at least some cells are TH+FOXA2+ by or on about day 25.

In the methods provided herein, the second incubation involves culturing cells produced by the first incubation under conditions to induce neural differentiation of the cells. In some embodiments, the second incubation begins on about day 11.

In some embodiments, the second incubation is from about day 11 until harvesting of the cells. In some embodiments, the cells are harvested on about day 16 or later. In some embodiments, the cells are harvested between about day 16 and about day 30. In some embodiments, the cells are harvested between about day 18 and about day 25. In some embodiments, the cells are harvested on about day 18. In some embodiments, the cells are harvested on about day 19. In some embodiments, the cells are harvested on about day 20. In some embodiments, the cells are harvested on about day 21. In some embodiments, the cells are harvested on about day 22. In some embodiments, the cells are harvested on about day 23. In some embodiments, the cells are harvested on about day 24. In some embodiments, the cells are harvested on about day 25. In some embodiments, the second incubation is from about day 11 until about day 18. In some embodiments, the second incubation is from about day 11 until about day 19. In some embodiments, the second incubation is from about day 11 until about day 20. In some embodiments, the second incubation is from about day 11 until about day 21. In some embodiments, the second incubation is from about day 11 until about day 22. In some embodiments, the second incubation is from about day 11 until about day 23. In some embodiments, the second incubation is from about day 11 until about day 24. In some embodiments, the second incubation is from about day 7 until about day 25.

In some embodiments, the second incubation involves culturing cells produced from the first incubation in a culture media (“media”).

In some embodiments, the second incubation involves culturing the cells in the media from about day 11 until harvest or collection. In some embodiments, cells are cultured in the media to produce determined dopamine (DA) neuron progenitor cells, or dopamine (DA) neurons.

In some embodiments, the media is further supplemented with small molecules. In some embodiments, the small molecules comprise a Rho-associated protein kinase (ROCK) inhibitor. In some embodiments, the small molecules comprise an inhibitor of GSK3β signaling.

In some embodiments, the small molecules comprise a Rho-associated protein kinase (ROCK) inhibitor and an inhibitor of GSK3β signaling.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 7, day 16, day 20, or a combination thereof. In some embodiments the media is supplemented with a ROCK inhibitor on day 7. In some embodiments the media is supplemented with a ROCK inhibitor on day 16.

In some embodiments the media is supplemented with a ROCK inhibitor on day 20. In some embodiments the media is supplemented with a ROCK inhibitor on day 7 and day 16. In some embodiments the media is supplemented with a ROCK inhibitor on day 16 and day 20. In some embodiments the media is supplemented with a ROCK inhibitor on day 7, day 16, and day 20.

In some embodiments, cells are exposed to the ROCK inhibitor at a concentration of between about 1 μM and about 20 μM, between about 5 μM and about 15 PM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 PM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is Fasudil, Ripasudil, Netarsudil, RKI-1447, Y-27632, GSK429286A, Y-30141, or a combination thereof. In some embodiments, the ROCK inhibitor is a small molecule. In some embodiments, the ROCK inhibitor selectively inhibits p160ROCK. In some embodiments, the ROCK inhibitor is Y-27632, having the formula:

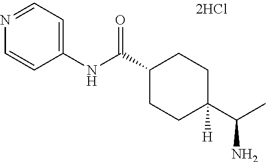 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 7, day 16, day 20, or a combination thereof. In some embodiments, cells are exposed to Y-27632 at a concentration of about 10 μM on day 7. In some embodiments, cells are exposed to Y-27632 at a concentration of about 10 μM on day 16. In some embodiments, cells are exposed to Y-27632 at a concentration of about 10 μM on day 20. In some embodiments, cells are exposed to Y-27632 at a concentration of about 10 μM on day 7 and day 16. In some embodiments, cells are exposed to Y-27632 at a concentration of about 10 μM on day 16 and day 20. In some embodiments, cells are exposed to Y-27632 at a concentration of about 10 μM on day 7, day 16, and day 20.

In some embodiments the media is supplemented with an inhibitor of GSK3β signaling. In some embodiments the media is supplemented with an inhibitor of GSK3β signaling from about day 11 up to about day 13 (e.g., day 12 or day 13). In some embodiments the media is supplemented with an inhibitor of GSK3β signaling from about day 11 through day 12, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of GSK3β signaling at a concentration of between about 1 μM and about 4 μM. In some embodiments, cells are exposed to the inhibitor of GSK3β signaling at a concentration of between about 2 μM and about 3 PM. In some embodiments, cells are exposed to the inhibitor of GSK3β signaling at a concentration of about 2 μM.

In some embodiments, the inhibitor of GSK3β signaling is selected from lithium ion, valproic acid, iodotubercidin, naproxen, famotidine, curcumin, olanzapine, CHIR99012, or a combination thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3β. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

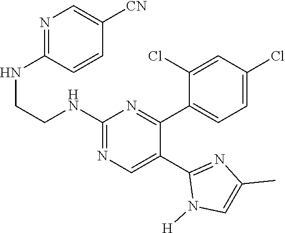 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM from about day 11 up to about day 13 (e.g., day 12 or day 13). In some embodiments, cells are exposed to CHIR99021 at a concentration of about 2.0 μM from about day 11 through about day 12, inclusive of each day.

In some embodiments the media is supplemented with brain-derived neurotrophic factor (BDNF). In some embodiments the media is supplemented with BDNF beginning on about day 11. In some embodiments the media is supplemented with BDNF from about day 11 until harvest or collection. In some embodiments the media is supplemented with BDNF from about day 11 through day 18. In some embodiments the media is supplemented with BDNF from about day 11 through day 20. In some embodiments the media is supplemented with BDNF from about day 11 through day 25.

In some embodiments, cells are exposed to BDNF at a concentration of between about 1 ng/mL and 100 ng/mL, between about 5 ng/mL and about 50 ng/mL, between about 10 ng/mL and about 30 ng/mL. In some embodiments, cells are exposed to BDNF at a concentration of between about 10 ng/mL and about 30 ng/mL. In some embodiments, cells are exposed to BDNF at a concentration of about 20 ng/mL.

In some embodiments, the media is supplemented with about 20 ng/mL BDNF beginning on about day 11. In some embodiments the media is supplemented with 20 ng/mL BDNF from about day 11 until harvest or collection. In some embodiments the media is supplemented with about 20 ng/mL BDNF from about day 11 through day 18. In some embodiments the media is supplemented with about 20 ng/mL BDNF from about day 11 through day 20. In some embodiments the media is supplemented with about 20 ng/mL BDNF from about day 11 through day 25.

In some embodiments the media is supplemented with glial cell-derived neurotrophic factor (GDNF). In some embodiments the media is supplemented with GDNF beginning on about day 11. In some embodiments the media is supplemented with GDNF from about day 11 until harvest or collection. In some embodiments the media is supplemented with GDNF from about day 11 through day 18. In some embodiments the media is supplemented with GDNF from about day 11 through day 20. In some embodiments the media is supplemented with GDNF from about day 11 through day 25.

In some embodiments, cells are exposed to GDNF at a concentration of between about 1 ng/mL and 100 ng/mL, between about 5 ng/mL and about 50 ng/mL, between about 10 ng/mL and about 30 ng/mL. In some embodiments, cells are exposed to GDNF at a concentration of between about 10 ng/mL and about 30 ng/mL. In some embodiments, cells are exposed to GDNF at a concentration of about 20 ng/mL.

In some embodiments, the media is supplemented with about 20 ng/mL GDNF beginning on about day 11. In some embodiments the media is supplemented with 20 ng/mL GDNF from about day 11 until harvest or collection. In some embodiments the media is supplemented with about 20 ng/mL GDNF from about day 11 through day 18. In some embodiments the media is supplemented with about 20 ng/mL GDNF from about day 11 through day 20. In some embodiments the media is supplemented with about 20 ng/mL GDNF from about day 11 through day 25.

In some embodiments the media is supplemented with ascorbic acid. In some embodiments the media is supplemented with ascorbic acid beginning on about day 11. In some embodiments the media is supplemented with ascorbic acid from about day 11 until harvest or collection. In some embodiments the media is supplemented with ascorbic acid from about day 11 through day 18. In some embodiments the media is supplemented with ascorbic acid from about day 11 through day 20. In some embodiments the media is supplemented with ascorbic acid from about day 11 through day 25.

In some embodiments, cells are exposed to ascorbic acid at a concentration of between about 0.05 mM and 5 mM, between about 0.1 mM and about 1 mM, between about 0.2 mM and about 0.5 mM, each inclusive. In some embodiments, cells are exposed to ascorbic acid at a concentration of between about 0.05 mM and about 5 mM, each inclusive. In some embodiments, cells are exposed to ascorbic acid at a concentration of between about 0.1 mM and about 1 mM, each inclusive. In some embodiments, cells are exposed to ascorbic acid at a concentration of about 0.2 mM.

In some embodiments, the media is supplemented with about 0.2 mM ascorbic acid beginning on about day 11. In some embodiments the media is supplemented with 0.2 mM ascorbic acid from about day 11 until harvest or collection. In some embodiments the media is supplemented with about 0.2 mM ascorbic acid from about day 11 through day 18. In some embodiments the media is supplemented with about 0.2 mM ascorbic acid from about day 11 through day 20. In some embodiments the media is supplemented with about 0.2 mM ascorbic acid from about day 11 through day 25.

In some embodiments the media is supplemented with dibutyryl cyclic AMP (dbcAMP). In some embodiments the media is supplemented with dbcAMP beginning on about day 11. In some embodiments the media is supplemented with dbcAMP from about day 11 until harvest or collection. In some embodiments the media is supplemented with dbcAMP from about day 11 through day 18. In some embodiments the media is supplemented with dbcAMP from about day 11 through day 20. In some embodiments the media is supplemented with dbcAMP from about day 11 through day 25.

In some embodiments, cells are exposed to dbcAMP at a concentration of between about 0.05 mM and 5 mM, between about 0.1 mM and about 3 mM, between about 0.2 mM and about 1 mM, each inclusive. In some embodiments, cells are exposed to dbcAMP at a concentration of between about 0.1 mM and about 3 mM, each inclusive. In some embodiments, cells are exposed to dbcAMP at a concentration of between about 0.2 mM and about 1 mM, each inclusive. In some embodiments, cells are exposed to dbcAMP at a concentration of about 0.5 mM.

In some embodiments, the media is supplemented with about 0.5 mM dbcAMP beginning on about day 11. In some embodiments the media is supplemented with 0.5 mM dbcAMP from about day 11 until harvest or collection. In some embodiments the media is supplemented with about 0.5 mM dbcAMP from about day 11 through day 18. In some embodiments the media is supplemented with about 0.5 mM dbcAMP from about day 11 through day 20. In some embodiments the media is supplemented with about 0.5 mM dbcAMP from about day 11 through day 25.

In some embodiments the media is supplemented with transforming growth factor beta 3 (TGF3β). In some embodiments the media is supplemented with TGFβ3 beginning on about day 11. In some embodiments the media is supplemented with TGFβ3 from about day 11 until harvest or collection. In some embodiments the media is supplemented with TGFβ3 from about day 11 through day 18. In some embodiments the media is supplemented with TGFβ3 from about day 11 through day 20. In some embodiments the media is supplemented with TGFβ3 from about day 11 through day 25.

In some embodiments, cells are exposed to TGFβ3 at a concentration of between about 0.1 ng/mL and 10 ng/mL, between about 0.5 ng/mL and about 5 ng/mL, or between about 1.0 ng/mL and about 2.0 ng/mL. In some embodiments, cells are exposed to TGFβ3 at a concentration of between about 1.0 ng/mL and about 2.0 ng/mL, each inclusive. In some embodiments, cells are exposed to TGFβ3 at a concentration of about 1 ng/mL.

In some embodiments, the media is supplemented with about 1 ng/mL TGFβ3 beginning on about day 11. In some embodiments the media is supplemented with 1 ng/mL TGFβ3 from about day 11 until harvest or collection. In some embodiments the media is supplemented with about 1 ng/mL TGFβ3 from about day 11 through day 18. In some embodiments the media is supplemented with about 1 ng/mL TGFβ3 from about day 11 through day 20. In some embodiments the media is supplemented with about 1 ng/mL TGFβ3 from about day 11 through day 25.

In some embodiments the media is supplemented with an inhibitor of Notch signaling. In some embodiments the media is supplemented with an inhibitor of Notch signaling beginning on about day 11. In some embodiments the media is supplemented with an inhibitor of Notch signaling from about day 11 until harvest or collection. In some embodiments the media is supplemented with an inhibitor of Notch signaling from about day 11 through day 18. In some embodiments the media is supplemented with an inhibitor of Notch signaling from about day 11 through day 20. In some embodiments the media is supplemented with an inhibitor of Notch signaling from about day 11 through day 25.

In some embodiments, an inhibitor of Notch signaling is selected from cowanin, PF-03084014, L685458, LY3039478, DAPT, or a combination thereof. In some embodiments, the inhibitor of Notch signaling inhibits gamma secretase. In some embodiments, the inhibitor of Notch signaling is a small molecule. In some embodiments, the inhibitor of Notch signaling is DAPT, having the following formula:

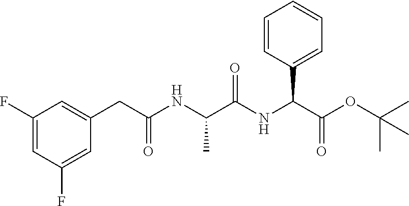 [SMILES: C[C@H](NC(=O)Cc1cc(F)cc(F)c1)C(=O)N[C@H](C(=O)OC(C)(C)C)c1ccccc1]

In some embodiments, cells are exposed to DAPT at a concentration of between about 1 μM and about 20 μM, between about 5 μM and about 15 μM, or between about 8 μM and about 12 μM. In some embodiments, cells are exposed to DAPT at a concentration of between about 1 μM and about 20 μM. In some embodiments, cells are exposed to DAPT at a concentration of between about 5 μM and about 15 μM. In some embodiments, cells are exposed to DAPT at a concentration of between about 8 μM and about 12 μM. In some embodiments, cells are exposed to DAPT at a concentration of about 10 PM.

In some embodiments, the media is supplemented with about 10 μM DAPT beginning on about day 11. In some embodiments the media is supplemented with 10 μM DAPT from about day 11 until harvest or collection. In some embodiments the media is supplemented with about 10 μM DAPT from about day 11 through day 18. In some embodiments the media is supplemented with about 10 μM DAPT from about day 11 through day 20. In some embodiments the media is supplemented with about 10 μM DAPT from about day 11 through day 25.

In some embodiments, beginning on about day 11, the media is supplemented with about 20 ng/mL BDNF, about 20 ng/mL GDNF, about 0.2 mM ascorbic acid, about 0.5 mM dbcAMP, about 1 ng/mL TGF3β, and about 10 μM DAPT. In some embodiments, from about day 11 until harvest or collection, the media is supplemented with about 20 ng/mL BDNF, about 20 ng/mL GDNF, about 0.2 mM ascorbic acid, about 0.5 mM dbcAMP, about 1 ng/mL TGF3β, and about 10 μM DAPT. In some embodiments, from about day 11 until day 18, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until day 20, the media is supplemented with about 20 ng/mL BDNF, about 20 ng/mL GDNF, about 0.2 mM ascorbic acid, about 0.5 mM dbcAMP, about 1 ng/mL TGF3β, and about 10 μM DAPT. In some embodiments, from about day 11 until day 25, the media is supplemented with about 20 ng/mL BDNF, about 20 ng/mL GDNF, about 0.2 mM ascorbic acid, about 0.5 mM dbcAMP, about 1 ng/mL TGF3β, and about 10 μM DAPT.

In some embodiments, from day about 7 to about day 18 (i.e. day 17 or day 18), at least about 50% of the media is replaced daily. In some embodiments, from day about 7 to about day 18 (i.e. day 17 or day 18), at least about 75% of the media is replaced daily. In some embodiments, from day about 7 to about day 18 (i.e. day 17 or day 18), about 100% of the media is replaced daily. In some embodiments, on about day 19, at least about 50% of the media is replaced daily. In some embodiments, on about day 19, at least about 75% of the media is replaced daily. In some embodiments, on about day 19, about 100% of the media is replaced daily. In some embodiments, about 100% of the media is replaced daily from about day 11 to day 18, and again on day 19. In some embodiments, about 100% of the media is replaced daily on days 11-17 and 19.

In some embodiments, the second incubation involves culturing cells produced from the first incubation in a “maturation media.”

In some embodiments, the second incubation involves comprises culturing the cells in the maturation media beginning on about day 11. In some embodiments, cells are cultured in the maturation media to produce determined dopamine (DA) neuron progenitor cells, or dopamine (DA) neurons.

In some embodiments, the maturation media is formulated to contain Neurobasal™ media, supplemented with N-2 and B27 supplements, non-essential amino acids (NEAA), and GlutaMAX™. In some embodiments, the maturation media is further supplemented with any of the molecules described in Section II.

In some embodiments, the cells are cultured in the maturation media beginning on about day 11. In some embodiments, the cells are cultured in the maturation media from about day 11 until harvest or collection of the cells. In some embodiments, cells are harvested between day 16 and 27. In some embodiments, cells are harvested between day 18 and day 25. In some embodiments, cells are harvested on day 18. In some embodiments, cells are harvested on day 20. In some embodiments, the cells are cultured in the maturation media from about day 11 until day 20. In some embodiments, cells are harvested on day 25.

### D. Harvesting, Collecting, and Formulating Differentiated Cells

In embodiments of the provided methods, neurally differentiated cells produced by the methods provided herein can be harvested or collected, such as for formulation and use of the cells. In some embodiments, the provided methods for producing differentiated cells, such as for use as a cell therapy in the treatment of a neurodegenerative disease may include formulation of cells, such as formulation of differentiated cells resulting from the provided methods described herein. In some embodiments, the dose of cells comprising differentiated cells (e.g. determined DA neuron progenitor cells or DA neurons), is provided as a composition or formulation, such as a pharmaceutical composition or formulation. Such compositions can be used in accord with the provided methods, such as in the prevention or treatment of neurodegenerative disorders, including Parkinson's disease.

In some cases, the cells are processed in one or more steps for manufacturing, generating or producing a cell therapy and/or differentiated cells may include formulation of cells, such as formulation of differentiated cells resulting from the methods. In some cases, the cells can be formulated in an amount for dosage administration, such as for a single unit dosage administration or multiple dosage administration.

In certain embodiments, one or more compositions of differentiated cells are formulated. In particular embodiments, one or more compositions of differentiated cells are formulated after the one or more compositions have been produced. In some embodiments, the one or more compositions have been previously cryopreserved and stored, and are thawed prior to the administration.

In certain embodiments, the differentiated cells include determined DA neuron progenitor cells. In some embodiments, a formulated composition of differentiated cells is a composition enriched for determined DA neuron progenitor cells. In certain embodiments, the differentiated cells include DA neurons. In some embodiments, a formulated composition of differentiated cells is a composition enriched for DA neurons.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are differentiated for at least 16 days. In some embodiments, the cells are differentiated for between 16 day and 30 days. In some embodiments, the cells are differentiated for between 16 day and 27 days. In some embodiments, the cells are differentiated for between 18 and 25 day. In some embodiments, the cells are differentiated for about 16 days. In some embodiments, the cells are differentiated for about 17 days. In some embodiments, the cells are differentiated for about 18 days. In some embodiments, the cells are differentiated for about 19 days. In some embodiments, the cells are differentiated for about 20 days. In some embodiments, the cells are differentiated for about 21 days. In some embodiments, the cells are differentiated for about 22 days. In some embodiments, the cells are differentiated for about 23 days. In some embodiments, the cells are differentiated for about 24 days. In some embodiments, the cells are differentiated for about 25 days.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are harvested after at least 16 days of culture.

In some embodiments, the cells are harvested between 16 days and 30 days of culture. In some embodiments, the cells are harvested between 16 days and 27 days of culture. In some embodiments, the cells are harvested between 18 days and 25 days of culture. In some embodiments, the cells are harvested after about 16 days of culture. In some embodiments, the cells are harvested after about 17 days of culture. In some embodiments, the cells are harvested after about 18 days of culture. In some embodiments, the cells are harvested after about 19 days of culture. In some embodiments, the cells are harvested after about 20 days of culture. In some embodiments, the cells are harvested after about 21 days of culture. In some embodiments, the cells are harvested after about 22 days of culture. In some embodiments, the cells are harvested after about 23 days of culture. In some embodiments, the cells are harvested after about 24 days of culture. In some embodiments, the cells are harvested after about 25 days of culture.

In some of any embodiments, the harvested cells include determined dopaminergic neuronal progenitor cells (DDPCs).

In some of any embodiments, at least 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, or 90% of the harvested cells are DDPCs.

In some of any embodiments, the DDPCs express one or more genes selected from (a) ASPM; (b) AURKB; (c) BRINP1; (d) BUB1; (e) CCNB2; (f) CDC20; (g) CDC25C; (h) CDKN1A; (i) CENPF; (j) DLGAP5; (k) FAM83D; (1) FANCD2; (m) GEM; (n) HMMR; (o) IQGAP3; (p) KIF20A; (q) KIF2C; (r) KIFC1; (s) MKI67; (t) PIMREG; (u) PLK2; (v) PTTG1; (w) SAPCD2; (x) TOP2A; (y) TPX2; and (z) TTK.

In some of any embodiments, the DDPCs express one or more genes selected from (b) AURKB; (c) BRINP1; (d) BUB1; (e) CCNB2; (f) CDC20; (g) CDC25C; (h) CDKN1A; (i) CENPF; (j) DLGAP5; (k) FAM83D; (1) FANCD2; (m) GEM; (n) HMMR; (o) IQGAP3; (p) KIF20A; (q) KIF2C; (r) KIFC1; (s) MKI67; (t) PIMREG; (u) PLK2; (v) PTTG1; (w) SAPCD2; (y) TPX2; and (z) TTK.

In some of any embodiments, the DDPCs express one or more genes selected from (b) AURKB; (e) CCNB2; (g) CDC25C; (j) DLGAP5; (k) FAM83D; (o) IQGAP3; (p) KIF20A; (q) KIF2C; (r) KIFC1; (v) PTTG1; (w) SAPCD2; (x) TOP2A; and (y) TPX2.

In some of any embodiments, the DDPCs express one or more genes selected from (b) AURKB; (e) CCNB2; (g) CDC25C; (j) DLGAP5; (k) FAM83D; (o) IQGAP3; (p) KIF20A; (q) KIF2C; (r) KIFC1; (v) PTTG1; (w) SAPCD2; and (y) TPX2.

In some of any embodiments, expression of at least one of the one or more genes by the DDPCs is or is on average 1.1, 1.2, 1.3, 1.4, 1.5, 1.6, 1.7, 1.8, 1.9, or 2.0 fold greater than expression by a reference population of cells. In some of any embodiments, expression of each of the one or more genes by the DDPCs is or is on average 1.1, 1.2, 1.3, 1.4, 1.5, 1.6, 1.7, 1.8, 1.9, or 2.0 fold greater than expression by a reference population of cells.

In some embodiments, the reference population of cells includes reference cells. In some embodiments, the reference population of cells is enriched for reference cells. In some embodiments, at least 50, 55, 60, 65, 70, 75, 80, 85, 90, 95, 96, 97, 98, or 99 percent of the reference population of cells is reference cells. In some embodiments, the reference population of cells is reference cells.

In some embodiments, the reference cells are not determined dopaminergic neuronal progenitor cells. In some embodiments, the reference cells are pluripotent stem cells. In some embodiments, the reference cells are floor plate midbrain progenitor cells. In some embodiments, the reference cells are differentiated dopaminergic neurons.

In some embodiments, the reference cells are cells differentiated from pluripotent stem cells under conditions to neurally differentiate the cells. In some embodiments, the cells are differentiated according to any of the methods described herein.

In some embodiments, the reference cells are cells at a particular timepoint of the differentiation method. In some embodiments, the timepoint is before the timepoint at which the harvested cells are harvested. In some embodiments, the timepoint is after the timepoint at which the harvested cells are harvested. In some embodiments, the timepoint is day 13. In some embodiments, the timepoint is day 14. In some embodiments, the timepoint is day 15. In some embodiments, the timepoint is day 16. In some embodiments, the timepoint is day 17. In some embodiments, the timepoint is day 16. In some embodiments, the timepoint is day 19. In some embodiments, the timepoint is day 20. In some embodiments, the timepoint is day 21. In some embodiments, the timepoint is day 22. In some embodiments, the timepoint is day 23. In some embodiments, the timepoint is day 24. In some embodiments, the timepoint is day 25.

In some of any embodiments, expression of at least one of the one or more genes by the DDPCs is at a ratio with respect to GAPDH expression. In some of any embodiments, expression of each of the one or more genes by the DDPCs is at a ratio with respect to GAPDH expression.

In some of any embodiments, the DDPCs exhibit or exhibit on average one or more of: (a) a ratio of ASPM to GAPDH expression of greater than about 7×10⁻⁴; (b) a ratio of AURKB to GAPDH expression of greater than about 9×10⁻⁴; (c) a ratio of BRINP1 to GAPDH expression of greater than about 9×10⁻⁵; (d) a ratio of BUB1 to GAPDH expression of greater than about 3×10⁻³; (e) a ratio of CCNB2 to GAPDH expression of greater than about 3×10⁻³; (f) a ratio of CDC20 to GAPDH expression of greater than about 3×10⁻³; (g) a ratio of CDC25C to GAPDH expression of greater than about 5×10⁻⁴; (h) a ratio of CDKN1A to GAPDH expression of greater than about 1×10⁻³; (i) a ratio of CENPF to GAPDH expression of greater than about 7×10⁻³; (j) a ratio of DLGAP5 to GAPDH expression of greater than about 2×10⁻³; (k) a ratio of FAM83D to GAPDH expression of greater than about 6×10⁻⁴; (1) a ratio of FANCD2 to GAPDH expression of greater than about 3×10⁻³; (m) a ratio of GEM to GAPDH expression of greater than about 6×10⁻⁴; (n) a ratio of HMMR to GAPDH expression of greater than about 8×10⁻⁴; (o) a ratio of IQGAP3 to GAPDH expression of greater than about 1×10⁻³; (p) a ratio of KIF20A to GAPDH expression of greater than about 1×10⁻³; (q) a ratio of KIF2C to GAPDH expression of greater than about 3×10⁻³; (r) a ratio of KIFC1 to GAPDH expression of greater than about 2×10⁻³; (s) a ratio of MKI67 to GAPDH expression of greater than about 2×10⁻³; (t) a ratio of PIMREG to GAPDH expression of greater than about 1×10⁻³; (u) a ratio of PLK2 to GAPDH expression of greater than about 4×10⁻³; (v) a ratio of PTTG1 to GAPDH expression of greater than about 3×10⁻³; (w) a ratio of SAPCD2 to GAPDH expression of greater than about 1×10⁻³; (x) a ratio of TOP2A to GAPDH expression of greater than about 3×10⁻²; (y) a ratio of TPX2 to GAPDH expression of greater than about 7×10⁻³; and (z) a ratio of TTK to GAPDH expression of greater than about 2×10⁻³.

In some of any embodiments, (a) the ratio of ASPM to GAPDH expression is between about 7×10⁻⁴ and about 2×10⁻¹; (b) the ratio of AURKB to GAPDH expression is between about 9×10⁻⁴ and about 4×10⁻²; (c) the ratio of BRINP1 to GAPDH expression is between about 9×10⁻⁵ and about 5×10⁻²; (d) the ratio of BUB1 to GAPDH expression is between about 3×10⁻³ and about 7×10⁻²; (e) the ratio of CCNB2 to GAPDH expression is between about 3×10⁻³ and about 7×10⁻²; (f) the ratio of CDC20 to GAPDH expression is between about 3×10⁻³ and about 1×10⁻¹; (g) the ratio of CDC25C to GAPDH expression is between about 5×10⁻⁴ and about 3×10⁻²; (h) the ratio of CDKN1A to GAPDH expression is between about 1×10⁻³ and about 9×10⁻²; (i) the ratio of CENPF to GAPDH expression is between about 7×10⁻³ and about 5×10⁻¹; (j) the ratio of DLGAP5 to GAPDH expression is between about 2×10⁻³ and about 9×10⁻²; (k) the ratio of FAM83D to GAPDH expression is between about 6×10⁻⁴ and about 3×10⁻²; (1) the ratio of FANCD2 to GAPDH expression is between about 3×10⁻³ and about 4×10⁻²; (m) the ratio of GEM to GAPDH expression is between about 6×10⁻⁴ and about 3×10⁻²; (n) the ratio of HMMR to GAPDH expression is between about 8×10⁻⁴ and about 6×10⁻²; (o) the ratio of IQGAP3 to GAPDH expression is between about 1×10⁻³ and about 6×10⁻²; (p) the ratio of KIF20A to GAPDH expression is between about 1×10⁻³ and about 8×10⁻²; (q) the ratio of KIF2C to GAPDH expression is between about 3×10⁻³ and about 7×10⁻²; (r) the ratio of KIFC1 to GAPDH expression is between about 2×10⁻³ and about 8×10⁻²; (s) the ratio of MKI67 to GAPDH expression is between about 2×10⁻³ and about 4×10⁻¹; (t) the ratio of PIMREG to GAPDH expression is between about 1×10⁻³ and about 4×10⁻²; (u) the ratio of PLK2 to GAPDH expression is between about 4×10⁻³ and about 6×10⁻²; (v) the ratio of PTTG1 to GAPDH expression is between about 3×10⁻³ and about 9×10⁻²; (w) the ratio of SAPCD2 to GAPDH expression is between about 1×10⁻³ and about 3×10⁻²; (x) the ratio of TOP2A to GAPDH expression is between about 3×10⁻² and about 7×10⁻¹; (y) the ratio of TPX2 to GAPDH expression is between about 7×10⁻³ and about 2×10⁻¹; and/or (z) the ratio of TTK to GAPDH expression is between about 2×10⁻³ and about 8×10⁻². In some of any embodiments, the ratio is on average across the DDPCs.

In some of any embodiments, the ratio of BRINP1 to GAPDH expression is between about 9×10⁻⁵ and about 5×10⁻²; the ratio of CDKN1A to GAPDH expression is between about 1×10⁻³ and about 9×10⁻²; the ratio of FAM83D to GAPDH expression is between about 6×10⁴ and about 3×10⁻²; the ratio of FANCD2 to GAPDH expression is between about 3×10⁻³ and about 4×10⁻²; the ratio of GEM to GAPDH expression is between about 6×10⁻⁴ and about 3×10⁻²; the ratio of PLK2 to GAPDH expression is between about 4×10⁻³ and about 6×10⁻²; and/or the ratio of SAPCD2 to GAPDH expression is between about 1×10⁻³ and about 3×10⁻². In some of any embodiments, the ratio is on average across the DDPCs.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the differentiated cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the neurodegenerative condition or disease (e.g. Parkinson's disease), such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cells are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the differentiated cells to replace the cells in a cryopreservative solution. In some embodiments, the cells are frozen, e.g., cryopreserved or cryoprotected, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryopreserved or cryoprotected, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the composition of differentiated cells are formulated, cryopreserved, and then stored for an amount of time. In certain embodiments, the formulated, cryopreserved cells are stored until the cells are released for administration. In particular embodiments, the formulated cryopreserved cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryopreserved and stored for, for about, or for less than 1 days, 2 days, 3 days, 4 days, 5 days, 6 days, or 7 days. In certain embodiments, the cells are thawed and administered to a subject after the storage.

In some embodiments, the formulation is carried out using one or more processing step including washing, diluting or concentrating the cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to differentiated cells. In some embodiments, the volume of formulation buffer is from or from about 1 μL to 5000 μL, such as at least or about at least or about or 5 μL, 10 μL, 20 μL, 50 μL, 100 μL, 200 μL, 300 μL, 400 μL, 500 μL, 1000 μL, 2000 μL, 3000 μL, 4000 μL, or 5000 μL.

A container may generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, such cells produced by the method, or a composition comprising such cells, are administered to a subject for treating a neurodegenerative disease or condition.

### E. Exemplary Processes

As described by the methods provided herein, pluripotent stem cells may be differentiated into lineage specific cell populations, including determined DA progenitors cells and DA neurons. These cells may then be used in cell replacement therapy. As described by the methods here, in some embodiments, the pluripotent stem cells are differentiated into floor plate midbrain progenitor cells, and the cells are further differentiated into determined dopamine (DA) neuron progenitor cells, and/or dopamine (DA) neurons. In some embodiments, the pluripotent stem cells are differentiated into determined DA neuron progenitor cells. In some embodiments, the pluripotent stem cells are differentiated into DA neurons. In some embodiments, pluripotent stem cells are embryonic stem cells. In some embodiments, pluripotent stem cells are induced pluripotent stem cells.

In some embodiments, embryonic stem cells are differentiated into floor plate midbrain progenitor cells, and then into determined dopamine (DA) neuron progenitor cells, and/or dopamine (DA) neurons. In some embodiments, embryonic stem cells are differentiated into determined DA neuron progenitor cells. In some embodiments, embryonic stem cells are differentiated into DA neurons.

In some embodiments, induced pluripotent stem cells are differentiated into floor plate midbrain progenitor cells, and then into determined dopamine (DA) neuron progenitor cells, and/or dopamine (DA) neurons. In some embodiments, induced pluripotent stem cells are differentiated into determined DA neuron progenitor cells. In some embodiments, induced pluripotent stem cells are differentiated into DA neurons.

In some embodiments, the method involves (a) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-3/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to induce neural differentiation the cells. In some embodiments, culturing the cells under conditions to induce neural differentiation of the cells involves exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF3β); and (vi) an inhibitor of Notch signaling.

In some embodiments, the method involves (a) performing a first incubation including culturing pluripotent stem cells in a plate having microwells under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; (iv) an inhibitor of glycogen synthase kinase 3β (GSK3β) signaling; and (v) a serum replacement; (b) dissociating the cells of the spheroid to produce a cell suspension; (c) transferring cells of the cell suspension to a laminin-coated culture vessel; (d) performing a second incubation including culturing cells of the spheroid in the laminin-coated culture vessel under conditions to induce neural differentiation of the cells; and (e) harvesting the neurally differentiated cells. In some embodiments, the second incubation involves culturing cells in the presence of a serum replacement. In some embodiments, culturing the cells under conditions to induce neural differentiation of the cells involves exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF3β); and (vi) an inhibitor of Notch signaling.

In some embodiments, the cells are exposed to the inhibitor of TGF-3/activin-Nodal (e.g., SB431542 or “SB”) from day 0 up to about day 7 (e.g., day 6 or day 7). In some embodiments, the cells are exposed to the inhibitor of TGF-β/activin-Nodal (e.g., SB431542 or “SB”) from day 0 through day 6, inclusive of each day. In some embodiments, the cells are exposed to the at least one activator of SHH signaling (e.g., SHH protein and purmorphamine, collectively “SHH/PUR”) from day 0 up to about day 7 (e.g. day 6 or day 7). In some embodiments, the cells are exposed to the at least one activator of SHH signaling (e.g., SHH protein and purmorphamine, collectively “SHH/PUR”) from day 0 through day 6, inclusive of each day. In some embodiments, the cells are exposed to the inhibitor of BMP signaling (e.g., LDN193189 or “LDN”) from day 0 up to about day 11 (e.g., day 10 or day 11). In some embodiments, the cells are exposed to the inhibitor of BMP signaling (e.g., LDN193189 or “LDN”) from day 0 through day 10, inclusive of each day. In some embodiments, the cells are exposed to the inhibitor of GSK3β signaling (e.g., CHIR99021 or “CHIR”) from day 0 up to about day 13 (e.g., day 12 or day 13). In some embodiments, the cells are exposed to the inhibitor of GSK3β signaling (e.g., CHIR99021 or “CHIR”) from day 0 through day 12.

In some embodiments, the cells are exposed to (i) an inhibitor of TGF-3/activin-Nodal signaling from day 0 up to about day 7 (e.g., day 6 or day 7); (ii) at least one activator of Sonic Hedgehog (SHH) signaling from day 0 up to about day 7 (e.g., day 6 or day 7); (iii) an inhibitor of bone morphogenetic protein (BMP) signaling from day 0 up to about day 11 (e.g., day 10 or day 11); and (iv) an inhibitor of glycogen synthase kinase 3β (GSK3β) signaling from day 0 up to about day 13 (e.g., day 12 or day 13). In some embodiments, the cells are exposed to (i) SB from day 0 up to about day 7 (e.g., day 6 or day 7); (ii) SHH/PUR from day 0 up to about day 7 (e.g., day 6 or day 8); (iii) LDN from day 0 up to about day 11 (e.g. day 10 or day 11); and (iv) CHIR from day 0 up to about day 13 (e.g. day 12 or day 13). In some embodiments, the cells are exposed to (i) an inhibitor of TGF-β/activin-Nodal signaling from day 0 through day 6, each day inclusive; (ii) at least one activator of Sonic Hedgehog (SHH) signaling from day 0 through day 6, each day inclusive; (iii) an inhibitor of bone morphogenetic protein (BMP) signaling from day 0 through day 10, each day inclusive; and (iv) an inhibitor of glycogen synthase kinase 3β (GSK3β) signaling from day 0 through day 12, each day inclusive. In some embodiments, the cells are exposed to (i) SB from day 0 through day 6, each day inclusive; (ii) SHH/PUR from day 0 through day 6, each day inclusive; (iii) LDN from day 0 through day 10, each day inclusive; and (iv) CHIR from day 0 through day 12, each day inclusive.

In some embodiments, the cells are exposed to brain-derived neurotrophic factor (BDNF) beginning on day 11. In some embodiments, the cells are exposed to ascorbic acid. In some embodiments, the cells are exposed to glial cell-derived neurotrophic factor (GDNF) beginning on day 11. In some embodiments, the cells are exposed to dibutyryl cyclic AMP (dbcAMP) beginning on day 11. In some embodiments, the cells are exposed to transforming growth factor beta-3 (TGF3β) beginning on day 11. In some embodiments, the cells are exposed to the inhibitor of Notch signaling (e.g., DAPT) beginning on day 11. In some embodiments, beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF3β); and (vi) the inhibitor of Notch signaling (e.g., DAPT) (collectively “BAGCT/DAPT”). In some embodiments, the cells are exposed to BAGCT/DAPT beginning on day 11 until harvest or collection. In some embodiments, the cells are exposed to BAGCT/DAPT from day 11 through day 18. In some embodiments, the cells are exposed to BAGCT/DAPT from day 11 through day 25.

In some embodiments, the cells are exposed to a Rho-associated protein kinase (ROCK) inhibitor on day 0. In some embodiments, the cells are exposed to a Rho-associated protein kinase (ROCK) inhibitor on day 7. In some embodiments, the cells are exposed to a Rho-associated protein kinase (ROCK) inhibitor on day 16. In some embodiments, the cells are exposed to a Rho-associated protein kinase (ROCK) inhibitor on day 20. In some embodiments, the cells are exposed to a Rho-associated protein kinase (ROCK) inhibitor on day 0, day 7, day 16, and day 20. In some embodiments, the cells are exposed to a ROCK inhibitor on the day on which the cells are passaged. In some embodiments, the cells are passaged on day 0, 7, 16, 20, or combinations thereof. In some embodiments, the cells are passaged on day 0, 7, 16, and 20.

In some embodiments, the cells are cultured in a basal induction medium comprising DMEM/F-12 and Neurobasal media (e.g., at a 1:1 ratio), supplemented with N2, B27, non-essential amino acids (NEAA), Glutamax, L-glutamine, β-mercaptoethanol, and insulin. In some embodiments, the cells are cultured in the basal induction media from about day 0 through about day 10. In some embodiments, the basal induction media is for differentiating pluripotent stem cells into floor plate midbrain progenitor cells.

In some embodiments, the cells are cultured in a maturation medium comprising Neurobasal media, supplemented with N2, B27, non-essential amino acids (NEAA), and Glutamax. In some embodiments, the cells are cultured in the basal induction media from about day 11 until harvest or collection. In some embodiments, the cells are cultured in the basal induction media from about day 11 through day 18. In some embodiments, the cells are cultured in the basal induction media from about day 11 through day 20. In some embodiments, the maturation media is for differentiating floor plate midbrain progenitor cells into determined dopamine (DA) neuron progenitor cells. In some embodiments, the cells are cultured in the basal induction media from about day 11 through day 25. In some embodiments, the maturation media is for differentiating floor plate midbrain progenitor cells into dopamine (DA) neurons.

In some embodiments, the media is supplemented with small molecules as described above, including SB, SHH/PUR, LDN, CHIR, BAGCT/DAPT, and ROCKi. In some embodiments, the media is changed every day or every other day. In some embodiments the media is changed every day. In some embodiments the media is changed every other day. In some embodiments, the media is changed every day from about day 0 up to about day 17 (e.g., day 16 or day 18). In some embodiments, the media is changed every other day from about day 18 until harvest or collection. In some embodiments, the media is changed every day from about day 0 up to about day 17 (e.g., day 16 or day 18), and then every other day from about day 18 until harvest or collection. In some embodiments, harvest or collection is on day 20.

In some embodiments, a serum replacement is provided in the media from about day 0 up to about day 10 (e.g. day 9 or day 11). In some embodiments, the serum replacement is provided at 5% (v/v) in the media on day 0 and day 1. In some embodiments, the serum replacement is provided at 2% (v/v) in the media on day 2 through day 10. In some embodiments, the serum replacement is provided at 5% (v/v) in the media on day 0 and day 1 and at 2% (v/v) in the media on day 2 through day 10. In some embodiments, serum replacement is not provided in the media after day 10.

In some embodiments, at least about 50% or at least about 75% of the media is changed. In some embodiments, at least about 50% of the media is changed. In some embodiments, at least about 75% of the media is changed. In some embodiments about 100% of the media is changed.

In some embodiments, about 50% or about 75% of the media is changed. In some embodiments, about 50% of the media is changed. In some embodiments, about 75% of the media is changed. In some embodiments about 100% of the media is changed.

In some embodiments, the media is supplemented with small molecules selected from SB, SHH/PUR, LDN, CHIR, BAGCT/DAPT, ROCKi, or a combination thereof. In some embodiments, when about 50% of the media is changed, the concentration of each small molecule is doubled as compared to its concentration on day 0.

In some embodiments, the method involves (a) performing a first incubation, wherein, beginning at the initiation of the first incubation (day 0) the cells are exposed to (i) an inhibitor of TGF-3/activin-Nodal signaling; and (ii) an inhibitor of bone morphogenetic protein (BMP) signaling; beginning on day 1, the cells are exposed to at least one activator of Sonic Hedgehog (SHH) signaling; and beginning on day 2, the cells are exposed to an inhibitor of bone morphogenetic protein (BMP) signaling; and (iv) an inhibitor of glycogen synthase kinase 3β (GSK3β) signaling; and (b) performing a second incubation, wherein, beginning on day 11, the cells are exposed to (i) brain-derived neurotrophic factor (BDNF); (ii) glial cell-derived neurotrophic factor (GDNF); (iii) transforming growth factor beta-3 (TGF3β); (iv) an inhibitor of Notch signaling; (v) ascorbic acid; and (vi) dibutyryl cyclic AMP (dbcAMP).

In some embodiments, the cells are exposed to the inhibitor of TGF-β/activin-Nodal (e.g., SB431542 or “SB”) from day 0 up to about day 5 (e.g., day 4 or day 5). In some embodiments, the cells are exposed to the inhibitor of TGF-β/activin-Nodal (e.g., SB431542 or “SB”) from day 0 through day 4, inclusive of each day. In some embodiments, the cells are exposed to the at least one activator of SHH signaling (e.g., SHH protein and purmorphamine, collectively “SHH/PUR”) from day 1 up to about day 7 (e.g. day 6 or day 7). In some embodiments, the cells are exposed to the at least one activator of SHH signaling (e.g., SHH protein and purmorphamine, collectively “SHH/PUR”) from day 1 through day 6, inclusive of each day. In some embodiments, the cells are exposed to the inhibitor of BMP signaling (e.g., LDN193189 or “LDN”) from day 0 up to about day 11 (e.g., day 10 or day 11). In some embodiments, the cells are exposed to the inhibitor of BMP signaling (e.g., LDN193189 or “LDN”) from day 0 through day 10, inclusive of each day. In some embodiments, the cells are exposed to the inhibitor of GSK3β signaling (e.g., CHIR99021 or “CHIR”) from day 2 up to about day 13 (e.g., day 12 or day 13). In some embodiments, the cells are exposed to the inhibitor of GSK3β signaling (e.g., CHIR99021 or “CHIR”) from day 2 through day 12.

In some embodiments, the cells are exposed to (i) an inhibitor of TGF-3/activin-Nodal signaling from day 0 up to about day 5 (e.g., day 4 or day 5); (ii) at least one activator of Sonic Hedgehog (SHH) signaling from day 1 up to about day 7 (e.g., day 6 or day 7); (iii) an inhibitor of bone morphogenetic protein (BMP) signaling from day 0 up to about day 11 (e.g., day 10 or day 11); and (iv) an inhibitor of glycogen synthase kinase 3β (GSK3β) signaling from day 2 up to about day 13 (e.g., day 12 or day 13). In some embodiments, the cells are exposed to (i) SB from day 0 up to about day 5 (e.g., day 4 or day 5); (ii) SHH/PUR from dayl up to about day 7 (e.g., day 6 or day 8); (iii) LDN from day 0 up to about day 11 (e.g. day 10 or day 11); and (iv) CHIR from day 2 up to about day 13 (e.g. day 12 or day 13). In some embodiments, the cells are exposed to (i) an inhibitor of TGF-3/activin-Nodal signaling from day 0 through day 4, each day inclusive; (ii) at least one activator of Sonic Hedgehog (SHH) signaling from day 1 through day 6, each day inclusive; (iii) an inhibitor of bone morphogenetic protein (BMP) signaling from day 0 through day 10, each day inclusive; and (iv) an inhibitor of glycogen synthase kinase 3β (GSK3β) signaling from day 2 through day 12, each day inclusive. In some embodiments, the cells are exposed to (i) SB from day 0 through day 4, each day inclusive; (ii) SHH/PUR from day 2 through day 6, each day inclusive; (iii) LDN from day 0 through day 10, each day inclusive; and (iv) CHIR from day 2 through day 12, each day inclusive.

In some embodiments, the cells are exposed to brain-derived neurotrophic factor (BDNF) beginning on day 11. In some embodiments, the cells are exposed to ascorbic acid. In some embodiments, the cells are exposed to glial cell-derived neurotrophic factor (GDNF) beginning on day 11. In some embodiments, the cells are exposed to dibutyryl cyclic AMP (dbcAMP) beginning on day 11. In some embodiments, the cells are exposed to transforming growth factor beta-3 (TGF3β) beginning on day 11. In some embodiments, the cells are exposed to the inhibitor of Notch signaling (e.g., DAPT) beginning on day 11. In some embodiments, beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF3β); and (vi) the inhibitor of Notch signaling (e.g., DAPT) (collectively “BAGCT/DAPT”). In some embodiments, the cells are exposed to BAGCT/DAPT beginning on day 11 until harvest or collection. In some embodiments, the cells are exposed to BAGCT/DAPT from day 11 through day 18. In some embodiments, the cells are exposed to BAGCT/DAPT from day 11 through day 20. In some embodiments, the cells are exposed to BAGCT/DAPT from day 11 through day 25.

In some embodiments, the cells are exposed to a Rho-associated protein kinase (ROCK) inhibitor on day 0. In some embodiments, the cells are exposed to a Rho-associated protein kinase (ROCK) inhibitor on day 7. In some embodiments, the cells are exposed to a Rho-associated protein kinase (ROCK) inhibitor on day 16. In some embodiments, the cells are exposed to a Rho-associated protein kinase (ROCK) inhibitor on day 20. In some embodiments, the cells are exposed to a Rho-associated protein kinase (ROCK) inhibitor on day 0, day 7, day 16, and day 20. In some embodiments, the cells are exposed to a ROCK inhibitor on the day on which the cells are passaged. In some embodiments, the cells are passaged on day 0, 7, 16, 20, or combinations thereof. In some embodiments, the cells are passaged on day 0, 7, 16, and 20.

In some embodiments, the cells are cultured in a basal induction medium comprising DMEM/F-12 and Neurobasal media (e.g., at a 1:1 ratio), supplemented with N2, B27, non-essential amino acids (NEAA), Glutamax, L-glutamine, β-mercaptoethanol, and insulin. In some embodiments, the cells are cultured in the basal induction media from about day 0 through about day 10. In some embodiments, the basal induction media is for differentiating pluripotent stem cells into floor plate midbrain progenitor cells.

In some embodiments, the cells are cultured in a maturation medium comprising Neurobasal media, supplemented with N2, B27, non-essential amino acids (NEAA), and Glutamax. In some embodiments, the cells are cultured in the basal induction media from about day 11 until harvest or collection. In some embodiments, the cells are cultured in the basal induction media from about day 11 through day 18. In some embodiments, the cells are cultured in the basal induction media from about day 11 through day 20. In some embodiments, the maturation media is for differentiating floor plate midbrain progenitor cells into determined dopamine (DA) neuron progenitor cells. In some embodiments, the cells are cultured in the basal induction media from about day 11 through day 25. In some embodiments, the maturation media is for differentiating floor plate midbrain progenitor cells into dopamine (DA) neurons.

In some embodiments, the media is supplemented with small molecules as described above, including SB, SHH/PUR, LDN, CHIR, BAGCT/DAPT, and ROCKi. In some embodiments, the media is changed every day or every other day. In some embodiments the media is changed every day. In some embodiments the media is changed every other day. In some embodiments, the media is changed every day from about day 0 up to about day 17 (e.g., day 16 or day 18). In some embodiments, the media is changed every other day from about day 18 until harvest or collection. In some embodiments, the media is changed every day from about day 0 up to about day 17 (e.g., day 16 or day 18), and then every other day from about day 18 until harvest or collection. In some embodiments, collection or havest is on about day 20.

In some embodiments, a serum replacement is provided in the media from about day 0 up to about day 10 (e.g. day 9 or day 11). In some embodiments, the serum replacement is provided at 5% (v/v) in the media on day 0 and day 1. In some embodiments, the serum replacement is provided at 2% (v/v) in the media on day 2 through day 10. In some embodiments, the serum replacement is provided at 5% (v/v) in the media on day 0 and day 1 and at 2% (v/v) in the media on day 2 through day 10. In some embodiments, serum replacement is not provided in the media after day 10.

In some embodiments, at least about 50% or at least about 75% of the media is changed. In some embodiments, at least about 50% of the media is changed. In some embodiments, at least about 75% of the media is changed. In some embodiments about 100% of the media is changed.

In some embodiments, about 50% or about 75% of the media is changed. In some embodiments, about 50% of the media is changed. In some embodiments, about 75% of the media is changed. In some embodiments about 100% of the media is changed.

In some embodiments, the media is supplemented with small molecules selected from SB, SHH/PUR, LDN, CHIR, BAGCT/DAPT, ROCKi, or a combination thereof.

In some embodiments, cells are harvested between about day 16 and about day 30. In some embodiments, cells are harvested between about day 16 and about day 27. In some embodiments, cells are harvested between about day 18 and about day 25. In some embodiments, cells are harvested on about day 18. In some embodiments, cells are harvested on about day 20. In some embodiments, cells are harvested on about day 25. In some embodiments, the harvested cells are formulated with a cryopreservant, e.g. DMSO. In some embodiments, the harvested cells produced by the method are cryopreserved before use. In some embodiments, such cryopreserved cells are thawed before use or administration to a subject, e.g. a human patient with a neurodegenerative disease or condition, such as Parkinson's disease.

In some embodiments, compositions comprising cells generated by the methods provided herein are used for the treatment of a neurodegenerative disease or condition, such as Parkinson's disease. In some embodiments, a composition of cells generated by any of the methods described herein are administered to a subject who has Parkinson's disease. In some embodiments, a composition of cells generated by any of the methods described herein are administered by stereotactic injection, such as with a catheter. In some embodiments, a composition of cells generated by any of the methods described herein are administered to the striatum of a subject with Parkinson's disease.

### III. Methods of Predicting Engraftment

Provided herein are methods of predicting cell engraftment of a population of neuronal progenitor cells. Also provided herein are methods of assessing a population of neuronal progenitor cells for implantation in a subject to treat a neurodegenerative disease. Also provided herein are methods of selecting a population of neuronal progenitor cells for implantation in a subject to treat a neurodegenerative disease. Also provided herein are methods of assessing engraftment fitness of a population of neuronal progenitor cells.

In some embodiments, the population of neuronal progenitor cells are for implantation in a brain region of the subject if the population of neuronal progenitor cells is predicted to engraft. In some embodiments, the provided methods include predicting if the population of neuronal progenitor cells will engraft in the brain region of the subject following implantation of the population of neuronal progenitor cells into the brain region. In some embodiments, the provided methods include selecting the population of neuronal progenitor cells for implantation in the subject if the population of neuronal progenitor cells is predicted to engraft. In some embodiments, the provided methods include selecting the population of neuronal progenitor cells as a population of neuronal progenitor cells that is predicted to engraft.

In some embodiments, the brain region is the substantia nigra. In some embodiments, the neurodegenerative disease is a Parkinsonism. In some embodiments, the neurodegenerative disease is Parkinson's disease.

In some embodiments, the population of neuronal progenitor cells is from a culture of cells differentiated from pluripotent stem cells. Exemplary types and sources of pluripotent stem cells are described in Section II. For instance, in some embodiments, the pluripotent stem cells are embryonic stem (ES) cells, induced pluripotent stem cells (iPSCs), or a combination thereof. In some embodiments, the pluripotent stem cells are induced pluripotent stem cells. In some embodiments, the pluripotent stem cells are human induced pluripotent stem cells. In some embodiments, the pluripotent stem cells are autologous to the subject. In some embodiments, the pluripotent stem cells are allogeneic to the subject. In some embodiments, the pluripotent stem cells are from a healthy human subject. In some embodiments, the pluripotent stem cells are from a human subject with a neurodegenerative disease or condition. In some embodiments, the neurodegenerative disease or condition includes the loss of dopaminergic neurons. In some embodiments, the neurodegenerative disease or condition is a Parkinsonism. In some embodiments, the neurodegenerative disease or condition is Parkinson's disease. In some embodiments, the pluripotent stem cells are hypoimmunogenic. In some embodiments, the pluripotent stem cells are engineered to remove genes encoding one or more of polymorphic HLA-A/-B/-C and HLA class II molecules. In some embodiments, the pluripotent stem cells are engineered to provide genes encoding one or more of PD-L1, HLA-G, and CD47. In some embodiments, the genes encoding one or more of PD-L1, HLA-G, and CD47 are provided into a AAVS1 safe harbor locus. In some embodiments, the pluripotent stem cells are any as described herein.

In some embodiments, the culture of cells is differentiated from pluripotent stem cells under conditions to neurally differentiate the cells. In some embodiments, the provided methods further include previously differentiating the culture of cells that includes the population of neuronal progenitor cells. In some embodiments, the culture of cells is cultured to differentiate the cells to determined dopaminergic neuron progenitor cells. In some embodiments, the population of neuronal progenitor cells includes determined dopaminergic neuron progenitor cells. Exemplary methods of neurally differentiating cells in order to form the population of neuronal progenitor cells are described in Section II. For instance, in some embodiments, the culture of cells that includes the population of neuronal progenitor cells is differentiated from pluripotent stem cells by a process involving performing a first incubation that includes culturing the pluripotent stem cells (PSCs) in a first culture vessel, wherein beginning at the initiation of the first incubation (day 0), the cells are exposed to (i) an inhibitor of TGF-3/activin-Nodal signaling and (ii) an inhibitor of bone morphogenetic protein (BMP) signaling. In some embodiments, the process further involves performing a second incubation that includes culturing cells produced by the first incubation in a second culture vessel under conditions to neurally differentiate the cells. In some embodiments, the second culture vessel is an adherent culture vessel. In some embodiments, the adherent culture vessel is coated with laminin or a fragment thereof. In some embodiments, the population of neuronal progenitor cells are formed according to any of the methods described herein.

In some embodiments, the provided methods include using gene expression levels of a plurality of genes for one or more cells of the population of neuronal progenitor cells in order to predict if the population of neuronal progenitor cells will engraft following implantation. In some embodiments, the provided methods include obtaining gene expression levels of a plurality of genes for one or more cells of the population of neuronal progenitor cells. In some embodiments, the provided methods include measuring gene expression levels of a plurality of genes for one or more cells of the population of neuronal progenitor cells. Exemplary methods of obtaining or measuring gene expression levels are described in Section III-A.

In some embodiments, the gene expression levels of the plurality of genes are gene expression levels of genes associated with the ability of a population of neuronal progenitor cells to engraft in a brain region of a subject. In some embodiments, the plurality of genes includes one or more cell cycle genes and/or one or more maturity genes. Exemplary cell cycle genes and maturity genes are described in Section III-A-1 and III-A-2, respectively. In some embodiments, the plurality of genes includes one or more cell cycle genes. In some embodiments, the plurality of genes includes only cell cycle genes. In some embodiments, the plurality of genes includes one or more maturity genes. In some embodiments, the plurality of genes includes only maturity genes. In some embodiments, the plurality of genes includes one or more cell cycle genes and one or more maturity genes.

In some embodiments, the provided methods include comparing one or more of the gene expression levels or one or more combinations thereof to one or more predetermined first threshold levels. In some embodiments, gene expression levels or combinations thereof that are greater than the first threshold levels are associated with a population of neuronal progenitor cells that is predicted to engraft in a brain region of a subject following implantation of the population of neuronal progenitor cells in the brain region.

In some embodiments, the provided methods include comparing one or more of the gene expression levels or one or more combinations thereof to one or more predetermined second threshold levels. In some embodiments, gene expression levels or combinations thereof that are less than the second threshold levels are associated with a population of neuronal progenitor cells that is predicted to engraft in a brain region of a subject following implantation of the population of neuronal progenitor cells in the brain region.

In some embodiments, the provided methods include applying the gene expression levels as input to a process configured to predict if the population of neuronal progenitor cells will engraft in the brain region of the subject following implantation of the population of neuronal progenitor cells into the brain region. In some embodiments, the predicting is based on gene expression levels of one or more of the plurality of genes.

In some embodiments, the process includes a machine learning model. Exemplary machine learning models are described in Section III-B. In some embodiments, the machine learning model is trained using gene expression levels of the one or more of the plurality of genes for a plurality of reference populations of neuronal progenitor cells. In some embodiments, the machine learning model is trained also using engraftment fitness of the plurality of reference populations.

Also provided herein are methods of training a machine learning model. In some embodiments, the provided methods include obtaining gene expression levels of one or more of a plurality of genes for a plurality of reference populations of neuronal progenitor cells. In some embodiments, the provided methods include applying the gene expression levels of the plurality of reference populations as input to train a machine learning model. Exemplary machine learning models are described in Section III-B. In some embodiments, the provided methods further include receiving engraftment fitness of the plurality of reference populations. In some embodiments, the provided methods further include applying the engraftment fitness of the plurality of reference populations as input to train the machine learning model. In some embodiments, the machine learning model is trained to predict based on gene expression levels of one or more of the plurality of genes if a population of neuronal progenitor cells will engraft in a brain region of a subject following implantation of the population of neuronal progenitor cells into the brain region. In some embodiments, the population of neuronal progenitor cells is from a culture of cells differentiated from pluripotent stem cells under conditions to neurally differentiate the cells.

In some embodiments, the engraftment fitness of a reference population indicates whether or not the reference population engrafted in a brain region of a subject following implantation of the reference population into the brain region. In some embodiments, the engraftment fitness of a reference population indicates the degree to which the reference population engrafted in a brain region of a subject following implantation of the reference population into the brain region.

In some embodiments, the engraftment fitness of a reference population is determined based on the number of cells of the reference population that are present in the brain region following the implantation. In some embodiments, the number of cells is counted at, about, at least, or at least about 7 days, 14 days, or 21 days following the implantation. In some embodiments, the number of cells is counted at, about, at least, or at least about 7 days following the implantation. In some embodiments, the number of cells is counted at, about, at least, or at least about 14 days following the implantation. In some embodiments, the number of cells is counted at, about, at least, or at least about 21 days following the implantation.

In some embodiments, the predetermined number of cells is greater than or greater than about 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9%, or 10% of the number of cells implanted in the brain region. In some embodiments, a reference population is considered fit for engraftment if at least a predetermined number of cells are present in the brain region following the implantation.

In some embodiments, the predetermined number of cells is greater than or greater than about 0.5% of the number of cells implanted in the brain region. In some embodiments, the predetermined number of cells is greater than or greater than about 0.6% of the number of cells implanted in the brain region. In some embodiments, the predetermined number of cells is greater than or greater than about 0.7% of the number of cells implanted in the brain region. In some embodiments, the predetermined number of cells is greater than or greater than about 0.8% of the number of cells implanted in the brain region. In some embodiments, the predetermined number of cells is greater than or greater than about 0.9% of the number of cells implanted in the brain region. In some embodiments, the predetermined number of cells is greater than or greater than about 1% of the number of cells implanted in the brain region. In some embodiments, the predetermined number of cells is greater than or greater than about 1.1% of the number of cells implanted in the brain region. In some embodiments, the predetermined number of cells is greater than or greater than about 1.2% of the number of cells implanted in the brain region. In some embodiments, the predetermined number of cells is greater than or greater than about 1.3% of the number of cells implanted in the brain region. In some embodiments, the predetermined number of cells is greater than or greater than about 1.4% of the number of cells implanted in the brain region. In some embodiments, the predetermined number of cells is greater than or greater than about 1.5% of the number of cells implanted in the brain region. In some embodiments, the predetermined number of cells is greater than or greater than about 1.6% of the number of cells implanted in the brain region. In some embodiments, the predetermined number of cells is greater than or greater than about 1.7% of the number of cells implanted in the brain region. In some embodiments, the predetermined number of cells is greater than or greater than about 1.8% of the number of cells implanted in the brain region. In some embodiments, the predetermined number of cells is greater than or greater than about 1.9% of the number of cells implanted in the brain region. In some embodiments, the predetermined number of cells is greater than or greater than about 2% of the number of cells implanted in the brain region. In some embodiments, the predetermined number of cells is greater than or greater than about 3% of the number of cells implanted in the brain region. In some embodiments, the predetermined number of cells is greater than or greater than about 4% of the number of cells implanted in the brain region. In some embodiments, the predetermined number of cells is greater than or greater than about 5% of the number of cells implanted in the brain region. In some embodiments, the predetermined number of cells is greater than or greater than about 6% of the number of cells implanted in the brain region. In some embodiments, the predetermined number of cells is greater than or greater than about 7% of the number of cells implanted in the brain region. In some embodiments, the predetermined number of cells is greater than or greater than about 8% of the number of cells implanted in the brain region. In some embodiments, the predetermined number of cells is greater than or greater than about 9% of the number of cells implanted in the brain region. In some embodiments, the predetermined number of cells is greater than or greater than about 10% of the number of cells implanted in the brain region.

In some embodiments, the reference populations have been differentiated from pluripotent stem cells. Exemplary types and sources of pluripotent stem cells are described in Section II. In some embodiments, the pluripotent stem cells are any as described herein.

In some embodiments, the reference populations have been differentiated from pluripotent stem cells from a plurality of subjects. In some embodiments, the plurality of subjects includes at least or at least about 5, 10, 15, 20, 25, 30, 35, or 40 subjects. In some embodiments, the plurality of subjects includes at least or at least about 5 subjects. In some embodiments, the plurality of subjects includes at least or at least about 10 subjects. In some embodiments, the plurality of subjects includes at least or at least about 15 subjects. In some embodiments, the plurality of subjects includes at least or at least about 20 subjects. In some embodiments, the plurality of subjects includes at least or at least about 25 subjects. In some embodiments, the plurality of subjects includes at least or at least about 30 subjects. In some embodiments, the plurality of subjects includes at least or at least about 35 subjects. In some embodiments, the plurality of subjects includes at least or at least about 40 subjects.

In some embodiments, the reference populations include populations of neuronal progenitor cells that engrafted following implantation. In some embodiments, the reference populations include populations of neuronal progenitor cells that did not engraft following implantation. In some embodiments, the reference populations include populations of neuronal progenitor cells that engrafted following implantation as well as populations of neuronal progenitor cells that did not engraft following implantation.

In some embodiments, the reference populations have been differentiated from pluripotent stem cells under conditions to neurally differentiate the cells. In some embodiments, the reference populations have been cultured to differentiate cells to determined dopaminergic neuron progenitor cells. In some embodiments, the reference populations includes determined dopaminergic neuron progenitor cells. Exemplary methods of neurally differentiating cells in order to form the reference populations are described in Section II. In some embodiments, the reference populations are formed according to any of the methods described herein. In some embodiments, the reference populations are all formed using the same method of neurally differenting cells, e.g., using any of the methods described herein. In some embodiments, the reference populations are formed using a number of different methods of neurally differenting cells, e.g., using a number of any of the methods described herein.

In some embodiments, if the population of neuronal progenitor cells is predicted to not engraft, the method further includes repeating the obtaining and applying steps. In some embodiments, the steps are repeated for the same population of neuronal progenitor cells. In some embodiments, the steps are repeated until the population of neuronal progenitor cells is predicted to engraft. In some embodiments, the steps are repeated between or between about 1 and 10 days after the previous iteration of the steps. In some embodiments, the steps are repeated between or between about 1 and 9 days after the previous iteration of the steps. In some embodiments, the steps are repeated between or between about 1 and 8 days after the previous iteration of the steps. In some embodiments, the steps are repeated between or between about 1 and 7 days after the previous iteration of the steps. In some embodiments, the steps are repeated between or between about 1 and 6 days after the previous iteration of the steps. In some embodiments, the steps are repeated between or between about 1 and 5 days after the previous iteration of the steps. In some embodiments, the steps are repeated between or between about 1 and 4 days after the previous iteration of the steps. In some embodiments, the steps are repeated between or between about 1 and 3 days after the previous iteration of the steps. In some embodiments, the steps are repeated between or between about 1 and 2 days after the previous iteration of the steps. In some embodiments, the steps are repeated between or between about 2 and 10 days after the previous iteration of the steps. In some embodiments, the steps are repeated between or between about 2 and 9 days after the previous iteration of the steps. In some embodiments, the steps are repeated between or between about 2 and 8 days after the previous iteration of the steps. In some embodiments, the steps are repeated between or between about 2 and 7 days after the previous iteration of the steps. In some embodiments, the steps are repeated between or between about 2 and 6 days after the previous iteration of the steps. In some embodiments, the steps are repeated between or between about 2 and 5 days after the previous iteration of the steps. In some embodiments, the steps are repeated between or between about 2 and 4 days after the previous iteration of the steps. In some embodiments, the steps are repeated between or between about 2 and 3 days after the previous iteration of the steps. In some embodiments, the steps are repeated between or between about 3 and 10 days after the previous iteration of the steps. In some embodiments, the steps are repeated between or between about 3 and 9 days after the previous iteration of the steps. In some embodiments, the steps are repeated between or between about 3 and 8 days after the previous iteration of the steps. In some embodiments, the steps are repeated between or between about 3 and 7 days after the previous iteration of the steps. In some embodiments, the steps are repeated between or between about 3 and 6 days after the previous iteration of the steps. In some embodiments, the steps are repeated between or between about 3 and 5 days after the previous iteration of the steps. In some embodiments, the steps are repeated between or between about 3 and 4 days after the previous iteration of the steps.

In some embodiments, the steps are repeated for a different population of neuronal progenitor cells. In some embodiments, the different population is also from a culture of cells that have been differented from pluripotent stem cells, e.g., differentiated according to any of the methods described herein. In some embodiments, the pluripotent stem cells are from the same subject. In some embodiments, the steps are repeated until the different population of neuronal progenitor cells is predicted to engraft.

In some embodiments, the provided methods further include harvesting the selected population of neuronal progenitor cells. Exemplary methods of harvesting cells are described in Section II-D. In some embodiments, the selected population is harvested according to any of the methods described herein. In some embodiments, the harvesting is carried out at about day 16 or later. In some embodiments, the selected population at harvest is at about day 16 or later of culture. In some embodiments, the harvesting is carried out between about day 18 and about day 23. In some embodiments, the selected population at harvest is at between about day 18 and about day 23 of culture. In some embodiments, the harvesting is carried out at or about at day 18, day 19, day 20, day 21, day 22, or day 23. In some embodiments, the selected population at harvest is at or about at day 18, day 19, day 20, day 21, day 22, or day 23 of culture. In some embodiments, the harvesting is carried out at or about at day 20. In some embodiments, the selected population at harvest is at or about day 20 of culture.

In some embodiments, the provided methods further include formulating the harvested cells with a cryoprotectant. In some embodiments, the harvested cells are formulated according to any of the methods described herein. In some embodiments, the provided methods further include cryopreserving the formulated cells. In some embodiments, the formulated cells are cryopreserved according to any of the methods described herein. In some embodiments, the cryopreserving includes controlled rate freezing.

### A. Gene Expression Levels

In some embodiments, the provided methods include measuring or obtaining gene expression levels of a plurality of genes. In some embodiments, the gene expression levels are obtained or measured for one or more cells of a population of neuronal progenitor cells. In some embodiments, the gene expression levels are used to predict engraftment of the population of neuronal progenitor cells. In some embodiments, the gene expression levels are measured or obtained for a plurality of reference populations of neuronal progenitor cells. In some embodiments, the gene expression levels are used to train a process, e.g., a machine learning model, for instance to predict cell engraftment.

In some embodiments, gene expression is or includes a process by which information of the gene is used in the synthesis of a gene product. Thus, in some embodiments, a gene product is any biomolecule that is assembled, generated, and/or synthesized with information encoded by a gene, and may include polynucleotides and/or polypeptides. In particular embodiments, assessing, measuring, and/or determining gene expression is or includes determining or measuring the level, amount, or concentration of the gene product. In certain embodiments, the level, amount, or concentration of the gene product may be transformed (e.g., normalized) or directly analyzed (e.g., raw).

In some embodiments, the gene product is or includes a protein, i.e., a polypeptide, that is encoded by and/or expressed by the gene. In particular embodiments, the gene product encodes a protein that is localized and/or exposed on the surface of a cell. In some embodiments, the protein is a soluble protein. In certain embodiments, the protein is secreted by a cell. In particular embodiments, the gene expression is the amount, level, and/or concentration of a protein that is encoded by the gene. In certain embodiments, one or more protein gene products are measured by any suitable means known in the art. Suitable methods for assessing, measuring, determining, and/or quantifying the level, amount, or concentration or more or more protein gene products include, but are not limited to detection with immunoassays, nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays). In some embodiments, the immunoassay is or includes methods or assays that detect proteins based on an immunological reaction, e.g., by detecting the binding of an antibody or antigen binding antibody fragment to a gene product. Immunoassays include, but are not limited to, quantitative immunocytochemisty or immunohistochemisty, ELISA (including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), enzyme immunoassay (EIA), RIA (radioimmunoassay), and SPR (surface plasmon resonance).

In certain embodiments, the gene product is a polynucleotide, e.g., an mRNA or a protein, that is encoded by the gene. In some embodiments, the gene product is a polynucleotide that is expressed by and/or encoded by the gene. In certain embodiments, the polynucleotide is an RNA. In some embodiments, the gene product is a messenger RNA (mRNA), a transfer RNA (tRNA), a ribosomal RNA, a small nuclear RNA, a small nucleolar RNA, an antisense RNA, long non-coding RNA, a microRNA, a Piwi-interacting RNA, a small interfering RNA, and/or a short hairpin RNA. In particular embodiments, the gene product is an mRNA.

In particular embodiments, assessing, measuring, determining, and/or quantifying amount or level of an RNA gene product includes a step of generating, polymerizing, and/or deriving a cDNA polynucleotide and/or a cDNA oligonucleotide from the RNA gene product. In certain embodiments, the RNA gene product is assessed, measured, determined, and/or quantified by directly assessing, measuring, determining, and/or quantifying a cDNA polynucleotide and/or a cDNA oligonucleotide that is derived from the RNA gene product.

In particular embodiments, the amount or level of a polynucleotide in a sample may be assessed, measured, determined, and/or quantified by any suitable means known in the art.

For example, in some embodiments, the amount or level of a polynucleotide gene product can be assessed, measured, determined, and/or quantified by polymerase chain reaction (PCR), including reverse transcriptase (rt) PCR, droplet digital PCR, real-time and quantitative PCR (qPCR) methods (including, e.g., TAQMAN®, molecular beacon, LIGHTUP™, SCORPION™ SIMPLEPROBES®; see, e.g., U.S. Pat. Nos. 5,538,848; 5,925,517; 6,174,670; 6,329,144; 6,326,145 and 6,635,427); northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or any other methods known in the art, such as discussed in Shendure et al., Nat. Rev. Genet. 5:335-44 (2004) or Nowrousian, Euk. Cell 9(9): 1300-1310 (2010), including such specific platforms as HELICOS®, ROCHE@ 454, ILLUMINA®/SOLEXA®, ABI SOLiD®, and POLONATOR® sequencing. In particular embodiments, the levels of nucleic acid gene products are measured by quantitative PCR (qPCR) methods, such qRT-PCR. In some embodiments, the qRT-PCR uses three nucleic acid sets for each gene, where the three nucleic acids comprise a primer pair together with a probe that binds between the regions of a target nucleic acid where the primers bind-known commercially as a TAQMAN® assay.

In particular embodiments, the expression of two or more of the genes are measured or assessed simultaneously. In certain embodiments, a multiplex PCR, e.g., a multiplex rt-PCR assessing or a multiplex quantitative PCR (qPCR) for, measuring, determining, and/or quantifying the level, amount, or concentration of two or more gene products. In some embodiments, microarrays (e.g., AFFYMETRIX®, AGILENT® and ILLUMINA®-style arrays) are used for assessing, measuring, determining, and/or quantifying the level, amount, or concentration of two or more gene products. In some embodiments, microarrays are used for assessing, measuring, determining, and/or quantifying the level, amount, or concentration of a cDNA polynucleotide that is derived from an RNA gene product. In some embodiments, the expression of one or more gene products, e.g., polynucleotide gene products, is determined by sequencing the gene product and/or by sequencing a cDNA polynucleotide that is derived from the from the gene product. In some embodiments, the sequencing is performed by a non-Sanger sequencing method and/or a next generation sequencing (NGS) technique. Examples of Next Generation Sequencing techniques include, but are not limited to Massively Parallel Signature Sequencing (MPSS), Polony sequencing, pyrosequencing, Reversible dye-terminator sequencing, SOLiD sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing.

In some embodiments, the NGS technique is RNA sequencing (RNA-Seq). In particular embodiments, the expression of the one or more polynucleotide gene products is measured, determined, and/or quantified by RNA-Seq. RNA-Seq, also called whole transcriptome shotgun sequencing determines the presence and quantity of RNA in a sample. RNA sequencing methods have been adapted for the most common DNA sequencing platforms [HiSeq systems (Illumina), 454 Genome Sequencer FLX System (Roche), Applied Biosystems SOLiD (Life Technologies), IonTorrent (Life Technologies). These platforms require initial reverse transcription of RNA into cDNA. Conversely, the single molecule sequencer HeliScope (Helicos BioSciences) is able to use RNA as a template for sequencing. A proof of principle for direct RNA sequencing on the PacBio RS platform has also been demonstrated (Pacific Bioscience). In some embodiments, the one or more RNA gene products are assessed, measured, determined, and/or quantified by RNA-seq. In some embodiments, the RNA-seq is a tag-based RNA-seq. In tag-based methods, each transcript is represented by a unique tag. Initially, tag-based approaches were developed as a sequence-based method to measure transcript abundance and identify differentially expressed genes, assuming that the number of tags (counts) directly corresponds to the abundance of the mRNA molecules. The reduced complexity of the sample, obtained by sequencing a defined region, was essential to make the Sanger-based methods affordable. When NGS technology became available, the high number of reads that could be generated facilitated differential gene expression analysis. A transcript length bias in the quantification of gene expression levels, such as observed for shotgun methods, is not encountered in tag-based methods. All tag-based methods are by definition strand specific. In particular embodiments, the one or more RNA gene products are assessed, measured, determined, and/or quantified by tag-based RNA-seq.

In some embodiments, the RNA-seq is a shotgun RNA-seq. Numerous protocols have been described for shotgun RNA-seq, but they have many steps in common: fragmentation (which can occur at RNA level or cDNA level, conversion of the RNA into cDNA (performed by oligo dT or random primers), second-strand synthesis, ligation of adapter sequences at the 3′ and 5′ ends (at RNA or DNA level) and final amplification. In some embodiments, RNA-seq can focus only on polyadenylated RNA molecules (mainly mRNAs but also some lncRNAs, snoRNAs, pseudogenes and histones) if poly(A)+ RNAs are selected prior to fragmentation, or may also include non-polyadenylated RNAs if no selection is performed. In the latter case, ribosomal RNA (more than 80% of the total RNA pool) needs to be depleted prior to fragmentation. It is, therefore, clear that differences in capturing of the mRNA part of the transcriptome lead to a partial overlap in the type of detected transcripts. Moreover, different protocols may affect the abundance and the distribution of the sequenced reads. This makes it difficult to compare results from experiments with different library preparation protocols.

In some embodiments, RNA from each sample is obtained, fragmented and used to generate complementary DNA (cDNA) samples, such as cDNA libraries for sequencing. Reads may be processed and aligned to the human genome and the expected number of mappings per gene/isoform are estimated and used to determine read counts. In some embodiments, read counts are normalized by the length of the genes/isoforms and number of reads in a library to yield FPKM normalized, e.g., by length of the genes/isoforms and number of reads in the library, to yield fragments per kilobase of exon per million mapped reads (FPKM) according to the gene length and total mapped reads. In some aspects, between-sample normalization is achieved by normalization, such as 75th quantile normalization, where each sample is scaled by the median of 75th quantiles from all samples, e.g., to yield quantile-normalized FPKM (FPKQ) values. The FPKQ values may be log-transformed (log 2).

In some embodiments, RNA from each sample is obtained, fragmented and used to generate complementary DNA (cDNA) samples, such as cDNA libraries for sequencing. Reads may be processed and aligned to the human genome and the expected number of mappings per gene/isoform are estimated and used to determine read counts. In some embodiments, read counts are normalized by the length of the genes/isoforms and number of reads in a library. In some embodiments, read counts are provided as counts per million (CPM).

In some embodiments, relative gene expression is measured by comparing the CPM of a target gene to the CPM of a housekeeping gene. In some embodiments, the housekeeping gene is GAPDH. In some embodiments, the relative gene expression of a target gene is determined as the ratio of the CPM of the target gene to CPM of a housekeeping gene (e.g. GAPDH).

In some embodiments, the gene expression levels are obtained using microarray analysis. In some embodiments, the gene expression levels are obtained using RNA sequencing. In some embodiments, the gene expression levels are obtained using both microarray analysis and RNA sequencing. In some embodiments, the RNA sequencing is performed on bulk RNA from a plurality of cells. In some embodiments, bulk RNA sequencing data is obtained from pooled RNA from the plurality of cells. In some embodiments, the RNA sequencing is performed on single cells. In some embodiments, the RNA sequencing is performed on bulk RNA from a plurality of cells and on single cells.

Any known and available methods for obtaining bulk RNA sequencing data can be used (for example, see Chao et al., 2019, BMC Genomics 20: 571, incorporated by reference herein in its entirety). For instance, total RNA from a sample, e.g., a plurality of cells from a population of cells, can be isolated using TRIZOL, treated with DNase I, and purified. Concentration and quality of isolated RNA can be measured and checked prior to library preparation for total RNA or mRNA. For library preparation, total RNA or mRNA can be fragmented and converted to cDNA using reverse transcription. After construction, amplification, and optional barcoding of double-stranded cDNA, libraries can be processed for next generation sequencing using any known and available library preparation techniques, sequencing platforms, and genomic-alignment tools.

In some embodiments, the gene expression levels are obtained using single-cell RNA sequencing. In some embodiments, the use of single-cell RNA sequencing data affords certain advantages. In some embodiments, the use of single-cell RNA sequencing data allows for characterization of subpopulations of cells, for instance of determined dopaminergic precursor cells within a larger population of cells. In some embodiments, the use of single-cell RNA sequencing data reduces the number of cells required for use in the methods provided herein, e.g., reduces the number of cells needed to obtain data for training a machine learning model, or reduces the number of cells needed to predict engraftment of a population of neuronal progenitor cells. In some embodiments, the use of single-cell RNA sequencing data improves characteriziation of biological variability across cells. In some embodiments, the use of single-cell RNA sequencing data allows for easier validation and interpretation of gene expression levels.

Any known and available methods for single-cell RNA sequencing can be used (for example, see Zheng et al., 2017 (Nature Communications 8: 14049), and Haque et al., 2017 (Genome Medicine 9: 75, incorporated by reference herein in their entirety). For single-RNA sequencing, single cells from a sample, for instance an in vitro population of cells, can be isolated using flow cytometric cell-sorting, microfluidic platform, or droplet-based methods. Isolated cells are lysed to allow capture of RNA molecules. Poly[T]-primers can be used for the analysis of polyadenylated mRNA molecules specifically, and primed mRNA molecules are converted to cDNA using reverse transcription. In some instances, unique molecular identifiers can be used to mark single mRNA molecules based on cellular origin. The cDNA pool can then amplified, optionally barcoded, and sequenced, for instance using next-generation sequencing (NGS) and with library preparation techniques, sequencing platforms, and genomic-alignment tools similar to those used for bulk RNA samples. In some instances, unbiased cell-type classification witin a mixed population of distinct cell types can be achieved with as few as 10,000 to 50,000 reads per cell, and single-cell libraries from various common protocols can be close to saturation when sequenced to a depth of 1,000,000 reads.

In some embodiments, the gene expression levels include bulk RNA sequencing data and single-cell RNA sequencing data. In some embodiments, the bulk RNA sequencing data and the single-cell RNA sequencing data are obtained from the same population of cells. In some embodiments, the single-cell RNA sequencing data can be used to approximate the bulk RNA sequencing data obtained from the same population of cells. In some embodiments, approximated bulk RNA sequencing data is obtained by averaging single-cell RNA sequencing data from cells in the same population of cells. In some embodiments, the gene expression levels include approximated bulk RNA sequencing data.

In some embodiments, the plurality of genes include one or more of AC104083.1, ACE, ACSL1, ACSS3, ADSS, AFAP1, ANLN, ANP32A, ANXA11, APBA1, ARHGDIG, ARL8A, ASPH, ASPM, AURKA, AURKB, BDNF, BICDL1, BIRC5, BRINP1, BUB1, BUB1B, CAMK2B, CCDC112, CCDC160, CCDC60, CCNA2, CCNB1, CCNB2, CDC20, CDC25C, CDCA2, CDCA8, CDK1, CDK5R2, CDKN1A, CENPE, CENPF, CEP170B, CEP55, CHGB, CIT, COL23A1, CTSC, CYFIP1, DAAM2, DIRAS1, DLGAP5, DMTN, DNAJB5, DPY19L1, DUSP26, ECT2, ESPL1, FABP7, FAM71E2, FAM83D, FAM86C2P, FANCD2, FBXL16, FNBP1L, FOXM1, FZD2, GEM, GFOD2, GTSE1, GUCY1A1, HAPLN3, HCN3, HJURP, HLA-E, HMMR, HTATIP2, IKZF2, IL4R, IQGAP3, ITGA5, JPT1, KCNB1, KCNC1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MGST1, MIR100HG, MKI67, MRVI1, MYCBP, NAALAD2, NACAD, NAV2, NCAM1, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SALL4, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TOP2A, TPH1, TPX2, TRIM46, TTK, TUBA1C, UBE2C, and YBX3.

In some embodiments, the plurality of genes includes one or more of AC104083.1, ACE, ACSL1, ACSS3, ADSS, AFAP1, ANLN, ANP32A, ANXA11, ARHGDIG, ARL8A, ASPH, AURKA, AURKB, BICDL1, BIRC5, BRINP1, BUB1, BUB1B, CCDC112, CCDC160, CCDC60, CCNA2, CCNB1, CCNB2, CDC20, CDC25C, CDCA2, CDCA8, CDK1, CDKN1A, CENPE, CENPF, CEP170B, CEP55, CHGB, CIT, CYFIP1, DAAM2, DIRAS1, DLGAP5, DNAJB5, DPY19L1, DUSP26, ECT2, ESPL1, FAM71E2, FAM83D, FAM86C2P, FANCD2, FBXL16, FNBP1L, FOXM1, FZD2, GEM, GFOD2, GTSE1, GUCY1A1, HCN3, HJURP, HLA-E, HMMR, HTATIP2, IKZF2, IQGAP3, ITGA5, JPT1, KCNB1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MIR100HG, MKI67, MRVI1, MYCBP, NAALAD2, NACAD, NAV2, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TPH1, TPX2, TRIM46, TTK, TUBA1C, and UBE2C.

In some embodiments, the plurality of genes includes one or more of ACE, ACSL1, ANP32A, ARL8A, AURKB, BDNF, BIRC5, CCDC112, CCDC160, CCDC60, CCNB1, CCNB2, CDC20, CDC25C, CDCA8, CDK1, CEP170B, CHGB, COL23A1, DAAM2, DLGAP5, DMTN, ESPL1, FABP7, FAM71E2, FAM83D, FNBP1L, GTSE1, HAPLN3, HJURP, HTATIP2, IQGAP3, KCNB1, KIF1A, KIF20A, KIF2C, KIFC1, LINC01128, LRIG1, MAP3K9, MGST1, MIR100HG, MRVI1, NAALAD2, NACAD, NDC80, NEK2, NFIC, NFIX, NR6A1, NT5DC1, NUF2, NUSAP1, PLAG1, PRKACB, PRTG, PTCH1, PTPN13, PTTG1, SALL4, SAPCD2, SEMA5B, SLC35D2, SLC6A17, STOX1, SUCLG2, SYT13, TACC3, TGFBR3, TOP2A, TPH1, TPX2, UBE2C, and YBX3.

In some embodiments, the plurality of genes includes one or more of ACE, ACSL1, ANP32A, ARL8A, AURKB, BIRC5, CCDC112, CCDC160, CCDC60, CCNB1, CCNB2, CDC20, CDC25C, CDCA8, CDK1, CEP170B, CHGB, COL23A1, DAAM2, DLGAP5, ESPL1, FABP7, FAM71E2, FAM83D, FNBP1L, GTSE1, HAPLN3, HJURP, HTATIP2, IQGAP3, KCNB1, KIF1A, KIF20A, KIF2C, KIFC1, LINC01128, LRIG1, MAP3K9, MGST1, MIR100HG, MRVI1, NAALAD2, NACAD, NDC80, NEK2, NFIC, NFIX, NR6A1, NT5DC1, NUF2, NUSAP1, PLAG1, PRKACB, PRTG, PTCH1, PTPN13, PTTG1, SALL4, SAPCD2, SEMA5B, SLC35D2, SLC6A17, STOX1, SUCLG2, SYT13, TACC3, TGFBR3, TOP2A, TPH1, TPX2, UBE2C, and YBX3.

In some embodiments, the plurality of genes includes, includes about, includes greater than, or includes greater than about 2 genes. In some embodiments, the plurality of genes includes, includes about, includes greater than, or includes greater than about 3 genes. In some embodiments, the plurality of genes includes, includes about, includes greater than, or includes greater than about 4 genes. In some embodiments, the plurality of genes includes, includes about, includes greater than, or includes greater than about 5 genes. In some embodiments, the plurality of genes includes, includes about, includes greater than, or includes greater than about 6 genes. In some embodiments, the plurality of genes includes, includes about, includes greater than, or includes greater than about 7 genes. In some embodiments, the plurality of genes includes, includes about, includes greater than, or includes greater than about 8 genes. In some embodiments, the plurality of genes includes, includes about, includes greater than, or includes greater than about 9 genes. In some embodiments, the plurality of genes includes, includes about, includes greater than, or includes greater than about 10 genes. In some embodiments, the plurality of genes includes, includes about, includes greater than, or includes greater than about 12 genes. In some embodiments, the plurality of genes includes, includes about, includes greater than, or includes greater than about 14 genes. In some embodiments, the plurality of genes includes, includes about, includes greater than, or includes greater than about 16 genes. In some embodiments, the plurality of genes includes, includes about, includes greater than, or includes greater than about 18 genes. In some embodiments, the plurality of genes includes, includes about, includes greater than, or includes greater than about 20 genes. In some embodiments, the plurality of genes includes, includes about, includes greater than, or includes greater than about 25 genes. In some embodiments, the plurality of genes includes, includes about, includes greater than, or includes greater than about 30 genes. In some embodiments, the plurality of genes includes, includes about, includes greater than, or includes greater than about 35 genes. In some embodiments, the plurality of genes includes, includes about, includes greater than, or includes greater than about 40 genes. In some embodiments, the plurality of genes includes, includes about, includes greater than, or includes greater than about 45 genes. In some embodiments, the plurality of genes includes, includes about, includes greater than, or includes greater than about 55 genes. In some embodiments, the plurality of genes includes, includes about, includes greater than, or includes greater than about 60 genes. In some embodiments, the plurality of genes includes, includes about, includes greater than, or includes greater than about 62 genes. In some embodiments, the plurality of genes includes, includes about, includes greater than, or includes greater than about 64 genes. In some embodiments, the plurality of genes includes, includes about, includes greater than, or includes greater than about 66 genes. In some embodiments, the plurality of genes includes, includes about, includes greater than, or includes greater than about 68 genes. In some embodiments, the plurality of genes includes, includes about, includes greater than, or includes greater than about 70 genes. In some embodiments, the plurality of genes includes, includes about, includes greater than, or includes greater than about 80 genes. In some embodiments, the plurality of genes includes, includes about, includes greater than, or includes greater than about 90 genes. In some embodiments, the plurality of genes includes, includes about, includes greater than, or includes greater than about 100 genes. In some embodiments, the plurality of genes includes, includes about, includes greater than, or includes greater than about 110 genes. In some embodiments, the plurality of genes includes, includes about, includes greater than, or includes greater than about 120 genes. In some embodiments, the plurality of genes includes, includes about, includes greater than, or includes greater than about 130 genes.

In some embodiments, the plurality of genes includes, includes about, includes greater than, or includes greater than about 140 genes. In some embodiments, the plurality of genes includes, includes about, includes greater than, or includes greater than about 150 genes. In some embodiments, the plurality of genes includes, includes about, includes greater than, or includes greater than about 160 genes. In some embodiments, the plurality of genes includes, includes about, includes greater than, or includes greater than about 170 genes. In some embodiments, the plurality of genes includes, includes about, includes greater than, or includes greater than about 180 genes. In some embodiments, the plurality of genes includes, includes about, includes greater than, or includes greater than about 190 genes. In some embodiments, the plurality of genes includes, includes about, includes greater than, or includes greater than about 200 genes.

In some embodiments, the plurality of genes includes between about 2 and 200 genes. In some embodiments, the plurality of genes includes between about 2 and 190 genes. In some embodiments, the plurality of genes includes between about 2 and 180 genes. In some embodiments, the plurality of genes includes between about 2 and 170 genes. In some embodiments, the plurality of genes includes between about 2 and 160 genes. In some embodiments, the plurality of genes includes between about 2 and 150 genes. In some embodiments, the plurality of genes includes between about 2 and 140 genes. In some embodiments, the plurality of genes includes between about 2 and 130 genes. In some embodiments, the plurality of genes includes between about 2 and 120 genes. In some embodiments, the plurality of genes includes between about 2 and 110 genes. In some embodiments, the plurality of genes includes between about 2 and 100 genes. In some embodiments, the plurality of genes includes between about 2 and 90 genes. In some embodiments, the plurality of genes includes between about 2 and 80 genes. In some embodiments, the plurality of genes includes between about 2 and 70 genes. In some embodiments, the plurality of genes includes between about 2 and 60 genes. In some embodiments, the plurality of genes includes between about 2 and 50 genes. In some embodiments, the plurality of genes includes between about 2 and 40 genes. In some embodiments, the plurality of genes includes between about 2 and 30 genes. In some embodiments, the plurality of genes includes between about 2 and 20 genes. In some embodiments, the plurality of genes includes between about 2 and 10 genes. In some embodiments, the plurality of genes includes between about 2 and 5 genes. In some embodiments, the plurality of genes includes between about 5 and 200 genes. In some embodiments, the plurality of genes includes between about 5 and 190 genes. In some embodiments, the plurality of genes includes between about 5 and 180 genes. In some embodiments, the plurality of genes includes between about 5 and 170 genes. In some embodiments, the plurality of genes includes between about 5 and 160 genes. In some embodiments, the plurality of genes includes between about 5 and 150 genes. In some embodiments, the plurality of genes includes between about 5 and 140 genes. In some embodiments, the plurality of genes includes between about 5 and 130 genes. In some embodiments, the plurality of genes includes between about 5 and 120 genes. In some embodiments, the plurality of genes includes between about 5 and 110 genes. In some embodiments, the plurality of genes includes between about 5 and 100 genes. In some embodiments, the plurality of genes includes between about 5 and 90 genes. In some embodiments, the plurality of genes includes between about 5 and 80 genes. In some embodiments, the plurality of genes includes between about 5 and 70 genes. In some embodiments, the plurality of genes includes between about 5 and 60 genes. In some embodiments, the plurality of genes includes between about 5 and 50 genes. In some embodiments, the plurality of genes includes between about 5 and 40 genes. In some embodiments, the plurality of genes includes between about 5 and 30 genes. In some embodiments, the plurality of genes includes between about 5 and 20 genes. In some embodiments, the plurality of genes includes between about 5 and 10 genes. In some embodiments, the plurality of genes includes between about 10 and 200 genes. In some embodiments, the plurality of genes includes between about 10 and 190 genes. In some embodiments, the plurality of genes includes between about 10 and 180 genes. In some embodiments, the plurality of genes includes between about 10 and 170 genes. In some embodiments, the plurality of genes includes between about 10 and 160 genes. In some embodiments, the plurality of genes includes between about 10 and 150 genes. In some embodiments, the plurality of genes includes between about 10 and 140 genes. In some embodiments, the plurality of genes includes between about 10 and 130 genes. In some embodiments, the plurality of genes includes between about 10 and 120 genes. In some embodiments, the plurality of genes includes between about 10 and 110 genes. In some embodiments, the plurality of genes includes between about 10 and 100 genes. In some embodiments, the plurality of genes includes between about 10 and 90 genes. In some embodiments, the plurality of genes includes between about 10 and 80 genes. In some embodiments, the plurality of genes includes between about 10 and 70 genes. In some embodiments, the plurality of genes includes between about 10 and 60 genes. In some embodiments, the plurality of genes includes between about 10 and 50 genes. In some embodiments, the plurality of genes includes between about 10 and 40 genes. In some embodiments, the plurality of genes includes between about 10 and 30 genes. In some embodiments, the plurality of genes includes between about 10 and 20 genes. In some embodiments, the plurality of genes includes between about 20 and 200 genes. In some embodiments, the plurality of genes includes between about 20 and 190 genes. In some embodiments, the plurality of genes includes between about 20 and 180 genes. In some embodiments, the plurality of genes includes between about 20 and 170 genes. In some embodiments, the plurality of genes includes between about 205 and 160 genes. In some embodiments, the plurality of genes includes between about 20 and 150 genes. In some embodiments, the plurality of genes includes between about 20 and 140 genes. In some embodiments, the plurality of genes includes between about 20 and 130 genes. In some embodiments, the plurality of genes includes between about 20 and 120 genes. In some embodiments, the plurality of genes includes between about 20 and 110 genes. In some embodiments, the plurality of genes includes between about 20 and 100 genes. In some embodiments, the plurality of genes includes between about 20 and 90 genes. In some embodiments, the plurality of genes includes between about 20 and 80 genes. In some embodiments, the plurality of genes includes between about 20 and 70 genes. In some embodiments, the plurality of genes includes between about 20 and 60 genes. In some embodiments, the plurality of genes includes between about 20 and 50 genes. In some embodiments, the plurality of genes includes between about 20 and 40 genes. In some embodiments, the plurality of genes includes between about 20 and 30 genes. In some embodiments, the plurality of genes includes between about 30 and 200 genes. In some embodiments, the plurality of genes includes between about 30 and 190 genes. In some embodiments, the plurality of genes includes between about 30 and 180 genes. In some embodiments, the plurality of genes includes between about 30 and 170 genes. In some embodiments, the plurality of genes includes between about 30 and 160 genes. In some embodiments, the plurality of genes includes between about 30 and 150 genes. In some embodiments, the plurality of genes includes between about 30 and 140 genes. In some embodiments, the plurality of genes includes between about 30 and 130 genes. In some embodiments, the plurality of genes includes between about 30 and 120 genes. In some embodiments, the plurality of genes includes between about 30 and 110 genes. In some embodiments, the plurality of genes includes between about 30 and 100 genes. In some embodiments, the plurality of genes includes between about 30 and 90 genes. In some embodiments, the plurality of genes includes between about 30 and 80 genes. In some embodiments, the plurality of genes includes between about 30 and 70 genes. In some embodiments, the plurality of genes includes between about 30 and 60 genes. In some embodiments, the plurality of genes includes between about 30 and 50 genes. In some embodiments, the plurality of genes includes between about 30 and 40 genes. In some embodiments, the plurality of genes includes between about 40 and 200 genes. In some embodiments, the plurality of genes includes between about 40 and 190 genes. In some embodiments, the plurality of genes includes between about 40 and 180 genes. In some embodiments, the plurality of genes includes between about 40 and 170 genes. In some embodiments, the plurality of genes includes between about 40 and 160 genes. In some embodiments, the plurality of genes includes between about 40 and 150 genes. In some embodiments, the plurality of genes includes between about 40 and 140 genes. In some embodiments, the plurality of genes includes between about 40 and 130 genes. In some embodiments, the plurality of genes includes between about 40 and 120 genes. In some embodiments, the plurality of genes includes between about 40 and 110 genes. In some embodiments, the plurality of genes includes between about 40 and 100 genes. In some embodiments, the plurality of genes includes between about 40 and 90 genes. In some embodiments, the plurality of genes includes between about 40 and 80 genes. In some embodiments, the plurality of genes includes between about 40 and 70 genes. In some embodiments, the plurality of genes includes between about 40 and 60 genes. In some embodiments, the plurality of genes includes between about 40 and 50 genes. In some embodiments, the plurality of genes includes between about 50 and 200 genes. In some embodiments, the plurality of genes includes between about 50 and 190 genes. In some embodiments, the plurality of genes includes between about 50 and 180 genes. In some embodiments, the plurality of genes includes between about 50 and 170 genes. In some embodiments, the plurality of genes includes between about 50 and 160 genes. In some embodiments, the plurality of genes includes between about 50 and 150 genes. In some embodiments, the plurality of genes includes between about 50 and 140 genes. In some embodiments, the plurality of genes includes between about 50 and 130 genes. In some embodiments, the plurality of genes includes between about 50 and 120 genes. In some embodiments, the plurality of genes includes between about 50 and 110 genes. In some embodiments, the plurality of genes includes between about 50 and 100 genes. In some embodiments, the plurality of genes includes between about 50 and 90 genes. In some embodiments, the plurality of genes includes between about 50 and 80 genes. In some embodiments, the plurality of genes includes between about 50 and 70 genes. In some embodiments, the plurality of genes includes between about 50 and 60 genes. In some embodiments, the plurality of genes includes between about 60 and 200 genes. In some embodiments, the plurality of genes includes between about 60 and 190 genes. In some embodiments, the plurality of genes includes between about 60 and 180 genes. In some embodiments, the plurality of genes includes between about 60 and 170 genes. In some embodiments, the plurality of genes includes between about 60 and 160 genes. In some embodiments, the plurality of genes includes between about 60 and 150 genes. In some embodiments, the plurality of genes includes between about 60 and 140 genes. In some embodiments, the plurality of genes includes between about 60 and 130 genes. In some embodiments, the plurality of genes includes between about 60 and 120 genes. In some embodiments, the plurality of genes includes between about 60 and 110 genes. In some embodiments, the plurality of genes includes between about 60 and 100 genes. In some embodiments, the plurality of genes includes between about 60 and 90 genes. In some embodiments, the plurality of genes includes between about 60 and 80 genes. In some embodiments, the plurality of genes includes between about 60 and 70 genes. In some embodiments, the plurality of genes includes between about 70 and 200 genes. In some embodiments, the plurality of genes includes between about 70 and 190 genes. In some embodiments, the plurality of genes includes between about 70 and 180 genes. In some embodiments, the plurality of genes includes between about 70 and 170 genes. In some embodiments, the plurality of genes includes between about 70 and 160 genes. In some embodiments, the plurality of genes includes between about 70 and 150 genes. In some embodiments, the plurality of genes includes between about 70 and 140 genes. In some embodiments, the plurality of genes includes between about 70 and 130 genes. In some embodiments, the plurality of genes includes between about 70 and 120 genes. In some embodiments, the plurality of genes includes between about 70 and 110 genes. In some embodiments, the plurality of genes includes between about 70 and 100 genes. In some embodiments, the plurality of genes includes between about 70 and 90 genes. In some embodiments, the plurality of genes includes between about 70 and 80 genes. In some embodiments, the plurality of genes includes between about 80 and 200 genes. In some embodiments, the plurality of genes includes between about 80 and 190 genes. In some embodiments, the plurality of genes includes between about 80 and 180 genes. In some embodiments, the plurality of genes includes between about 80 and 170 genes. In some embodiments, the plurality of genes includes between about 80 and 160 genes. In some embodiments, the plurality of genes includes between about 80 and 150 genes. In some embodiments, the plurality of genes includes between about 80 and 140 genes. In some embodiments, the plurality of genes includes between about 80 and 130 genes. In some embodiments, the plurality of genes includes between about 80 and 120 genes. In some embodiments, the plurality of genes includes between about 80 and 110 genes. In some embodiments, the plurality of genes includes between about 80 and 100 genes. In some embodiments, the plurality of genes includes between about 80 and 90 genes. In some embodiments, the plurality of genes includes between about 90 and 200 genes. In some embodiments, the plurality of genes includes between about 90 and 190 genes. In some embodiments, the plurality of genes includes between about 90 and 180 genes. In some embodiments, the plurality of genes includes between about 90 and 170 genes. In some embodiments, the plurality of genes includes between about 90 and 160 genes. In some embodiments, the plurality of genes includes between about 90 and 150 genes. In some embodiments, the plurality of genes includes between about 90 and 140 genes. In some embodiments, the plurality of genes includes between about 90 and 130 genes. In some embodiments, the plurality of genes includes between about 90 and 120 genes. In some embodiments, the plurality of genes includes between about 90 and 110 genes. In some embodiments, the plurality of genes includes between about 90 and 100 genes. In some embodiments, the plurality of genes includes between about 100 and 200 genes. In some embodiments, the plurality of genes includes between about 100 and 190 genes. In some embodiments, the plurality of genes includes between about 100 and 180 genes. In some embodiments, the plurality of genes includes between about 100 and 170 genes. In some embodiments, the plurality of genes includes between about 100 and 160 genes. In some embodiments, the plurality of genes includes between about 100 and 150 genes. In some embodiments, the plurality of genes includes between about 100 and 140 genes. In some embodiments, the plurality of genes includes between about 100 and 130 genes. In some embodiments, the plurality of genes includes between about 100 and 120 genes. In some embodiments, the plurality of genes includes between about 100 and 110 genes. In some embodiments, the plurality of genes includes between about 110 and 200 genes. In some embodiments, the plurality of genes includes between about 110 and 190 genes. In some embodiments, the plurality of genes includes between about 110 and 180 genes. In some embodiments, the plurality of genes includes between about 110 and 170 genes. In some embodiments, the plurality of genes includes between about 110 and 160 genes. In some embodiments, the plurality of genes includes between about 110 and 150 genes. In some embodiments, the plurality of genes includes between about 110 and 140 genes. In some embodiments, the plurality of genes includes between about 110 and 130 genes. In some embodiments, the plurality of genes includes between about 110 and 120 genes. In some embodiments, the plurality of genes includes between about 120 and 200 genes. In some embodiments, the plurality of genes includes between about 120 and 190 genes. In some embodiments, the plurality of genes includes between about 120 and 180 genes. In some embodiments, the plurality of genes includes between about 120 and 170 genes. In some embodiments, the plurality of genes includes between about 120 and 160 genes. In some embodiments, the plurality of genes includes between about 120 and 150 genes. In some embodiments, the plurality of genes includes between about 120 and 140 genes. In some embodiments, the plurality of genes includes between about 120 and 130 genes. In some embodiments, the plurality of genes includes between about 130 and 200 genes. In some embodiments, the plurality of genes includes between about 130 and 190 genes. In some embodiments, the plurality of genes includes between about 130 and 180 genes. In some embodiments, the plurality of genes includes between about 130 and 170 genes. In some embodiments, the plurality of genes includes between about 130 and 160 genes. In some embodiments, the plurality of genes includes between about 130 and 150 genes. In some embodiments, the plurality of genes includes between about 130 and 140 genes. In some embodiments, the plurality of genes includes between about 140 and 200 genes. In some embodiments, the plurality of genes includes between about 140 and 190 genes. In some embodiments, the plurality of genes includes between about 140 and 180 genes. In some embodiments, the plurality of genes includes between about 140 and 170 genes. In some embodiments, the plurality of genes includes between about 140 and 160 genes. In some embodiments, the plurality of genes includes between about 140 and 150 genes. In some embodiments, the plurality of genes includes between about 150 and 200 genes. In some embodiments, the plurality of genes includes between about 150 and 190 genes. In some embodiments, the plurality of genes includes between about 150 and 180 genes. In some embodiments, the plurality of genes includes between about 150 and 170 genes. In some embodiments, the plurality of genes includes between about 150 and 160 genes. In some embodiments, the plurality of genes includes between about 160 and 200 genes. In some embodiments, the plurality of genes includes between about 160 and 190 genes. In some embodiments, the plurality of genes includes between about 160 and 180 genes. In some embodiments, the plurality of genes includes between about 160 and 170 genes. In some embodiments, the plurality of genes includes between about 170 and 200 genes. In some embodiments, the plurality of genes includes between about 170 and 190 genes. In some embodiments, the plurality of genes includes between about 170 and 180 genes. In some embodiments, the plurality of genes includes between about 180 and 200 genes. In some embodiments, the plurality of genes includes between about 180 and 190 genes. In some embodiments, the plurality of genes includes between about 190 and 200 genes.

### 1. Cell Cycle Genes

In some embodiments, the plurality of genes include one or more cell cycle genes. In some embodiments, the plurality of genes include only cell cycle genes. In some embodiments, the one or more cell cycle genes include one or more of ANLN, ASPM, AURKA, AURKB, BIRC5, BRINP1, BUB1, BUB1B, CCNA2, CCNB1, CCNB2, CDC20, CDC25C, CDCA2, CDCA8, CDK1, CDKN1A, CENPE, CENPF, CEP55, CIT, DLGAP5, ECT2, ESPL1, FAM83D, FANCD2, FOXM1, GEM, GTSE1, HJURP, HMMR, IQGAP3, KIF11, KIF14, KIF15, KIF18A, KIF20A, KIF23, KIF2C, KIF4A, KIFC1, KNL1, MELK, MKI67, NCAPG, NCAPH, NDC80, NEK2, NUF2, NUSAP1, PBK, PIMREG, PLK1, PLK2, POC1A, PRC1, PRR11, PTTG1, RACGAP1, SAPCD2, SKA3, SPAG5, TACC3, TOP2A, TPX2, TTK, TUBA1C, and UBE2C. In some embodiments, the one or more cell cycle genes include BRINP1, CDKN1A, FAM83D, FANCD2, GEM, PLK2, and SAPCD2. In some embodiments, the one or more cell cycle genes include only BRINP1, CDKN1A, FAM83D, FANCD2, GEM, PLK2, and SAPCD2. In some embodiments, the plurality of genes include only BRINP1, CDKN1A, FAM83D, FANCD2, GEM, PLK2, and SAPCD2.

In some embodiments, the plurality of genes includes one or more of ANLN, ASPM, AURKA, AURKB, BIRC5, BRINP1, BUB1, BUB1B, CCNA2, CCNB1, CCNB2, CDC20, CDC25C, CDCA2, CDCA8, CDK1, CDKN1A, CENPE, CENPF, CEP55, CIT, DLGAP5, ECT2, ESPL1, FAM83D, FANCD2, FOXM1, GEM, GTSE1, HJURP, HMMR, IQGAP3, KIF11, KIF14, KIF15, KIF18A, KIF20A, KIF23, KIF2C, KIF4A, KIFC1, KNL1, MELK, MKI67, NCAPG, NCAPH, NDC80, NEK2, NUF2, NUSAP1, PBK, PIMREG, PLK1, PLK2, POC1A, PRC1, PRR11, PTTG1, RACGAP1, SAPCD2, SKA3, SPAG5, TACC3, TPX2, TTK, TUBA1C, and UBE2C.

In some embodiments, the plurality of genes includes one or more of AURKB, BIRC5, CCNB1, CCNB2, CDC20, CDC25C, CDCA8, CDK1, DLGAP5, ESPL1, FAM83D, GTSE1, HJURP, IQGAP3, KIF20A, KIF2C, KIFC1, NDC80, NEK2, NUF2, NUSAP1, PTTG1, SAPCD2, TACC3, TOP2A, TPX2, and UBE2C.

In some embodiments, the plurality of genes includes one or more of AURKB, BIRC5, CCNB1, CCNB2, CDC20, CDC25C, CDCA8, CDK1, DLGAP5, ESPL1, FAM83D, GTSE1, HJURP, IQGAP3, KIF20A, KIF2C, KIFC1, NDC80, NEK2, NUF2, NUSAP1, PTTG1, SAPCD2, TACC3, TPX2, and UBE2C.

### 2. Maturity Genes

In some embodiments, the plurality of genes include one or more maturity genes. In some embodiments, the plurality of genes include only maturity genes. In some embodiments, the one or more maturity genes include genes whose gene expression levels increase or decrease during differentiation. In some embodiments, the one or more maturity genes include genes whose gene expression levels increase or decrease during days 17-22 of differentiation, e.g., differentiation of cells according to any of the methods described herein. In some embodiments, the one or more maturity genes include genes whose gene expression levels increase or decrease substantially monotonically during differentiation. In some embodiments, the one or more maturity genes include one or more of AC104083.1, ACE, ACSL1, AFAP1, APBA1, ARHGDIG, ARL8A, BDNF, BICDL1, CAMK2B, CCDC112, CDK5R2, CEP170B, CHGB, DIRAS1, DMTN, DNAJB5, DUSP26, FBXL16, FNBP1L, GFOD2, GUCY1A1, HCN3, JPT1, KCNB1, KCNC1, KCNH6, KIF1A, KLF7, LINC01128, MACO1, MAP3K9, MAPRE3, MIR100HG, NACAD, NCAM1, NFIC, NFIX, PARP6, PRKACB, RIMS1, SBK1, SHISA7, SLC6A17, SPTBN1, SRGAP2, SYT13, TMEM151B, TPH1, TRIM46, ACSS3, ADSS, ANP32A, ANXA11, ASPH, CCDC160, CCDC60, COL23A1, CTSC, CYFIP1, DAAM2, DPY19L1, FABP7, FAM71E2, FAM86C2P, FZD2, HAPLN3, HLA-E, HTATIP2, IKZF2, IL4R, ITGA5, KCNJ2-AS1, LPIN3, LRIG1, LRIG3, MGST1, MRVI1, MYCBP, NAALAD2, NAV2, NR6A1, NT5DC1, PLAG1, POFUT2, PRTG, PTCH1, PTPN13, SALL4, SAV1, SELENOP, SEMA5B, SLC35D2, SLC66A3, STOX1, SUCLG2, TGFBR3, TM6SF2, TOB1, and YBX3.

In some embodiments, the plurality of genes includes one or more of AC104083.1, ACE, ACSL1, ACSS3, ADSS, AFAP1, ANP32A, ANXA11, ARHGDIG, ARL8A, ASPH, BICDL1, CCDC112, CCDC160, CCDC60, CEP170B, CHGB, CYFIP1, DAAM2, DIRAS1, DNAJB5, DPY19L1, DUSP26, FAM71E2, FAM86C2P, FBXL16, FNBP1L, FZD2, GFOD2, GUCY1A1, HCN3, HLA-E, HTATIP2, IKZF2, ITGA5, JPT1, KCNB1, KCNH6, KCNJ2-AS1, KIF1A, KLF7, LINC01128, LPIN3, LRIG1, LRIG3, MACO1, MAP3K9, MAPRE3, MIR100HG, MRVI1, MYCBP, NAALAD2, NACAD, NAV2, NFIC, NFIX, NR6A1, NT5DC1, PARP6, PLAG1, POFUT2, PRKACB, PRTG, PTCH1, PTPN13, RIMS1, SAV1, SBK1, SELENOP, SEMA5B, SHISA7, SLC35D2, SLC66A3, SLC6A17, SPTBN1, SRGAP2, STOX1, SUCLG2, SYT13, TGFBR3, TM6SF2, TMEM151B, TOB1, TPH1, and TRIM46.

In some embodiments, the plurality of genes includes one or more of ACE, ACSL1, ANP32A, ARL8A, BDNF, CCDC112, CCDC160, CCDC60, CEP170B, CHGB, COL23A1, DAAM2, DMTN, FABP7, FAM71E2, FNBP1L, HAPLN3, HTATIP2, KCNB1, KIF1A, LINC01128, LRIG1, MAP3K9, MGST1, MIR100HG, MRVI1, NAALAD2, NACAD, NFIC, NFIX, NR6A1, NT5DC1, PLAG1, PRKACB, PRTG, PTCH1, PTPN13, SALL4, SEMA5B, SLC35D2, SLC6A17, STOX1, SUCLG2, SYT13, TGFBR3, TPH1, and YBX3.

In some embodiments, the plurality of genes includes one or more of ACE, ACSL1, ANP32A, ARL8A, CCDC112, CCDC160, CCDC60, CEP170B, CHGB, DAAM2, FAM71E2, FNBP1L, HTATIP2, KCNB1, KIF1A, LINC01128, LRIG1, MAP3K9, MIR100HG, MRVI1, NAALAD2, NACAD, NFIC, NFIX, NR6A1, NT5DC1, PLAG1, PRKACB, PRTG, PTCH1, PTPN13, SEMA5B, SLC35D2, SLC6A17, STOX1, SUCLG2, SYT13, TGFBR3, and TPH1.

a. Upregulated Maturity Genes

In some embodiments, the one or more maturity genes include genes whose gene expression levels increase during differentiation. In some embodiments, the one or more maturity genes include genes whose gene expression levels increase during days 17-22 of differentiation, e.g., differentiation of cells according to any of the methods described herein. In some embodiments, the one or more maturity genes include genes whose gene expression levels increase substantially monotonically during differentiation. In some embodiments, the one or more maturity genes only include genes whose gene expression levels increase during differentiation. In some embodiments, the one or more maturity genes include one or more of AC104083.1, ACE, ACSL1, AFAP1, APBA1, ARHGDIG, ARL8A, BDNF, BICDL1, CAMK2B, CCDC112, CDK5R2, CEP170B, CHGB, DIRAS1, DMTN, DNAJB5, DUSP26, FBXL16, FNBP1L, GFOD2, GUCY1A1, HCN3, JPT1, KCNB1, KCNC1, KCNH6, KIF1A, KLF7, LINC01128, MACO1, MAP3K9, MAPRE3, MIR100HG, NACAD, NCAM1, NFIC, NFIX, PARP6, PRKACB, RIMS1, SBK1, SHISA7, SLC6A17, SPTBN1, SRGAP2, SYT13, TMEM151B, TPH1, and TRIM46.

In some embodiments, the plurality of genes includes one or more of AC104083.1, ACE, ACSL1, AFAP1, ARHGDIG, ARL8A, BICDL1, CCDC112, CEP170B, CHGB, DIRAS1, DNAJB5, DUSP26, FBXL16, FNBP1L, GFOD2, GUCY1A1, HCN3, JPT1, KCNB1, KCNH6, KIF1A, KLF7, LINC01128, MACO1, MAP3K9, MAPRE3, MIR100HG, NACAD, NFIC, NFIX, PARP6, PRKACB, RIMS1, SBK1, SHISA7, SLC6A17, SPTBN1, SRGAP2, SYT13, TMEM151B, TPH1, and TRIM46.

In some embodiments, the plurality of genes includes one or more of ACE, ACSL1, ARL8A, BDNF, CCDC112, CEP170B, CHGB, DMTN, FNBP1L, KCNB1, KIF1A, LINC01128, MAP3K9, MIR100HG, NACAD, NFIC, NFIX, PRKACB, SLC6A17, SYT13, and TPH1.

In some embodiments, the plurality of genes includes one or more of ACE, ACSL1, ARL8A, CCDC112, CEP170B, CHGB, FNBP1L, KCNB1, KIF1A, LINC01128, MAP3K9, MIR100HG, NACAD, NFIC, NFIX, PRKACB, SLC6A17, SYT13, and TPH1.

b. Downregulated Maturity Genes

In some embodiments, the one or more maturity genes include genes whose gene expression levels decrease during differentiation. In some embodiments, the one or more maturity genes include genes whose gene expression levels decrease during days 17-22 of differentiation, e.g., differentiation of cells according to any of the methods described herein. In some embodiments, the one or more maturity genes include genes whose gene expression levels decrease substantially monotonically during differentiation. In some embodiments, the one or more maturity genes only include genes whose gene expression levels decrease during differentiation. In some embodiments, the one or more maturity genes include one or more of ACSS3, ADSS, ANP32A, ANXA11, ASPH, CCDC160, CCDC60, COL23A1, CTSC, CYFIP1, DAAM2, DPY19L1, FABP7, FAM71E2, FAM86C2P, FZD2, HAPLN3, HLA-E, HTATIP2, IKZF2, IL4R, ITGA5, KCNJ2-AS1, LPIN3, LRIG1, LRIG3, MGST1, MRVI1, MYCBP, NAALAD2, NAV2, NR6A1, NT5DC1, PLAG1, POFUT2, PRTG, PTCH1, PTPN13, SALL4, SAV1, SELENOP, SEMA5B, SLC35D2, SLC66A3, STOX1, SUCLG2, TGFBR3, TM6SF2, TOB1, and YBX3.

In some embodiments, the plurality of genes includes one or more of ACSS3, ADSS, ANP32A, ANXA11, ASPH, CCDC160, CCDC60, CYFIP1, DAAM2, DPY19L1, FAM71E2, FAM86C2P, FZD2, HLA-E, HTATIP2, IKZF2, ITGA5, KCNJ2-AS1, LPIN3, LRIG1, LRIG3, MRVI1, MYCBP, NAALAD2, NAV2, NR6A1, NT5DC1, PLAG1, POFUT2, PRTG, PTCH1, PTPN13, SAV1, SELENOP, SEMA5B, SLC35D2, SLC66A3, STOX1, SUCLG2, TGFBR3, TM6SF2, and TOB1.

In some embodiments, the plurality of genes includes one or more of ANP32A, CCDC160, CCDC60, COL23A1, DAAM2, FABP7, FAM71E2, HAPLN3, HTATIP2, LRIG1, MGST1, MRVI1, NAALAD2, NR6A1, NT5DC1, PLAG1, PRTG, PTCH1, PTPN13, SALL4, SEMA5B, SLC35D2, STOX1, SUCLG2, TGFBR3, and YBX3.

In some embodiments, the plurality of genes includes one or more of ANP32A, CCDC160, CCDC60, DAAM2, FAM71E2, HTATIP2, LRIG1, MRVI1, NAALAD2, NR6A1, NT5DC1, PLAG1, PRTG, PTCH1, PTPN13, SEMA5B, SLC35D2, STOX1, SUCLG2, and TGFBR3.

### B. Machine Learning Models

In some embodiments, the provided methods include predicting if a population of neuronal progenitor cells will engraft in a brain region of a subject following implantation of the population of neuronal progenitor cells into the brain region. In some embodiments, the predicting is by applying gene expression levels of a plurality of genes for one or more cells of the population of neuronal progenitor cells as input to a process configured to predict if the population of neuronal progenitor cells will engraft in the brain region of the subject following implantation of the population of neuronal progenitor cells into the brain region. In some embodiments, the process includes a machine learning model. In some embodiments, the process, e.g., machine learning model, is trained using gene expression levels of one or more of the plurality of genes for a plurality of reference populations of neuronal progenitor cells. In some embodiments, the process, e.g., machine learning model, is trained also using engraftment fitness of the plurality of reference populations.

Various machine learning models that are suitable for predicting engraftment based on gene expression levels are familiar to those skilled in the art and are within the scope of the disclosure. Methods of training such machine learning models using gene expression levels and/or engraftment fitness of reference populations are also familiar to those skilled in the art and are within the scope of the disclosure. Machine learning models that can be used in accordance with the provided methods include supervised, unsupervised, and semi-supervised machine learning models. In some embodiments, the process, e.g., machine learning model, is or includes a supervised machine learning model. In some embodiments, the process, e.g., machine learning model, is or includes an unsupervised machine learning model. In some embodiments, the process, e.g., machine learning model, is or includes a semi-supervised machine learning model. In some embodiments, the process, e.g., machine learning model, includes performance of one or more data preprocessing techniques. In some embodiments, the process, e.g., machine learning model, includes performance of one or more dimensionality reduction methods.

In some embodiments, the process, e.g., machine learning model, is or includes a regression model. In some embodiments, the process, e.g., machine learning model, is or includes a classification model. In some embodiments, the process, e.g., machine learning model, is or includes a binary classification model. In some embodiments, the process, e.g., machine learning model, is or includes a multiclass classification model.

In some embodiments, the process, e.g., machine learning model, is or includes a logistic regression model. In some embodiments, the process, e.g., machine learning model, is or includes a linear regression model. In some embodiments, the process, e.g., machine learning model, is or includes a multiple linear regression model. In some embodiments, the process, e.g., machine learning model, is or includes a polynomial regression model. In some embodiments, the process, e.g., machine learning model, is or includes a quantile regression model. In some embodiments, the process, e.g., machine learning model, is or includes a principle components regression model. In some embodiments, the process, e.g., machine learning model, is or includes a partial least regression model. In some embodiments, the process, e.g., machine learning model, is or includes a support vector regression model. In some embodiments, the process, e.g., machine learning model, is or includes an ordinal regression model. In some embodiments, the process, e.g., machine learning model, is or includes a Poisson regression model. In some embodiments, the process, e.g., machine learning model, is or includes a negative binomial regression model. In some embodiments, the process, e.g., machine learning model, is or includes a quasi Poisson regression model. In some embodiments, the process, e.g., machine learning model, is or includes a linear discriminant analysis (LDA) model. In some embodiments, the process, e.g., machine learning model, is or includes a Naive Bayes classifier. In some embodiments, the process, e.g., machine learning model, is or includes a perceptron. In some embodiments, the process, e.g., machine learning model, is or includes a support vector machine (SVM). In some embodiments, the process, e.g., machine learning model, is or includes a quadratic classifier. In some embodiments, the process, e.g., machine learning model, is or includes a decision tree. In some embodiments, the process, e.g., machine learning model, is or includes a random forest. In some embodiments, the process, e.g., machine learning model, is or includes a neural network. In some embodiments, the process, e.g., machine learning model, is or includes an ensemble model comprising any of the foregoing models.

In some embodiments, the process, e.g., machine learning model, is or includes a penalized machine learning model. A penalized machine learning model is one in which coefficient estimates are regularized or constrained towards zero. In some embodiments, the process, e.g., machine learning model, is or includes a ridge regression model. In some embodiments, the process, e.g., machine learning model, is or includes a lasso regression model. In some embodiments, the process, e.g., machine learning model, is or includes an elastic net regression model. In some embodiments, the process, e.g., machine learning model, is or includes a lasso logistic regression model.

In some embodiments, the process, e.g., machine learning model, is or includes a clustering method. In some embodiments, the process, e.g., machine learning model, is or includes a connectivity-based clustering method. In some embodiments, the process, e.g., machine learning model, is or includes hierarchical clustering. In some embodiments, the process, e.g., machine learning model, is or includes a centroid-based clustering method. In some embodiments, the process, e.g., machine learning model, is or includes k-means clustering. In some embodiments, the process, e.g., machine learning model, is or includes a distribution-based clustering method. In some embodiments, the process, e.g., machine learning model, is or includes Gaussian mixture modeling. In some embodiments, the process, e.g., machine learning model, is or includes a density-based clustering method. In some embodiments, the process, e.g., machine learning model, is or includes DBSCAN. In some embodiments, the process, e.g., machine learning model, is or includes OPTICS. In some embodiments, the process, e.g., machine learning model, is or includes a grid-based clustering method. In some embodiments, the process, e.g., machine learning model, is or includes STING. In some embodiments, the process, e.g., machine learning model, is or includes CLIQUE.

In some embodiments, the process, e.g., machine learning model, is or includes factor analysis. In some embodiments, the process, e.g., machine learning model, is or includes network component analysis. In some embodiments, the process, e.g., machine learning model, is or includes linear discriminant analysis. In some embodiments, the process, e.g., machine learning model, is or includes independent component analysis (ICA). In some embodiments, the process, e.g., machine learning model, is or includes principal component analysis (PCA). In some embodiments, the process, e.g., machine learning model, is or includes sparse PCA. In some embodiments, the process, e.g., machine learning model, is or includes robust PCA.

In some embodiments, the process, e.g., machine learning model, is or includes non-negative matrix factorization (NMF). In some embodiments, the process, e.g., machine learning model, is or includes conventional NMF. In some embodiments, the process, e.g., machine learning model, is or includes discriminant NMF. In some embodiments, the process, e.g., machine learning model, is or includes regularized NMF. In some embodiments, the process, e.g., machine learning model, is or includes graph regularized NMF. In some embodiments, the process, e.g., machine learning model, is or includes bootstrapping sparse NMF.

In some embodiments, the process, e.g., machine learning model, is or includes kernel PCA. In some embodiments, the process, e.g., machine learning model, is or includes generalized discriminant analysis (GDA). In some embodiments, the process, e.g., machine learning model, is or includes an autoencoder. In some embodiments, the process, e.g., machine learning model, is or includes T-distributed Stochastic Neighbor Embedding (t-SNE). In some embodiments, the process, e.g., machine learning model, is or includes a manifold learning technique. In some embodiments, the process, e.g., machine learning model, is or includes Isomap. In some embodiments, the process, e.g., machine learning model, is or includes locally linear embedding (LLE). In some embodiments, the process, e.g., machine learning model, is or includes Hessian LLE. In some embodiments, the process, e.g., machine learning model, is or includes Laplacian eigenmaps. In some embodiments, the process, e.g., machine learning model, is or includes graph-based kernel PCA. In some embodiments, the process, e.g., machine learning model, is or includes uniform manifold approximation and projection (UMAP).

In some embodiments, the methods include selecting based on an output of the process, e.g., machine learning model, the population of neuronal progenitor cells as a population of neuronal progenitor cells that is predicted to engraft. Those skilled in the art are familiar with methods by which an output of a process trained using gene expression levels can be used to predict engraftment. In some embodiments, the process, e.g., machine learning model, is configured to predict the presence or absence of engraftment, and the population of neuronal progenitor cells is predicted to engraft if the process, e.g., machine learning model, predicts the presence of engraftment. In some embodiments, the process is configured to predict the degree of engraftment, and the population of neuronal progenitor cells is predicted to engraft if the predicted degree of engraftment exceeds a predetermined threshold level. In some embodiments, the process is configured to predict the probability of engraftment, and the population of neuronal progenitor cells is predicted to engraft if the predicted probability of engraftment exceeds a predetermined threshold level. In some embodiments, the predetermined probability threshold level is, is about, is greater than, or is greater than about 0.5. In some embodiments, the predetermined probability threshold level is, is about, is greater than, or is greater than about 0.55. In some embodiments, the predetermined probability threshold level is, is about, is greater than, or is greater than about 0.6. In some embodiments, the predetermined probability threshold level is, is about, is greater than, or is greater than about 0.65. In some embodiments, the predetermined probability threshold level is, is about, is greater than, or is greater than about 0.7. In some embodiments, the predetermined probability threshold level is, is about, is greater than, or is greater than about 0.75. In some embodiments, the predetermined probability threshold level is, is about, is greater than, or is greater than about 0.8. In some embodiments, the predetermined probability threshold level is, is about, is greater than, or is greater than about 0.85. In some embodiments, the predetermined probability threshold level is, is about, is greater than, or is greater than about 0.9. In some embodiments, the predetermined probability threshold level is, is about, is greater than, or is greater than about 0.91. In some embodiments, the predetermined probability threshold level is, is about, is greater than, or is greater than about 0.92. In some embodiments, the predetermined probability threshold level is, is about, is greater than, or is greater than about 0.93. In some embodiments, the predetermined probability threshold level is, is about, is greater than, or is greater than about 0.94. In some embodiments, the predetermined probability threshold level is, is about, is greater than, or is greater than about 0.95. In some embodiments, the predetermined probability threshold level is, is about, is greater than, or is greater than about 0.96. In some embodiments, the predetermined probability threshold level is, is about, is greater than, or is greater than about 0.97. In some embodiments, the predetermined probability threshold level is, is about, is greater than, or is greater than about 0.98. In some embodiments, the predetermined probability threshold level is, is about, is greater than, or is greater than about 0.99.

### IV. Compositions and Formulations

Provided herein are therapeutic compositions containing differentiated cells that are determined dopamine (DA) neuron progenitor cells. Also provided herein are therapeutic compositions containing differentiated cells produced by any of the provided methods, such as any methods described in Section II. Also provided herein are therapeutic compositions containing differentiated cells selected as predicted to engraft by any of the provided methods, such as any methods described in Section III. In some embodiments, the differentiated cells produced by any of the methods described herein are determined dopamine (DA) neuron progenitor cells.

In some embodiments, the differentiated cells in the provided therapeutic compositions, including those produced by any of the methods described herein, are capable of producing dopamine (DA). In some embodiments, the differentiated cells in the provided therapeutic compositions, including those produced by any of the methods described herein, do not produce or do not substantially produce norepinephrine (NE). Thus, in some embodiments, the differentiated cells in the therapeutic compositions provided herein, including those produced by any of the methods described herein, are capable of producing DA but do not produce or do not substantially produce NE.

In some embodiments, determined dopamine neuronal progenitor cells (DDPCs) of the therapeutic composition express one or more genes selected from (a) ASPM; (b) AURKB; (c) BRINP1; (d) BUB1; (e) CCNB2; (f) CDC20; (g) CDC25C; (h) CDKN1A; (i) CENPF; (j) DLGAP5; (k) FAM83D; (1) FANCD2; (m) GEM; (n) HMMR; (o) IQGAP3; (p) KIF20A; (q) KIF2C; (r) KIFC1; (s) MKI67; (t) PIMREG; (u) PLK2; (v) PTTG1; (w) SAPCD2; (x) TOP2A; (y) TPX2; and (z) TTK. In some of any embodiments, the DDPCs express one or more genes selected from (b) AURKB; (c) BRINP1; (d) BUB1; (e) CCNB2; (f) CDC20; (g) CDC25C; (h) CDKN1A; (i) CENPF; (j) DLGAP5; (k) FAM83D; (1) FANCD2; (m) GEM; (n) HMMR; (o) IQGAP3; (p) KIF20A; (q) KIF2C; (r) KIFC1; (s) MKI67; (t) PIMREG; (u) PLK2; (v) PTTG1; (w) SAPCD2; (y) TPX2; and (z) TTK.

In some embodiments, the DDPCs express one or more genes selected from (b) AURKB; (e) CCNB2; (g) CDC25C; (j) DLGAP5; (k) FAM83D; (o) IQGAP3; (p) KIF20A; (q) KIF2C; (r) KIFC1; (v) PTTG1; (w) SAPCD2; (x) TOP2A; and (y) TPX2.

In some embodiments, the DPPCs express one or more genes selected from (b) AURKB; (e) CCNB2; (g) CDC25C; (j) DLGAP5; (k) FAM83D; (o) IQGAP3; (p) KIF20A; (q) KIF2C; (r) KIFC1; (v) PTTG1; (w) SAPCD2; and (y) TPX2.

In some of any embodiments, expression of at least one of the one or more genes by the DDPCs is or is on average 1.1, 1.2, 1.3, 1.4, 1.5, 1.6, 1.7, 1.8, 1.9, or 2.0 fold greater than expression by a reference population of cells. In some of any embodiments, expression of each of the one or more genes by the DDPCs is or is on average 1.1, 1.2, 1.3, 1.4, 1.5, 1.6, 1.7, 1.8, 1.9, or 2.0 fold greater than expression by a reference population of cells.

In some embodiments, the reference population of cells comprises reference cells. In some embodiments, the reference population of cells is enriched for reference cells. In some embodiments, at least 50, 55, 60, 65, 70, 75, 80, 85, 90, 95, 96, 97, 98, or 99 percent of the reference population of cells is reference cells. In some embodiments, the reference population of cells is reference cells.

In some embodiments, the reference cells are not determined dopaminergic neuronal progenitor cells. In some embodiments, the reference cells are pluripotent stem cells. In some embodiments, the reference cells are floor plate midbrain progenitor cells. In some embodiments, the reference cells are differentiated dopaminergic neurons.

In some embodiments, the reference cells are cells differentiated from pluripotent stem cells under conditions to neurally differentiate the cells. In some embodiments, the cells are differentiated according to any of the methods described herein.

In some embodiments, the reference cells are cells at a particular timepoint of the differentiation method. In some embodiments, the timepoint is before the timepoint at which the harvested cells are harvested. In some embodiments, the timepoint is after the timepoint at which the harvested cells are harvested. In some embodiments, the timepoint is day 13. In some embodiments, the timepoint is day 14. In some embodiments, the timepoint is day 15. In some embodiments, the timepoint is day 16. In some embodiments, the timepoint is day 17. In some embodiments, the timepoint is day 16. In some embodiments, the timepoint is day 19. In some embodiments, the timepoint is day 20. In some embodiments, the timepoint is day 21. In some embodiments, the timepoint is day 22. In some embodiments, the timepoint is day 23. In some embodiments, the timepoint is day 24. In some embodiments, the timepoint is day 25.

In some of any embodiments, expression of at least one of the one or more genes by the DDPCs is at a ratio with respect to GAPDH expression. In some of any embodiments, expression of each of the one or more genes by the DDPCs is at a ratio with respect to GAPDH expression.

In some embodiments, the therapeutic composition exhibits one or more of (a) a ratio of ASPM to GAPDH expression of greater than about 7×10⁻⁴; (b) a ratio of AURKB to GAPDH expression of greater than about 9×10⁻⁴; (c) a ratio of BRINP1 to GAPDH expression of greater than about 9×10⁻⁵; (d) a ratio of BUB1 to GAPDH expression of greater than about 3×10⁻³; (e) a ratio of CCNB2 to GAPDH expression of greater than about 3×10⁻³; (f) a ratio of CDC20 to GAPDH expression of greater than about 3×10⁻³; (g) a ratio of CDC25C to GAPDH expression of greater than about 5×10⁻⁴; (h) a ratio of CDKN1A to GAPDH expression of greater than about 1×10⁻³; (i) a ratio of CENPF to GAPDH expression of greater than about 7×10⁻³; (j) a ratio of DLGAP5 to GAPDH expression of greater than about 2×10⁻³; (k) a ratio of FAM83D to GAPDH expression of greater than about 6×10⁻⁴; (1) a ratio of FANCD2 to GAPDH expression of greater than about 3×10⁻³; (m) a ratio of GEM to GAPDH expression of greater than about 6×10⁻⁴; (n) a ratio of HMMR to GAPDH expression of greater than about 8×10⁻⁴; (o) a ratio of IQGAP3 to GAPDH expression of greater than about 1×10⁻³; (p) a ratio of KIF20A to GAPDH expression of greater than about 1×10⁻³; (q) a ratio of KIF2C to GAPDH expression of greater than about 3×10⁻³; (r) a ratio of KIFC1 to GAPDH expression of greater than about 2×10⁻³; (s) a ratio of MKI67 to GAPDH expression of greater than about 2×10⁻³; (t) a ratio of PIMREG to GAPDH expression of greater than about 1×10⁻³; (u) a ratio of PLK2 to GAPDH expression of greater than about 4×10⁻³; (v) a ratio of PTTG1 to GAPDH expression of greater than about 3×10⁻³; (w) a ratio of SAPCD2 to GAPDH expression of greater than about 1×10⁻³; (x) a ratio of TOP2A to GAPDH expression of greater than about 3×10⁻²; (y) a ratio of TPX2 to GAPDH expression of greater than about 7×10⁻³; and (z) a ratio of TTK to GAPDH expression of greater than about 2×10⁻³.

In some embodiments, (a) the ratio of ASPM to GAPDH expression is between about 7×10⁻⁴ and about 2×10⁻¹; (b) the ratio of AURKB to GAPDH expression is between about 9×10⁻⁴ and about 4×10⁻²; (c) the ratio of BRINP1 to GAPDH expression is between about 9×10⁻⁵ and about 5×10⁻²; (d) the ratio of BUB1 to GAPDH expression is between about 3×10⁻³ and about 7×10⁻²; (e) the ratio of CCNB2 to GAPDH expression is between about 3×10⁻³ and about 7×10⁻²; (f) the ratio of CDC20 to GAPDH expression is between about 3×10⁻³ and about 1×10⁻¹; (g) the ratio of CDC25C to GAPDH expression is between about 5×10⁻⁴ and about 3×10⁻²; (h) the ratio of CDKN1A to GAPDH expression is between about 1×10⁻³ and about 9×10⁻²; (i) the ratio of CENPF to GAPDH expression is between about 7×10⁻³ and about 5×10⁻¹; (j) the ratio of DLGAP5 to GAPDH expression is between about 2×10⁻³ and about 9×10⁻²; (k) the ratio of FAM83D to GAPDH expression is between about 6×10⁻⁴ and about 3×10⁻²; (1) the ratio of FANCD2 to GAPDH expression is between about 3×10⁻³ and about 4×10⁻²; (m) the ratio of GEM to GAPDH expression is between about 6×10⁻⁴ and about 3×10⁻²; (n) the ratio of HMMR to GAPDH expression is between about 8×10⁻⁴ and about 6×10⁻²; (o) the ratio of IQGAP3 to GAPDH expression is between about 1×10⁻³ and about 6×10⁻²; (p) the ratio of KIF20A to GAPDH expression is between about 1×10⁻³ and about 8×10⁻²; (q) the ratio of KIF2C to GAPDH expression is between about 3×10⁻³ and about 7×10⁻²; (r) the ratio of KIFC1 to GAPDH expression is between about 2×10⁻³ and about 8×10⁻²; (s) the ratio of MKI67 to GAPDH expression is between about 2×10⁻³ and about 4×10⁻¹; (t) the ratio of PIMREG to GAPDH expression is between about 1×10⁻³ and about 4×10⁻²; (u) the ratio of PLK2 to GAPDH expression is between about 4×10⁻³ and about 6×10⁻²; (v) the ratio of PTTG1 to GAPDH expression is between about 3×10⁻³ and about 9×10⁻²; (w) the ratio of SAPCD2 to GAPDH expression is between about 1×10⁻³ and about 3×10⁻²; (x) the ratio of TOP2A to GAPDH expression is between about 3×10⁻² and about 7×10⁻¹; (y) the ratio of TPX2 to GAPDH expression is between about 7×10⁻³ and about 2×10⁻¹; and/or (z) the ratio of TTK to GAPDH expression is between about 2×10⁻³ and about 8×10⁻².

In some embodiments, the composition exhibits between 2 and 26 of (a)-(z). In some embodiments, the composition exhibits between 2 and 22 of (a)-(z). In some embodiments, the composition exhibits between 2 and 18 of (a)-(z). In some embodiments, the composition exhibits between 2 and 14 of (a)-(z). In some embodiments, the composition exhibits between 2 and 10 of (a)-(z). In some embodiments, the composition exhibits between 2 and 6 of (a)-(z). In some embodiments, the composition exhibits between 2 and 4 of (a)-(z). In some embodiments, the composition exhibits between 4 and 26 of (a)-(z). In some embodiments, the composition exhibits between 4 and 22 of (a)-(z). In some embodiments, the composition exhibits between 4 and 18 of (a)-(z). In some embodiments, the composition exhibits between 4 and 14 of (a)-(z). In some embodiments, the composition exhibits between 4 and 10 of (a)-(z). In some embodiments, the composition exhibits between 4 and 6 of (a)-(z). In some embodiments, the composition exhibits between 6 and 26 of (a)-(z). In some embodiments, the composition exhibits between 6 and 22 of (a)-(z). In some embodiments, the composition exhibits between 6 and 18 of (a)-(z). In some embodiments, the composition exhibits between 6 and 14 of (a)-(z). In some embodiments, the composition exhibits between 6 and 10 of (a)-(z). In some embodiments, the composition exhibits between 10 and 26 of (a)-(z). In some embodiments, the composition exhibits between 10 and 22 of (a)-(z). In some embodiments, the composition exhibits between 10 and 18 of (a)-(z). In some embodiments, the composition exhibits between 10 and 14 of (a)-(z). In some embodiments, the composition exhibits between 14 and 26 of (a)-(z). In some embodiments, the composition exhibits between 14 and 22 of (a)-(z). In some embodiments, the composition exhibits between 14 and 18 of (a)-(z). In some embodiments, the composition exhibits between 18 and 26 of (a)-(z). In some embodiments, the composition exhibits between 18 and 22 of (a)-(z). In some embodiments, the composition exhibits between 22 and 26 of (a)-(z).

In some embodiments, the ratio of BRINP1 to GAPDH expression is between about 9×10⁻⁵ and about 5×10⁻²; the ratio of CDKN1A to GAPDH expression is between about 1×10⁻³ and about 9×10⁻²; the ratio of FAM83D to GAPDH expression is between about 6×10⁻⁴ and about 3×10⁻²; the ratio of FANCD2 to GAPDH expression is between about 3×10⁻³ and about 4×10⁻²; the ratio of GEM to GAPDH expression is between about 6×10⁻⁴ and about 3×10⁻²; the ratio of PLK2 to GAPDH expression is between about 4×10⁻³ and about 6×10⁻²; and/or the ratio of SAPCD2 to GAPDH expression is between about 1×10⁻³ and about 3×10⁻².

In some embodiments, the ratio of BRINP1 to GAPDH expression is between about 9×10⁻⁵ and about 5×10⁻²; the ratio of CDKN1A to GAPDH expression is between about 1×10⁻³ and about 9×10⁻²; the ratio of FAM83D to GAPDH expression is between about 6×10⁻⁴ and about 3×10²; the ratio of FANCD2 to GAPDH expression is between about 3×10⁻³ and about 4×10⁻²; the ratio of GEM to GAPDH expression is between about 6×10⁻⁴ and about 3×10⁻²; the ratio of PLK2 to GAPDH expression is between about 4×10⁻³ and about 6×10⁻²; and the ratio of SAPCD2 to GAPDH expression is between about 1×10⁻³ and about 3×10⁻².

In some embodiments, the ratio is on average across the DDPCs.

In some of any embodiments, at least 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, or 90% of cells of the therapeutic composition is DDPCs.

In some embodiments, the expression is RNA expression. In some embodiments, the RNA expression is measured using RNA sequencing.

In some embodiments, the determined dopamine (DA) neuron progenitor cells express EN1. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express EN1. In some embodiments, at least about 20% of the cells of the therapeutic composition express EN1. In some embodiments, at least about 25% of the cells of the therapeutic composition express EN1. In some embodiments, at least about 30% of the cells of the therapeutic composition express EN1. In some embodiments, at least about 35% of the cells of the therapeutic composition express EN1. In some embodiments, at least about 40% of the cells of the therapeutic composition express EN1. In some embodiments, at least about 45% of the cells of the therapeutic composition express EN1. In some embodiments, at least about 50% of the cells of the therapeutic composition express EN1. In some embodiments, at least about 55% of the cells of the therapeutic composition express EN1. In some embodiments, at least about 60% of the cells of the therapeutic composition express EN1. In some embodiments, at least about 65% of the cells of the therapeutic composition express EN1. In some embodiments, at least about 70% of the cells of the therapeutic composition express EN1. In some embodiments, at least about 75% of the cells of the therapeutic composition express EN1. In some embodiments, at least about 80% of the cells of the therapeutic composition express EN1.

In some embodiments, the therapeutic composition exhibits a ratio of counts per million (CPM) EN1 to CPM GAPDH of greater than about 1×10⁻⁴. In some embodiments, the ratio of CPM EN1 to CPM GAPDH is between about 1.5×10⁻³ and 1×10⁻².

In some embodiments, the determined dopamine (DA) neuron progenitor cells express CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express CORIN. In some embodiments, at least about 20% of the cells of the therapeutic composition express CORIN. In some embodiments, at least about 25% of the cells of the therapeutic composition express CORIN. In some embodiments, at least about 30% of the cells of the therapeutic composition express CORIN. In some embodiments, at least about 35% of the cells of the therapeutic composition express CORIN. In some embodiments, at least about 40% of the cells of the therapeutic composition express CORIN. In some embodiments, at least about 45% of the cells of the therapeutic composition express CORIN. In some embodiments, at least about 50% of the cells of the therapeutic composition express CORIN. In some embodiments, at least about 55% of the cells of the therapeutic composition express CORIN. In some embodiments, at least about 60% of the cells of the therapeutic composition express CORIN. In some embodiments, at least about 65% of the cells of the therapeutic composition express CORIN. In some embodiments, at least about 70% of the cells of the therapeutic composition express CORIN. In some embodiments, at least about 75% of the cells of the therapeutic composition express CORIN. In some embodiments, at least about 80% of the cells of the therapeutic composition express CORIN.

In some embodiments, the therapeutic composition exhibits a ratio of counts per million (CPM) CORIN to CPM GAPDH of greater than about 1×10⁻⁴. In some embodiments, the ratio of CPM CORIN to CPM GAPDH is between about 5×10⁻² and 5×10⁻¹.

In In some embodiments, the determined dopamine (DA) neuron progenitor cells express EN1 and CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express EN1 and CORIN. In some embodiments, at least about 20% of the cells of the therapeutic composition express EN1 and CORIN. In some embodiments, at least about 25% of the cells of the therapeutic composition express EN1 and CORIN. In some embodiments, at least about 30% of the cells of the therapeutic composition express EN1 and CORIN. In some embodiments, at least about 35% of the cells of the therapeutic composition express EN1 and CORIN. In some embodiments, at least about 40% of the cells of the therapeutic composition express EN1 and CORIN. In some embodiments, at least about 45% of the cells of the therapeutic composition express EN1 and CORIN. In some embodiments, at least about 50% of the cells of the therapeutic composition express EN1 and CORIN. In some embodiments, at least about 55% of the cells of the therapeutic composition express EN1 and CORIN. In some embodiments, at least about 60% of the cells of the therapeutic composition express EN1 and CORIN. In some embodiments, at least about 65% of the cells of the therapeutic composition express EN1 and CORIN. In some embodiments, at least about 70% of the cells of the therapeutic composition express EN1 and CORIN. In some embodiments, at least about 75% of the cells of the therapeutic composition express EN1 and CORIN. In some embodiments, at least about 80% of the cells of the therapeutic composition express EN1 and CORIN.

In some embodiments, the therapeutic composition exhibits (a) a ratio of counts per million (CPM) EN1 to CPM GAPDH of greater than about 1×10⁻⁴; and (b) a ratio of CPM CORIN to CPM GAPDH of greater than about 2×10⁻². In some embodiments, the ratio of CPM EN1 to CPM GAPDH isbetween about 1.5×10⁻³ and 1×10⁻²; and the ratio of CPM CORIN to CPM GAPDH of between about 5×10⁻² and 5×10⁻¹.

In some embodiments, less than 10% of determined dopamine (DA) neuron progenitor cells express TH. In some embodiments, the determined dopamine (DA) neuron progenitor cells express low levels of TH. In some embodiments, the determined dopamine (DA) neuron progenitor cells do not express TH. In some embodiments, the determined dopamine (DA) neuron progenitor cells express TH at lower levels than cells harvested or collected on other days. In some embodiments, some of the determined dopamine (DA) neuron progenitor cells express EN1 and CORIN and less than 10% of the cells express TH.

In some embodiments, less than 8% of the cells express TH. In some embodiments, less than 5% of the cells express TH. In some embodiments, between about 2% and 10%, between about 2% and 8%, between about 2% and 6%, between about 2% and 4%, between about 4% and 10%, between about 4% and 8%, between about 4% and 6%, between about 6% and 10%, between about 6% and 8%, or between aobut 8% and 10% of the total cells in the composition express TH.

In some embodiments the therapeutic composition exhibits a ratio of counts per million (CPM) TH to CPM GAPDH of less than about 3×10⁻². In some embodiments, the ratio of CPM TH to CPM GAPDH is between about 1×10⁻³ and 2.5×10⁻².

In some embodiments, less than 10% of the total cells in the composition express TH, and at least about 20% of the cells of the therapeutic composition express EN1. In some embodiments, less than 10% of the total cells in the composition express TH, and at least about 25% of the cells of the therapeutic composition express EN1. In some embodiments, less than 10% of the total cells in the composition express TH, and at least about 30% of the cells of the therapeutic composition express EN1. In some embodiments, less than 10% of the total cells in the composition express TH, and at least about 35% of the cells of the therapeutic composition express EN1. In some embodiments, less than 10% of the total cells in the composition express TH, and at least about 40% of the cells of the therapeutic composition express EN1. In some embodiments, less than 10% of the total cells in the composition express TH, and at least about 45% of the cells of the therapeutic composition express EN1. In some embodiments, less than 10% of the total cells in the composition express TH, and at least about 50% of the cells of the therapeutic composition express EN1. In some embodiments, less than 10% of the total cells in the composition express TH, and at least about 55% of the cells of the therapeutic composition express EN1. In some embodiments, less than 10% of the total cells in the composition express TH, and at least about 60% of the cells of the therapeutic composition express EN1. In some embodiments, less than 10% of the total cells in the composition express TH, and at least about 65% of the cells of the therapeutic composition express EN1. In some embodiments, less than 10% of the total cells in the composition express TH, and at least about 70% of the cells of the therapeutic composition express EN1. In some embodiments, less than 10% of the total cells in the composition express TH, and at least about 75% of the cells of the therapeutic composition express EN1. In some embodiments, less than 10% of the total cells in the composition express TH, and at least about 80% of the cells of the therapeutic composition express EN1.

In some embodiments, less than 10% of the total cells in the composition express TH, and at least about 20% of the cells of the therapeutic composition express CORIN. In some embodiments, less than 10% of the total cells in the composition express TH, and at least about 25% of the cells of the therapeutic composition express CORIN. In some embodiments, less than 10% of the total cells in the composition express TH, and at least about 30% of the cells of the therapeutic composition express CORIN. In some embodiments, less than 10% of the total cells in the composition express TH, and at least about 35% of the cells of the therapeutic composition express CORIN. In some embodiments, less than 10% of the total cells in the composition express TH, and at least about 40% of the cells of the therapeutic composition express CORIN. In some embodiments, less than 10% of the total cells in the composition express TH, and at least about 45% of the cells of the therapeutic composition express CORIN.

In some embodiments, less than 10% of the total cells in the composition express TH, and at least about 50% of the cells of the therapeutic composition express CORIN. In some embodiments, less than 10% of the total cells in the composition express TH, and at least about 55% of the cells of the therapeutic composition express CORIN. In some embodiments, less than 10% of the total cells in the composition express TH, and at least about 60% of the cells of the therapeutic composition express CORIN. In some embodiments, less than 10% of the total cells in the composition express TH, and at least about 65% of the cells of the therapeutic composition express CORIN. In some embodiments, less than 10% of the total cells in the composition express TH, and at least about 70% of the cells of the therapeutic composition express CORIN. In some embodiments, less than 10% of the total cells in the composition express TH, and at least about 75% of the cells of the therapeutic composition express CORIN. In some embodiments, less than 10% of the total cells in the composition express TH, and at least about 80% of the cells of the therapeutic composition express CORIN.

In some embodiments, less than 10% of the total cells in the composition express TH, and at least about 20% of the cells of the therapeutic composition express EN1 and CORIN. In some embodiments, less than 10% of the total cells in the composition express TH, and at least about 25% of the cells of the therapeutic composition express EN1 and CORIN. In some embodiments, less than 10% of the total cells in the composition express TH, and at least about 30% of the cells of the therapeutic composition express EN1 and CORIN. In some embodiments, less than 10% of the total cells in the composition express TH, and at least about 35% of the cells of the therapeutic composition express EN1 and CORIN. In some embodiments, less than 10% of the total cells in the composition express TH, and at least about 40% of the cells of the therapeutic composition express EN1 and CORIN. In some embodiments, less than 10% of the total cells in the composition express TH, and at least about 45% of the cells of the therapeutic composition express EN1 and CORIN. In some embodiments, less than 10% of the total cells in the composition express TH, and at least about 50% of the cells of the therapeutic composition express EN1 and CORIN. In some embodiments, less than 10% of the total cells in the composition express TH, and at least about 55% of the cells of the therapeutic composition express EN1 and CORIN. In some embodiments, less than 10% of the total cells in the composition express TH, and at least about 60% of the cells of the therapeutic composition express EN1 and CORIN. In some embodiments, less than 10% of the total cells in the composition express TH, and at least about 65% of the cells of the therapeutic composition express EN1 and CORIN. In some embodiments, less than 10% of the total cells in the composition express TH, and at least about 70% of the cells of the therapeutic composition express EN1 and CORIN. In some embodiments, less than 10% of the total cells in the composition express TH, and at least about 75% of the cells of the therapeutic composition express EN1 and CORIN. In some embodiments, less than 10% of the total cells in the composition express TH, and at least about 80% of the cells of the therapeutic composition express EN1 and CORIN.

In some embodiments, among any of the provided compositions are pharmaceutical compositions containing a pharmaceutically acceptable carrier. In some embodiments, the dose of cells comprising cells produced by any of the methods disclosed herein, is provided as a composition or formulation, such as a pharmaceutical composition or formulation. Such compositions can be used in accord with the provided methods, articles of manufacture, and/or with the provided compositions, such as in the prevention or treatment of diseases, conditions, and disorders, such as neurodegenerative disorders.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The formulation or composition may also be administered in combination with another form of treatment useful for the particular indication, disease, or condition being prevented or treated with the cells or agents, where the respective activities do not adversely affect one another. Thus, in some embodiments, the pharmaceutical composition is administered in combination with deep brain stimulation (DBS).

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by stereotactic injection (e.g., using a catheter). In some embodiments, a given dose is administered by a single bolus administration of the cells or agent. In some embodiments, it is administered by multiple bolus administrations of the cells or agent, for example, over a period of months or years. In some embodiments, the agents or cells can be administered by stereotactic injection into the brain, such as in the striatum.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous. For example, non-pluripotent cells (e.g., fibroblasts) can be obtained from a subject, and administered to the same subject following reprogramming and differentiation. When administering a therapeutic composition (e.g., a pharmaceutical composition containing a genetically reprogrammed and/or differentiated cell or an agent that treats or ameliorates symptoms of a disease or disorder, such as a neurodegenerative disorder), it will generally be formulated in a unit dosage injectable form (solution, suspension, emulsion). Formulations include those for stereotactic administration, such as into the brain (e.g. the striatum).

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### V. Methods of Treatment

Provided herein are methods of using any of the provided compositions for treating a disease or condition in a subject in need thereof. In particular embodiments, the composition is produced by the methods provided herein. Such methods and uses include therapeutic methods and uses, for example, involving administration of the therapeutic cells, or compositions containing the same, to a subject having a disease, condition, or disorder. In some embodiments the disease or condition is a neurodegenerative disease or condition. In some embodiments, the cells or pharmaceutical composition thereof is administered in an effective amount to effect treatment of the disease or disorder. Uses include uses of the cells or pharmaceutical compositions thereof in such methods and treatments, and in the preparation of a medicament in order to carry out such therapeutic methods. In some embodiments, the methods thereby treat the disease or condition or disorder in the subject.

The present disclosure relates to methods of lineage specific differentiation of pluripotent stem cells (PSCs), including embryonic stem (ES) cells and induced pluripotent stem cells (iPSCs), for use in neurodegenerative diseases. Specifically, the methods, compositions, and uses thereof provided herein contemplate differentiation of pluripotent stem cells for administration to subjects exhibiting a loss of dopamine (DA) neurons, including Parkinson's disease.

Parkinson's disease (PD) is the second most common neurodegenerative, estimated to affect 4-5 million patients worldwide. This number is predicted to more than double by 2030. PD is the second most common neurodegenerative disorder after Alzheimer's disease, affecting approximately 1 million patients in the US with 60,000 new patients diagnosed each year. Currently there is no cure for PD, which is characterized pathologically by a selective loss of midbrain DA neurons in the substantia nigra. A fundamental characteristic of PD is therefore progressive, severe and irreversible loss of midbrain dopamine (DA) neurons resulting in ultimately disabling motor dysfunction.

In some embodiments, a subject has a neurodegenerative disease. In some embodiments, the neurodegenerative disease comprises the loss of dopamine neurons in the brain. In some embodiments, the subject has lost dopamine neurons in the substantia nigra (SN). In some embodiments, the subject has lost dopamine neurons in the substantia nigra pas compacta (SNc). In some embodiments, the subject exhibits rigidity, bradykinesia, postural reflect impairment, resting tremor, or a combination thereof. In some embodiments, the subject exhibits abnormal [18F]-L-DOPA PET scan. In some embodiments, the subject exhibits [18F]-DG-PET evidence for a Parkinson's Disease Related Pattern (PDRP).

In some embodiments, the neurodegenerative disease is Parkinsonism. In some embodiments, the neurodegenerative disease is Parkinson's disease. In some embodiments, the neurodegenerative disease is idiopathic Parkinson's disease. In some embodiments, the neurodegenerative disease is a familial form of Parkinson's disease. In some embodiments, the subject has mild Parkinson's disease. In some embodiments, the subject has a Movement Disorder Society-Unified Parkinson's Disease Rating Scale (MDS-UPDRS) motor score of less than or equal to 32. In some embodiments, the subject has moderate or advanced Parkinson's disease. In some embodiments, the subject has mild Parkinson's disease. In some embodiments, the subject has a MDS-UPDRS motor score of between 3β and 60.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells is administered to the striatum of the subject. In some embodiments, the dose of cells is administered to one hemisphere of the subject's striatum. In some embodiments, the dose of cells is administered to both hemispheres of the subject's.

In some embodiments, the dose of cells administered to the subject is about 5×10⁶ cells. In some embodiments, the dose of cells administered to the subject is about 10×10⁶ cells. In some embodiments, the dose of cells administered to the subject is about 15×10⁶ cells. In some embodiments, the dose of cells administered to the subject is about 20×10⁶ cells. In some embodiments, the dose of cells administered to the subject is about 25×10⁶ cells. In some embodiments, the dose of cells administered to the subject is about 30×10⁶ cells.

In some embodiments, the dose of cells comprises between at or about 250,000 cells per hemisphere and at or about 20 million cells per hemisphere, between at or about 500,000 cells per hemisphere and at or about 20 million cells per hemisphere, between at or about 1 million cells per hemisphere and at or about 20 million cells per hemisphere, between at or about 5 million cells per hemisphere and at or about 20 million cells per hemisphere, between at or about 10 million cells per hemisphere and at or about 20 million cells per hemisphere, between at or about 15 million cells per hemisphere and at or about 20 million cells per hemisphere, between at or about 250,000 cells per hemisphere and at or about 15 million cells per hemisphere, between at or about 500,000 cells per hemisphere and at or about 15 million cells per hemisphere, between at or about 1 million cells per hemisphere and at or about 15 million cells per hemisphere, between at or about 5 million cells per hemisphere and at or about 15 million cells per hemisphere, between at or about 10 million cells per hemisphere and at or about 15 million cells per hemisphere, between at or about 250,000 cells per hemisphere and at or about 10 million cells per hemisphere, between at or about 500,000 cells per hemisphere and at or about 10 million cells per hemisphere, between at or about 1 million cells per hemisphere and at or about 10 million cells per hemisphere, between at or about 5 million cells per hemisphere and at or about 10 million cells per hemisphere, between at or about 250,000 cells per hemisphere and at or about 5 million cells per hemisphere, between at or about 500,000 cells per hemisphere and at or about 5 million cells per hemisphere, between at or about 1 million cells per hemisphere and at or about 5 million cells per hemisphere, between at or about 250,000 cells per hemisphere and at or about 1 million cells per hemisphere, between at or about 500,000 cells per hemisphere and at or about 1 million cells per hemisphere, or between at or about 250,000 cells per hemisphere and at or about 500,00 cells per hemisphere.

In some embodiments, the dose of cells is between at or about 1 million cells per hemisphere and at or about 30 million cells per hemisphere. In some embodiments, the dose of cells is between at or about 5 million cells per hemisphere and at or about 20 million cells per hemisphere. In some embodiments, the dose of cells is between at or about 10 million cells per hemisphere and at or about 15 million cells per hemisphere.

In some embodiments, the dose of cells is between about 3×10⁶ cells/hemisphere and 15×10⁶ cells/hemisphere. In some embodiments, the dose of cells is about about 3×10⁶ cells/hemisphere. In some embodiments, the dose of cells is about 4×10⁶ cells/hemisphere. In some embodiments, the dose of cells is about 5×10⁶ cells/hemisphere. In some embodiments, the dose of cells is about 6×10⁶ cells/hemisphere. In some embodiments, the dose of cells is about 7×10⁶ cells/hemisphere. In some embodiments, the dose of cells is about 8×10⁶ cells/hemisphere. In some embodiments, the dose of cells is about 9×10⁶ cells/hemisphere. In some embodiments, the dose of cells is about 10×10⁶ cells/hemisphere. In some embodiments, the dose of cells is about 11×10⁶ cells/hemisphere. In some embodiments, the dose of cells is about 12×10⁶ cells/hemisphere. In some embodiments, the dose of cells is about 13×10⁶ cells/hemisphere. In some embodiments, the dose of cells is about 14×10⁶ cells/hemisphere. In some embodiments, the dose of cells is about 15×10⁶ cells/hemisphere.

In some embodiments, the number of cells administered to the subject is between about 0.25×10⁶ total cells and about 20×10⁶ total cells, between about 0.25×10⁶ total cells and about 15×10⁶ total cells, between about 0.25×10⁶ total cells and about 10×10⁶ total cells, between about 0.25×10⁶ total cells and about 5×10⁶ total cells, between about 0.25×10⁶ total cells and about 1×10⁶ total cells, between about 0.25×10⁶ total cells and about 0.75×10⁶ total cells, between about 0.25×10⁶ total cells and about 0.5×10⁶ total cells, between about 0.5×10⁶ total cells and about 20×10⁶ total cells, between about 0.5×10⁶ total cells and about 15×10⁶ total cells, between about 0.5×10⁶ total cells and about 10×10⁶ total cells, between about 0.5×10⁶ total cells and about 5×10⁶ total cells, between about 0.5×10⁶ total cells and about 1×10⁶ total cells, between about 0.5×10⁶ total cells and about 0.75×10⁶ total cells, between about 0.75×10⁶ total cells and about 20×10⁶ total cells, between about 0.75×10⁶ total cells and about 15×10⁶ total cells, between about 0.75×10⁶ total cells and about 10×10⁶ total cells, between about 0.75×10⁶ total cells and about 5×10⁶ total cells, between about 0.75×10⁶ total cells and about 1×10⁶ total cells, between about 1×10⁶ total cells and about 20×10⁶ total cells, between about 1×10⁶ total cells and about 15×10⁶ total cells, between about 1×10⁶ total cells and about 10×10⁶ total cells, between about 1×10⁶ total cells and about 5×10⁶ total cells, between about 5×10⁶ total cells and about 20×10⁶ total cells, between about 5×10⁶ total cells and about 15×10⁶ total cells, between about 5×10⁶ total cells and about 10×10⁶ total cells, between about 10×10⁶ total cells and about 20×10⁶ total cells, between about 10×10⁶ total cells and about 15×10⁶ total cells, or between about 15×10⁶ total cells and about 20×10⁶ total cells.

In certain embodiments, the cells, or individual populations of sub-types of cells, are administered to the subject at a range of about 5 million cells per hemisphere to about 20 million cells per hemisphere or any value in between these ranges. Dosages may vary depending on attributes particular to the disease or disorder and/or patient and/or other treatments.

In some embodiments, the patient is administered multiple doses, and each of the doses or the total dose can be within any of the foregoing values. In some embodiments, the dose of cells comprises the administration of from or from about 5 million cells per hemisphere to about 20 million cells per hemisphere, each inclusive.

In some embodiments, the dose of cells, e.g. differentiated cells, is administered to the subject as a single dose or is administered only one time within a period of two weeks, one month, three months, six months, 1 year or more.

In the context of stem cell transplant, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as a day. Thus, in some contexts, the dose is a single or continuous administration of the specified number of cells, given or initiated at a single point in time. In some contexts, however, the dose is administered in multiple injections or infusions in a single period, such as by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time.

In some embodiments, the administration of the composition or dose, e.g., administration of the plurality of cell compositions, involves administration of the cell compositions separately. In some aspects, the separate administrations are carried out simultaneously, or sequentially, in any order.

In some embodiments, the subject receives multiple doses, e.g., two or more doses or multiple consecutive doses, of the cells. In some embodiments, two doses are administered to a subject. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. disease stage and/or likelihood or incidence of the subject developing adverse outcomes, e.g., dyskinesia.

In some embodiments, the dose of cells is generally large enough to be effective in improving symptoms of the disease.

In some embodiments, the cells are administered at a desired dosage, which in some aspects includes a desired dose or number of cells or cell type(s) and/or a desired ratio of cell types. In some embodiments, the dosage of cells is based on a desired total number (or number per kg of body weight) of cells in the individual populations or of individual cell types (e.g., TH+ or TH−). In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In other embodiments, the numbers and/or concentrations of cells refer to the number or concentration of all cells administered.

In some embodiments, the cells are administered at a desired dosage, which in some aspects includes a desired dose or number of cells or cell type(s) and/or a desired ratio of cell types. Thus, the dosage of cells in some embodiments is based on a total number of cells and a desired ratio of the individual populations or sub-typesIn some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In other embodiments, the numbers and/or concentrations of cells refer to the number or concentration of all cells administered.

In some aspects, the size of the dose is determined based on one or more criteria such as response of the subject to prior treatment, e.g. disease type and/or stage, and/or likelihood or incidence of the subject developing toxic outcomes, e.g., dyskinesia.

### VI. Articles of Manufacture and Kits

Also provided are articles of manufacture, systems, apparatuses, and kits useful in performing the provided methods. Also provided are articles of manufacture, including: (i) one or more reagents for differentiation of pluripotent stem cells into floor plate midbrain progenitor cells, determined dopamine (DA) neuron progenitor cells, and/or dopamine (DA) neurons; and (ii) instructions for use of the one or more reagents for performing any methods described herein.

In some of any such embodiments, the reagent for differentiation is or includes a small molecule, capable of inhibiting TGF-β/activin-Nodal signaling. In some of any such embodiments, the reagent for differentation is or includes SB431542. In some of any such embodiments, the reagent for differentiation is or includes a small molecule, capable of activating SHH signaling. In some of any such embodiments, the reagent for activating SHH signaling is or includes SHH. In some of any such embodiments, the reagent for activating SHH signaling is or includes purmorphamine. In some of any such embodiments, the reagent for activating SHH signaling is or includes SHH and purmorphamine. In some of any such embodiments, the reagent for differentiation is or includes a small molecule, capable of inhibiting BMP signaling. In some of any such embodiments, the reagent for inhibiting BMP signaling is LDN193189. In some of any such embodiments, the reagent for differentiation is or includes a small molecule, capable of inhibiting GSK3β signaling. In some of any of such embodiments, the reagent is or includes CHIR99021. In some of any of such embodiments, the reagent for differentiation is or includes one or more of BDNF, GDNF, dbcAMP, ascorbic acid, TGF3β, and DAPT. The reagents in the kit in one embodiment may be in solution, may be frozen, or may be lyophilized.

Also provided are articles of manufacture, including (i) any composition described herein; and (ii) instructions for administering the composition to a subject.

In some embodiments, the articles of manufacture or kits include one or more containers, typically a plurality of containers, packaging material, and a label or package insert on or associated with the container or containers and/or packaging, generally including instructions for use, e.g., instructions for reagents for differentiation of pluripotent cells, e.g., differentiation of iPSCs into floor plate midbrain progenitor cells, determined dopamine (DA) neuron progenitor cells, and/or dopamine (DA) neurons, and instructions to carry out any of the methods provided herein. In some aspects, the provided articles of manufacture contain reagents for differentiation and/or maturation of cells, for example, at one or more steps of the manufacturing process, such as any reagents described in any steps of Sections II and III.

Also provided are articles of manufacture and kits containing differentiated cells, such as those generated using the methods provided herein, and optionally instructions for use, for example, instructions for administering. In some embodiments, the instructions provide directions or specify methods for assessing if a subject, prior to receiving a cell therapy, is likely or suspected of being likely to respond and/or the degree or level of response following administration of differentiated cells expressing a recombinant receptor for treating a disease or disorder. In some aspects, the articles of manufacture can contain a dose or a composition of differentiated cells.

The articles of manufacture provided herein contain packaging materials. Packaging materials for use in packaging the provided materials are well known to those of skill in the art. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the reagents and/or cell compositions are packaged separately. In some embodiments, each container can have a single compartment. In some embodiments, other components of the articles of manufacture or kits are packaged separately, or together in a single compartment.

### VII. Exemplary Embodiments

Among the provided embodiments are:

1. A method of predicting cell engraftment of a population of neuronal progenitor cells, the method comprising:
(a) obtaining gene expression levels of a plurality of genes for one or more cells of a population of neuronal progenitor cells, wherein:
the population of neuronal progenitor cells is from a culture of cells differentiated from pluripotent stem cells under conditions to neurally differentiate the cells; and
the plurality of genes comprise one or more cell cycle genes and/or one or more maturity genes; and
(b) applying the gene expression levels as input to a process configured to predict if the population of neuronal progenitor cells will engraft in a brain region of a subject following implantation of the population of neuronal progenitor cells into the brain region, wherein the predicting is based on gene expression levels of one or more of the plurality of genes.

2. A method of assessing a population of neuronal progenitor cells for implantation in a subject to treat a neurodegenerative disease, the method comprising:
(a) obtaining gene expression levels of a plurality of genes for one or more cells of a population of neuronal progenitor cells, wherein:
the population of neuronal progenitor cells is from a culture of cells differentiated from pluripotent stem cells under conditions to neurally differentiate the cells; and
the plurality of genes comprise one or more cell cycle genes and/or one or more maturity genes; and
(b) applying the gene expression levels as input to a process configured to predict if the population of neuronal progenitor cells will engraft in a brain region of a subject following implantation of the population of neuronal progenitor cells into the brain region, wherein the predicting is based on gene expression levels of one or more of the plurality of genes.

3. The method of embodiment 2, wherein the population of neuronal progenitor cells are for implantation in a brain region of the subject if the population of neuronal progenitor cells is predicted to engraft.

4. A method of selecting a population of neuronal progenitor cells for implantation in a subject for treating a neurodegenerative disease, the method comprising:
(a) obtaining gene expression levels of a plurality of genes for one or more cells of a population of neuronal progenitor cells, wherein:
the population of neuronal progenitor cells is from a culture of cells differentiated from pluripotent stem cells under conditions to neurally differentiate the cells; and
the plurality of genes comprise one or more cell cycle genes and/or one or more maturity genes; and
(b) applying the gene expression levels as input to a process configured to predict if the population of neuronal progenitor cells will engraft in a brain region of a subject following implantation of the population of neuronal progenitor cells into the brain region, wherein the predicting is based on gene expression levels of one or more of the plurality of genes; and
(c) selecting the population of neuronal progenitor cells for implantation in the subject if the population of neuronal progenitor cells are predicted to engraft.

5. The method of any of embodiments 1-4, wherein the process comprises a machine learning model.

6. The method of embodiment 5, wherein the machine learning model is trained using gene expression levels of the one or more of the plurality of genes for a plurality of reference populations of neuronal progenitor cells that have been differentiated from pluripotent stem cells under conditions to neurally differentiate the cells.

7. The method of embodiment 5 or embodiment 6, wherein the machine learning model is or comprises a supervised machine learning model.

8. The method of any of embodiments 5-7, wherein the machine learning model is trained using (i) gene expression levels of the one or more of the plurality of genes for a plurality of reference populations of neuronal progenitor cells that have been differentiated from pluripotent stem cells under conditions to neurally differentiate the cells and (ii) engraftment fitness of the plurality of reference populations, wherein the engraftment fitness of a reference population indicates whether or not, or the degree to which, the reference population engrafted in a brain region of a subject following implantation of the reference population into the brain region.

9. A method of training a machine learning model, comprising:
(a) obtaining gene expression levels of one or more of a plurality of genes for a plurality of reference populations of neuronal progenitor cells that are from cultures of cells that have been differentiated from pluripotent stem cells under conditions to neurally differentiate the cells, wherein the plurality of genes comprise one or more cell cycle genes and/or one or more maturity genes; and
(b) applying the gene expression levels of the plurality of reference populations as input to train a machine learning model.

10. The method of embodiment 9, wherein the machine learning model is or comprises a supervised machine learning model.

11. The method of embodiment 9 or embodiment 10, further comprising:
(a) receiving engraftment fitness of the plurality of reference populations, wherein the engraftment fitness of a reference population indicates whether or not, or the degree to which, the reference population engrafted in a brain region of a subject following implantation of the reference population into the brain region; and
(b) applying the engraftment fitness of the plurality of reference populations as input to train the machine learning model, wherein the machine learning model is trained to predict based on gene expression levels of one or more of the plurality of genes if a population of neuronal progenitor cells that is from a culture of cells differentiated from pluripotent stem cells under conditions to neurally differentiate the cells will engraft in a brain region of a subject following implantation of the population of neuronal progenitor cells into the brain region.

12. The method of embodiment 8 or embodiment 11, wherein the engraftment fitness of a reference population is determined based on the number of cells of the reference population that are present in the brain region following the implantation.

13. The method of embodiment 12, wherein the number of cells is counted at, about, at least, or at least about 7 days, 14 days, or 21 days following the implantation.

14. The method of any of embodiments 8 and 11-13, wherein a reference population is considered fit for engraftment if at least a predetermined number of cells are present in the brain region following the implantation.

15. The method of embodiment 14, wherein the predetermined number of cells is greater than or greater than about 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9%, or 10% of the number of cells implanted in the brain region.

16. The method of any of embodiments 5-15, wherein the machine learning model is or comprises a classification model.

17. The method of any of embodiments 5-16, wherein the machine learning model is or comprises a regression model.

18. The method of any of embodiments 5-17, wherein the machine learning model is or comprises a logistic regression model.

19. The method of any of embodiments 5-18, wherein the machine learning model is or comprises a penalized model.

20. The method of any of embodiments 5-19, wherein the machine learning model is or comprises a ridge regression model, a lasso regression model, and/or an elastic net regression model.

21. The method of any of embodiments 5-20, wherein the machine learning model is or comprises a lasso regression model.

22. The method of any of embodiments 5-21, wherein the machine learning model is or comprises a lasso logistic regression model.

23. The method of any of embodiments 1-22, wherein the plurality of genes comprises, comprises about, comprises greater than, or comprises greater than about 2, 3, 4, 5, 6, 7, 8, 9, 10, 12, 14, 16, 18, 20, 25, 30, 35, 40, 45, 50, 55, 60, 62, 64, 66, 68, or 70 genes.

24. The method of any of embodiments 1-23, wherein the plurality of genes comprises between or between about 2 and 100, 2 and 95, 2 and 90, 2 and 85, 2 and 80, 2 and 75, or 2 and 70 genes.

25. The method of any of embodiments 1-24, wherein the plurality of genes comprises between or between about 5 and 100, 5 and 95, 5 and 90, 5 and 85, 5 and 80, 5 and 75, or 5 and 70 genes.

26. The method of any of embodiments 1-25, wherein the gene expression levels of the plurality of genes are gene expression levels of genes associated with the ability of a population of neuronal progenitor cells to engraft in a brain region of a subject.

27. The method of any of embodiments 1-26, wherein the plurality of genes comprise one or more cell cycle genes.

28. The method of any of embodiments 1-27, wherein the one or more cell cycle genes comprise one or more of ANLN, ASPM, AURKA, AURKB, BIRC5, BRINP1, BUB1, BUB1B, CCNA2, CCNB1, CCNB2, CDC20, CDC25C, CDCA2, CDCA8, CDK1, CDKN1A, CENPE, CENPF, CEP55, CIT, DLGAP5, ECT2, ESPL1, FAM83D, FANCD2, FOXM1, GEM, GTSE1, HJURP, HMMR, IQGAP3, KIF11, KIF14, KIF15, KIF18A, KIF20A, KIF23, KIF2C, KIF4A, KIFC1, KNL1, MELK, MKI67, NCAPG, NCAPH, NDC80, NEK2, NUF2, NUSAP1, PBK, PIMREG, PLK1, PLK2, POC1A, PRC1, PRR11, PTTG1, RACGAP1, SAPCD2, SKA3, SPAG5, TACC3, TOP2A, TPX2, TTK, TUBA1C, and UBE2C.

29. The method of any of embodiments 1-28, wherein the one or more cell cycle genes comprise BRINP1, CDKN1A, FAM83D, FANCD2, GEM, PLK2, and SAPCD2.

30. The method of any of embodiments 1-29, wherein the one or more cell cycle genes consist of BRINP1, CDKN1A, FAM83D, FANCD2, GEM, PLK2, and SAPCD2.

31. The method of any of embodiments 1-30, wherein the plurality of genes comprise one or more maturity genes.

32. The method of any of embodiments 1-31, wherein the one or more maturity genes comprise genes whose gene expression levels increase during differentiation, optionally during days 17-22 of differentiation of the culture of cells.

33. The method of any of embodiments 1-32, wherein the one or more maturity genes comprise genes whose gene expression levels increase substantially monotonically during differentiation, optionally during days 17-22 of differentiation of the culture of cells.

34. The method of any of embodiments 1-33, wherein the one or more maturity genes comprise one or more of AC104083.1, ACE, ACSL1, AFAP1, APBA1, ARHGDIG, ARL8A, BDNF, BICDL1, CAMK2B, CCDC112, CDK5R2, CEP170B, CHGB, DIRAS1, DMTN, DNAJB5, DUSP26, FBXL16, FNBP1L, GFOD2, GUCY1A1, HCN3, JPT1, KCNB1, KCNC1, KCNH6, KIF1A, KLF7, LINC01128, MACO1, MAP3K9, MAPRE3, MIR100HG, NACAD, NCAM1, NFIC, NFIX, PARP6, PRKACB, RIMS1, SBK1, SHISA7, SLC6A17, SPTBN1, SRGAP2, SYT13, TMEM151B, TPH1, and TRIM46.

35. The method of any of embodiments 1-34, wherein the one or more maturity genes comprise genes whose gene expression levels decrease during differentiation, optionally during days 17-22 of differentiation of the culture of cells.

36. The method of any of embodiments 1-35, wherein the one or more maturity genes comprise genes whose gene expression levels decrease substantially monotonically during differentiation, optionally during days 17-22 of differentiation of the culture of cells.

37. The method any of embodiments 1-36, wherein the one or more maturity genes comprise one or more of ACSS3, ADSS, ANP32A, ANXA11, ASPH, CCDC160, CCDC60, COL23A1, CTSC, CYFIP1, DAAM2, DPY19L1, FABP7, FAM71E2, FAM86C2P, FZD2, HAPLN3, HLA-E, HTATIP2, IKZF2, IL4R, ITGA5, KCNJ2-AS1, LPIN3, LRIG1, LRIG3, MGST1, MRVI1, MYCBP, NAALAD2, NAV2, NR6A1, NT5DC1, PLAG1, POFUT2, PRTG, PTCH1, PTPN13, SALL4, SAV1, SELENOP, SEMA5B, SLC35D2, SLC66A3, STOX1, SUCLG2, TGFBR3, TM6SF2, TOB1, and YBX3.

38. The method of any of embodiments 1-8 and 12-37, wherein if the population of neuronal progenitor cells is predicted to not engraft, the method further comprises repeating steps (a) and (b) for the same or a different population of neuronal progenitor cells.

39. The method of any of embodiments 1-8 and 12-38, further comprising selecting based on an output of the process the population of neuronal progenitor cells as a population of neuronal progenitor cells that is predicted to engraft.

40. The method of any of embodiments 1-8 and 11-39, wherein the process is configured to predict the presence or absence of engraftment, and the population of neuronal progenitor cells is predicted to engraft if the process predicts the presence of engraftment.

41. The method of any of embodiments 1-8 and 11-39, wherein the process is configured to predict the probability of engraftment, and the population of neuronal progenitor cells is predicted to engraft if the predicted probability of engraftment exceeds a predetermined threshold level.

42. The method of embodiment 41, wherein the predetermined probability threshold level is, is about, is greater than, or is greater than about 0.5.

43. The method of embodiment 41 or embodiment 42, wherein the predetermined probability threshold level is, is about, is greater than, or is greater than about 0.55, 0.6, 0.65, 0.7, 0.75, 0.8, 0.85, 0.9, 0.91, 0.92, 0.93, 0.94, 0.95, 0.96, 0.97, 0.98, or 0.99.

44. The method of any of embodiments 1-8 and 11-39, wherein the process is configured to predict the degree of engraftment, and the population of neuronal progenitor cells is predicted to engraft if the predicted degree of engraftment exceeds a predetermined threshold level.

45. The method of any one of embodiments 39-44, further comprising harvesting the selected population of neuronal progenitor cells.

46. A method of assessing engraftment fitness of a population of neuronal progenitor cells, the method comprising (a) measuring gene expression levels of a plurality of genes for one or more cells of a population of neuronal progenitor cells, wherein:
the population of neuronal progenitor cells is from a culture of cells differentiated from pluripotent stem cells under conditions to neurally differentiate the cells; and
the plurality of genes comprise one or more of AC104083.1, ACE, ACSL1, ACSS3, ADSS, AFAP1, ANLN, ANP32A, ANXA11, APBA1, ARHGDIG, ARL8A, ASPH, ASPM, AURKA, AURKB, BDNF, BICDL1, BIRC5, BRINP1, BUB1, BUB1B, CAMK2B, CCDC112, CCDC160, CCDC60, CCNA2, CCNB1, CCNB2, CDC20, CDC25C, CDCA2, CDCA8, CDK1, CDK5R2, CDKN1A, CENPE, CENPF, CEP170B, CEP55, CHGB, CIT, COL23A1, CTSC, CYFIP1, DAAM2, DIRAS1, DLGAP5, DMTN, DNAJB5, DPY19L1, DUSP26, ECT2, ESPL1, FABP7, FAM71E2, FAM83D, FAM86C2P, FANCD2, FBXL16, FNBP1L, FOXM1, FZD2, GEM, GFOD2, GTSE1, GUCY1A1, HAPLN3, HCN3, HJURP, HLA-E, HMMR, HTATIP2, IKZF2, IL4R, IQGAP3, ITGA5, JPT1, KCNB1, KCNC1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MGST1, MIR100HG, MKI67, MRVI1, MYCBP, NAALAD2, NACAD, NAV2, NCAM1, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SALL4, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TOP2A, TPH1, TPX2, TRIM46, TTK, TUBA1C, UBE2C, and YBX3.

47. The method of embodiment 46, wherein the plurality of genes comprise one or more of ANLN, ASPM, AURKA, AURKB, BIRC5, BRINP1, BUB1, BUB1B, CCNA2, CCNB1, CCNB2, CDC20, CDC25C, CDCA2, CDCA8, CDK1, CDKN1A, CENPE, CENPF, CEP55, CIT, DLGAP5, ECT2, ESPL1, FAM83D, FANCD2, FOXM1, GEM, GTSE1, HJURP, HMMR, IQGAP3, KIF11, KIF14, KIF15, KIF18A, KIF20A, KIF23, KIF2C, KIF4A, KIFC1, KNL1, MELK, MKI67, NCAPG, NCAPH, NDC80, NEK2, NUF2, NUSAP1, PBK, PIMREG, PLK1, PLK2, POC1A, PRC1, PRR11, PTTG1, RACGAP1, SAPCD2, SKA3, SPAG5, TACC3, TOP2A, TPX2, TTK, TUBA1C, and UBE2C.

48. The method of embodiment 46 or embodiment 47, wherein the plurality of genes comprise one or more of AC104083.1, ACE, ACSL1, AFAP1, APBA1, ARHGDIG, ARL8A, BDNF, BICDL1, CAMK2B, CCDC112, CDK5R2, CEP170B, CHGB, DIRAS1, DMTN, DNAJB5, DUSP26, FBXL16, FNBP1L, GFOD2, GUCY1A1, HCN3, JPT1, KCNB1, KCNC1, KCNH6, KIF1A, KLF7, LINC01128, MACO1, MAP3K9, MAPRE3, MIR100HG, NACAD, NCAM1, NFIC, NFIX, PARP6, PRKACB, RIMS1, SBK1, SHISA7, SLC6A17, SPTBN1, SRGAP2, SYT13, TMEM151B, TPH1, and TRIM46.

49. The method of any of embodiments 46-48, wherein the plurality of genes comprise one or more of ACSS3, ADSS, ANP32A, ANXA11, ASPH, CCDC160, CCDC60, COL23A1, CTSC, CYFIP1, DAAM2, DPY19L1, FABP7, FAM71E2, FAM86C2P, FZD2, HAPLN3, HLA-E, HTATIP2, IKZF2, IL4R, ITGA5, KCNJ2-AS1, LPIN3, LRIG1, LRIG3, MGST1, MRVI1, MYCBP, NAALAD2, NAV2, NR6A1, NT5DC1, PLAG1, POFUT2, PRTG, PTCH1, PTPN13, SALL4, SAV1, SELENOP, SEMA5B, SLC35D2, SLC66A3, STOX1, SUCLG2, TGFBR3, TM6SF2, TOB1, and YBX3.

50. The method of any of embodiments 46-49, further comprising comparing one or more of the gene expression levels or one or more combinations thereof to one or more predetermined first threshold levels, wherein gene expression levels or combinations thereof that are greater than the first threshold levels are associated with a population of neuronal progenitor cells that is predicted to engraft in a brain region of a subject following implantation of the population of neuronal progenitor cells in the brain region.

51. The method of any of embodiments 46-50, further comprising comparing one or more of the gene expression levels or one or more combinations thereof to one or more predetermined second threshold levels, wherein gene expression levels or combinations thereof that are less than the second threshold levels are associated with a population of neuronal progenitor cells that is predicted to engraft in a brain region of a subject following implantation of the population of neuronal progenitor cells in the brain region.

52. The method of one of embodiments 1-51, wherein the gene expression levels are obtained by RNA sequencing.

53. The method of any of embodiments 1-8, 11-45, and 50-52, wherein the brain region is the substantia nigra.

54. The method of any one of embodiments 1-8 and 11-53, wherein the population of neuronal progenitor cells comprises determined dopaminergic neuron progenitor cells.

55. The method of any of embodiments 1-8 and 12-54, wherein prior to (a), the method further comprises differentiating the culture of cells comprising the population of neuronal progenitor cells.

56. The method of any of embodiments 1-8 and 11-55, wherein the culture of cells comprising the population of neuronal progenitor cells is differentiated from pluripotent stem cells by a process comprising:
(a) performing a first incubation comprising culturing the pluripotent stem cells (PSCs) in a first culture vessel, wherein beginning at the initiation of the first incubation (day 0) the cells are exposed to (i) an inhibitor of TGF-3/activin-Nodal signaling; and (ii) an inhibitor of bone morphogenetic protein (BMP) signaling; and
(b) performing a second incubation comprising culturing cells produced by the first incubation in a second culture vessel under conditions to neurally differentiate the cells.

57. The method of embodiment 56, wherein the second culture vessel is an adherent culture vessel.

58. The method of embodiment 57, wherein the adherent culture vessel is coated with laminin or a fragment thereof.

59. A method of differentiating neural cells, the method comprising:
(a) performing a first incubation comprising culturing pluripotent stem cells (PSCs) in a first culture vessel, wherein beginning at the initiation of the first incubation (day 0) the cells are exposed to (i) an inhibitor of TGF-β/activin-Nodal signaling; and (ii) an inhibitor of bone morphogenetic protein (BMP) signaling; and
(b) performing a second incubation comprising culturing cells produced by the first incubation in a second culture vessel under conditions to neurally differentiate the cells,
wherein the second culture vessel is an adherent culture vessel coated with laminin or a fragment thereof.

60. The method of embodiment 58 or embodiment 59, wherein the laminin is or comprises Laminin-111, Laminin-211, Laminin-121, Laminin-221, Laminin-332, Laminin-3A32, Laminin-3B32, Laminin-311, Laminin-3A11, Laminin-321, Laminin-3A21, Laminin-411, Laminin-421, Laminin-511, Laminin-521, Laminin-213, Laminin-423, Laminin-522, Laminin-523, or a fragment of any of the foregoing, optionally wherein the laminin is or comprises Laminin-521, Laminin-111, Laminin-511, or a fragment of any of the foregoing.

61. The method of any of embodiments 58-60, wherein the laminin is or comprises Laminin-511 or a fragment thereof.

62. The method of any of embodiments 58-61, wherein the laminin is or comprises a Laminin-511 E8 fragment.

63. The method of any of embodiments 56-62, wherein the first culture vessel is a non-adherent culture vessel.

64. The method of any of embodiments 56-63, wherein, beginning on day 0, the cells are also exposed to (iii) at least one activator of Sonic Hedgehog (SHH) signaling and (iv) an inhibitor of glycogen synthase kinase 3β(GSK3β).

65. The method of any of embodiments 56-64, wherein the second incubation begins on about day 7.

66. The method of any of embodiments 56-65, wherein the cells are exposed to the inhibitor of TGF-3/activin-Nodal signaling up to a day at or before day 7.

67. The method of any of embodiments 56-66, wherein the cells are exposed to the inhibitor of TGF-β/activin-Nodal beginning at day 0 and through day 6, inclusive of each day.

68. The method of any of embodiments 64-67, wherein the cells are exposed to the at least one activator of SHH signaling up to a day at or before day 7.

69. The method of any of embodiments 64-68, wherein the cells are exposed to the at least one activator of SHH signaling beginning at day 0 and through day 6, inclusive of each day.

70. The method of any of embodiments 56-69, wherein the cells are exposed to the inhibitor of BMP signaling up to a day at or before day 11.

71. The method of any of embodiments 56-70, wherein the cells are exposed to the inhibitor of BMP signaling beginning at day 0 and through day 10, inclusive of each day.

72. The method of any of embodiments 64-71, wherein the cells are exposed to the inhibitor of GSK3β signaling up to a day at or before day 13.

73. The method of any of embodiments 64-72, wherein the cells are exposed to the inhibitor of GSK3β signaling beginning at day 0 and through day 12, inclusive of each day.

74. The method of any of embodiments 63-73, wherein the first incubation produces a spheroid of cells, and prior to performing the second incubation, the spheroid is dissociated to produce a cell suspension, and cells of the cell suspension are cultured in the second culture vessel.

75. The method of embodiment 74, wherein the spheroid is dissociated by enzymatic dissociation.

76. The method of embodiment 74 or embodiment 75, wherein the spheroid is dissociated by enzymatic dissociation comprising use of an enzyme selected from among the group consisting of accutase, dispase, collagenase, and combinations thereof.

77. The method of any of embodiments 74-76, wherein the spheroid is dissociated by enzymatic dissociation comprising use of accutase.

78. The method of any of embodiments 74-77, wherein the dissociating is carried out at a time when the cells of the spheroid express at least one of PAX6 and OTX2.

79. The method of any of embodiments 74-78, wherein the dissociating is carried out on about day 7.

80. The method of any of embodiments 56-62, wherein the first culture vessel is an adherent culture vessel coated with laminin or a fragment thereof, optionally wherein the laminin or a fragment thereof is or comprises: Laminin-111, Laminin-211, Laminin-121, Laminin-221, Laminin-332, Laminin-3A32, Laminin-3B32, Laminin-311, Laminin-3A11, Laminin-321, Laminin-3A21, Laminin-411, Laminin-421, Laminin-511, Laminin-521, Laminin-213, Laminin-423, Laminin-522, Laminin-523, or a fragment of any of the foregoing, further optionally wherein the laminin is or comprises Laminin-511 or Laminin-511 E8 fragment.

81. The method of embodiment 80, wherein beginning on day 1, the cells are exposed to (iii) at least one activator of Sonic Hedgehog (SHH) signaling; and beginning on day 2, the cells are exposed to an (iv) an inhibitor of glycogen synthase kinase 3β (GSK3β) signaling.

82. The method of embodiment 80 or embodiment 81, wherein the second incubation begins on about day 11.

83. The method of any one of embodiments 80-82, wherein the cells are exposed to the inhibitor of TGF-3/activin-Nodal signaling up to a day at or before day 5.

84. The method of any one of embodiments 80-83, wherein the cells are exposed to the inhibitor of TGF-β/activin-Nodal beginning at day 0 and through day 4, inclusive of each day.

85. The method of any one of embodiments 81-84, wherein the cells are exposed to the at least one activator of SHH signaling up to a day at or before day 7.

86. The method of any one of embodiments 81-85, wherein the cells are exposed to the at least one activator of SHH signaling beginning at day 0 and through day 6, inclusive of each day.

87. The method of any one of embodiments 80-86, wherein the cells are exposed to the inhibitor of BMP signaling up to a day at or before day 11.

88. The method of any one of embodiments 80-87, wherein the cells are exposed to the inhibitor of BMP signaling beginning at day 0 and through day 10, inclusive of each day.

89. The method of any one of embodiments 81-88, wherein the cells are exposed to the inhibitor of GSK3β signaling up to a day at or before day 13.

90. The method of any one of embodiments 81-89, wherein the cells are exposed to the inhibitor of GSK3β signaling beginning at day 0 and through day 12, inclusive of each day.

91. The method of any of embodiments 1-90, wherein the cells are cultured to differentiate the cells to determined dopaminergic neuron progenitor cells.

92. The method of any one of embodiments 1-91, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF3β) (collectively, “BAGCT”); and (vi) an inhibitor of Notch signaling.

93. The method of embodiment 92, wherein the cells are exposed to BAGCT and the inhibitor of Notch signaling beginning on day 11.

94. The method of embodiment 92 or embodiment 93, wherein the cells are exposed to BAGCT and the inhibitor of Notch signaling beginning at day 11 and until harvest of the neurally differentiated cells, optionally until day 20.

95. The method of any one of embodiments 56-94, wherein the inhibitor of TGF-β/activin-Nodal signaling is SB431542.

96. The method of embodiment 95, wherein the cells are exposed to SB431542 at a concentration of between about 1 μM and about 20 μM, between about 5 μM and about 15 μM, or between about 8 μM and about 12 μM, optionally about 10 μM.

97. The method of any one of embodiments 64-79 and 81-96, wherein the at least one activator of SHH signaling is SHH or purmorphamine.

98. The method of any one of embodiments 64-79 and 81-97, wherein the at least one activator of SHH signaling comprises two activators of SHH signaling selected from SHH protein and purmorphamine.

99. The method of embodiment 97 or embodiment 98, wherein the cells are exposed to SHH at a concentration of between about 10 ng/mL and 500 ng/mL, between about 20 ng/mL and about 400 ng/mL, between about 50 ng/mL and about 200 ng/mL, or between about 75 ng/mL and about 150 ng/mL, optionally about 100 ng/mL.

100. The method of any one of embodiments 97-99, wherein the cells are exposed to purmorphamine at a concentration of between about 1 μM and about 20 PM, between about 5 μM and about 15 μM, or between about 8 μM and about 12 μM, optionally about 10 μM.

101. The method of any one of embodiments 56-100, wherein the inhibitor of BMP signaling is LDN193189.

102. The method of embodiment 101, wherein the cells are exposed to LDN193189 at a concentration of between about 10 nM and 500 nM, between about 20 nM and about 400 nM, between about 50 nM and about 200 nM, or between about 75 nM and about 150 nM, optionally about 100 nM.

103. The method of any one of embodiments 64-79 and 81-102, wherein the inhibitor of GSK3β signaling is CHIR99021.

104. The method of embodiment 103, wherein the cells are exposed to CHIR99021 at a concentration of between about 0.1 μM and about 5 μM, between about 0.5 μM and about 4 μM, or between about 1 μM and about 3 μM, optionally about 2 PM.

105. The method of any one of embodiments 92-104, wherein the cells are exposed to GDNF at a concentration of between about 1 ng/mL and about 100 ng/mL, between about 5 ng/mL and about 80 ng/mL, between about 10 ng/mL and about 60 ng/mL, or between about 15 ng/mL and about 30 ng/mL, optionally about 20 ng/mL.

106. The method of any one of embodiments 92-105, wherein the cells are exposed to BDNF at a concentration of between about 1 ng/mL and about 100 ng/mL, between about 5 ng/mL and about 80 ng/mL, between about 10 ng/mL and about 60 ng/mL, or between about 15 ng/mL and about 30 ng/mL, optionally about 20 ng/mL.

107. The method of any one of embodiments 92-106, wherein the cells are exposed to dbcAMP at a concentration of between about 0.1 mM and 5 mM, between about 0.2 mM and about 4 mM, between about 0.3 mM and about 3 mM, or between about 0.4 mM and about 2 mM, optionally about 0.5 mM.

108. The method of any one of embodiments 92-107, wherein the cells are exposed to ascorbic acid at a concentration of between about 0.05 mM and about 5 mM, between about 0.1 mM and about 1 mM, or between about 0.2 mM and about 0.5 mM, optionally about 0.2 mM.

109. The method of any one of embodiments 92-108, wherein the cells are exposed to TGFβ3 at a concentration of between about 0.1 ng/mL and about 5 ng/mL, between about 0.3 ng/mL and about 3 ng/mL, or between about 0.5 ng/mL and about 2 ng/mL, optionally about 1 ng/mL.

110. The method of any one of embodiments 92-109, wherein the inhibitor of Notch signaling is DAPT.

111. The method of any one of embodiments 92-110, wherein the cells are exposed to DAPT at a concentration of between about 1 μM and about 20 μM, between about 5 μM and about 15 μM, or between about 8 μM and about 12 μM, optionally about 10 μM.

112. The method of any one of embodiments 56-111, wherein the culturing in the first incubation and/or the second incubation is carried out in media comprising serum or a serum replacement.

113. The method of any one of embodiments 56-112, wherein the cells are cultured in a media comprising serum or a serum replacement from day about 0 to about day 10.

114. The method of embodiment 112 or embodiment 113, wherein the serum or serum replacement comprises about 5% of the media (v/v) or about 2% of the media (v/v).

115. The method of any one of embodiments 112-114, wherein the media comprises about 5% serum or serum replacement (v/v) from about day 0 to about day 1 about 2% serum replacement (v/v) from about day 2 to about day 10.

116. The method of any one of embodiments 59-115, further comprising harvesting the neurally differentiated cells, optionally wherein the harvesting comprises enzymatic dissociation comprising use of Accutase.

117. The method of any of embodiments 45, 52-58, and 60-116, wherein the harvesting is carried out at about day 16 or later, optionally between about day 18 and about day 23, optionally wherein the harvesting comprises enzymatic dissociation comprising use of Accutase.

118. The method of any of embodiments 45, 52-58, and 60-117, wherein the harvesting is carried out at or about at day 18, day 19, day 20, day 21, day 22, or day 23, optionally wherein the harvesting comprises enzymatic dissociation comprising use of Accutase.

119. The method of any of embodiments 45, 52-58, and 60-118, wherein the harvesting is carried out at or about at day 20, optionally wherein the harvesting comprises enzymatic dissociation comprising use of Accutase.

120. The method of any of embodiments 45-119, wherein the cells are passaged during the first incubation and/or during the second incubation by enzymatic dissociation comprising use of Accutase.

121. The method of any of embodiments 45, 52-58, and 60-120, further comprising formulating the harvested cells with a cryoprotectant.

122. The method of embodiment 121, wherein the cryoprotectant is selected from among the group consisting of glycerol, propylene glycol, and dimethyl sulfoxide (DMSO).

123. The method of embodiment 121 or embodiment 122, further comprising cryopreserving the formulated cells.

124. The method of embodiment 123, wherein the cryopreserving comprises controlled rate freezing.

125. The method of any one of embodiments 1-124, wherein the pluripotent stem cells are embryonic stem (ES) cells, induced pluripotent stem cells (iPSCs), or a combination thereof.

126. The method of any one of embodiments 1-125, wherein the pluripotent stem cells are induced pluripotent stem cells, optionally human induced pluripotent stem cells.

127. The method of any one of embodiments 1-8, 11-45, 50-58, and 60-126, wherein the pluripotent stem cells are autologous to the subject.

128. The method of any one of embodiments 1-8, 11-45, 50-58, and 60-126, wherein the pluripotent stem cells are allogeneic to the subject.

129. The method of any one of embodiments 1-128, wherein the pluripotent stem cells are from a healthy human subject.

130. The method of any one of embodiments 1-128, wherein the pluripotent stem cells are from a human subject with a neurodegenerative disease or condition.

131. The method of embodiment 130, wherein the neurodegenerative disease or condition comprises the loss of dopaminergic neurons.

132. The method of embodiment 130 or embodiment 131, wherein the neurodegenerative disease or condition is a Parkinsonism.

133. The method of embodiment 130 or embodiment 131, wherein the neurodegenerative disease or condition is Parkinson's disease.

134. The method of any one of embodiments 1-133, wherein the pluripotent stem cells are hypoimmunogenic.

135. The method of embodiment 134, wherein the pluripotent stem cells are engineered to (a) remove genes encoding one or more of polymorphic HLA-A/-B/-C and HLA class II molecules; and (b) to provide genes encoding one or more of PD-L1, HLA-G, and CD47, optionally into a AAVS1 safe harbor locus.

136. A therapeutic composition produced by the method of any of embodiments 59-135.

137. A population of neuronal progenitor cells that is selected as a population of neuronal progenitor cells that is predicted to engraft by the method of any of embodiments 39-45, 52-58, and 60-135.

138. A therapeutic composition comprising the population of neuronal progenitor cells of embodiment 137.

139. A therapeutic composition comprising determined dopaminergic neuronal progenitor cells (DDPCs) derived from pluripotent stem cells, wherein the therapeutic composition exhibits one or more of:
(a) a ratio of ASPM to GAPDH expression of greater than about 7×10⁻⁴;
(b) a ratio of AURKB to GAPDH expression of greater than about 9×10⁻⁴;
(c) a ratio of BRINP1 to GAPDH expression of greater than about 9×10⁻⁵;
(d) a ratio of BUB1 to GAPDH expression of greater than about 3×10⁻³;
(e) a ratio of CCNB2 to GAPDH expression of greater than about 3×10⁻³;
(f) a ratio of CDC20 to GAPDH expression of greater than about 3×10⁻³;
(g) a ratio of CDC25C to GAPDH expression of greater than about 5×10⁻⁴;
(h) a ratio of CDKN1A to GAPDH expression of greater than about 1×10⁻³;
(i) a ratio of CENPF to GAPDH expression of greater than about 7×10⁻³;
(j) a ratio of DLGAP5 to GAPDH expression of greater than about 2×10⁻³;
(k) a ratio of FAM83D to GAPDH expression of greater than about 6×10⁻⁴;
(l) a ratio of FANCD2 to GAPDH expression of greater than about 3×10⁻³;
(m) a ratio of GEM to GAPDH expression of greater than about 6×10⁻⁴;
(n) a ratio of HMMR to GAPDH expression of greater than about 8×10⁻⁴;
(o) a ratio of IQGAP3 to GAPDH expression of greater than about 1×10⁻³;
(p) a ratio of KIF20A to GAPDH expression of greater than about 1×10⁻³;
(q) a ratio of KIF2C to GAPDH expression of greater than about 3×10⁻³;
(r) a ratio of KIFC1 to GAPDH expression of greater than about 2×10⁻³;
(s) a ratio of MKI67 to GAPDH expression of greater than about 2×10⁻³;
(t) a ratio of PIMREG to GAPDH expression of greater than about 1×10⁻³;
(u) a ratio of PLK2 to GAPDH expression of greater than about 4×10⁻³;
(v) a ratio of PTTG1 to GAPDH expression of greater than about 3×10⁻³;
(w) a ratio of SAPCD2 to GAPDH expression of greater than about 1×10⁻³;
(x) a ratio of TOP2A to GAPDH expression of greater than about 3×10⁻²;
(y) a ratio of TPX2 to GAPDH expression of greater than about 7×10⁻³; and
(z) a ratio of TTK to GAPDH expression of greater than about 2×10⁻³.

140. The therapeutic composition of any of embodiments 136, 138, and 139, wherein:
(a) the ratio of ASPM to GAPDH expression is between about 7×10⁻⁴ and about 2×10⁻¹;
(b) the ratio of AURKB to GAPDH expression is between about 9×10⁻⁴ and about 4×10⁻²;
(c) the ratio of BRINP1 to GAPDH expression is between about 9×10⁻⁵ and about 5×10⁻²;
(d) the ratio of BUB1 to GAPDH expression is between about 3×10⁻³ and about 7×10⁻²;
(e) the ratio of CCNB2 to GAPDH expression is between about 3×10⁻³ and about 7×10⁻²;
(f) the ratio of CDC20 to GAPDH expression is between about 3×10⁻³ and about 1×10⁻¹;
(g) the ratio of CDC25C to GAPDH expression is between about 5×10⁻⁴ and about 3×10⁻²;
(h) the ratio of CDKN1A to GAPDH expression is between about 1×10⁻³ and about 9×10⁻²;
(i) the ratio of CENPF to GAPDH expression is between about 7×10⁻³ and about 5×10⁻¹;
(j) the ratio of DLGAP5 to GAPDH expression is between about 2×10⁻³ and about 9×10⁻²;
(k) the ratio of FAM83D to GAPDH expression is between about 6×10⁻⁴ and about 3×10⁻²;
(1) the ratio of FANCD2 to GAPDH expression is between about 3×10⁻³ and about 4×10⁻²;
(m) the ratio of GEM to GAPDH expression is between about 6×10⁻⁴ and about 3×10⁻²;
(n) the ratio of HMMR to GAPDH expression is between about 8×10⁻⁴ and about 6×10⁻²;
(o) the ratio of IQGAP3 to GAPDH expression is between about 1×10⁻³ and about 6×10⁻²;
(p) the ratio of KIF20A to GAPDH expression is between about 1×10⁻³ and about 8×10⁻²;
(q) the ratio of KIF2C to GAPDH expression is between about 3×10⁻³ and about 7×10⁻²;
(r) the ratio of KIFC1 to GAPDH expression is between about 2×10⁻³ and about 8×10⁻²;
(s) the ratio of MKI67 to GAPDH expression is between about 2×10⁻³ and about 4×10⁻¹;
(t) the ratio of PIMREG to GAPDH expression is between about 1×10⁻³ and about 4×10⁻²;
(u) the ratio of PLK2 to GAPDH expression is between about 4×10⁻³ and about 6×10⁻²;
(v) the ratio of PTTG1 to GAPDH expression is between about 3×10⁻³ and about 9×10⁻²;
(w) the ratio of SAPCD2 to GAPDH expression is between about 1×10⁻³ and about 3×10⁻²;
(x) the ratio of TOP2A to GAPDH expression is between about 3×10⁻² and about 7×10⁻¹;
(y) the ratio of TPX2 to GAPDH expression is between about 7×10⁻³ and about 2×10⁻¹; and/or
(z) the ratio of TTK to GAPDH expression is between about 2×10⁻³ and about 8×10⁻².

141. The therapeutic composition of embodiment 139 or embodiment 140, wherein the composition exhibits between two and 26 of (a)-(z).

142. The therapeutic composition of any of embodiments 136 and 138-141, wherein:
the ratio of BRINP1 to GAPDH expression is between about 9×10⁻⁵ and about 5×10⁻²;
the ratio of CDKN1A to GAPDH expression is between about 1×10⁻³ and about 9×10⁻²;
the ratio of FAM83D to GAPDH expression is between about 6×10⁻⁴ and about 3×10⁻²;
the ratio of FANCD2 to GAPDH expression is between about 3×10⁻³ and about 4×10⁻²;
the ratio of GEM to GAPDH expression is between about 6×10⁻⁴ and about 3×10⁻²;
the ratio of PLK2 to GAPDH expression is between about 4×10⁻³ and about 6×10⁻²; and/or
the ratio of SAPCD2 to GAPDH expression is between about 1×10⁻³ and about 3×10⁻².

143. The therapeutic composition of any one of embodiments 136 and 138-142, wherein cells in the therapeutic composition express EN1 and CORIN.

144. The therapeutic composition of any one of embodiments 136 and 138-143, wherein the composition exhibits:
(a) a ratio of EN1 to GAPDH expression of greater than about 1×10⁻⁴; and/or
(b) a ratio of CORIN to GAPDH expression of greater than about 2×10⁻².

145. The therapeutic composition of any one of embodiments 136 and 138-144, wherein the composition exhibits:
a ratio of EN1 to GAPDH expression of between about 1.5×10⁻³ and 1×10⁻²; and/or
a ratio of CORIN to GAPDH expression of between about 5×10⁻² and 5×10⁻¹.

146. The therapeutic composition of any one of embodiments 136 and 138-145, wherein cells in the composition express TH.

147. The therapeutic composition of any one of embodiments 136 and 138-146, wherein less than 10% of the total cells in the composition express TH.

148. The therapeutic composition of any one of embodiments 136 and 138-147, wherein the composition exhibits a ratio of TH to GAPDH expression of less than about 3×10⁻².

149. The therapeutic composition of any one of embodiments 139-148, wherein the expression is RNA expression.

150. The therapeutic composition of embodiment 149, wherein the RNA expression is measured by RNA sequencing.

151. The therapeutic composition of any one of embodiments 136 and 138-150, wherein between about 2% and about 10%, between about 2% and about 8%, between about 2% and about 6%, between about 2% and about 4%, between about 4% and about 10%, between about 4% and about 8%, between about 4% and about 6%, between about 6% and about 10%, between about 6% and about 8%, or between about 8% and 10% of the total cells in the composition express TH.

152. The therapeutic composition of any one of embodiments 136 and 138-151, wherein cells in the composition are capable of engrafting in and innervating other cells in vivo.

153. The therapeutic composition of any one of embodiments 136 and 138-152, wherein cells in the composition are capable of producing dopamine and optionally do not produce or do not substantially produce norepinephrine.

154. The therapeutic composition of any one of embodiments 136 and 138-153, wherein the composition comprises at least 5 million total cells, at least 10 million total cells, at least 15 million total cells, at least 20 million total cells, at least 30 million total cells, at least 40 million total cells, at least 50 million total cells, at least 100 million total cells, at least 150 million total cells, or at least 200 million total cells.

155. The therapeutic composition of any one of embodiments 136 and 138-154, wherein the composition comprises between at or about 5 million total cells and at or about 200 million total cells, between at or about 5 million total cells and at or about 150 million total cells, between at or about 5 million total cells and at or about 100 million total cells, between at or about 5 million total cells and at or about 50 million total cells, between at or about 5 million total cells and at or about 25 million total cells, between at or about 5 million total cells and at or about 10 million total cells, between at or about 10 million total cells and at or about 200 million total cells, between at or about 10 million total cells and at or about 150 million total cells, between at or about 10 million total cells and at or about 100 million total cells, between at or about 10 million total cells and at or about 50 million total cells, between at or about 10 million total cells and at or about 25 million total cells, between at or about 25 million total cells and at or about 200 million total cells, between at or about 25 million total cells and at or about 150 million total cells, between at or about 25 million total cells and at or about 100 million total cells, between at or about 25 million total cells and at or about 50 million total cells, between at or about 50 million total cells and at or about 200 million total cells, between at or about 50 million total cells and at or about 150 million total cells, between at or about 50 million total cells and at or about 100 million total cells, between at or about 100 million total cells and at or about 200 million total cells, between at or about 100 million total cells and at or about 150 million total cells, or between at or about 150 million total cells and at or about 200 million total cells.

156. The therapeutic composition of any one of embodiments 136 and 138-155, wherein at least about 70%, 75%, 80%, 85%, 90%, or 95% of the total cells in the composition are viable.

157. The therapeutic composition of any one of embodiments 136 and 138-156, wherein the composition comprises a cryoprotectant.

158. The therapeutic composition of embodiment 157, wherein the cryoprotectant is selected from among the group consisting of glycerol, propylene glycol, and dimethyl sulfoxide (DMSO).

159. The therapeutic composition of any one of embodiments 136 and 138-158, wherein the composition is for use in treatment of a neurodegenerative disease or condition in a subject, optionally wherein the neurodegenerative disease or condition comprises a loss of dopaminergic neurons.

160. The therapeutic composition of any one of embodiments 136 and 138-159, wherein the neurodegenerative disease or condition comprises a loss of dopaminergic neurons in the substantia nigra, optionally in the SNc.

161. The therapeutic composition of any of embodiments 158-160, wherein the neurodegenerative disease or condition is Parkinson's disease.

162. The therapeutic composition of any of embodiments 158-160, wherein the neurodegenerative disease or condition is a Parkinsonism.

163. A method of treatment, comprising implanting in a brain region of a subject in need thereof a therapeutically effective amount of the therapeutic composition of any one of embodiments 136 and 138-162.

164. The method of embodiment 163, wherein the number of cells implanted in the subject is between about 0.25×10⁶ cells and about 20×10⁶ cells, between about 0.25×10⁶ cells and about 15×10⁶ cells, between about 0.25×10⁶ cells and about 10×10⁶ cells, between about 0.25×10⁶ cells and about 5×10⁶ cells, between about 0.25×10⁶ cells and about 1×10⁶ cells, between about 0.25×10⁶ cells and about 0.75×10⁶ cells, between about 0.25×10⁶ cells and about 0.5×10⁶ cells, between about 0.5×10⁶ cells and about 20×10⁶ cells, between about 0.5×10⁶ cells and about 15×10⁶ cells, between about 0.5×10⁶ cells and about 10×10⁶ cells, between about 0.5×10⁶ cells and about 5×10⁶ cells, between about 0.5×10⁶ cells and about 1×10⁶ cells, between about 0.5×10⁶ cells and about 0.75×10⁶ cells, between about 0.75×10⁶ cells and about 20×10⁶ cells, between about 0.75×10⁶ cells and about 15×10⁶ cells, between about 0.75×10⁶ cells and about 10×10⁶ cells, between about 0.75×10⁶ cells and about 5×10⁶ cells, between about 0.75×10⁶ cells and about 1×10⁶ cells, between about 1×10⁶ cells and about 20×10⁶ cells, between about 1×10⁶ cells and about 15×10⁶ cells, between about 1×10⁶ cells and about 10×10⁶ cells, between about 1×10⁶ cells and about 5×10⁶ cells, between about 5×10⁶ cells and about 20×10⁶ cells, between about 5×10⁶ cells and about 15×10⁶ cells, between about 5×10⁶ cells and about 10×10⁶ cells, between about 10×10⁶ cells and about 20×10⁶ cells, between about 10×10⁶ cells and about 15×10⁶ cells, or between about 15×10⁶ cells and about 20×10⁶ cells.

165. The method of embodiment 163 or embodiment 164, wherein the subject has a neurodegenerative disease or condition.

166. The method of any one of embodiments 163-165, wherein the neurodegenerative disease or condition comprises the loss of dopaminergic neurons.

167. The method of any one of embodiments 163-166, wherein the subject has lost at least 50%, at least 60%, at least 70%, or at least 80% of dopaminergic neurons.

168. The method of any one of embodiments 163-167, wherein the subject has lost at least 50%, at least 60%, at least 70%, or at least 80% of dopaminergic neurons in the substantia nigra (SN), optionally in the SN pars compacta (SNc).

169. The method of any one of 163-168, wherein the neurodegenerative disease or condition is a Parkinsonism.

170. The method of any one of embodiments 163-168, wherein the neurodegenerative disease or condition is Parkinson's disease.

171. The method of any of embodiments 163-170, wherein the brain region is the substantia nigra.

172. The method of any one of embodiments 163-171, wherein the implanting is by stereotactic injection.

173. The method of any one of embodiments 163-172, wherein the cells of the therapeutic composition are autologous to the subject.

174. The method of any one of embodiments 163-172, wherein the cells of the therapeutic composition are allogeneic to the subject.

175. The method of any one of embodiments 163-174, wherein the cells of the therapeutic composition are hypoimmunogenic.

176. The method of embodiment 175, wherein the cells of the therapeutic composition are engineered to (a) remove genes encoding one or more of polymorphic HLA-A/-B/-C and HLA class II molecules; and (b) to provide genes encoding one or more of PD-L1, HLA-G, and CD47, optionally into a AAVS1 safe harbor locus.

### VIII. EXAMPLES

The following examples are included for illustrative purposes only and are not intended to limit the scope of the invention.

### Example 1: Method of Differentiatingg iPSCs into Dopamineric (DA) Neurons

The wells of tissue culture plates were coated with laminin 511-e8. On Day −1, patient-derived induced pluripotent stem cells (iPSC) were seeded in the wells of the laminin 511-e8 coated plates at a density of 3.05×10⁵ cells/cm² in mTeSR (STEMCELL Technologies) medium supplemented with 10 μM of the Y-27632 ROCK inhibitor.

On Day 0, approximately 24 hours after cell seeding, differentiation medium comprised of a 1:1 mixture of N2 and B27 media was prepared to contain 5% knockout serum replacement (KSR), 10 μM of the BMP inhibitor LDN193189, and 100 nM of the TGF-β inhibitor SB431542. N2 media consisted of DMEM/F12, N2, Sug/mL insulin, GlutaMax, 100 μM non-essential amino acids, 100 μM β-mercaptoethanol, 50 U/mL Penicillin, and 50 mg/mL streptomycin. B27 medium consisted of neurobasal, B27 with vitamin A, 200 mM glutamine, 50 U/mL penicillin, and 50 mg/mL streptomycin. The mTeSR medium from the plates of iPSCs seeded approximately 24 hours earlier was aspirated and replaced with the differentiation medium.

On Day 1, the medium was replaced with new differentiation medium containing 5% KSR, 10 μM SB431541, 100 nM LDN193189, 100 ng/mL recombinant Sonic Hedgehog (rSHH), and 2 μM purmorphamine.

On each of Days 2, 3, and 4, the medium was replaced with new differentiation medium comprising 2% KSR, 10 μM SB431541, 100 nM LDN193189, 100 ng/mL rSHH, 2 μM purmorphamine, and 2 μM of the GSK3β inhibitor CHIR99021.

On each of Days 5 and 6, the medium was replaced with new differentiation medium comprising 2% KSR, 100 nM LDN193189, 100 ng/mL rSHH, 2 μM purmorphamine, and 2 μM CHIR99021.

On each of Days 7, 8, 9, and 10, the medium was replaced with new differentiation medium comprising 2% KSR, 100 nM LDN193189, and 2 μM CHIR99021.

Beginning at Day 11, the cells were cultured in maturation medium containing Neurobasal, B27, N2, 100 μM non-essential amino acids, GlutaMax, 20 ng/mL recombinant human brain-derived neurotrophic factor (rhBDNF), 20 ng/mL recombinant human glial cell-derived neurotrophic factor (rhGDNF), 0.2 mM ascorbic acid (Vitamin C), 0.5 mM dibutyryl cyclic-AMP (dbcAMP), 1 ng/mL recombinant human transforming growth factor Beta 3 (rhTGFβ3), 10 μM of the gamma secretase inhibitor DAPT. On each of days 11 and 12, the medium is replaced with new maturation medium and supplemented with 2 μM CHIR99021.

On Days 13, 14, and 15, the medium is replaced daily with new maturation medium, without CHIR99021.

In some cases, an alternative method (“non-adherent method”) may be used in which iPSC are seeded on a non-adherent vessel to produce a spheroid of cells, in which prior to performing the second incubation in a second culture vessel, the spheroid is dissociated to produce a cell suspension (e.g. by enzymatic dissociation), and cells of the cell suspension are cultured in the second culture vessel. The non-adherent method may also include adding the small molecules on different schedules, such as described herein.

On Day 16, the cells were passaged at 1:2 ratio by removing the previous maturation medium, washing the cells once with PBS, dissociating the cells with Accutase, washing followed by centrifuging the cells three times, and then resuspending half of the cells in a second culture vessel that is a laminin 511-e8 coated 6-well plates in the maturation medium supplemented with 10 μM Y-27632. On each of Days 17 and 19, the medium is replaced with new maturation media, without CHIR99021 or Y-27632.

On Day 20, the previous maturation medium was aspirated, the cells were washed once with PBS, and the cells were dissociated with Accutase. In some experiments, the cells are dissociated and collected with Accutase on an earlier or later day of the method, such as on day 16, 17, 18, 19, 21, 22, 23, 24, or 25. Cell counts were obtained, and then the cells were cryopreserved in CryoStor CS10 solution at 1×10⁷ cells/mL using a controlled cooling protocol (approximately −1 degree Celsius per minute) until achieving a temperature of −130 degrees Celsius. The cells were then transferred to a liquid nitrogen freezer for longer-term storage.

### Example 2: Efficacy Study Following Transplantation of Differentiated Neural Cells

Differentiated dopaminergic neurons that were differentiated in accordance with the method described in Example 1, with cells collected, i.e., harvested, on either day 18 or day 25, were obtained. These cells were derived from two different iPSC cell lines (PDA410 and PD411).

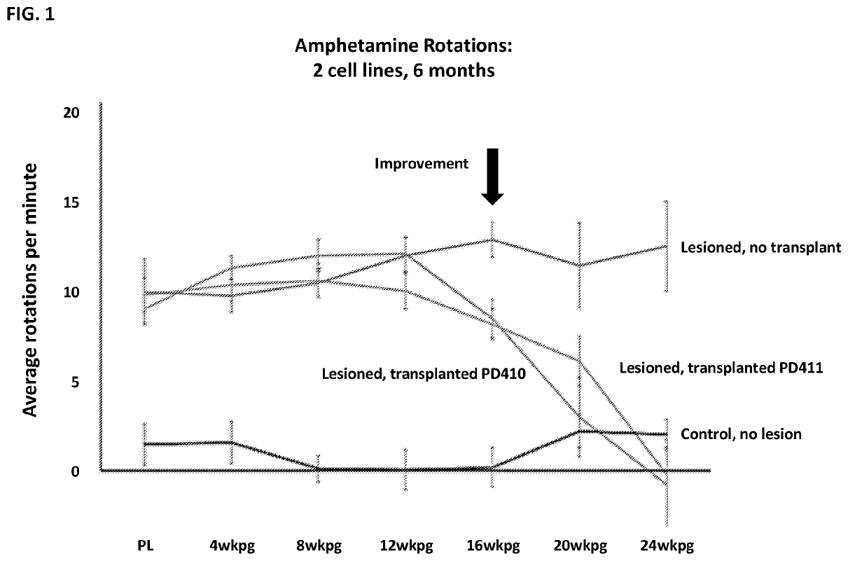

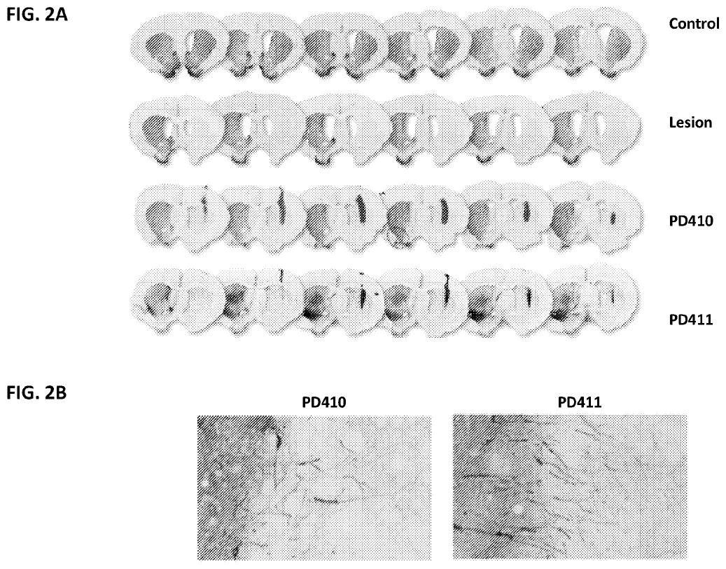

The differentiated dopaminergic neurons were transplanted into Lister-Hooded rats (Cardiff University) that were lesioned via unilateral stereotaxic injection of 6-hydroxydopamine (6-OHDA). Amphetamine-induced rotation was measured over the course of 24 weeks. Control rats that were lesioned but received no transplantation were also tested, and control rats that were not lesioned and received no transplantation were also tested. As shown in FIG. 1, cells that were harvested on day 18 were efficacious over the course of 24 weeks, as shown by the average number of rotations per minute. Cells that were harvested on day 25 were less efficacious over the course of 24 weeks (data not shown). Fiber outgrowth was also assessed. Rats transplanted with the cells harvested on day 18 also exhibited fiber outgrowth (FIG. 2A-2B).

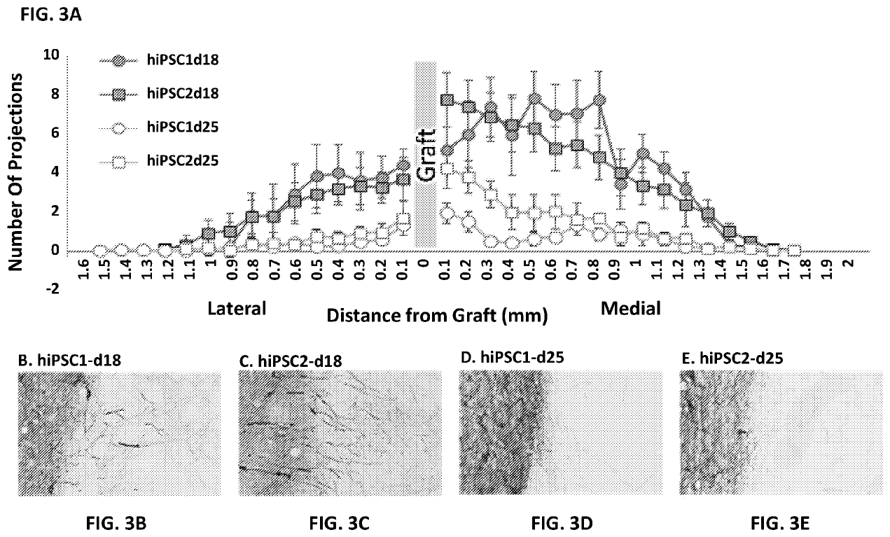

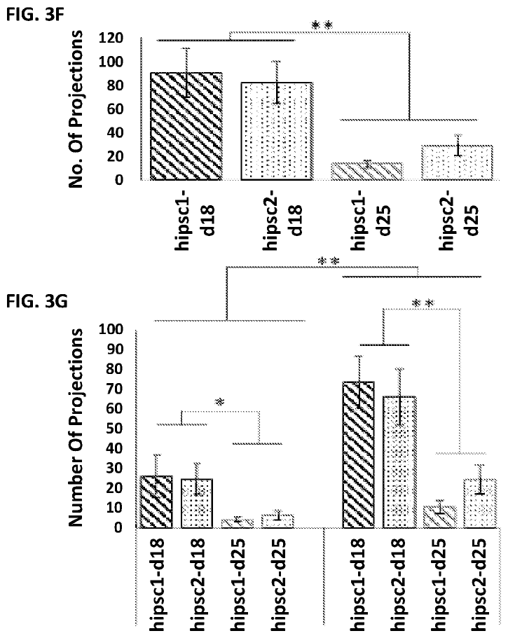

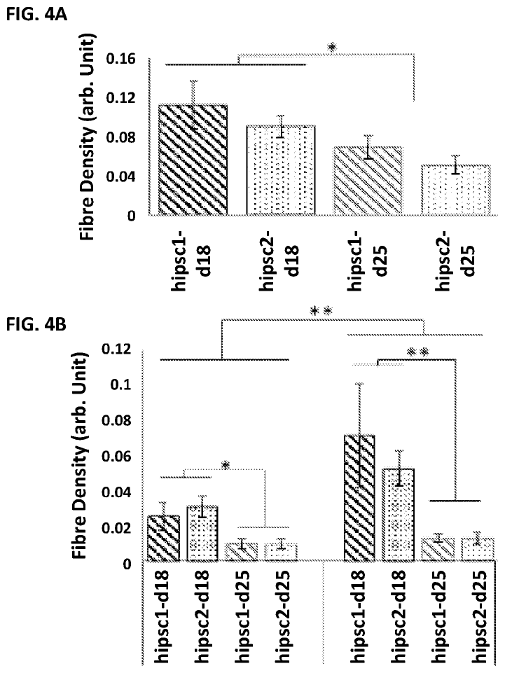

The lateral and medial fiber outgrowth was also assessed in the rats, as well as fiber density. As shown in FIG. 3A-3E, the cells harvested on day 18 (d18) resulted in increased lateral and medial fiber outgrowth as compared to the cells harvested on day 25 (d25). As shown in FIG. 3F-3G, the cells harvested on day 18 resulted in a statistically significant increase in the number of projections as compared to the cells harvested on day 25. The cells harvested on day 18 also resulted in increased fiber density as compared to the cells harvested on day 25 (FIG. 4A-4B).

Collectively, this data demonstrates that differentiated dopaminergic neurons that are harvested on day 18 provide greater efficacy in repairing a lesion when transplanted into a rat model, as compared to cells harvested on day 25.

### Example 3: Analysis of Cell Survival and Fiber Outgrowth in Day 20 vs Day 18

Cells harvested on day 18 or day 20 of different differentiation methods were tested for cell survival and fiber outgrowth following transplantation into lesioned rats. Machine learning was used to identify healthy human nuclei and fiber outgrowth, with over 85% accuracy with cross-validation being achieved.

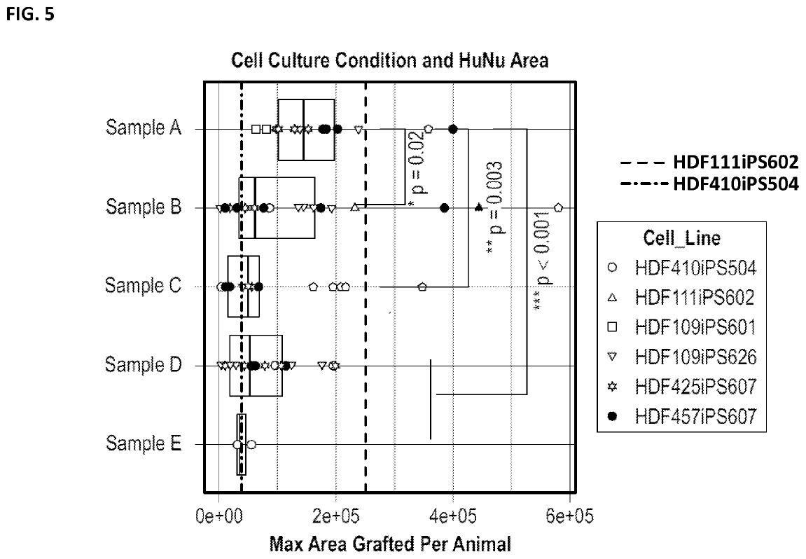

Cells harvested on day 20 of a differentiation method as described in Example 1 in which cells were cultured on a laminin substrate and passaged on day 16 using accutase (Sample A) exhibited increased cell survival, as shown by staining for human nuclei, compared to cells harvested on day 18 of a differentiation method in which cells were cultured on a laminin substrate and passaged on day 16 using accutase (Sample B); cells harvested on day 18 of a differentiation method in which cells were cultured on a laminin substrate and passaged on day 16 using dispase (Sample C); and cells harvested on day 18 of a differentiation method in which cells were cultured on a Geltrex substrate and passaged on day 16 onto a poly-ornithine, laminin, and fibronectin-coated matrix using dispase (Samples D and E). As shown in FIG. 5, cells harvested on day 20 exhibited significantly increased cell survival as compared to each of the cell samples harvested on day 18. For comparison, cell survival of exemplary cells harvested on day 18 of a differentiation method in which cells were cultured on a laminin substrate and passaged on day 16 using accutase (HDF111iPS602) and exemplary cells harvested on day 18 of a differentiation method in which cells were cultured on a Geltrex substrate and passaged on day 16 using dispase (HDF410iPS504) are also shown in FIG. 5.

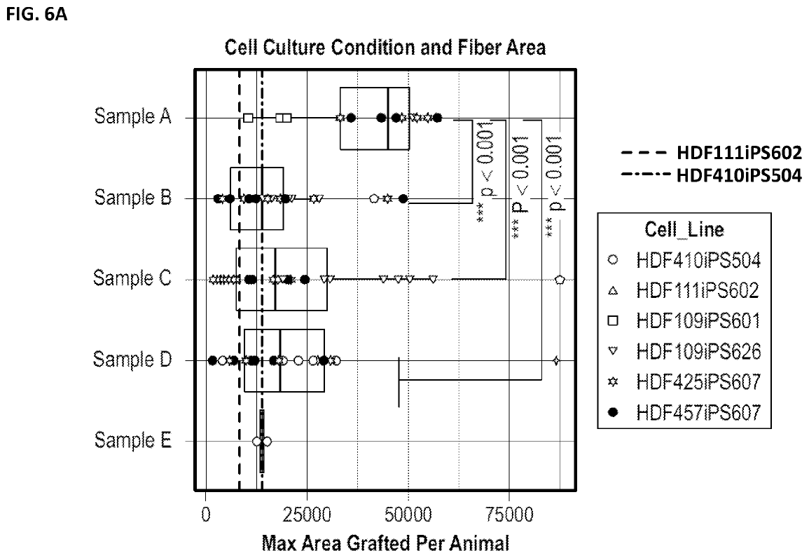

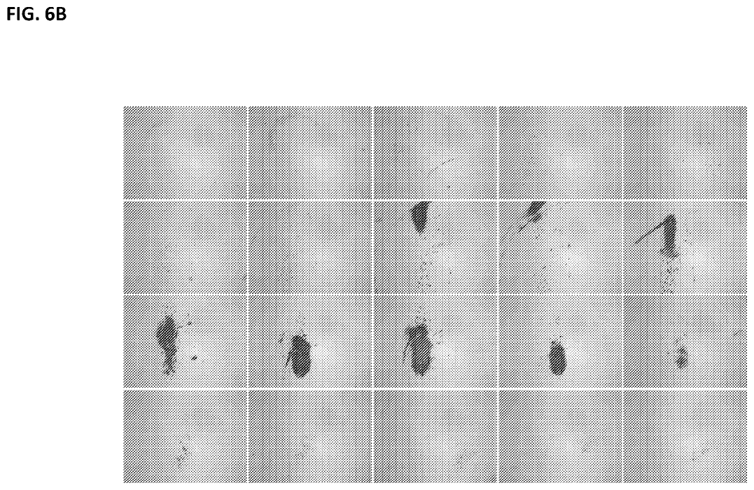

Cells harvested on day 20 also exhibited increased fiber outgrowth as shown by staining for human cytoplasm. As shown in FIG. 6A, cells harvested on day 20 exhibited significantly increased fiber outgrowth as compared to each of the cell samples harvested on day 18. For comparison, fiber outgrowth of HDF111iPS602 and HDF410iPS504 cells are also shown in FIG. 6A. Extensive fiber outgrowth was observed after 21 days following transplantation with cells harvested on day 20 of differentiation (FIG. 6B).

### Example 4: Analysis of Gene Expression to Predict Engraftment

A reference set of samples of differentiated neural cells with known engraftment status was used to identify a set of differentially expressed genes that differed between samples that did engraft and those that did not engraft after being transplanted into rat brains.

Gene expression was determined using RNA sequencing (RNA-Seq).

68 differentially expressed genes were identified that were upregulated in the samples known to engraft compared to those that were known to not engraft. These genes are identified in Table E1. Among the differentially expressed genes, cell cycle genes were strongly overrepresented.

TABLE E1
Genes Upregulated in Engrafting Samples

| | | | | | | | |
|---|---|---|---|---|---|---|---|
| ANLN | CCNB1 | CENPF | GEM | KIF20A | NCAPH | POC1A | TOP2A |
| ASPM | CCNB2 | CEP55 | GTSE1 | KIF23 | NDC80 | PRC1 | TPX2 |
| AURKA | CDC20 | CIT | HJURP | KIF2C | NEK2 | PRR11 | TTK |
| AURKB | CDC25C | DLGAP5 | HMMR | KIF4A | NUF2 | PTTG1 | TUBA1C |
| BIRC5 | CDCA2 | ECT2 | IQGAP3 | KIFC1 | NUSAP1 | RACGAP1 | UBE2C |
| BRINP1 | CDCA8 | ESPL1 | KIF11 | KNL1 | PBK | SAPCD2 | |
| BUB1 | CDK1 | FAM83D | KIF14 | MELK | PIMREG | SKA3 | |
| BUB1B | CDKN1A | FANCD2 | KIF15 | MKI67 | PLK1 | SPAG5 | |
| CCNA2 | CENPE | FOXM1 | KIF18A | NCAPG | PLK2 | TACC3 | |

Using the set of 68 identified genes, a model was developed to predict engraftment.

The expression levels of the 68 differentially expressed genes were used to generate a sparse model by Lasso regression (also known as an L1 regularized regression). Lasso regression resulted in a 7-gene model that included the following genes: BRINP1, CDKNIA, FAM83D, FANCD2, GEM, PLK2, and SAPCD2. Samples that include differentiated neural cells that exhibit upregulated gene expression for these 7 genes were associated with an increased likelihood of engraftment.

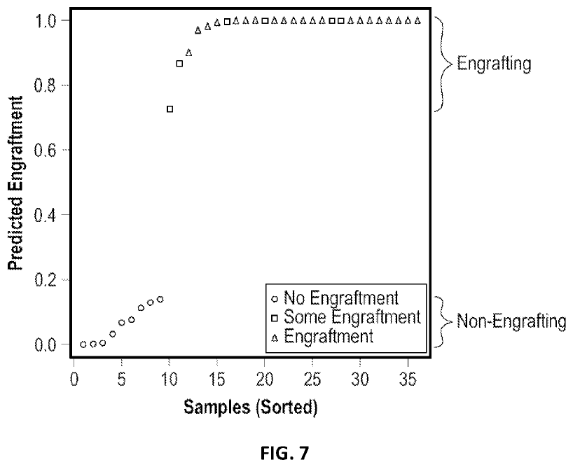

This 7-gene model was used to predict engraftment of 36 samples. Each of the 36 samples received a prediction score within a range of 0.00 to 1.0, with a value of 0.00 being associated with the lowest likelihood of engraftment, i.e., a prediction of no engraftment, and a value of 1.0 being associated with the highest likelihood of engraftment, i.e., a prediction of engraftment. The results as shown in FIG. 7. As shown in FIG. 7, each of the 9 samples that did not engraft were assigned a prediction score below 0.2, with 3 of the samples being assigned a prediction score of approximately 0.00. In contrast, each of the 21 samples that were shown to engraft were assigned a prediction score of approximately 0.9 or higher, with 18 of the samples being assigned a prediction score of approximately 1.0 (FIG. 7). Each of the 6 samples that were shown to exhibit some engraftment were assigned a prediction score of approximately 0.7 or higher, with 4 of the samples being assigned a prediction score of approximately 1.0 (FIG. 7). In this example, a cut-off of, for instance, 0.75 could be set to predict engraftment, with values at or above 0.75 being predicted to engraft, and values below 0.75 being predicted to not engraft.

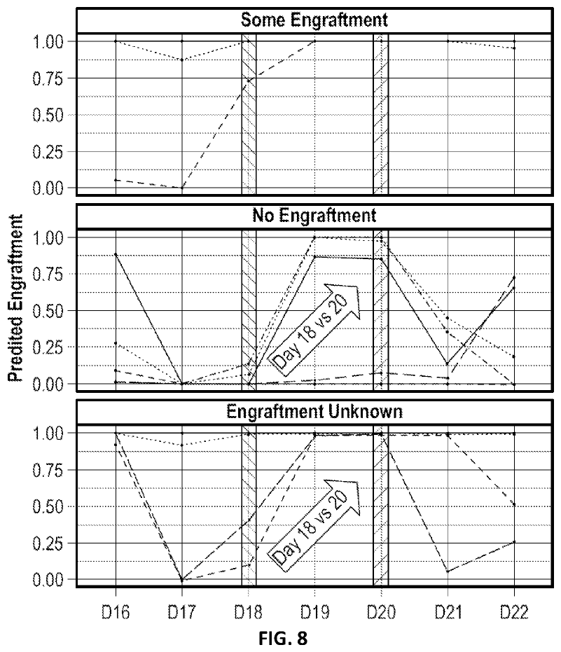

Predicted engraftment was plotted relative to the day of differentiation (days 16, 17, 18, 19, 20, 21, and 22) for samples with cells that demonstrated some engraftment, no engraftment, or unknown engraftment on day 18 (FIG. 8). A pronounced difference in engraftment was noted between day 18 and day 20. As shown in FIG. 8, on day 18, cells that were known to exhibit some engraftment had a prediction score at or above approximately 0.75. Cells that were known to not exhibit engraftment on day 18 had a prediction score below 0.25 on day 18; however, these cells were predicted to engraft on day 20 by exhibiting a prediction score above 0.75 on day 20. This data demonstrates that the likelihood of a successful engraftment can be predicted using this 7-gene model and that the ideal duration of differentiation can differ among samples.

### Example 5: Analysis of Gene Expression Associated with Differentiation Time

In order to develop a point estimate of how much development cells have undergone at a given time point during differentiation, the transcript abundance of genes (a proxy of gene expression) was used as a measure of development, or maturity. To capture an accurate representation of transcriptional maturity, transcript abundance of multiple genes that either increased or decreased with time during differentiation were measured. Multiple (10⁻⁵⁰) genes with a similar expression pattern were considered in order to increase the signal and dampen the noise that would be encountered if lower gene numbers were considered instead.

Genes with transcript abundance that changed unidirectionally, and significantly, with time were identified. To capture developmentally relevant time points, cells were differentiated by the protocol described in Example 1 and collected at various days of differentiation for analysis of gene expression by RNAseq. Gene expression in the cells was analyzed by RNAseq at days including day 17, day 18, day 19, day 20, day 21, and day 22 of differentiation.

### A. Identification of Maturity Genes

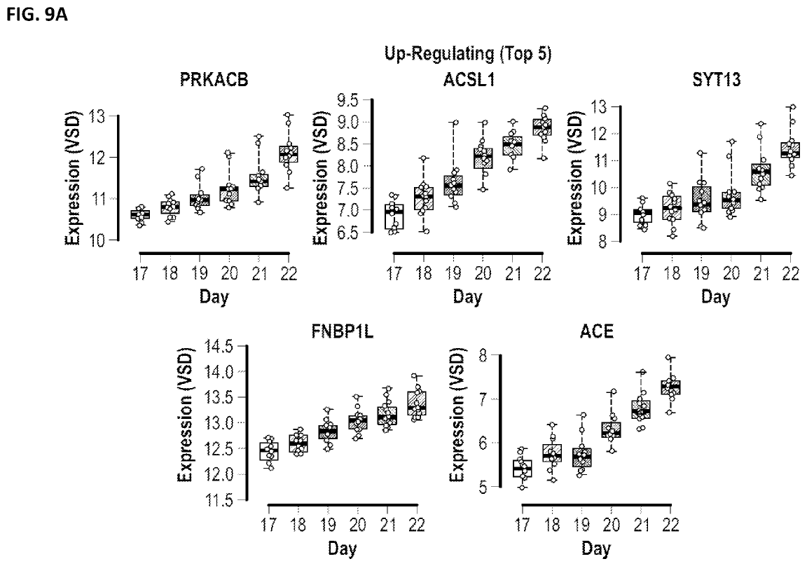

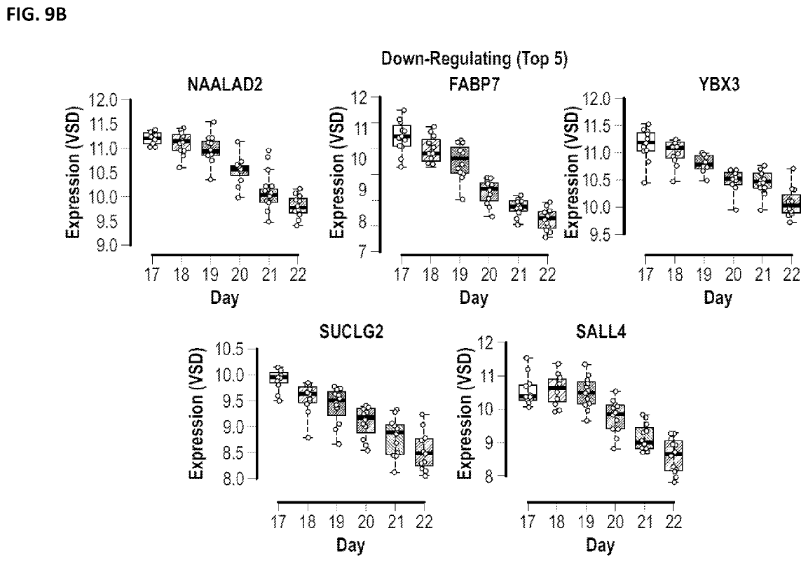

Genes whose expression was associated with differentiation time were identified using a linear regression model implemented in the edgeR Bioconductor package. Expression data were normalized using the DESeq2 Bioconductor package, and variance stabilized data (vsd) were used. The timepoints used were Day 17, Day 18, Day 19, Day 20, Day 21, and Day 22. In order to more accurately model over-dispersed RNAseq count data, one degree-of-freedom was specified (linear model), and the negative binomial error structure was assumed by the model. Exemplary genes whose expression was associated with differentiation time are depicted in FIG. 9A-9B. FIG. 9A shows the top 5 significantly up-regulated genes ranked by increasing P-value (Table E2). FIG. 9B shows the top 5 significant down-regulating genes ranked by increasing P-value.

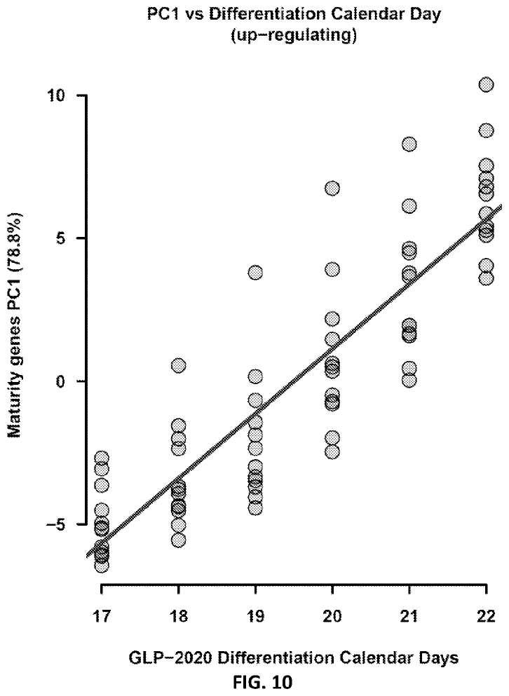

A Principal Component Analysis (PCA) was performed using the top 50 upregulated genes (Table E2) that were associated with the day of differentiation, hereby referred to as “maturity genes.” The result of this PCA analysis is depicted in FIG. 10, with Principal Component 1 (PC1) depicted for the maturity genes, which carried 78.8% of the variance of the transcript data. This demonstrates that PC1 was associated with a measure of transcriptional age (and calendar days), with the magnitude of PC1 also reflecting the magnitude of developmental maturity. The same process can also be applied to the top 50 down-regulated genes (Table E3).

TABLE E2
The top 50 up-regulating ‘maturity’ genes determined
by linear model (degree-of-freedom = 1) in edgeR.
The order from most significant to least significant
is from top left TO BOTTOM RIGHT (BY COLUMN).

| | | | | |
|---|---|---|---|---|
| PRKACB | NFIX | NCAM1 | CHGB | KCNC1 |
| ACSL1 | GFOD2 | FBXL16 | MACO1 | APBA1 |
| SYT13 | ARL8A | MAPRE3 | MAP3K9 | CCDC112 |
| FNBP1L | MIR100HG | SLC6A17 | CDK5R2 | SBK1 |
| ACE | DMTN | DIRAS1 | AFAP1 | RIMS1 |
| NACAD | KIF1A | BDNF | ARHGDIG | TRIM46 |
| CEP170B | TMEM151B | GUCY1A1 | DUSP26 | PARP6 |
| LINC01128 | KCNH6 | SHISA7 | KLF7 | AC104083.1 |
| TPH1 | CAMK2B | BICDL1 | SRGAP2 | DNAJB5 |
| NFIC | KCNB1 | HCN3 | JPT1 | SPTBN1 |

TABLE E3
The top 50 down-regulating ‘maturity’ genes
determined by linear model (degree-of-freedom =
1) in edgeR. The order from most significant to least
significant is from top left to bottom right (by column).

| | | | | |
|---|---|---|---|---|
| NAALAD2 | STOX1 | SLC35D2 | ANXA11 | LRIG1 |
| FABP7 | SEMA5B | TOB1 | KCNJ2-AS1 | CCDC160 |
| YBX3 | MGST1 | DPY19L1 | FZD2 | FAM71E2 |
| SUCLG2 | PLAG1 | TM6SF2 | MYCBP | CTSC |
| SALL4 | ACSS3 | NT5DC1 | FAM86C2P | COL23A1 |
| HAPLN3 | TGFBR3 | ADSS | CCDC60 | NAV2 |
| NR6A1 | MRVI1 | SELENOP | POFUT2 | IKZF2 |
| HTATIP2 | HLA-E | ANP32A | ASPH | SLC66A3 |
| PRTG | ITGA5 | PTCH1 | IL4R | CYFIP1 |
| PTPN13 | LRIG3 | SAV1 | DAAM2 | LPIN3 |

### B. Differences in Maturity Gene Expression Based on Differentiation Conditions

To test whether the set of 50 up-regulated maturity genes could reveal differences between differentiation experiments, the biological maturity of cells differentiated using two different sets of conditions that differed with respect to the substrate and dissociation agents used was assessed. Some cells were differentiated according to the method described in Example 1, with Geltrex used as the substrate from day 0-16, followed by poly-L-ornithine, laminin, and fibronectin used as the substrate from day 16-18, except that dispase/collagenase was used as the dissociation agent, hereby referred to in this example as “the Geltrex method.” Some cells were differentiated according to the method described in Example 1, except that laminin 511 E8 fragment was used as the substrate from day 0-16, hereby referred to as “the Laminin method.” For some cells harvested using the Laminin method, the method also differed from the method of Example 1 by using a Laminin 111 substrate on days 16-18 and/or by using dispase/collagenase as the dissociation agent.

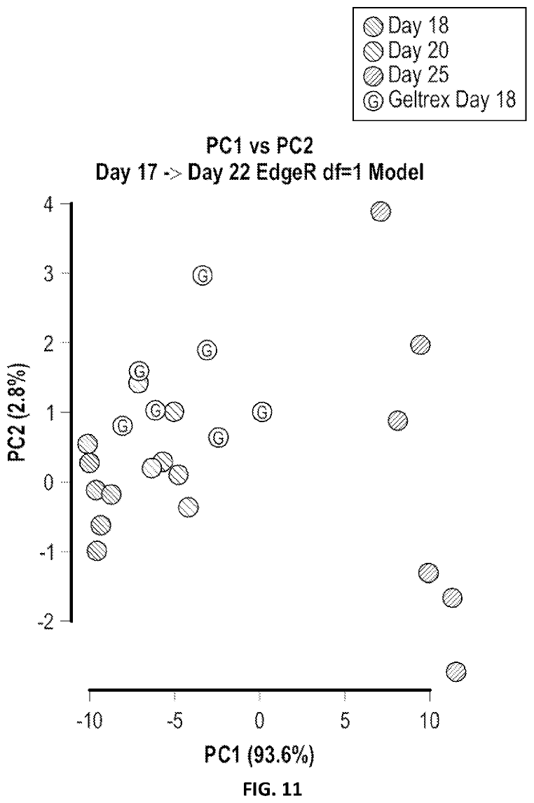

The maturity of the cells in samples harvested on day 18, day 20, or day 25 was assessed using the identified 50 up-regulated maturity genes. The results are depicted in FIG. 11. FIG. 11 shows the ordination of samples from the two different sets of differentiation conditions based on the 50 up-regulated maturity genes as measured using RNA-seq and analyzed using PCA. PC1 and PC2 carried 93.6% and 2.8% of the variance of the clock gene expression data, respectively.

As shown in FIG. 11, cells harvested on day 18 using the Geltrex method appeared to be more transcriptionally or biologically mature than cells harvested on day 18 using the Laminin method, based on the PCA analysis of the 50 up-regulated maturity genes (FIG. 11). As shown in FIG. 11, expression of the 50 up-regulated maturity genes for cells harvested on day 18 using the Geltrex method overlapped with cells harvested on day 20 using variations of the Laminin method.

This data demonstrates that the 50 up-regulated maturity genes distinguished large scale cell maturity differences that may be evident between, for example, differentiation protocols.

### C. Differences in Maturity Gene Expression Based on Engraftment Success

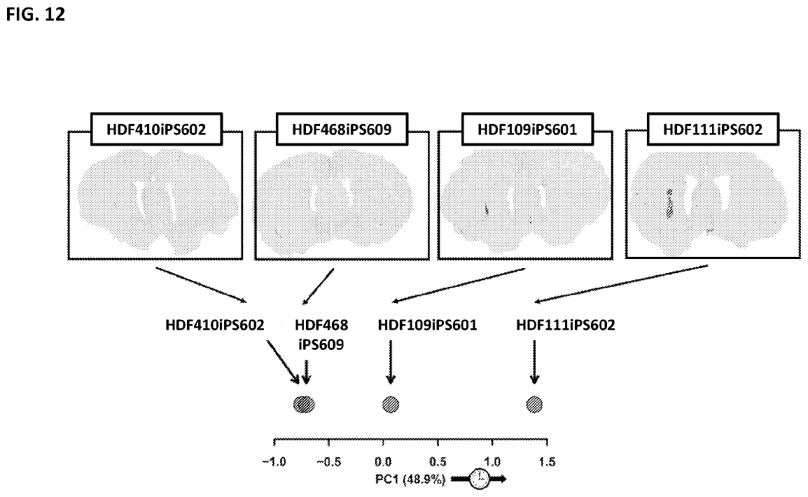

To test whether more fine scale differences could be detected within a timepoint using the identified maturity genes, the biological maturity of differentiated cells was compared in relation to the ability of the differentiated cells to engraft following neural implantation. Samples were plotted with assigned PC1 values, which were associated with biological maturity (FIG. 12). As shown in FIG. 12, cells having a more mature biological age, per the PC1 value, correlated with improved engraftment. For instance, of the four samples shown, HDF11liPS602 was the most biologically mature and exhibited the greatest engraftment area, and HDF109iPS601 was the second most biologically mature and exhibited the second greatest engraftment area (FIG. 12).

### D. Conceptual Model of Engraftment Success

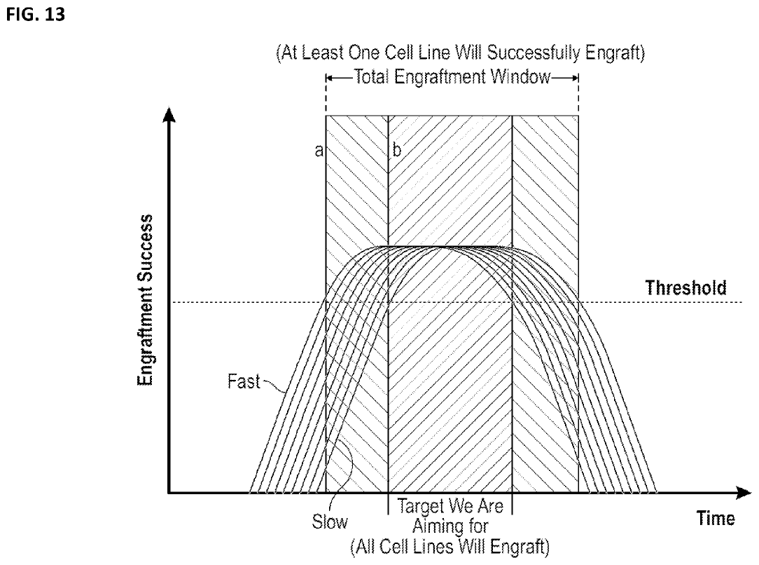

Using these results, a conceptual “maturity gene” model of cell maturity was developed to predict engraftment success (FIG. 13). Depending on the speed at which the differentiating cells reach biological maturity, the cells can reach the threshold for engraftment success by approximately day 18-20. According to the available data, time point (a) is reached on approximately day 18-19, while time point (b) is reached on approximately day 20, the time points possibly variable between cell lines. The window from the point at which time point (b) is reached to where the engraftment success returns below the threshold is the target window that is aimed for, in which all cells within that window are predicted to engraft. Given this window of engraftment success, the expression of maturity genes or surrogate biomarkers that reach time point (a) can be used to either (i) harvest the cells if they have surpassed the engraftment success threshold, and optionally freeze the cells for future use, or (ii) allow for extended differentiation to allow those cells to reach and surpass the engraftment success threshold.

The data shown herein demonstrates that cells can be ‘ready’ for transplanting at some point along the Day 17→Day 22 developmental timeframe, but that different differentiation protocols can confer alternative slopes or norms of reaction (shapes) of gene expression with time. The data shown herein also demonstrates that certain genes can be used as a ‘maturity estimator’ tool to measure if a population of cells is developing relatively fast or relatively slow. This procedure is not limited to dopaminergic neuron progenitors and could be applied to any differentiating cell type where differentiation time is an important variable.

### Example 6: Further Analysis of Gene Expression Associated with Differentiation Time

Further analysis of maturity genes with increasing or decreasing expression during differentiation was performed. The analysis was performed as described in Example 5, but with RNAseq gene expression data collected on day 17, day 18, day 19, day 20, day 21, day 22, and day 25 of differentiation. In this manner, genes with differential expression from Day 17→Day 25 were identified (Day 17→Day 25 maturity genes). The Day 17→Day 25 maturity genes were then compared to the genes identified in Example 5 that exhibited differential expression from Day 17→Day 22 (Day 17→Day 22 maturity genes).

Table E4 lists the top 50 up-regulating Day 17→Day 25 maturity genes that were also in the top 50 up-regulating Day 17→Day 22 maturity genes (Table E2). Table E5 lists the top 50 down-regulating Day 17→Day 25 maturity genes that were also in the top 50 down-regulating Day 17→Day 22 maturity genes (Table E3).

This analysis indicated that the genes listed in Table E4 and Table E5 were differentially expressed from Day 17→Day 25 as well as from Day 17→Day 22. These findings support that expression of these genes can be used to assess the maturity of cell samples during differentiation, as well as to predict engraftment of the cell samples following implantation.

TABLE E4
Intersect of Day 17→ Day 25 Up-Regulating Maturity Genes with
Day 17→ Day 22 Up-Regulating Maturity Genes (Table E3)

| | |
|---|---|
| PRKACB | NFIX |
| ACSL1 | ARL8A |
| SYT13 | MIR100HG |
| FNBP1L | DMTN |
| ACE | KIF1A |
| NACAD | KCNB1 |
| CEP170B | SLC6A17 |
| LINC01128 | CHGB |
| TPH1 | MAP3K9 |
| NFIC | CCDC112 |

TABLE E5
Intersect of Day 17→ Day 25 Down-Regulating Maturity Genes with
Day 17→ Day 22 Down-Regulating Maturity Genes (Table E4)

| | | |
|---|---|---|
| NAALAD2 | STOX1 | DAAM2 |
| FABP7 | SEMA5B | LRIG1 |
| YBX3 | MGST1 | CCDC160 |
| SUCLG2 | PLAG1 | |
| SALL4 | TGFBR3 | |
| HAPLN3 | MRVI1 | |
| NR6A1 | SLC35D2 | |
| HTATIP2 | NT5DC1 | |
| PRTG | ANP32A | |
| PTPN13 | PTCH1 | |

### Example 7: Further Analysis of Gene Expression to Predict Engraftment

Further analysis of genes differentially expressed between engrafting and non-engrafting cell samples was performed. To do so, 40 cell lines were differentiated using protocols similar to that described in Example 1, harvested at day 18 or day 20 of differentiation in culture, and implanted in both hemispheres of 239 animals using a procedure similar to that described in Example 2. Engraftment was scored based on immunohistochemical staining for human nuclei following implantation. Engraftment scores were averaged between hemispheres, then averaged across animals receiving cells from the same cell line. Gene expression levels collected at harvest were then compared between cell lines exhibiting above-median engraftment scores (n=21) and below-median engraftment scores (n=19). Expression data was analyzed using the DESeq2 Bioconductor package.

This analysis identified 237 genes up-regulated in engrafting cells with fold-change (FC) greater than or equal to 2 and adjusted p-value (false discovery rate) less than 0.05. This analysis also identified 146 genes down-regulated in engrafting cells with FC less than or equal to 0.5 and adjusted p-value (false discovery rate) less than 0.05. These genes are shown in Table E6-E8. Table E6 lists the up-regulated engraftment-associated genes that were also identified in Example 4 as up-regulated in cells shown to engraft following implantation (Table E1). Table E7 lists the up-regulated engraftment-associated genes that were also identified in Example 5 as in the top 50 up-regulating Day 17→Day 22 maturity genes (Table E2). Table E8 lists the down-regulated engraftment-associated genes that were also identified in Example 5 as in the top 50 down-regulating Day 17→Day 22 maturity genes (Table E3).

These findings support that expression of the genes in Table E6-E8 can be used to predict engraftment of cell samples following implantation.

TABLE E6
Intersect of Up-Regulated Engraftment-Associated Genes (p <
0.05, FC > 2) with Cell Cycle Genes (Table E1)

| | | |
|---|---|---|
| AURKB | FAM83D | NUSAP1 |
| BIRC5 | GTSE1 | PTTG1 |
| CCNB1 | HJURP | SAPCD2 |
| CCNB2 | IQGAP3 | TACC3 |
| CDC20 | KIF20A | TOP2A |
| CDC25C | KIF2C | TPX2 |
| CDCA8 | KIFC1 | UBE2C |
| CDK1 | NDC80 | |
| DLGAP5 | NEK2 | |
| ESPL1 | NUF2 | |

TABLE E7
Intersect of Up-Regulated Engraftment Associated
Genes (p < 0.05, FC > 2) with Day 17→ Day 22 Up-
Regulating Maturity Genes (Table E2)

MIR100HG
BDNF

TABLE E8
Intersect of Down-Regulated Engraftment Associated
Ganes (p < 0.05, FC < 0.5) with Day
17→ Day 22 Down-Regulating Maturity Genes (Table E3)

PRTG
CCDC60
FAM71E2
COL23A1

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of predicting cell engraftment of a population of neuronal progenitor cells, the method comprising:
(a) obtaining gene expression levels of a plurality of genes for one or more cells of a population of neuronal progenitor cells, wherein:
the population of neuronal progenitor cells is from a culture of cells differentiated from pluripotent stem cells under conditions to neurally differentiate the cells; and
the plurality of genes comprises one or more of AC104083.1, ACE, ACSL1, ACSS3, ADSS, AFAP1, ANLN, ANP32A, ANXA11, APBA1, ARHGDIG, ARL8A, ASPH, ASPM, AURKA, AURKB, BDNF, BICDL1, BIRC5, BRINP1, BUB1, BUB1B, CAMK2B, CCDC112, CCDC160, CCDC60, CCNA2, CCNB1, CCNB2, CDC20, CDC25C, CDCA2, CDCA8, CDK1, CDK5R2, CDKN1A, CENPE, CENPF, CEP170B, CEP55, CHGB, CIT, COL23A1, CTSC, CYFIP1, DAAM2, DIRAS1, DLGAP5, DMTN, DNAJB5, DPY19L1, DUSP26, ECT2, ESPL1, FABP7, FAM71E2, FAM83D, FAM86C2P, FANCD2, FBXL16, FNBP1L, FOXM1, FZD2, GEM, GFOD2, GTSE1, GUCY1A1, HAPLN3, HCN3, HJURP, HLA-E, HMMR, HTATIP2, IKZF2, IL4R, IQGAP3, ITGA5, JPT1, KCNB1, KCNC1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MGST1, MIR100HG, MKI67, MRVI1, MYCBP, NAALAD2, NACAD, NAV2, NCAM1, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SALL4, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TOP2A, TPH1, TPX2, TRIM46, TTK, TUBA1C, UBE2C, and YBX3; and
(b) applying the gene expression levels as input to a process configured to predict if the population of neuronal progenitor cells will engraft in a brain region of a subject following implantation of the population of neuronal progenitor cells into the brain region, wherein the predicting is based on the gene expression levels, and the process comprises a machine learning model trained using gene expression levels of the plurality of genes for a plurality of reference populations of neuronal progenitor cells that have been differentiated from pluripotent stem cells under conditions to neurally differentiate the cells.

2. A method of assessing a population of neuronal progenitor cells for implantation in a subject to treat a neurodegenerative disease, the method comprising:
(a) obtaining gene expression levels of a plurality of genes for one or more cells of a population of neuronal progenitor cells, wherein:
the population of neuronal progenitor cells is from a culture of cells differentiated from pluripotent stem cells under conditions to neurally differentiate the cells; and
the plurality of genes comprises one or more of AC104083.1, ACE, ACSL1, ACSS3, ADSS, AFAP1, ANLN, ANP32A, ANXA11, APBA1, ARHGDIG, ARL8A, ASPH, ASPM, AURKA, AURKB, BDNF, BICDL1, BIRC5, BRINP1, BUB1, BUB1B, CAMK2B, CCDC112, CCDC160, CCDC60, CCNA2, CCNB1, CCNB2, CDC20, CDC25C, CDCA2, CDCA8, CDK1, CDK5R2, CDKN1A, CENPE, CENPF, CEP170B, CEP55, CHGB, CIT, COL23A1, CTSC, CYFIP1, DAAM2, DIRAS1, DLGAP5, DMTN, DNAJB5, DPY19L1, DUSP26, ECT2, ESPL1, FABP7, FAM71E2, FAM83D, FAM86C2P, FANCD2, FBXL16, FNBP1L, FOXM1, FZD2, GEM, GFOD2, GTSE1, GUCY1A1, HAPLN3, HCN3, HJURP, HLA-E, HMMR, HTATIP2, IKZF2, IL4R, IQGAP3, ITGA5, JPT1, KCNB1, KCNC1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MGST1, MIR100HG, MKI67, MRVI1, MYCBP, NAALAD2, NACAD, NAV2, NCAM1, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SALL4, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TOP2A, TPH1, TPX2, TRIM46, TTK, TUBA1C, UBE2C, and YBX3; and
(b) applying the gene expression levels as input to a process configured to predict if the population of neuronal progenitor cells will engraft in a brain region of a subject following implantation of the population of neuronal progenitor cells into the brain region, wherein the predicting is based on the gene expression levels, and the process comprises a machine learning model trained using gene expression levels of the plurality of genes for a plurality of reference populations of neuronal progenitor cells that have been differentiated from pluripotent stem cells under conditions to neurally differentiate the cells.

3. A method of selecting a population of neuronal progenitor cells for implantation in a subject for treating a neurodegenerative disease, the method comprising:
(a) obtaining gene expression levels of a plurality of genes for one or more cells of a population of neuronal progenitor cells, wherein:
the population of neuronal progenitor cells is from a culture of cells differentiated from pluripotent stem cells under conditions to neurally differentiate the cells; and
the plurality of genes comprises one or more of AC104083.1, ACE, ACSL1, ACSS3, ADSS, AFAP1, ANLN, ANP32A, ANXA11, APBA1, ARHGDIG, ARL8A, ASPH, ASPM, AURKA, AURKB, BDNF, BICDL1, BIRC5, BRINP1, BUB1, BUB1B, CAMK2B, CCDC112, CCDC160, CCDC60, CCNA2, CCNB1, CCNB2, CDC20, CDC25C, CDCA2, CDCA8, CDK1, CDK5R2, CDKN1A, CENPE, CENPF, CEP170B, CEP55, CHGB, CIT, COL23A1, CTSC, CYFIP1, DAAM2, DIRAS1, DLGAP5, DMTN, DNAJB5, DPY19L1, DUSP26, ECT2, ESPL1, FABP7, FAM71E2, FAM83D, FAM86C2P, FANCD2, FBXL16, FNBP1L, FOXM1, FZD2, GEM, GFOD2, GTSE1, GUCY1A1, HAPLN3, HCN3, HJURP, HLA-E, HMMR, HTATIP2, IKZF2, IL4R, IQGAP3, ITGA5, JPT1, KCNB1, KCNC1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MGST1, MIR100HG, MKI67, MRVI1, MYCBP, NAALAD2, NACAD, NAV2, NCAM1, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SALL4, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TOP2A, TPH1, TPX2, TRIM46, TTK, TUBA1C, UBE2C, and YBX3;
(b) applying the gene expression levels as input to a process configured to predict if the population of neuronal progenitor cells will engraft in a brain region of a subject following implantation of the population of neuronal progenitor cells into the brain region, wherein the predicting is based on the gene expression levels, and the process comprises a machine learning model trained using gene expression levels of the plurality of genes for a plurality of reference populations of neuronal progenitor cells that have been differentiated from pluripotent stem cells under conditions to neurally differentiate the cells; and
(c) selecting the population of neuronal progenitor cells for implantation in the subject if the population of neuronal progenitor cells are predicted to engraft.

4. The method of any one of claims 1-3, wherein the machine learning model is trained using (i) the gene expression levels for the plurality of reference populations and (ii) engraftment fitness of the plurality of reference populations, wherein the engraftment fitness of a reference population indicates whether or not, or the degree to which, the reference population engrafted in a brain region of a subject following implantation of the reference population into the brain region.

5. A method of training a machine learning model, comprising:
(a) obtaining gene expression levels of a plurality of genes for a plurality of reference populations of neuronal progenitor cells that are from cultures of cells that have been differentiated from pluripotent stem cells under conditions to neurally differentiate the cells, wherein the plurality of genes comprises one or more of AC104083.1, ACE, ACSL1, ACSS3, ADSS, AFAP1, ANLN, ANP32A, ANXA11, APBA1, ARHGDIG, ARL8A, ASPH, ASPM, AURKA, AURKB, BDNF, BICDL1, BIRC5, BRINP1, BUB1, BUB1B, CAMK2B, CCDC112, CCDC160, CCDC60, CCNA2, CCNB1, CCNB2, CDC20, CDC25C, CDCA2, CDCA8, CDK1, CDK5R2, CDKN1A, CENPE, CENPF, CEP170B, CEP55, CHGB, CIT, COL23A1, CTSC, CYFIP1, DAAM2, DIRAS1, DLGAP5, DMTN, DNAJB5, DPY19L1, DUSP26, ECT2, ESPL1, FABP7, FAM71E2, FAM83D, FAM86C2P, FANCD2, FBXL16, FNBP1L, FOXM1, FZD2, GEM, GFOD2, GTSE1, GUCY1A1, HAPLN3, HCN3, HJURP, HLA-E, HMMR, HTATIP2, IKZF2, IL4R, IQGAP3, ITGA5, JPT1, KCNB1, KCNC1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MGST1, MIR100HG, MKI67, MRVI1, MYCBP, NAALAD2, NACAD, NAV2, NCAM1, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SALL4, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TOP2A, TPH1, TPX2, TRIM46, TTK, TUBA1C, UBE2C, and YBX3; and
(b) applying the gene expression levels of the plurality of reference populations as input to train a machine learning model.

6. The method of claim 5, further comprising:
(a) receiving engraftment fitness of the plurality of reference populations, wherein the engraftment fitness of a reference population indicates whether or not, or the degree to which, the reference population engrafted in a brain region of a subject following implantation of the reference population into the brain region; and
(b) applying the engraftment fitness of the plurality of reference populations as input to train the machine learning model, wherein the machine learning model is trained to predict based on the gene expression levels of the plurality of genes if a population of neuronal progenitor cells that is from a culture of cells differentiated from pluripotent stem cells under conditions to neurally differentiate the cells will engraft in a brain region of a subject following implantation of the population of neuronal progenitor cells into the brain region.

7. The method of claim 4 or claim 6, wherein a reference population is considered fit for engraftment if at least a predetermined number of cells are present in the brain region following the implantation.

8. The method of claim 7, wherein the number of cells is counted at, about, at least, or at least about 7 days, 14 days, or 21 days following the implantation.

9. The method of claim 7 or claim 8, wherein the predetermined number of cells is greater than or greater than about 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9%, or 10% of the number of cells implanted in the brain region.

10. The method of any one of claims 1-9, wherein the plurality of genes comprises one or more of AC104083.1, ACE, ACSL1, ACSS3, ADSS, AFAP1, ANLN, ANP32A, ANXA11, ARHGDIG, ARL8A, ASPH, AURKA, AURKB, BICDL1, BIRC5, BRINP1, BUB1, BUB1B, CCDC112, CCDC160, CCDC60, CCNA2, CCNB1, CCNB2, CDC20, CDC25C, CDCA2, CDCA8, CDK1, CDKN1A, CENPE, CENPF, CEP170B, CEP55, CHGB, CIT, CYFIP1, DAAM2, DIRAS1, DLGAP5, DNAJB5, DPY19L1, DUSP26, ECT2, ESPL1, FAM71E2, FAM83D, FAM86C2P, FANCD2, FBXL16, FNBP1L, FOXM1, FZD2, GEM, GFOD2, GTSE1, GUCY1A1, HCN3, HJURP, HLA-E, HMMR, HTATIP2, IKZF2, IQGAP3, ITGA5, JPT1, KCNB1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MIR100HG, MKI67, MRVI1, MYCBP, NAALAD2, NACAD, NAV2, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TPH1, TPX2, TRIM46, TTK, TUBA1C, and UBE2C.

11. The method of any one of claims 1-9, wherein the plurality of genes comprises one or more of ACE, ACSL1, ANP32A, ARL8A, AURKB, BDNF, BIRC5, CCDC112, CCDC160, CCDC60, CCNB1, CCNB2, CDC20, CDC25C, CDCA8, CDK1, CEP170B, CHGB, COL23A1, DAAM2, DLGAP5, DMTN, ESPL1, FABP7, FAM71E2, FAM83D, FNBP1L, GTSE1, HAPLN3, HJURP, HTATIP2, IQGAP3, KCNB1, KIF1A, KIF20A, KIF2C, KIFC1, LINC01128, LRIG1, MAP3K9, MGST1, MIR100HG, MRVI1, NAALAD2, NACAD, NDC80, NEK2, NFIC, NFIX, NR6A1, NT5DC1, NUF2, NUSAP1, PLAG1, PRKACB, PRTG, PTCH1, PTPN13, PTTG1, SALL4, SAPCD2, SEMA5B, SLC35D2, SLC6A17, STOX1, SUCLG2, SYT13, TACC3, TGFBR3, TOP2A, TPH1, TPX2, UBE2C, and YBX3.

12. The method of any one of claims 1-9 and 11, wherein the plurality of genes comprises one or more of ACE, ACSL1, ANP32A, ARL8A, AURKB, BIRC5, CCDC112, CCDC160, CCDC60, CCNB1, CCNB2, CDC20, CDC25C, CDCA8, CDK1, CEP170B, CHGB, COL23A1, DAAM2, DLGAP5, ESPL1, FABP7, FAM71E2, FAM83D, FNBP1L, GTSE1, HAPLN3, HJURP, HTATIP2, IQGAP3, KCNB1, KIF1A, KIF20A, KIF2C, KIFC1, LINC01128, LRIG1, MAP3K9, MGST1, MIR100HG, MRVI1, NAALAD2, NACAD, NDC80, NEK2, NFIC, NFIX, NR6A1, NT5DC1, NUF2, NUSAP1, PLAG1, PRKACB, PRTG, PTCH1, PTPN13, PTTG1, SALL4, SAPCD2, SEMA5B, SLC35D2, SLC6A17, STOX1, SUCLG2, SYT13, TACC3, TGFBR3, TOP2A, TPH1, TPX2, UBE2C, and YBX3.

13. The method of any one of claims 1-10, wherein the plurality of genes comprises BRINP1, CDKN1A, FAM83D, FANCD2, GEM, PLK2, and SAPCD2.

14. The method of any one of claims 1-10, wherein the plurality of genes comprises one or more of AURKB, BIRC5, CCNB1, CCNB2, CDC20, CDC25C, CDCA8, CDK1, DLGAP5, ESPL1, FAM83D, GTSE1, HJURP, IQGAP3, KIF20A, KIF2C, KIFC1, NDC80, NEK2, NUF2, NUSAP1, PTTG1, SAPCD2, TACC3, TPX2, and UBE2C.

15. The method any one of claims 1-10, wherein the plurality of genes comprises one or more of ACE, ACSL1, ANP32A, ARL8A, CCDC112, CCDC160, CCDC60, CEP170B, CHGB, DAAM2, FAM71E2, FNBP1L, HTATIP2, KCNB1, KIF1A, LINC01128, LRIG1, MAP3K9, MIR100HG, MRVI1, NAALAD2, NACAD, NFIC, NFIX, NR6A1, NT5DC1, PLAG1, PRKACB, PRTG, PTCH1, PTPN13, SEMA5B, SLC35D2, SLC6A17, STOX1, SUCLG2, SYT13, TGFBR3, and TPH1.

16. The method of any one of claims 1-15, further comprising selecting, based on an output of the process, the population of neuronal progenitor cells as a population of neuronal progenitor cells that is predicted to engraft, and harvesting the selected population of neuronal progenitor cells.

17. A method of assessing engraftment fitness of a population of neuronal progenitor cells, the method comprising (a) measuring gene expression levels of a plurality of genes for one or more cells of a population of neuronal progenitor cells, wherein:
the population of neuronal progenitor cells is from a culture of cells differentiated from pluripotent stem cells under conditions to neurally differentiate the cells; and
the plurality of genes comprises one or more of AC104083.1, ACE, ACSL1, ACSS3, ADSS, AFAP1, ANLN, ANP32A, ANXA11, ARHGDIG, ARL8A, ASPH, AURKA, AURKB, BICDL1, BIRC5, BRINP1, BUB1, BUB1B, CCDC112, CCDC160, CCDC60, CCNA2, CCNB1, CCNB2, CDC20, CDC25C, CDCA2, CDCA8, CDK1, CDKN1A, CENPE, CENPF, CEP170B, CEP55, CHGB, CIT, CYFIP1, DAAM2, DIRAS1, DLGAP5, DNAJB5, DPY19L1, DUSP26, ECT2, ESPL1, FAM71E2, FAM83D, FAM86C2P, FANCD2, FBXL16, FNBP1L, FOXM1, FZD2, GEM, GFOD2, GTSE1, GUCY1A1, HCN3, HJURP, HLA-E, HMMR, HTATIP2, IKZF2, IQGAP3, ITGA5, JPT1, KCNB1, KCNH6, KCNJ2-AS1, KIF11, KIF14, KIF15, KIF18A, KIF1A, KIF20A, KIF23, KIF2C, KIF4A, KIFC1, KLF7, KNL1, LINC01128, LPIN3, LRIG1, LRIG3, MACO1, MAP3K9, MAPRE3, MELK, MIR100HG, MKI67, MRVI1, MYCBP, NAALAD2, NACAD, NAV2, NCAPG, NCAPH, NDC80, NEK2, NFIC, NFIX, NR6A1, NT5DC1, NUF2, NUSAP1, PARP6, PBK, PIMREG, PLAG1, PLK1, PLK2, POC1A, POFUT2, PRC1, PRKACB, PRR11, PRTG, PTCH1, PTPN13, PTTG1, RACGAP1, RIMS1, SAPCD2, SAV1, SBK1, SELENOP, SEMA5B, SHISA7, SKA3, SLC35D2, SLC66A3, SLC6A17, SPAG5, SPTBN1, SRGAP2, STOX1, SUCLG2, SYT13, TACC3, TGFBR3, TM6SF2, TMEM151B, TOB1, TPH1, TPX2, TRIM46, TTK, TUBA1C, and UBE2C.

18. The method of claim 17, further comprising comparing one or more of the gene expression levels or one or more combinations thereof to one or more predetermined first threshold levels, wherein gene expression levels or combinations thereof that are greater than the first threshold levels are associated with a population of neuronal progenitor cells that is predicted to engraft in a brain region of a subject following implantation of the population of neuronal progenitor cells in the brain region.

19. The method of claim 17 or claim 18, further comprising comparing one or more of the gene expression levels or one or more combinations thereof to one or more predetermined second threshold levels, wherein gene expression levels or combinations thereof that are less than the second threshold levels are associated with a population of neuronal progenitor cells that is predicted to engraft in a brain region of a subject following implantation of the population of neuronal progenitor cells in the brain region.

20. The method of any one of claims 1-19, wherein the gene expression levels are obtained by RNA sequencing.

21. The method of any one of claims 1-4 and 7-20, wherein the population of neuronal progenitor cells comprises determined dopaminergic neuron progenitor cells.

22. The method of any one of claims 1-4 and 7-21, wherein prior to (a), the method further comprises differentiating the culture of cells comprising the population of neuronal progenitor cells.

23. The method of any one of claims 1-4 and 7-22, wherein the culture of cells comprising the population of neuronal progenitor cells is differentiated from pluripotent stem cells by a process comprising:
(a) performing a first incubation comprising culturing the pluripotent stem cells (PSCs) in a first culture vessel, wherein beginning at the initiation of the first incubation (day 0), the cells are exposed to (i) an inhibitor of TGF-β/activin-Nodal signaling; and (ii) an inhibitor of bone morphogenetic protein (BMP) signaling; and
(b) performing a second incubation comprising culturing cells produced by the first incubation in a second culture vessel under conditions to neurally differentiate the cells, optionally wherein the second culture vessel is an adherent culture vessel, optionally wherein the adherent culture vessel is coated with laminin or a fragment thereof.

24. A method of differentiating neural cells, the method comprising:
(a) performing a first incubation comprising culturing pluripotent stem cells (PSCs) in a first culture vessel, wherein beginning at the initiation of the first incubation (day 0), the cells are exposed to (i) an inhibitor of TGF-β/activin-Nodal signaling; and (ii) an inhibitor of bone morphogenetic protein (BMP) signaling; and
(b) performing a second incubation comprising culturing cells produced by the first incubation in a second culture vessel under conditions to neurally differentiate the cells;
wherein the second culture vessel is an adherent culture vessel coated with laminin or a fragment thereof; and
(c) harvesting the cells between at or about day 19 and day 24.

25. The method of claim 23 or claim 24, wherein the laminin is or comprises Laminin-111, Laminin-211, Laminin-121, Laminin-221, Laminin-332, Laminin-3A32, Laminin-3B32, Laminin-311, Laminin-3A11, Laminin-321, Laminin-3A21, Laminin-411, Laminin-421, Laminin-511, Laminin-521, Laminin-213, Laminin-423, Laminin-522, Laminin-523, or a fragment of any of the foregoing, optionally wherein the laminin is or comprises Laminin-521, Laminin-111, Laminin-511, or a fragment of any of the foregoing.

26. The method of any one of claims 23-25, wherein the laminin is or comprises Laminin-511 or a fragment thereof.

27. The method of any one of claims 23-26, wherein the laminin is or comprises a Laminin-511 E8 fragment.

28. The method of any one of claims 23-27, wherein the first culture vessel is a non-adherent culture vessel.

29. The method of any one of claims 23-28, wherein, beginning on day 0, the cells are also exposed to (iii) at least one activator of Sonic Hedgehog (SHH) signaling and (iv) an inhibitor of glycogen synthase kinase 3β(GSK3β).

30. The method of any one of claims 23-29, wherein the second incubation begins on about day 7.

31. The method of any one of claims 23-30, wherein the cells are exposed to the inhibitor of TGF-3/activin-Nodal signaling up to a day at or before day 7.

32. The method of any one of claims 23-31, wherein the cells are exposed to the inhibitor of TGF-β/activin-Nodal beginning at day 0 and through day 6, inclusive of each day.

33. The method of any one of claims 29-32, wherein the cells are exposed to the at least one activator of SHH signaling up to a day at or before day 7.

34. The method of any one of claims 29-33, wherein the cells are exposed to the at least one activator of SHH signaling beginning at day 0 and through day 6, inclusive of each day.

35. The method of any one of claims 23-34, wherein the cells are exposed to the inhibitor of BMP signaling up to a day at or before day 11.

36. The method of any one of claims 23-35, wherein the cells are exposed to the inhibitor of BMP signaling beginning at day 0 and through day 10, inclusive of each day.

37. The method of any one of claims 29-36, wherein the cells are exposed to the inhibitor of GSK3β signaling up to a day at or before day 13.

38. The method of any one of claims 29-37, wherein the cells are exposed to the inhibitor of GSK3β signaling beginning at day 0 and through day 12, inclusive of each day.

39. The method of any one of claims 23-38, wherein the first incubation produces a spheroid of cells, and prior to performing the second incubation, the spheroid is dissociated to produce a cell suspension, wherein cells of the cell suspension are cultured in the second culture vessel.

40. The method of claim 39, wherein the spheroid is dissociated by enzymatic dissociation.

41. The method of claim 39 or claim 40, wherein the spheroid is dissociated by enzymatic dissociation comprising use of an enzyme selected from the group consisting of accutase, dispase, collagenase, and combinations thereof.

42. The method of any of claims 39-41, wherein the spheroid is dissociated by enzymatic dissociation comprising use of accutase.

43. The method of any one of claims 39-42, wherein the dissociating is carried out at a time when the cells of the spheroid express at least one of PAX6 and OTX2.

44. The method of any one of claims 39-43, wherein the dissociating is carried out on about day 7.

45. The method of any one of claims 23-27, wherein the first culture vessel is an adherent culture vessel coated with laminin or a fragment thereof, optionally wherein the laminin or a fragment thereof is or comprises Laminin-111, Laminin-211, Laminin-121, Laminin-221, Laminin-332, Laminin-3A32, Laminin-3B32, Laminin-311, Laminin-3A11, Laminin-321, Laminin-3A21, Laminin-411, Laminin-421, Laminin-511, Laminin-521, Laminin-213, Laminin-423, Laminin-522, Laminin-523, or a fragment of any of the foregoing, further optionally wherein the laminin is or comprises Laminin-511 or Laminin-511 E8 fragment.

46. The method of claim 45, wherein beginning on day 1, the cells are exposed to (iii) at least one activator of Sonic Hedgehog (SHH) signaling; and beginning on day 2, the cells are exposed to an (iv) an inhibitor of glycogen synthase kinase 3β (GSK3β) signaling.

47. The method of claim 45 or claim 46, wherein the second incubation begins on about day 11.

48. The method of any one of claims 45-47, wherein the cells are exposed to the inhibitor of TGF-3/activin-Nodal signaling up to a day at or before day 5.

49. The method of any one of claims 45-48, wherein the cells are exposed to the inhibitor of TGF-β/activin-Nodal beginning at day 0 and through day 4, inclusive of each day.

50. The method of any one of claims 46-49, wherein the cells are exposed to the at least one activator of SHH signaling up to a day at or before day 7.

51. The method of any one of claims 46-50, wherein the cells are exposed to the at least one activator of SHH signaling beginning at day 0 and through day 6, inclusive of each day.

52. The method of any one of claims 45-51, wherein the cells are exposed to the inhibitor of BMP signaling up to a day at or before day 11.

53. The method of any one of claims 45-52, wherein the cells are exposed to the inhibitor of BMP signaling beginning at day 0 and through day 10, inclusive of each day.

54. The method of any one of claims 46-53, wherein the cells are exposed to the inhibitor of GSK3β signaling up to a day at or before day 13.

55. The method of any one of claims 46-54, wherein the cells are exposed to the inhibitor of GSK3β signaling beginning at day 0 and through day 12, inclusive of each day.

56. The method of any one of claims 1-55, wherein the cells are cultured to differentiate the cells to determined dopaminergic neuron progenitor cells.

57. The method of any one of claims 1-56, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGF3β) (collectively, “BAGCT”); and (vi) an inhibitor of Notch signaling.

58. The method of claim 57, wherein the cells are exposed to BAGCT and the inhibitor of Notch signaling beginning on day 11.

59. The method of claim 57 or claim 58, wherein the cells are exposed to BAGCT and the inhibitor of Notch signaling beginning at day 11 and until harvest of the neurally differentiated cells.

60. The method of any one of claims 23-59, wherein the inhibitor of TGF-β/activin-Nodal signaling is SB431542.

61. The method of any one of claims 29-44 and 46-60, wherein the at least one activator of SHH signaling is SHH or purmorphamine.

62. The method of any one of claims 29-44 and 46-61, wherein the at least one activator of SHH signaling comprises two activators of SHH signaling selected from SHH protein and purmorphamine.

63. The method of any one of claims 23-62, wherein the inhibitor of BMP signaling is LDN193189.

64. The method of any one of claims 29-44 and 46-63, wherein the inhibitor of GSK3β signaling is CHIR99021.

65. The method of any one of claims 57-64, wherein the inhibitor of Notch signaling is DAPT.

66. The method of any one of claims 23-65, wherein the culturing in the first incubation and/or the second incubation is carried out in media comprising serum or a serum replacement.

67. The method of any one of claims 23-66, wherein the cells are cultured in a media comprising serum or a serum replacement from about day 0 to about day 10.

68. The method of claim 66 or claim 67, wherein the media comprises about 5% serum or serum replacement (v/v) from about day 0 to about day 1 about 2% serum replacement (v/v) from about day 2 to about day 10.

69. The method of any one of claims 16 and 20-68, wherein the harvesting comprises enzymatic dissociation comprising use of Accutase.

70. The method of any one of claims 16 and 20-69, wherein the harvesting is carried out at or about at day 20.

71. The method of any one of claims 16 and 20-70, wherein the harvested cells exhibit one or more of:
(a) a ratio of ASPM to GAPDH expression of between about 7×10⁻⁴ and about 2×10⁻¹;
(b) a ratio of AURKB to GAPDH expression of between about 9×10⁻⁴ and about 4×10⁻²;
(c) a ratio of BRINP1 to GAPDH expression of between about 9×10⁻⁵ and about 5×10⁻²;
(d) a ratio of BUB1 to GAPDH expression of between about 3×10⁻³ and about 7×10⁻²;
(e) a ratio of CCNB2 to GAPDH expression of between about 3×10⁻³ and about 7×10⁻²;
(f) a ratio of CDC20 to GAPDH expression of between about 3×10⁻³ and about 1×10⁻¹;
(g) a ratio of CDC25C to GAPDH expression of between about 5×10⁻⁴ and about 3×10⁻²;
(h) a ratio of CDKN1A to GAPDH expression of between about 1×10⁻³ and about 9×10⁻²;
(i) a ratio of CENPF to GAPDH expression of between about 7×10⁻³ and about 5×10⁻¹;
(j) a ratio of DLGAP5 to GAPDH expression of between about 2×10⁻³ and about 9×10⁻²;
(k) a ratio of FAM83D to GAPDH expression of between about 6×10⁻⁴ and about 3×10⁻²;
(l) a ratio of FANCD2 to GAPDH expression of between about 3×10⁻³ and about 4×10⁻²;
(m) a ratio of GEM to GAPDH expression of between about 6×10⁻⁴ and about 3×10⁻²;
(n) a ratio of HMMR to GAPDH expression of between about 8×10⁻⁴ and about 6×10⁻²;
(o) a ratio of IQGAP3 to GAPDH expression of between about 1×10⁻³ and about 6×10⁻²;
(p) a ratio of KIF20A to GAPDH expression of between about 1×10⁻³ and about 8×10⁻²;
(q) a ratio of KIF2C to GAPDH expression of between about 3×10⁻³ and about 7×10⁻²;
(r) a ratio of KIFC1 to GAPDH expression of between about 2×10⁻³ and about 8×10⁻²;
(s) a ratio of MKI67 to GAPDH expression of between about 2×10⁻³ and about 4×10⁻¹;
(t) a ratio of PIMREG to GAPDH expression of between about 1×10⁻³ and about 4×10⁻²;
(u) a ratio of PLK2 to GAPDH expression of between about 4×10⁻³ and about 6×10⁻²;
(v) a ratio of PTTG1 to GAPDH expression of between about 3×10⁻³ and about 9×10⁻²;
(w) a ratio of SAPCD2 to GAPDH expression of between about 1×10⁻³ and about 3×10⁻²;
(x) a ratio of TOP2A to GAPDH expression of between about 3×10⁻² and about 7×10⁻¹;
(y) a ratio of TPX2 to GAPDH expression of between about 7×10⁻³ and about 2×10⁻¹; and/or
(z) a ratio of TTK to GAPDH expression of between about 2×10⁻³ and about 8×10⁻².

72. The method of claim 71, wherein:
the ratio of BRINP1 to GAPDH expression is between about 9×10⁻⁵ and about 5×10⁻²;
the ratio of CDKN1A to GAPDH expression is between about 1×10⁻³ and about 9×10⁻²;
the ratio of FAM83D to GAPDH expression is between about 6×10⁻⁴ and about 3×10⁻²;
the ratio of FANCD2 to GAPDH expression is between about 3×10⁻³ and about 4×10⁻²;
the ratio of GEM to GAPDH expression is between about 6×10⁻⁴ and about 3×10⁻²;
the ratio of PLK2 to GAPDH expression is between about 4×10⁻³ and about 6×10⁻²;
and/or
the ratio of SAPCD2 to GAPDH expression is between about 1×10⁻³ and about 3×10⁻².

73. The method of any one of claims 23-72, wherein the cells are passaged during the first incubation and/or during the second incubation by enzymatic dissociation comprising use of Accutase.

74. The method of any one of claims 16 and 20-73, further comprising formulating the harvested cells with a cryoprotectant.

75. The method of claim 74, wherein the cryoprotectant is selected from among the group consisting of glycerol, propylene glycol, and dimethyl sulfoxide (DMSO).

76. The method of claim 74 or claim 75, further comprising cryopreserving the formulated cells.

77. The method of claim 76, wherein the cryopreserving comprises controlled rate freezing.

78. The method of any one of claims 1-77, wherein the pluripotent stem cells are embryonic stem (ES) cells, induced pluripotent stem cells (iPSCs), or a combination thereof.

79. The method of any one of claims 1-78, wherein the pluripotent stem cells are induced pluripotent stem cells, optionally human induced pluripotent stem cells.

80. The method of any one of claims 1-79, wherein the pluripotent stem cells are from a healthy human subject.

81. The method of any one of claims 1-79, wherein the pluripotent stem cells are from a human subject with a neurodegenerative disease or condition.

82. The method of claim 81, wherein the neurodegenerative disease or condition comprises the loss of dopaminergic neurons.

83. The method of claim 81 or claim 82, wherein the neurodegenerative disease or condition is a Parkinsonism.

84. The method of claim 81 or claim 82, wherein the neurodegenerative disease or condition is Parkinson's disease.

85. The method of any one of claims 1-84, wherein the pluripotent stem cells are hypoimmunogenic.

86. The method of claim 85, wherein the pluripotent stem cells are engineered to (a) remove genes encoding one or more of polymorphic HLA-A/-B/-C and HLA class II molecules; and (b) to provide genes encoding one or more of PD-L1, HLA-G, and CD47, optionally into a AAVS1 safe harbor locus.

87. A therapeutic composition produced by the method of any one of claims 24-86.

88. A population of neuronal progenitor cells that is selected as a population of neuronal progenitor cells that is predicted to engraft by the method of any one of claims 15, 16, 20-23, and 25-86.

89. A therapeutic composition comprising the population of neuronal progenitor cells of claim 88.

90. A therapeutic composition comprising determined dopaminergic neuronal progenitor cells (DDPCs) derived from pluripotent stem cells, wherein the therapeutic composition exhibits one or more of:
(a) a ratio of ASPM to GAPDH expression of greater than about 7×10⁻⁴;
(b) a ratio of AURKB to GAPDH expression of greater than about 9×10⁻⁴;
(c) a ratio of BRINP1 to GAPDH expression of greater than about 9×10⁻⁵;
(d) a ratio of BUB1 to GAPDH expression of greater than about 3×10⁻³;
(e) a ratio of CCNB2 to GAPDH expression of greater than about 3×10⁻³;
(f) a ratio of CDC20 to GAPDH expression of greater than about 3×10⁻³;
(g) a ratio of CDC25C to GAPDH expression of greater than about 5×10⁻⁴;
(h) a ratio of CDKN1A to GAPDH expression of greater than about 1×10⁻³;
(i) a ratio of CENPF to GAPDH expression of greater than about 7×10⁻³;
(j) a ratio of DLGAP5 to GAPDH expression of greater than about 2×10⁻³;
(k) a ratio of FAM83D to GAPDH expression of greater than about 6×10⁻⁴;
(l) a ratio of FANCD2 to GAPDH expression of greater than about 3×10⁻³;
(m) a ratio of GEM to GAPDH expression of greater than about 6×10⁻⁴;
(n) a ratio of HMMR to GAPDH expression of greater than about 8×10⁻⁴;
(o) a ratio of IQGAP3 to GAPDH expression of greater than about 1×10⁻³;
(p) a ratio of KIF20A to GAPDH expression of greater than about 1×10⁻³;
(q) a ratio of KIF2C to GAPDH expression of greater than about 3×10⁻³;
(r) a ratio of KIFC1 to GAPDH expression of greater than about 2×10⁻³;
(s) a ratio of MKI67 to GAPDH expression of greater than about 2×10⁻³;
(t) a ratio of PIMREG to GAPDH expression of greater than about 1×10⁻³;
(u) a ratio of PLK2 to GAPDH expression of greater than about 4×10⁻³;
(v) a ratio of PTTG1 to GAPDH expression of greater than about 3×10⁻³;
(w) a ratio of SAPCD2 to GAPDH expression of greater than about 1×10⁻³;
(x) a ratio of TOP2A to GAPDH expression of greater than about 3×10⁻²;
(y) a ratio of TPX2 to GAPDH expression of greater than about 7×10⁻³; and
(z) a ratio of TTK to GAPDH expression of greater than about 2×10⁻³.

91. The therapeutic composition of any one of claims 87, 89, and 90, wherein:
(a) the ratio of ASPM to GAPDH expression is between about 7×10⁻⁴ and about 2×10⁻¹;
(b) the ratio of AURKB to GAPDH expression is between about 9×10⁻⁴ and about 4×10⁻²;
(c) the ratio of BRINP1 to GAPDH expression is between about 9×10⁻⁵ and about 5×10⁻²;
(d) the ratio of BUB1 to GAPDH expression is between about 3×10⁻³ and about 7×10⁻²;
(e) the ratio of CCNB2 to GAPDH expression is between about 3×10⁻³ and about 7×10⁻²;
(f) the ratio of CDC20 to GAPDH expression is between about 3×10⁻³ and about 1×10⁻¹;
(g) the ratio of CDC25C to GAPDH expression is between about 5×10⁻⁴ and about 3×10⁻²;
(h) the ratio of CDKN1A to GAPDH expression is between about 1×10⁻³ and about 9×10⁻²;
(i) the ratio of CENPF to GAPDH expression is between about 7×10⁻³ and about 5×10⁻¹;
(j) the ratio of DLGAP5 to GAPDH expression is between about 2×10⁻³ and about 9×10⁻²;
(k) the ratio of FAM83D to GAPDH expression is between about 6×10⁻⁴ and about 3×10⁻²;
(l) the ratio of FANCD2 to GAPDH expression is between about 3×10⁻³ and about 4×10⁻²;
(m) the ratio of GEM to GAPDH expression is between about 6×10⁻⁴ and about 3×10⁻²;
(n) the ratio of HMMR to GAPDH expression is between about 8×10⁻⁴ and about 6×10⁻²;
(o) the ratio of IQGAP3 to GAPDH expression is between about 1×10⁻³ and about 6×10⁻²;
(p) the ratio of KIF20A to GAPDH expression is between about 1×10⁻³ and about 8×10⁻²;
(q) the ratio of KIF2C to GAPDH expression is between about 3×10⁻³ and about 7×10⁻²;
(r) the ratio of KIFC1 to GAPDH expression is between about 2×10⁻³ and about 8×10⁻²;
(s) the ratio of MKI67 to GAPDH expression is between about 2×10⁻³ and about 4×10⁻¹;
(t) the ratio of PIMREG to GAPDH expression is between about 1×10⁻³ and about 4×10⁻²;
(u) the ratio of PLK2 to GAPDH expression is between about 4×10⁻³ and about 6×10⁻²;
(v) the ratio of PTTG1 to GAPDH expression is between about 3×10⁻³ and about 9×10⁻²;
(w) the ratio of SAPCD2 to GAPDH expression is between about 1×10⁻³ and about 3×10⁻²;
(x) the ratio of TOP2A to GAPDH expression is between about 3×10⁻² and about 7×10⁻¹;
(y) the ratio of TPX2 to GAPDH expression is between about 7×10⁻³ and about 2×10⁻¹; and/or
(z) the ratio of TTK to GAPDH expression is between about 2×10⁻³ and about 8×10⁻².

92. The therapeutic composition of any one of claims 87 and 89-91, wherein:
the ratio of BRINP1 to GAPDH expression is between about 9×10⁻⁵ and about 5×10⁻²;
the ratio of CDKN1A to GAPDH expression is between about 1×10⁻³ and about 9×10⁻²; the ratio of FAM83D to GAPDH expression is between about 6×10⁻⁴ and about 3×10⁻²;
the ratio of FANCD2 to GAPDH expression is between about 3×10⁻³ and about 4×10⁻²;
the ratio of GEM to GAPDH expression is between about 6×10⁻⁴ and about 3×10⁻²;
the ratio of PLK2 to GAPDH expression is between about 4×10⁻³ and about 6×10⁻²; and/or
the ratio of SAPCD2 to GAPDH expression is between about 1×10⁻³ and about 3×10⁻².

93. The therapeutic composition of any one of claims 90-92, wherein the expression is RNA expression.

94. The therapeutic composition of any one of claims 87 and 89-93, wherein the composition comprises a cryoprotectant.

95. The therapeutic composition of claim 94, wherein the cryoprotectant is selected from among the group consisting of glycerol, propylene glycol, and dimethyl sulfoxide (DMSO).

96. The therapeutic composition of any one of claims 87 and 89-95, wherein the composition is for use in treatment of a neurodegenerative disease or condition in a subject, optionally wherein the neurodegenerative disease or condition comprises a loss of dopaminergic neurons.

97. The therapeutic composition of any one of claims 87 and 89-95, wherein the composition is for use in the manufacture of a medicament for treatment of a neurodegenerative disease or condition in a subject, optionally wherein the neurodegenerative disease or condition comprises a loss of dopaminergic neurons.

98. A method of treatment, comprising implanting in a brain region of a subject having a neurodegenerative disease or condition a therapeutically effective amount of the therapeutic composition of any one of claims 87 and 89-95, optionally wherein the neurodegenerative disease or condition comprises a loss of dopaminergic neurons.
